SEQUENCE LISTING (27 sequences)

SEQ ID NO: 1 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 2 - protein, 955 aa, Homo sapiens [sequence omitted]

SEQ ID NO: 3 - protein, 344 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 4 - protein, 180 aa, Homo sapiens
MLRVLVGAVLPAMLLAAPPPINKLALFPDKSAWCEAKNITQIVGHSGCEAKSIQNRACLGQCFSYSVPNTFPQSTESLVHCDSCMPAQSMWEIVTLECPGHEEVPRVDKLVEKILHCSCQACGKEPSHEGLSVYVQGEDGPGSQPGTHPHPHPHPHPGGQTPEPEDPPGAPHTEEEGAED

SEQ ID NO: 5 - protein, 267 aa, Homo sapiens
MHLLLFQLLVLLPLGKTTRHQDGRQNQSSLSPVLLPRNQRELPTGNHEEAEEKPDLFVAVPHLVATSPAGEGQRQREKMLSRFGRFWKKPEREMHPSRDSDSEPFPPGTQSLIQPIDGMKMEKSPLREEAKKFWHHFMFRKTPASQGVILPIKSHEVHWETCRTVPFSQTITHEGCEKVVVQNNLCFGKCGSVHFPGAAQHSHTSCSHCLPAKFTTMHLPLNCTELSSVIKVVMLVEECQCKVKTEHEDGHILHAGSQDSFIPGVSA

SEQ ID NO: 6 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 7 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 8 - protein, 70 aa, Homo sapiens
MKATIILLLLAQVSWAGPFQQRGLFDFMLEDEASGIGPEVPDDRDFEPSLGPVCPFRCQCHLRVVQCSDL

SEQ ID NO: 9 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 10 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 11 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 12 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 13 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 14 - protein, 172 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQERSPGQKKGRKDRRPRKDRKLDRRLDVRPRQPGLQP

SEQ ID NO: 15 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 16 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 17 - protein, 338 aa, Homo sapiens
AVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 18 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 19 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 20 - protein, 208 aa, Homo sapiens
MWKWILTHCASAFPHLPGCCCCCFLLLFLVSSVPVTCQALGQVMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 21 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 22 - protein, 160 aa, Homo sapiens
MVPSAGQLALFALGIVLAACQALENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 23 - protein, 159 aa, Homo sapiens
MVPSAGQLALFALGIVLAACQALENSTSPLSDPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 24 - protein, 160 aa, Artificial Sequence
  Synthetic amino acid
MVPLAGQLALFALGIVLAACQALENSTSPLSDPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVVL

SEQ ID NO: 25 - protein, 50 aa, Artificial Sequence
  Synthetic amino acid
VVSHFNDCPDSHTQFCFHGTCRFLVQEDKPACVCHSGYVGARCEHADLLA

SEQ ID NO: 26 - protein, 246 aa, Homo sapiens
MTILFLTMVISYFGCMKAAPMKEAIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 27 - protein, 194 aa, Homo sapiens; identical to SEQ ID NO: 19

(19) United States
(12) Patent Application Publication
(10) Pub. No.: US 2024/0158756 A1
(43) Pub. Date: May 16, 2024

(54) FEEDER-BASED AND FEEDER-FREE STEM CELL CULTURE SYSTEMS FOR STRATIFIED EPITHELIAL STEM CELLS, AND USES RELATED THERETO

(71) Applicant: University of Houston System, Houston, TX (US)

(72) Inventors: Frank McKEON, Sugar Land, TX (US); Wa XIAN, Sugar Land, TX (US); Matthew P. VINCENT, Amesbury, MA (US); Jingzhong XIE, Houston, TX (US); Wei RAO, Houston, TX (US)

(73) Assignee: University of Houston System, Houston, TX (US)

(21) Appl. No.: 17/767,690

(22) PCT Filed: Oct. 9, 2020
(86) PCT No.: PCT/US2020/055043

§ 371 Date: Apr. 8, 2022

Related U.S. Application Data
(60) Provisional application No. 62/913,226, filed on Oct. 10, 2019.

(51) Int. Cl. C12N 5/071; C12N 5/095

(52) U.S. Cl. CPC C12N 5/0689; C12N 5/0685; C12N 5/0695; C12N 2501/119; C12N 2501/15; C12N 2501/155; C12N 2501/165; C12N 2501/33; C12N 2501/603; C12N 2501/727

(57) ABSTRACT

The present invention relates to a culture media system that is useful for the isolation and epigenetically stable propagation of normal stem cells in culture, wherein the stem cells are derived from stratified epithelial tissues and cancer stem cells from epithelial cancers. In certain embodiments, the culture system is a feeder-free system.

### PRIORITY CLAIM

This application claims benefit of priority to U.S. Provisional Application Ser. No. 62/913,226, filed Oct. 10, 2020, the entire contents of which are hereby incorporated by reference.

### BACKGROUND OF THE INVENTION

Stratified epithelium differs from simple epithelium in that it is multilayered. It is therefore often found where body linings have to withstand mechanical or chemical insult such that layers can be abraded and lost without exposing subepithelial layers. Cells flatten as the layers become more apical, though in their most basal layers the cells can be squamous or cuboidal.

Stratified epithelia include columnar, cuboidal and squamous types. Squamous epithelium is found lining surfaces such as the skin, and alveoli in the lung, enabling simple passive diffusion as also found in the alveolar epithelium in the lungs. Specialized squamous epithelium also forms the lining of cavities such as in blood vessels, as endothelium and in the pericardium, as mesothelium and in other body cavities.

Cuboidal epithelial cells have a cube-like shape and appear square in cross-section. The cell nucleus is large, spherical and is in the centre of the cell. Cuboidal epithelium is commonly found in secretive tissue such as the exocrine glands, or in absorptive tissue such as the pancreas, the lining of the kidney tubules as well as in the ducts of the glands. The germinal epithelium that covers the female ovary, and the germinal epithelium that lines the walls of the seminiferous tubules in the testes are also of the cuboidal type. Cuboidal cells provide protection and may be active in pumping material in or out of the lumen, or passive depending on their location and specialization. Simple cuboidal epithelium commonly differentiates to form the secretory and duct portions of glands. Stratified cuboidal epithelium protects areas such as the ducts of sweat glands, mammary glands, and salivary glands.

Columnar epithelial cells are elongated and column-shaped and have a height of at least four times their width. Their nuclei are elongated and are usually located near the base of the cells. Columnar epithelium forms the lining of the stomach and intestines. The cells here may possess microvilli for maximizing the surface area for absorption and these microvilli may form a brush border. Other cells may be ciliated to move mucus in the function of mucociliary clearance. Other ciliated cells are found in the fallopian tubes, the uterus and central canal of the spinal cord. Some columnar cells are specialized for sensory reception such as in the nose, ears and the taste buds. Hair cells in the inner ears have stereocilia which are similar to microvilli. Goblet cells are modified columnar cells and are found between the columnar epithelial cells of the duodenum. They secrete mucus, which acts as a lubricant. Single-layered non-ciliated columnar epithelium tends to indicate an absorptive function. Stratified columnar epithelium is rare but is found in lobar ducts in the salivary glands, the eye, pharynx and sex organs. This consists of a layer of cells resting on at least one other layer of epithelial cells which can be squamous, cuboidal, or columnar.

The isolation and long-term expansion of primary cells, particularly stem/progenitor populations, are fundamental and important basic techniques in various biological fields, including developmental biology and stem cell biology, and medical science. Cells in stratified epithelial tissues are highly regenerative and disproportionately accountable for many human cancers and inflammatory/autoimmune diseases; however, cloning epithelial stem cells is limited by difficulties in maintaining these cells in an immature state. However, the inability to maintain the immaturity of stem cell populations in vitro restricts the long-term expansion of various types of human epithelial stem cells.

For instance, the majority of human cancers are derived from epithelial tissues. Since the concept of cancer stem cells (“CSC”) was introduced in late 1990s, it has gained acceptance as the mechanism underlying tumor initiation, propagation and ultimately drug resistance; these stem cells have influenced all approaches to cancer research and therapy as they help to mechanistically explain the progression of more benign to more aggressive forms of cancers. The majority of cancer drugs, while killing the bulk of tumor cells, ultimately fail to induce durable clinical responses because they are not able to eliminate the critical CSCs that are resistant to existing cancer therapies including targeted drugs, chemo and radiation therapy. Surviving CSCs give rise to new tumors and metastases, causing relapse of the disease. The recurrent tumors become more malignant, fast spreading and resistant to radiotherapy and previously used drugs, making the prognosis for cancer patients dismal.

Compounding matters is that many tumors are believed to contain a heterogeneous population of CSCs, representing a range of tumor promoting activities and a range of drug sensitivity. Thus, the specific survival of CSCs, or subsets of CSCs from the heterogeneous CSC population, could provide an explanation for many therapeutic failures and highlight new directions for the enhancement of cancer therapy. In order to develop truly effective treatments that can create a durable clinical response it is important to develop drugs that can target and kill CSCs. CSC have only recently begun to be precisely identified due to technical advancements that facilitate identification, isolation, and interrogation of distinct tumor cell subpopulations with differing abilities to form and perpetuate tumors. There is therefore a need for methods and reagents for the isolation and stable passaging and expansion of stratified epithelial CSCs—so as to be useful in drug screening.

It is an object of the present invention to provide systems and reagents for the rapid isolation/cloning of stratified epithelial stem cells, particularly from small biopsies, under conditions which preserve the epigenetic memory and faithfully preserves the in vivo characteristics of the stem cells as they existed in the tissue biopsy through rounds of expansion and passaging in culture, so as to be scalable, efficient and ultimately affordable enough to be done on a patient-by-patient process for patient-specific diagnostic and treatment strategy purposes (inflammatory diseases and metaplasia/tumors as examples) or regenerative medicine purposes.

### SUMMARY OF THE INVENTION

In one aspect, the invention provides a method for isolating a stem cell from epithelial tissue, preferably stratified epithelial tissue, e.g., normal or diseased tissue, the method comprising:
(1) culturing dissociated epithelial cells from a stratified epithelial tissue sample to form stem cell colonies, wherein the dissociated cells and cell colonies are cultured in a medium comprising:
   (a) a ROCK (Rho Kinase) inhibitor; (c) a mitogenic growth factor; (d) insulin or IGF; (e) a TrkA Inhibitor (GW441756); and (h) an Oct4-activating agent;
      wherein the medium includes at least one of a VEGF Inhibitor, a tyrosine kinase inhibitor and/or a FGF10 or FGF10 agonist;
      wherein the medium optionally further comprises a TGFβ signaling pathway inhibitor (e.g., a TGFβ inhibitor or a TGFβ receptor inhibitor);
      wherein the medium optionally further comprising a Bone Morphogenetic Protein (BMP) antagonist;
      wherein the medium optionally further comprising a Wnt agonist;
      wherein the cells from the tissue sample are optionally in fluid or direct contact with mitotically inactive feeder cells, but preferably are cultured in the absence of feeder cells;
      wherein the cells from the tissue sample are optionally in contact with
      extracellular matrix (such as a basement membrane matrix) or other bio or synthetic matrix;
(2) isolating single stem cells from the cell colonies, and
(3) culturing isolated single stem cells from step (2) individually to form cultures purified stem cell clones, (optionally) in contact with feeder cells and/or a basement membrane matrix in the medium; wherein each of the stem cell clones represents a clonal expansion of an epithelial stem cell present in the stratified epithelial tissue sample, thereby isolating stratified epithelial stem cells.

In certain preferred embodiments, the culture media includes a VEGF inhibitor, preferably a VEGF inhibitor that is a small molecule tyrosine kinase inhibitor. In certain preferred embodiments, the culture media includes a VEGF inhibitor and lacks FGF10 or an FGF10 agonist. In certain preferred embodiments, the culture media includes both a VEGF Receptor kinase inhibitor and a Tyrosine Kinase inhibitor, which may be the same or different compounds. In certain preferred embodiments, the culture media includes a VEGF receptor kinase inhibitor and a Pan-ABL1 Kinase Inhibitor, which may be the same or different compound.

In feeder-free embodiments of the present invention, the medium further comprises (i) a SYK Inhibitor; (j) an LPA receptor antagonist; (k) a GSK3 inhibitor; and (l) a CK2 inhibitor.

In certain embodiments, the epithelial tissue from the patient having the disease, disorder, or abnormal condition is afflicted by the disease, disorder, or abnormal condition. In certain embodiments, the stratified epithelial stem cell is an adult stratified epithelial stem cell. In certain embodiments, the stratified epithelial stem cell is a fetal stratified epithelial stem cell.

In certain embodiments, the medium optionally further comprises nicotinamide and/or includes a Notch Agonist.

In certain other embodiments, the medium specifically lacks one or both of nicotinamide and/or includes a Notch Agonist.

In certain embodiments, in step (1), the (epithelial) cells are dissociated from the tissue through enzymatic digestion with an enzyme. For example, the enzyme may comprise collagenase, protease, dispase, pronase, elastase, hyaluronidase, accutase or trypsin.

In certain embodiments, in step (1), the (epithelial) cells are dissociated from the tissue through dissolving extracellular matrix surrounding the (epithelial) cells.

In certain embodiments involving fluid contact with feeder cells, the mitotically inactivated cells are mitotically-inactivated fibroblasts, preferably human or murine fibroblasts, such as 3T3-J2 cells. Mitotic inactivation can be accomplished by the administration of mitomycin C or other chemically-based mitotic inhibitors, irradiation with γ-rays, irradiation with X-rays, and/or irradiation with UV light.

In certain embodiments involving the contact with extracellular matrix, the extracellular matrix is a basement membrane matrix, such as a laminin-containing basement membrane matrix (e.g., MATRIGEL™ basement membrane matrix (BD Biosciences)) and is preferably growth factor-reduced. In other embodiments, the biopolymer is selected from the group consisting of collagen, chitosan; fibronectin, fibrin, and mixtures thereof.

In certain embodiments, the basement membrane matrix does not support 3-dimensional growth or does not form a 3-dimensional matrix necessary to support 3-dimensional growth.

In certain embodiments, the medium further comprises serum, preferably FBS (and even more preferably FBS that is not heat inactivated), such as in a concentration of 5%-15%, such as 10% FBS.

In certain embodiments, the ROCK inhibitor comprises Rho Kinase Inhibitor VI (Y-27632, (R)-(+)-trans-N-(4-Pyridyl)-4-(1-aminoethyl)-cyclohexanecarboxamide)), Fasudil or HA1077 (5-(1,4-diazepan-1-ylsulfonyl)isoquinoline), or HI 152 ((S)-(+)-2-methyl-1-[(4-methyl-5-isoquinolinyl)sulfonyl]-hexahydro-1H-1,4-diazepine dihydrochloride).

In certain embodiments, the BMP antagonist comprises Noggin, DAN, a DAN-like proteins comprising a DAN cysteine-knot domain (e.g., Cerberus and Gremlin), Chordin, a chordin-like protein comprising a chordin domain, Follistatin, a follistatin-related protein comprising a follistatin domain, sclerostin/SOST, decorin, or a-2 macro globulin. In certain preferred embodiments, the BMP antagonist is Noggin.

In certain embodiments, the Medium includes a Wnt agonist, such as R-spondin 1, R-spondin 2, R-spondin 3, R-spondin 4, an R-spondin mimic, a Wnt family protein (e.g., Wnt-3a, Wnt 5, Wnt-6a), Norrin, or a GSK-inhibitor (e.g., CHIR99021).

In certain embodiments, the mitogenic growth factor comprises EGF, Keratinocyte Growth Factor (KGF), TGFa, BDNF, HGF, and/or FGF (e.g., FGF7 or FGF10).

In certain embodiments, the TGF-beta receptor inhibitor comprises SB431542 (4-(4-(5-benzo[1,3]dioxol-5-yl)-4-(pyridin-2-yl)-1H-imidazol-2-yl)benzamide), A83-01, SB505124, SB-525334, LY 364947, SD-208, or SJN 2511.

In certain embodiments, the TGF-beta (signaling) inhibitor binds to and reduces the activity of one or more serine/threonine protein kinases selected from the group consisting of ALK5, ALK4, TGF-beta receptor kinase 1 and ALK7.

In certain embodiments, the TGF-beta (signaling) inhibitor is added at a concentration of between 1 nM and 100 μM, between 10 nM and 100 μM, between 100 nM and 10 μM, or approximately 1 μM.

In certain embodiments, the VEGF inhibitor is selected from aflibercept, pegaptanib, tivozanib, 3-(4-Bromo-2,6-difluoro-benzyloxy)-5-[3-(4-pyrrolidin-1-yl-butyl)-ureido]-isothiazole-4-carboxylic acid amide hydrochloride, axitinib, N-(4-bromo-2-fluorophenyl)-6-methoxy-7-[(1-methylpiperidin-4-yl-)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyrid-in-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl) methoxy]quinazol in-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, 5-((7-Benzyloxyquinazolin-4-yl)amino)-4-fluoro-2-methyl phenol hydrochloride, any derivatives thereof and any combinations thereof.

In certain preferred embodiments, the VEGF inhibitor is a VEGF Receptor inhibitor, and even more preferably a VEGF Receptor kinase inhibitor such as Tivozanib (AV-951), AZD2932, Midostaurin (pkc412), BAW2881 (NVP-BAW2881), Nintedanib (BIBF 1120), SU5402, SU1498, BFH772, Sorafenib, Sunitinib, Dovitinib (TK1258), Semaxanib (SU5416), hypericin, vatalanib, ZM306416, AAL993, SU4312, DMXAA or Foretinib.

In certain embodiments, the Medium includes a tyrosine kinase inhibitor, such as nilotinib, ponatinib, and dasatinib (but not bosutinib or imatinib). In certain preferred embodiments, the tyrosine kinase inhibitor is a Pan-ABL1 Kinase Inhibitor such as Ponatinib or Dasatinib.

In certain embodiments, the Medium includes both a VEGF Receptor kinase inhibitor and a Tyrosine Kinase inhibitor, which may be the same or different compounds, such as a combination of Ponatinib and Tivozanib.

In certain embodiments, the TrkA inhibitors is selected from BMS-754807, GW441756, PF-06273340, Sitravatinib (MGCD516), ANA-12, GNF-5837, Belizatinib (TSR-011), Larotrectinib (LOXO-101) sulfate, Lestaurtinib, Entrectinib (RXDX-101), GNF 5837 and AG-879. Preferably the TrkA inhibitor is selective for TrkA relative to TrkB or TrkC, such as GW441756 and Sitravatinib (MGCD516). Preferably the TrkA inhibitor is a potent, selective inhibitor of TrkA with IC50 of 10 nM or less, with an IC50 for inhibiting c-Raf1 and CDK2 at least 100-fold greater than the IC50 for inhibiting TrkA. such as GW441756.

In certain embodiments, the Oct4-activating agent is an agent that can activate Oct4 promoter-driven reporter genes, such as a luciferase gene under the transcriptional control of an Oct4-promoter, and more preferably is an able to activate both Oct4 and Nanog promoter-driven reporter genes. Furthermore, when added to the reprogramming mixture along with the quartet reprogramming factors (Oct4, Sox2, c-Myc, and Klf4), an Oct4-activating agent enhances the iPSC reprogramming efficiency and accelerated the reprogramming process. Exemplary Oct4-activating Agents are taught in, for example, US Patent Application 20150191701 and Li et al. (2012) “Identification of Oct4-activating compounds that enhance reprogramming efficiency”. PNAS 109(51):20853-8.

In certain embodiments, the Oct4-activating agent is represented in formula

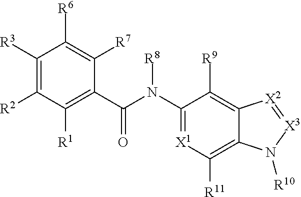

wherein,
X¹ is C(R¹²) or N;
X² is C(R⁴) or N;
X³ is C(R⁵) or N;
R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl, wherein R² and R³ are optionally joined to form a substituted or unsubstituted heterocycloalkyl or substituted or unsubstituted heteroaryl.

Particularly in those embodiments which are feeder-free, the medium may also include a SYK (Spleen Tyrosine Kinase) inhibitor. Representative SYK inhibitor can be is selected from the group consisting of Entospletinib (GS-9973), Fostamatinib (R788), R406, cerdulatinib (PRT062070) and TAK-659.

Particularly in those embodiments which are feeder-free, the medium may also include an LPA receptor antagonist, such as an antagonist that inhibits LPA1 and LPA3-induced inositol phosphate production with Ki's for each of 1000 μM or less, and is a substantially weaker inhibition for LPA2, LPA4, LPA5, LPA6, i.e., with Ki's for each of 5000 μM or less. Ki16198 is a preferred LPA receptor antagonist and is the methyl ester of Ki16425.

Particularly in those embodiments which are feeder-free, the medium may also include a GSK3 Inhibitor. Exemplary GSK3 inhibitors include CHIR-99021 (CT99021) HCl, SB216763, CHIR-98014, TWS119, Tideglusib, SB415286, CHIR-99021 (CT99021), AZD2858, AZD1080, AR-A014418, TDZD-8, LY2090314, BIO-acetoxime, IM-12, 1-Azakenpaullone, Indirubin and 6-BIO.

Particularly in those embodiments which are feeder-free, the medium may also include a CK2 inhibitor, such as CX-4945 (Silmitasertib), CX-8184, DMAT, ellagic acid or TTP22.

In another aspect, the invention provides a single cell clone of an epithelial stem cell, or an in vitro culture thereof, such as one comprising a subject medium, wherein the epithelial stem cell substantially lacks expression of marker(s) associated with differentiated cell types in the epithelial tissue from which it was derived.

In another aspect, the invention provides a single cell clone of a non-embryonic epithelial stem cell, or an in vitro culture thereof, such as one comprising a subject medium, wherein the non-embryonic epithelial stem cell has an immature, undifferentiated morphology characterized by small round cell shape with high nucleus to cytoplasm ratio.

In a related aspect, the invention also provides a library or collection of the subject single cell clone, or in vitro culture (such as one comprising a subject medium) thereof. In certain embodiments, the library or collection may comprise single cell clones from the same tissue/organ type. In certain embodiments, the library or collection may comprise single cell clones isolated from the same type of tissue/organ type, but from different members of a population. In certain embodiments, one or more (preferably each) member of the population are homozygous across at least one tissue typing locus (such as HLA-A, HLA-B, and HLAD). In certain embodiments, at least one tissue typing locus (e.g., the HLA loci above) is engineered in the cloned stem cells via, for example, TALEN or CRISPR technologies (see below) to generate universal donor cell lines (e.g., liver cells) lacking tissue antigens encode by the tissue typing locus (e.g., HLA-A, HLA-B, and HLA-D, etc.). See Torikai et al. (Blood, 122(8): 1341-1349, 2013, incorporated by reference). In certain embodiments, the population may be defined by ethnic group, age, gender, disease status, or any common characteristics of a population. The library or collection may have at least about 20, 30, 40, 50, 60, 70, 80, 90, 100, 120, 150, 180, 200, 250, 300 or more members.

In another aspect, the invention provides a method of treating a subject having a disease, a disorder, or an abnormal condition and in need of treatment, comprising: (1) using any of the subject method, isolating an epithelial stem cell from a tissue corresponding to a tissue affected by the disease, disorder, or abnormal condition in the subject; (2) optionally, altering the expression of at least one gene in the epithelial stem cell to produce an altered epithelial stem cell; (3) reintroducing the isolated epithelial stem cell or altered epithelial stem cell, or a clonal expansion thereof, into the subject, wherein at least one adverse effect or symptom of the disease, disorder, or abnormal condition is alleviated in the subject.

In certain embodiments, the expression of at least one gene in the epithelial stem cell is genetically, recombinantly and/or epigenetically altered to produce an altered epithelial stem cell.

In certain embodiments, the tissue from which the epithelial stem cell is isolated is from a healthy adult or fetal (i.e., non-embryonic) subject.

In certain embodiments, the tissue from which the epithelial stem cell is isolated is from the subject. In certain embodiments, the tissue from which the epithelial stem cell is isolated is an affected tissue affected by the disease, disorder, or abnormal condition.

In certain embodiments, the tissue from which the epithelial stem cell is isolated is adjacent to an affected tissue affected by the disease, disorder, or abnormal condition.

In certain embodiments, the at least one gene is under-expressed in the tissue affected by the disease, disorder, or abnormal condition in the subject, and expression of the at least one gene is enhanced in the altered epithelial stem cell.

In certain embodiments, the at least one gene is over-expressed in the tissue affected by the disease, disorder, or abnormal condition in the subject, and expression of the at least one gene is reduced in the altered epithelial stem cell.

In certain embodiments, step (2) is effected by introducing into the epithelial stem cell an exogenous DNA or RNA.

In yet another aspect, the invention provides a method of screening for a compound, the method comprising: (1) using any of the methods of the invention, isolating an epithelial stem cell from a subject; (2) producing a cell line of the epithelial stem cell via single cell clonal expansion; (3) contacting test cells from the cell line with a plurality of candidate compounds; and, (4) identifying one or more compounds that produces a pre-determined phenotype change in the test cells.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Representative images of cloned human epithelial stem cells derived from Epidermis, Upper airway, Distal airway, Bladder, Esophagus, Ovarian Tumor. The human epithelial tissues were digested and seeded on the irradiated 3T3-J2 feeder in the presence of SQM medium.

FIG. 2A. Single-cell derived pedigree of human bladder stem cells were seeded in air-liquid interface system. A single bladder stem cell can differentiate into all cell types existing in bladder epithelium including basal cells, transitional epithelium and basal cells.

FIG. 2B. One thousand single bladder epithelium stem cells were seeded on top of irradiated 3T3-J2 feeder and formed over 500 colonies in the presence of SQM medium. The clonogenic ability was not changed after 7 passages, around 100 days culturing and 200 cell divisions.

FIG. 2C. CNV, BAF (B allele frequency) and LRR (log R ratio) profiles of pedigrees at P1 to P7 showed genomic stability during passaging.

FIG. 3A. Representative images of human distal airway stem cells at passage 5 and passage 25. CNV, BAF (B allele frequency) and LRR (log R ratio) profiles of pedigrees at P5 to P25 of human DASCs showed the stability of the genome during passaging.

FIG. 3B. Single cell derived pedigree of human DASCs were seeded in ALI culture and differentiate into Club cell (CC10), Type I (AQP4) and Type II (SEPTB) pneumocytes.

FIG. 3C. Generation of DASCs by the methods of the present invention produces a high degree of clonogenicity (clonogenic ability) which, like the observed genetic and epigenetic stability of these stem cell clones, is maintained over multiple passages (compare passage 5 to 25).

FIG. 4A. Single cell derived pedigree of human upper airway stem cells were seeded in air-liquid interface for in vitro differentiation. A single cell was differentiated into ciliated cell (Tubulin) and goblet cell (MUC5AC).

FIG. 4B. Left, representative image of human upper airway stem cell pedigree growing on top of irradiated 3T3-J2 feeder in the presence of SQM medium. Right, The cells from the pedigree were transplanted into NSG mouse and formed upper airway epithelium comprising ciliated cells and goblet cells.

FIG. 5. Stem cells of single-cell derived pedigree of human skin were seeded in air-liquid interface differentiation system and induced to differentiate into squamous epithelium resembling human skin.

FIG. 6. Representative images of cloned human epithelial stem cells derived from Epidermis, Upper airway, Distal airway, Bladder, Esophagus, Ovarian Tumor. The human epithelial tissues were digested and seeded in the presence of SGM-63+ medium without any mouse feeder support.

FIG. 7. Stem cells of single cell derived pedigree of human upper airway epithelium were transplanted into the NSG mice and produced structure resembling normal human upper airway epithelium based on histology and immunostaining using markers specific for ciliated cells (Tubulin), goblet cells (MUC5AC) and club cells (CC10).

FIGS. 8A-E. Representative images of cloned human epithelial stem cells derived from skin using the B1 Media system. The human skin tissue was digested and seeded on the irradiated 3T3-J2 feeder in the presence of specialized medium. A. Bright field image of human skin epithelial stem cells. B. The stem cell colonies were stained positively with anti p63 antibody. C. The stem cell colonies were stained positively with anti Krt5 antibody. D. The stem cell colonies were stained positively with Ki67 antibody, suggesting the cells are highly proliferative. E. Single human skin stem cell was sorted into each well of 384-well cell culture dish. More than 60% of the cells are clonogenic based on the rhodamine staining.

FIGS. 9A-E. Representative images of cloned human epithelial stem cells derived from bladder. The human bladder tissue was digested and seeded on the irradiated 3T3-J2 feeder in the presence of specialized medium. A. Bright field image of human bladder epithelial stem cells. B. The stem cell colonies were stained positively with anti p63 antibody. C. The stem cell colonies were stained positively with anti Krt5 antibody. D. The stem cell colonies were stained positively with Ki67 antibody, suggesting the cells are highly proliferative. E. Single human bladder stem cell was sorted into each well of 384-well cell culture dish. More than 60% of the cells are clonogenic based on the rhodamine staining.

FIGS. 10A-E. Representative images of cloned human epithelial stem cells derived from salivary gland. The human salivary gland tissue was digested and seeded on the irradiated 3T3-J2 feeder in the presence of specialized medium. A. Bright field image of human salivary gland epithelial stem cells. B. The stem cell colonies were stained positively with anti p63 antibody. C. The stem cell colonies were stained positively with anti Krt5 antibody. D. The stem cell colonies were stained positively with Ki67 antibody, suggesting the cells are highly proliferative. E. Single human salivary gland stem cell was sorted into each well of 384-well cell culture dish. More than 60% of the cells are clonogenic based on the rhodamine staining.

FIGS. 11A-D. Representative images of cloned human epithelial stem cells derived from airway. The human airway tissue was digested and seeded on the irradiated 3T3-J2 feeder in the presence of specialized medium. A. Bright field image of human airway epithelial stem cells. B. The stem cell colonies were stained positively with anti p63 antibody. C. The stem cell colonies were stained positively with anti Krt5 antibody. D. Single human airway stem cell was sorted into each well of 384-well cell culture dish. More than 70% of the cells are clonogenic based on the rhodamine staining.

FIGS. 12A-12B. Single-cell derived pedigree of human upper or distal airway stem cells were induced to differentiate in air-liquid interface system. 12A. Single cell derived pedigree of human upper airway stem cells was differentiated into ciliated cell (Tubulin) and goblet cell (MUCSAC). 12B. Single cell derived pedigree of human DASCs were seeded in ALI culture and differentiate into Club cell (CC10), Type I (AQP4) and Type II (SEPTB) pneumocytes.

### DETAILED DESCRIPTION OF EXEMPLARY EMBODIMENTS

### 1. Overview

The invention described herein relates to methods of isolating and/or maintaining in culture non-embryonic (e.g., adult or fetal) epithelial stem cells from the stratified epithelia of organs. Epithelial stem cells thus isolated from the various tissues or organs can self-renew or propagate indefinitely in vitro, are multipotent and can differentiate into the various differentiated cell types normally found within the tissue or organ from which the stem cells are isolated. Cultures (including in vitro cultures) comprising the epithelial stem cells thus isolated are also within the scope of the invention.

In addition, the isolated epithelial stem cells can be propagated through clonal expansion of a single isolated stem cell, to produce a clone (e.g., as an in vitro culture) of which at least about 40%, 70%, or 90% or more cells within the clone can be further passaged as single cell originated clones. Thus, the stem cells isolated using the methods of the invention are uniquely capable of being manipulated in vitro through standard molecular biology techniques, such as introduction of exogenous genetic materials through infection or transfection.

As used herein, “epithelial stem cell” includes adult stem cell isolated from an adult tissue or organ, and fetal stem cell isolated from prenatal tissue or organ.

In a related embodiment, the methods of the invention described herein isolate fetal stem cells from a fetal or prenatal tissue or organ. In certain embodiments, when fetal tissue or organ is the source of the stem cell, the methods of the invention do not destroy the fetus or otherwise impair the normal development of the fetus, especially when the fetus is a human fetus. In other embodiments, the source of the fetal tissue is obtained from aborted fetus, dead fetus, macerated fetal material, or cell, tissue or organs excised therefrom.

The methods of the invention is applicable to any animal stratified epithelial tissue containing epithelial stem cells, including tissues from human, non-human mammal, non-human primate, rodent (including but not limited to mouse, rat, ferret, hamster, guinea pig, rabbit), livestock animals (including but not limited to pig, cattle, sheep, goat, horse, camel), bird, reptile, fish, pet or other companion animals (e.g., cat, dog, bird) or other vertebrates, etc.

The classification of stratified epithelium is based on the cell shape of the superficial layer. If, for example, the superficial layer consists of flat cells, it is part of a stratified squamous epithelium. The stratified epithelium is classified into three different forms.

“Stratified, non-keratinized squamous epithelium”. The cell shape of the stratified, non-keratinized squamous epithelium changes from the basal toward the free surface and is divided into four sections:
Stratum basale: Prismatic dark-colored cells with round nucleus;
Stratum parabasale: Polygonal dark-colored cells arranged in stratified tissue;
Stratum spinosum: Polyhedral, polygonal cells connected by desmosomes;
Stratum superficiale: Flattened cells degraded and desquamated in the outermost layers.

This type of epithelium is found in the mucosa of the oral cavity and esophagus, as well as the vagina and in the eye (corneal epithelium).

“Stratified, keratinized squamous epithelium”. The outermost cell layers of the epithelium consist of flattened cells with no nuclei, converting into scales. They are called stratum corneum, and their purpose is to mechanically protect underlying tissue from dehydration. The stratified, keratinized squamous epithelium is divided into five sections:
Stratum basale
Stratum spinosum
Stratum granulosum: Flattened cells with keratohyalin granules
Stratum lucidum: Conversion area
Stratum corneum

“Transitional epithelium (urothelium)”. The urothelium consists of a basal layer, several intermediate cells layers and an umbrella cell layer. Umbrella cells (superficial cells) are large and often have two nuclei. The crusta, a very dense network of cytoplasm, is located beneath its apical membrane. The plasma membrane consists predominantly of rigid plaques containing uroplakin (transmembrane proteins). Transitional epithelium is primarily found in the efferent urinary tract, i.e., in the renal pelvis, ureter, urinary bladder and the initial part of the urethra.

“Pseudostratified epithelium”. The important characteristic of this epithelium type is that their cells do touch the basal membrane, but not all of them reach the free surface. The cells that reach the free surface belong to the columnar type. The cells that do not reach the free surface rest on the basal lamina and have a round nucleus. The term pseudostratified is derived from the appearance of this epithelium. Because the cell nuclei appear at different heights, it conveys the erroneous impression that there is more than one layer of cells. Non-ciliated pseudostratified epithelium is found, e.g., in the epidydimal duct and vas deferens, and ciliated pseudostratified epithelium with kinocilium is found in the respiratory tract (nasal cavity and bronchi).

In certain embodiments, the epithelial tissue is isolated from a healthy or normal individual.

In certain embodiments, the epithelial tissue is isolated from a disease tissue (e.g., a tissue affected by a disease), a disorder tissue (e.g., a tissue affected by a disorder), or a tissue otherwise have an abnormal condition.

As used herein, the term “disease” includes an abnormal or medical condition that affects the body of an organism and is usually associated with specific symptoms and signs. The disease may be caused by external factors (such as infectious disease, including papilloma virus infection or a sexually transmitted disease), or by internal dysfunctions (such as autoimmune diseases or cancer). In a broad sense, “disease” may also include any condition that causes pain, dysfunction, distress, social problems, or death to the person afflicted, or similar problems for those in contact with the person. In this broader sense, it may include injuries, disabilities, disorders, syndromes, infections, isolated symptoms, deviant behaviors, and atypical variations of structure and function, while in other contexts and for other purposes these may be considered distinguishable categories. In certain preferred embodiments, the stem cells are isolated from a tumor biopsy.

In certain embodiments, the epithelial tissue is isolated from an individual having a disease, disorder, or otherwise abnormal condition, although the epithelial tissue itself may not have been inflicted with the disease, disorder, or abnormal condition. For example, the epithelial tissue may be isolated from a patient having inflammatory bowel disease or gastric cancer, but from a healthy portion of the bowel (in the case of IBD) or stomach (in the case of the tumor) not already inflicted with the inflammatory condition or cancer. In certain embodiments, the epithelial tissue may be nearby or distant from the disease, disorder, or abnormal tissue.

In certain embodiments, the epithelial tissue is isolated from an individual predisposed to develop a disease, disorder, or otherwise abnormal condition, or in high risk of developing the disease, disorder, or otherwise abnormal condition, based on, for example, genetic composition, family history, life style choice (e.g., smoking, diet, exercise habit), previous viral infection or the like of the individual, although the individual has not yet developed the disease, disorder, or otherwise abnormal condition, or displayed a detectable symptom of the disease, disorder, or otherwise abnormal condition.

Another aspect of the invention provides an epithelial stem cell isolated according to any one of the methods of the invention, or an in vitro culture thereof.

In yet another aspect, the invention further provides a single cell clone of an isolated epithelial stem cell, or an in vitro culture thereof, wherein at least about 40%, 50%, 60%, 70%, or about 80% of cells within the single cell clone, when isolated as single cell, is capable of proliferation to produce single cell clone.

Each single cell clone, depending on stages of growth and other growth conditions, may comprise at least about 10, 100, 10³, 10⁴, 10⁵, 10⁶ or more cells.

In a related aspect, the invention provides a single cell clone of an isolated epithelial stem cell, or an in vitro culture thereof, wherein the epithelial stem cell, when isolated as single cell, is capable of self-renewal for greater than about 50 generations, 70 generations, 100 generations, 150 generations, 200 generations, 250 generations, 300 generations, 350 generations, or about 400 or more generations.

In certain embodiments, the in vitro culture comprises a medium of the invention (e.g., a modified medium of the invention as described below). See section below describing the medium of the invention, each medium described therein is incorporated herein by reference. In certain embodiments, the epithelial stem cell is capable of differentiating into a differentiated cell type of the epithelial tissue from which it was originally biopsied, or in the case of a cancer stem cell, a tumor of that tissue origin. For example, the isolated epithelial stem cell of the invention may differentiate into one or more cell types normally found in epithelial tissue of the biopsy from which it was derived.

In certain embodiments, the epithelial stem cell is capable of differentiating into organized structures resembling the structure or substructures found in the tissue from which such epithelial stem cell originates. For example, an isolated liver stem cell of the invention may differentiate into liver-tissue-like structure that resembles liver epithelia, and an isolated gastrointestinal stem cell of the invention may differentiate into GI-tissue-like structure that resembles gastrointestinal epithelia.

In certain embodiments, the epithelial stem cell has an immature, undifferentiated morphology characterized by small round cell shape with high nucleus to cytoplasm ratio.

A further aspect of the invention provides a method of treating a subject having a disease, a disorder, or an abnormal condition and in need of treatment, comprising: (1) using any of the methods of the invention to isolate a non-embryonic (e.g., an adult) stem cell from a regenerative tissue corresponding to a tissue affected by the disease, disorder, or abnormal condition in the subject; (2) altering the expression of at least one gene in the epithelial stem cell to produce an altered epithelial stem cell; (3) reintroducing the altered epithelial stem cell or a clonal expansion or a culture derived tissue transplant thereof into the subject, wherein at least one adverse effect or symptom of the disease, disorder, or abnormal condition is alleviated in the subject, or as a means of regenerating/replacing damaged reproductive tissue. In other instances, the transplanted cells/tissue can be genetically engineered to be resistant to viral infection, such as papillomavirus infection.

For example, step (2) of the method may be effected by introducing into the epithelial stem cell an exogenous DNA or RNA that either increases or decreases the expression of a target gene in the isolated epithelial stem cell. Any of the art-recognized molecular biology techniques can be used to alter gene expression in a cell, e.g., in vitro or ex vivo. Such methods may include, without limitation, transfection or infection by a viral or nonviral based vector, which may encode the coding sequence of a protein or functional fragments thereof that is dysfunctional or deficient in the target cell, or may encode an RNA (antisense RNA, siRNA, miRNA, shRNA, ribozyme, etc.) that disrupts the function of a target gene.

In certain embodiments, the tissue from which the epithelial stem cell is isolated is from a healthy subject. Preferably, the healthy subject is HLA-type matched with the subject in need of treatment.

In certain embodiments, the tissue from which the epithelial stem cell is isolated is from the subject, and the isolated epithelial stem cell is autologous with respect to the subject.

In certain embodiments, the tissue from which the epithelial stem cell is isolated is an affected tissue affected by the disease, disorder, or abnormal condition.

In certain embodiments, the tissue from which the epithelial stem cell is isolated is adjacent to an affected tissue affected by the disease, disorder, or abnormal condition. In certain embodiments, at least one gene is under-expressed in the tissue affected by the disease, disorder, or abnormal condition in the subject, and expression of the at least one gene is enhanced in the altered epithelial stem cell.

In certain embodiments, at least one gene is over-expressed in the tissue affected by the disease, disorder, or abnormal condition in the subject, and expression of the at least one gene is reduced in the altered epithelial stem cell.

In another aspect, the invention also provides a method of screening for agents or conditions that alter the “phenotype” of the cells—such as the differentiation, epigenetics, survival or the like of a reproductive tissue stem cells—whether normal or from a cancer/disease state. In an exemplary embodiment, the method comprises: (1) using any of the methods of the invention to isolate epithelial stem cells (including a cancer stem cell) from the reproductive tissue of a subject; (2) producing one or more stem cell lines from the epithelial stem cells via single cell clonal expansion; (3) contacting test cells from the cell line with one or more candidate compounds; and, (4) identifying compounds that produces a predetermined phenotype change in the test cells. This screening method of the invention may be used for target identification and validation. For example, a potential target gene in an epithelial stem cell isolated from a patient in need of treatment may functional abnormally (either over-expression or under-expression) to cause a phenotype associated with a disease, disorder, or abnormal condition. A clonal expansion of the epithelial stem cell isolated using the method of the invention may be subject to the screening method of the invention to test an array of potential compounds (small molecule compounds, etc.) to identify one or more compounds that can correct, alleviate, or reverse the phenotype.

In another embodiment, an epithelial stem cell may be isolated from regenerative tissue of a patient in need of treatment, such as from the reproductive tissue affected by a disease, disorder, or abnormal condition. A clonal expansion of the epithelial stem cell isolated using the method of the invention may be subject to the screening method of the invention to test an array of potential compounds (small molecule compounds, or any RNA-based antagonists such as library of siRNA, etc.) to identify one or more compounds that can correct, alleviate, or reverse the phenotype. The affected target gene by an effective compound may be further identified by, for example, microarray, RNA-Seq, or PCR based expression profile analysis.

The epithelial stem cell isolated using the methods of the invention and clonal expansion thereof may be further useful for toxicology screens or studies such that any toxicology analysis and test can be tailored to individual patients set to receive a certain medicine or medical intervention.

The epithelial stem cell isolated using the methods of the invention and clonal expansion thereof may also be useful for regenerative medicine, where either autologous stem cells or stem cells isolated from HLA-type matched healthy donor can be induced to differentiate into reproductive tissues or organs in vitro, ex vivo, or in vivo to treat an existing condition or prevent/delay such a condition from developing. Such stem cells may be genetically manipulated prior to induced differentiation.

### 2. Methods for Obtaining and/or Culturing Stem Cells

One aspect of the invention relates to a method for isolating a epithelial stem cell from a epithelial tissue, as generally described above.

To illustrate, one step of the method comprises culturing dissociated epithelial cells from the epithelial tissue, (optionally) in contact with a first population of mitotically inactive feeder cells and/or an extracellular matrix, e.g., a basement membrane matrix, to form epithelial cell clones.

In certain embodiments, the (epithelial) cells are dissociated from the tissue through enzymatic digestion with an enzyme, including, without limitation, any one or more of collagenase, protease, dispase, pronase, elastase, hyaluronidase, Accutase and/or trypsin.

These enzymes or functional equivalents are well known in the art, and in almost all cases are commercially available.

In other embodiments, the (epithelial) cells may be dissociated from the tissue sample through dissolving extracellular matrix surrounding the (epithelial) cells. One reagent suitable for this embodiment of the invention include a non-enzymatic proprietary solution marketed by BD Biosciences (San Jose, CA) as the BD™ Cell Recovery Solution (BD Catalog No. 354253), which allows for the recovery of cells cultured on BD MATRIGEL™ Basement Membrane Matrix for subsequent biochemical analyses.

In certain embodiments, the feeder cells may comprise certain lethally irradiated fibroblast, such as the murine 3T3-J2 cells. The feeder cells may form a feeder cell layer on top of the basement membrane matrix.

A suitable 3T3-J2 cell clone is well known in the art (see, for example, Todaro and Green, “Quantitative studies of the growth of mouse embryo cells in culture and their development into established lines.”/. Cell Biol. 17: 299-313, 1963), and is readily available to the public. For example, Waisman Biomanufacturing (Madison, Wisconsin) sells irradiated 3T3-J2 feeder cells produced and tested according to cGMP guidelines. These cells were originally obtained from Dr. Howard Green's laboratory under a material transfer agreement, and according to the vender, are of the quality sufficient to support, for example, skin gene therapy and wound healing clinical trials. Also, according to the vender, each vial of the 3T3 cells contains a minimum of 3×10⁶ cells that have been manufactured in fully compliant cleanrooms, and are certified mycoplasma free and low endotoxin. In addition, the cell bank has been fully tested for adventitious agents, including murine viruses. These cells have been screened for keratinocyte culture support and do not contain mitomycin C.

The method of the invention provides the use of feeder cells, such as the murine 3T3J2 clone of fibroblasts. In general, without being limited to any particular phenotype, feeder cell layers are often used to support the culture of stem cells, and/or to inhibit their differentiation. A feeder cell layer is generally a monolayer of cells that is co-cultured with, and which provides a surface suitable for growth of, the cells of interest. The feeder cell layer provides an environment in which the cells of interest can grow. Feeder cells are often mitotically inactivated (e.g., by (lethal) irradiation or treatment with mitomycin C) to prevent their proliferation.

In certain embodiments, the feeder cells are appropriately screened and GMP-grade human feeder cells, e.g., those sufficient to support clinical-grade stem cell of the invention. See Crook et al. (Cell Stem Cell I(5):490-494, 2007, incorporated by reference), for GMPgrade human feeder cells grown in medium with GMP-quality FBS.

In certain embodiments, the feeder cells can be labeled by a marker that is lacking in the stem cells, such that the stem cells can be readily distinguished and isolated from the feeder cells. For example, the feeder cells can be engineered to express a fluorescent marker, such as GFP or other similar fluorescent markers. The fluorescent-labeled feeder cells can be isolated from the stem cells using, for example, FACS sorting.

Any one of a number of physical methods of separation known in the art may be used to separate the stem cells of the invention from the feeder cells. Such physical methods, other than FACS, may include various immuno-affinity methods based upon specifically expressed makers. For example, the stem cells of the invention can be isolated based on the specific stem cell markers they express, using antibodies specific for such markers.

In one embodiment, the stem cells of the invention may be isolated by FACS utilizing an antibody, for example, against one of these markers. Fluorescent activated cell sorting (FACS) can be used to detect markers characteristic of a particular cell type or lineage. As will be apparent to one skilled in the art, this may be achieved through a fluorescent labeled antibody, or through a fluorescent labeled secondary antibody with binding specificity for the primary antibody. Examples of suitable fluorescent labels includes, but is not limited to, FITC, Alexa Fluor® 488, GFP, CFSE, CFDA-SE, DyLight 488, PE, PerCP, PE-Alexa Fluor® 700, PE-Cy5 (TRI-COLOIT), PE-Cy5.5, PI, PE-Alexa Fluor* 750, and PE-Cy7. The list of fluorescent markers is provided by way of example only and is not intended to be limiting.

It will be apparent to a person skilled in the art that FACS analysis using, for example, an antibody specific for stem cell will provide a purified stem cell population. However, in some embodiments, it may be preferable to purify the cell population further by performing a further round of FACS analysis using one or more of the other identifiable markers, such as one that select against the feeders.

The use of feeder cells is considered undesirable for certain competing methods, because the presence of feeders may complicate passaging of the cells in those competing methods. For example, the cells must be separated from the feeder cells at each passage, and new feeder cells are required at each passage. In addition, the use of feeder cells may lead to contamination of the desired cells by the feeder cells.

Use of feeder layer, however, is not necessarily a disadvantage of the present invention, since the isolated stem cells of the invention are capable of being passaged as single cells, and are in fact preferably passaged as single cell clones. Thus the potential risk of contamination by the feeders during passaging is minimized, if not eliminated.

In certain embodiments, the basement membrane matrix is a laminin-containing basement membrane matrix (e.g., MATRIGEL™ basement membrane matrix (BD Biosciences)), preferably growth factor-reduced.

In certain embodiments, the basement membrane matrix does not support 3-dimensional growth, or does not form a 3-dimensional matrix necessary to support 3-dimensional growth. Thus when plating the basement membrane matrix, it is usually not required to deposit the basement membrane matrix in a specific shape or form on a support, such as forming a dome shape or form and maintaining such shape or form after solidification, which shape or form may be required to support 3-dimensional growth. In certain embodiments, the basement membrane matrix is evenly distributed or spread out on a flat surface or supporting structure (such as a flat bottom tissue culture dish or well).

In certain embodiments, the basement membrane matrix is first thawed and diluted in cold (e.g., about 0-4° C.) feeder cell growth medium to a proper concentration (e.g., 10%), and plated and solidified on a flat surface, such as by warming up to 37° C. in a tissue culture incubator with appropriate C0₂ content (e.g., about 5%). Lethally irradiated feeder cells are then plated on top of the solidified basement membrane matrix at a proper density such that settled feeder cells forms a subconfluent or confluent feeder cell layer overnight on top of the basement membrane matrix. The feeder cells are cultured in feeder cell medium, such as a medium (e.g., 3T3-J2 growth medium) comprising: a base tissue culture medium that preferably has high glucose (e.g., about 4.5 g/L), no L-glutamine, and no sodium pyruvate (e.g., DMEM (Invitrogen cat. no. 11960; high glucose (4.5 g/L), no L-glutamine, no sodium pyruvate), 10% bovine calf serum (not heat inactivated), one or more antibiotics (e.g., 1% penicillin-streptomycin), and L-glutamine (e.g., about 1.5 mM, or 1-2 mM, or 0.5-5 mM, or 0.210 mM, or 0.1-20 mM).

According to the methods of the invention, epithelial cell colonies becomes detectable after a few days (e.g., 3-4 days, or about 10 days) of culturing the dissociated cells from the source tissue in the subject stem cell medium.

In certain embodiments, single cells may be isolated from these epithelial cell colonies by, for example, enzyme digestion. Suitable enzymes for this purpose include trypsin, such as warm 0.25% trypsin (Invitrogen, cat. no 25200056). In certain embodiments, the enzyme digestion is substantially complete such that essentially all cells in the epithelial cell clones becomes dissociated from other cells and becomes single cells. In certain embodiments, the method comprises culturing the isolated single cells (preferably after washing and resuspending the single cells) in the modified growth medium in contact with a second population of lethally irradiated feeder cells and a second basement membrane matrix in the modified growth medium. Optionally, the isolated single cells may be passed through a cell strainer of proper size (e.g., 40 micron), before the single cells are plated on the feeder cells and basement membrane matrix.

In certain embodiments, the modified growth medium is changed periodically (e.g., once every day, once every 2, 3, or 4 days, etc.) till single cell clones or clonal expansion of the isolated single stem cells form.

In certain embodiments, a single colony of the stem cell can be isolated using, for example, a cloning ring. The isolated stem cell clone can be expanded to develop a pedigree cell line, i.e., a cell line that has been derived from a single stem cell.

In certain embodiments, single stem cells can be isolated from the clonal expansion of the single stem cell, and can be passaged again as single stem cells.

### 3. Medium

The invention provides various cell culture media for isolating, culturing, and/or differentiation of the subject stem cells, comprising a base medium to which a number of factors are added to produce the stem cell culture medium for reproductive tissue stem cells. The factors that may be added to the base medium or the modified medium are first described below. Several exemplary base media and modified media of the invention are then described with further details to illustrate specific non-limiting embodiments of the invention.

Rock (Rho-kinase) Inhibitor. While not wishing to be bound by any particular theory, the addition of a Rock inhibitor may prevent anoikis, especially when culturing single stem cells. The Rock inhibitor may be (R)-(+)-trans-N-(4-Pyridyl)-4-(1-aminoethyl)-cyclohexanecarboxamide) dihydrochloride monohydrate (Y-27632, SigmaAldrich), 5-(1,4-diazepan-1-ylsulfonyl)isoquinoline (fasudil or HA1077, Cayman Chemical), (1S)-(+)-2-methyl-1-[(4methyl-5-isoquinolinyl)sulfonyl]-hexahydro-1H-1,4-diazepine dihydrochloride (HI 152, Tocris Bioscience), and N-(6-fluoro-1H-indazol-5-yl)-2-methyl-6-oxo-4-(4-(trifluoromethyl)phenyl)-1,4,5,6-tetrahydropyridine-3-carboxamide (GSK429286A, Stemgent). In certain embodiments, the final concentration for Y27632 is about 1-5 μM, or 2.5 μM. The Rho-kinase inhibitor, e.g., Y-21632, may be added to the culture medium every 1, 2, 3, 4, 5, 6, or 7 days during the first seven days of culturing the stem cells.

Wnt Agonist. The Wnt signaling pathway is defined by a series of events that occur when a Wnt protein ligand binds to a cellsurface receptor of a Frizzled receptor family member. This results in the activation of Disheveled (Dsh) family proteins which inhibit a complex of proteins that includes axin, GSK-3, and the protein APC to degrade intracellular β-catenin. The resulting enriched nuclear β-catenin enhances transcription by TCF/LEF family of transcription factors. A “Wnt agonist” as used herein includes an agent that directly or indirectly activates TCF/LEF-mediated transcription in a cell, such as through modulating the activity of any one of the proteins/genes in the Wnt signaling cascade (e.g., enhancing the activity of a positive regulator of the Wnt signaling pathway, or inhibiting the activity of a negative regulator of the Wnt signaling pathway).

Wnt agonists are selected from true Wnt agonists that bind and activate a Frizzled receptor family member including any and all of the Wnt family proteins, an inhibitor of intracellular β-catenin degradation, and activators of TCF/LEF. The Wnt agonist may stimulate a Wnt activity in a cell by at least about 10%, at least about 20%, at least about 30%, at least about 50%, at least about 70%, at least about 90%, at least about 100%, at least about 2-fold, 3-fold, 5-fold, 10-fold, 20-fold, 50-fold, 100-fold, 200-fold, 500-fold, or 1000 fold or more relative to a level of the Wnt activity in the absence of the Wnt agonist. As is known to a person of skill in the art, a Wnt activity can be determined by measuring the transcriptional activity of Wnt, for example by pTOPFLASH and pFOPFLASH Tcf luciferase reporter constructs (see Korinek et ah, Science 275: 1784-1787, 1997, incorporated herein by reference).

Representative Wnt agonist may comprise a secreted glycoprotein including Wnt1/Int-1, Wnt-2/Irp (Int-1-related Protein), Wnt-2b/13, Wnt-3/Int-4, Wnt-3a (R&D systems), Wnt4, Wnt-5a, Wnt-5b, Wnt-6 (Kirikoshi et al, Biochem. Biophys. Res. Com., 283:798805, 2001), Wnt-7a (R&D systems), Wnt-7b, Wnt-8a/8d, Wnt-8b, Wnt-9a/14, Wnt9b/14b/15, Wnt10a, Wnt10b/12, Wnt11, and Wnt16. An overview of human Wnt proteins is provided in “The Wnt Family of Secreted Proteins,” R&D Systems Catalog, 2004 (incorporated herein by reference).

Further Wnt agonists include the R-spondin family of secreted proteins, which is implicated in the activation and regulation of Wnt signaling pathway, and which comprises at least 4 members, namely R-spondin 1 (NU206, Nuvelo, San Carlos, CA), R-spondin 2 (R&D systems), R-spondin 3, and R-spondin 4. Wnt agonists also include Norrin (also known as Norrie Disease Protein or NDP) (R&D systems), which is a secreted regulatory protein that functions like a Wnt protein in that it binds with high affinity to the Frizzled-4 receptor and induces activation of the Wnt signaling pathway (Kestutis Planutis et ah, BMC Cell Biol. 8:12, 2007).

Wnt agonists further include a small-molecule agonist of the Wnt signaling pathway, an aminopyrimidine derivative (N⁴-[(2H-1,3-benzodioxol-5-yl)methyl)-6-(3methoxyphenyl)pyrimidine-2,4-diamine) of the following structure, as described in Liu et al. (Angew Chem. Int. Ed. Engl. 44 13): 1987-1990, 2005, incorporated herein by reference).

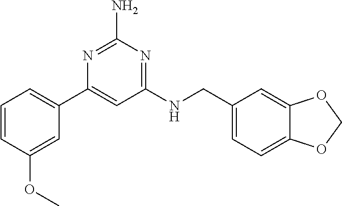 [SMILES: COc1cccc(-c2cc(NCc3ccc4c(c3)OCO4)nc(N)n2)c1]

GSK-inhibitors comprise small-interfering RNAs (siRNA, Cell Signaling), lithium (Sigma), kenpaullone (Biomol International, Leost et al., Eur. J. Biochem. 267:5983-5994, 2000), 6-Bromoindirubin-30-acetoxime (Meyer et al., Chem. Biol. 10:1255-1266, 2003), SB 216763, and SB 415286 (Sigma-Aldrich), and FRAT-family members and FRAT-derived peptides that prevent interaction of GSK-3 with axin. An overview is provided by Meijer et al. (Trends in Pharmacological Sciences 25:471-480, 2004, incorporated herein by reference). Methods and assays for determining a level of GSK-3 inhibition are known in the art, and may comprise, for example, the methods and assay as described in Liao et al. (Endocrinology 145(6):2941-2949, 2004, incorporated herein by reference).

In certain embodiments, Wnt agonist is selected from: one or more of a Wnt family member, R-spondin 1-4 (such as R-spondin 1), Norrin, Wnt3a, Wnt6, and a GSK-inhibitor.

In certain embodiments, the Wnt agonist comprises or consists of R-spondin 1. Rspondin 1 may be added to the subject culture medium at a concentration of at least about 50 ng/mL, at least about 75 ng/mL, at least about 100 ng/mL, at least about 125 ng/mL, at least about 150 ng/mL, at least about 175 ng/mL, at least about 200 ng/mL, at least about 300 ng/mL, at least about 500 ng/mL. In certain embodiments, R-spondin 1 is about 125 ng/mL.

In certain embodiments, any of the specific protein-based Wnt agonist referenced herein, such as R-spondin 1 to R-spondin 4, any Wnt family member, etc. may be replaced by a natural, synthetic, or recombinantly produced homologs or fragments thereof that retain at least about 80%, 85%, 90%, 95%, 99% of the respective Wnt agonist activity, and/or homologs or fragments thereof that share at least about 60%, 70%, 80%, 90%, 95%, 97%, 99% amino acid sequence identity as measured by any art recognized sequence alignment software based on either a global alignment technique (e.g., the Needleman-Wunsch algorithm) or a local alignment technique (e.g., the Smith-Waterman algorithm). The sequences of the representative Wnt agonist referenced herein are represented in SEQ ID NOs. 10 17.

During culturing of the subject stem cells, the Wnt family member may be added to the medium every day, every second day, every third day, while the medium is refreshed, e.g., every 1, 2, 3, 4, 5, or more days.

In certain embodiments, a Wnt agonist is selected from the group consisting of: an Rspondin, Wnt-3a and Wnt-6, or combinations thereof. In certain embodiments, an R-spondin and Wnt-3a are used together as Wnt agonist. In certain embodiments, R-spondin concentration is about 125 ng/mL, and Wnt3a concentration is about 100 ng/mL.

Mitogenic Growth Factor. Mitogenic growth factors suitable for the invention may include a family of growth factors comprising epidermal growth factor (EGF) (Peprotech), Transforming Growth Factorα (TGFa, Peprotech), basic Fibroblast Growth Factor (bFGF, Peprotech), brain-derived neurotrophic factor (BDNF, R&D Systems), and Keratinocyte Growth Factor (KGF, Peprotech).

EGF is a potent mitogenic factor for a variety of cultured ectodermal and mesodermal cells, and has a profound effect on the differentiation of specific cells in vivo and in vitro, and of some fibroblasts in cell culture. The EGF precursor exists as a membrane-bound molecule, which is proteolytically cleaved to generate the 53-amino acid peptide hormone that stimulates cells. EGF may be added to the subject culture medium at a concentration of between 1-500 ng/mL. In certain embodiments, final EGF concentration in the medium is at least about 1, 2, 5, 10, 20, 25, 30, 40, 45, or 50 ng/mL, and is not higher than about 500, 450, 400, 350, 300, 250, 200, 150, 100, 50, 30, 20 ng/mL. In certain embodiments, final EGF concentration is about 1-50 ng/mL, or about 2-50 ng/mL, or about 5-30 ng/mL, or about 5-20 ng/mL, or about 10 ng/mL.

The same concentrations may be used for an FGF, such as FGF10 or FGF7. If more than one FGF is used, for example FGF7 and FGF 10, the concentration of FGF above may refer to the total concentration of all FGF used in the medium.

In certain embodiments, any of the specific mitogenic growth factors referenced herein, such as EGF, TGFa, bFGF, BDNF, KGF, etc. may be replaced by a natural, synthetic, or recombinantly produced homologs or fragments thereof that retain at least about 80%, 85%, 90%, 95%, 99% of the respective mitogenic growth factor activity, and/or homologs or fragments thereof that share at least about 60%, 70%, 80%, 90%, 95%, 97%, 99% amino acid sequence identity as measured by any art recognized sequence alignment software based on either a global alignment technique (e.g., the Needleman-Wunsch algorithm) or a local alignment technique (e.g., the Smith-Waterman algorithm).

The sequences of the representative mitogenic growth factors referenced herein are represented in SEQ ID NOs. 18-27.

During culturing of the subject stem cells, the mitogenic growth factor may be added to the culture medium every day, every 2nd day, while the culture medium is refreshed, e.g., every day.

Any member of the bFGF family may be used. In certain embodiments, FGF7 and/or FGF10 is used. FGF7 is also known as KGF (Keratinocyte Growth Factor). In certain embodiments, a combination of mitogenic growth factors, such as EGF and KGF, or EGF and BDNF, is added to the subject culture medium. In certain embodiments, a combination of mitogenic growth factors, such as EGF and KGF, or EGF and FGF10, is added to the subject culture medium.

BMP Inhibitor. Bone Morphogenetic Proteins (BMPs) bind as a dimeric ligand to a receptor complex consisting of two different receptor serine/threonine kinases, type I and type II receptors. The type II receptor phosphorylates the type I receptor, resulting in the activation of this receptor kinase. The type I receptor subsequently phosphorylates specific receptor substrates (such as SMAD), resulting in a signal transduction pathway leading to transcriptional activity.

A BMP inhibitor as used herein includes an agent that inhibits BMP signaling through its receptors. In one embodiment, a BMP inhibitor binds to a BMP molecule to form a complex such that BMP activity is neutralized, for example, by preventing or inhibiting the binding of the BMP molecule to a BMP receptor. Examples of such BMP inhibitors may include an antibody specific for the BMP ligand, or an antigen-binding portion thereof. Other examples of such BMP inhibitors include a dominant negative mutant of a BMP receptor, such as a soluble BMP receptor that binds the BMP ligand and prevents the ligand from binding to the natural BMP receptor on the cell surface.

Alternatively, the BMP inhibitor may include an agent that acts as an antagonist or reverse agonist. This type of inhibitor binds with a BMP receptor and prevents binding of a BMP to the receptor. An example of such an agent is an antibody that specifically binds a BMP receptor and prevents binding of BMP to the antibody-bound BMP receptor.

In certain embodiments, the BMP inhibitor inhibits a BMP-dependent activity in a cell to at most 90%, at most 80%, at most 70%, at most 50%, at most 30%, at most 10%, or about 0% (near complete inhibition), relative to a level of a BMP activity in the absence of the inhibitor. As is known to one of skill in the art, a BMP activity can be determined by, for example, measuring the transcriptional activity of BMP as exemplified in Zilberberg et al. (“A rapid and sensitive bioassay to measure bone morphogenetic protein activity,” BMC Cell Biology 8:41, 2007, incorporated herein by reference).

Several classes of natural BMP-binding proteins are known, including Noggin (Peprotech), Chordin, and chordin-like proteins comprising a chordin domain (R&D systems) comprising chordin domains, Follistatin and follistatin-related proteins comprising a follistatin domain (R&D systems) comprising a follistatin domain, DAN and DAN-like proteins comprising a DAN Cystine-knot domain (e.g., Cerberus and Gremlin) (R&D systems), sclerostin/SOST (R&D systems), decorin (R&D systems), and alpha-2 macroglobulin (R&D systems) or as described in U.S. Pat. No. 8,383,349. An exemplary BMP inhibitor for use in a method of the invention is selected from Noggin, DAN, and DAN-like proteins including Cerberus and Gremlin (R&D systems). These diffusible proteins are able to bind a BMP ligand with varying degrees of affinity and inhibit BMPs' access to their signaling receptors.

Any of the above-described BMP inhibitors may be added either alone or in combination to the subject culture medium when desirable.

In certain embodiments, the BMP inhibitor is Noggin. Noggin may be added to the respective culture medium at a concentration of at least about 10 ng/mL, or at least about 20 ng/mL, or at least about 50 ng/mL, or at least about 100 ng/mL (e.g., 100 ng/mL).

In certain embodiments, any of the specific BMP inhibitors referenced herein, such as Noggin, Chordin, Follistatin, DAN, Cerberus, Gremlin, sclerostin/SOST, decorin, and alpha-2 macroglobulin may be replaced by a natural, synthetic, or recombinantly produced homologs or fragments thereof that retain at least about 80%, 85%, 90%, 95%, 99% of the respective BMP inhibiting activity, and/or homologs or fragments thereof that share at least about 60%, 70%, 80%, 90%, 95%, 97%, 99% amino acid sequence identity as measured by any art recognized sequence alignment software based on either a global alignment technique (e.g., the Needleman-Wunsch algorithm) or a local alignment technique (e.g., the SmithWaterman algorithm).

The sequences of the representative BMP inhibitors referenced herein are represented in SEQ ID NOs. 1 9.

During culturing of the subject stem cells, the BMP inhibitor may be added to the culture medium every day, every 2nd day, every 3rd day, or every 4th day, while the culture medium is refreshed every day, every second day, every third day, or every fourth day as appropriate.

VEGF Inhibitor. In certain embodiments, the VEGF inhibitor is selected from aflibercept, pegaptanib, tivozanib, 3-(4-Bromo-2,6-difluoro-benzyloxy)-5-[3-(4-pyrrolidin-1-yl-butyl)-ureido]-isothiazole-4-carboxylic acid amide hydrochloride, axitinib, N-(4-bromo-2-fluorophenyl)-6-methoxy-7-[(1-methylpiperidin-4-0)methoxy]quinazolin-4-amine, an inhibitor of VEGF-R2 and VEGF-R1, axitinib, N,2-dimethyl-6-(2-(1-methyl-1H-imidazol-2-yl)thieno[3,2-b]pyrid-in-7-yloxy)benzo[b]thiophene-3-carboxamide, tyrosine kinase inhibitor of the RET/PTC oncogenic kinase, N-(4-bromo-2-fluorophenyl)-6-methoxy-7-[(1-methylpiperidin-4-yl)methoxy]quinazolin-4-amine, pan-VEGF-R-kinase inhibitor; protein kinase inhibitor, multitargeted human epidermal receptor (HER) 1/2 and vascular endothelial growth factor receptor (VEGFR) 1/2 receptor family tyrosine kinases inhibitor, cediranib, sorafenib, vatalanib, glufanide disodium, VEGFR2-selective monoclonal antibody, angiozyme, an siRNA-based VEGFR1 inhibitor, 5-((7-Benzyloxyquinazolin-4-yl)amino)-4-fluoro-2-methyl phenol hydrochloride, any derivatives thereof and any combinations thereof.

In certain preferred embodiments, the VEGF inhibitor is a VEGF Receptor inhibitor, and even more preferably a VEGF Receptor kinase inhibitor such as Tivozanib (AV-951), AZD2932, Midostaurin (pkc412), BAW2881 (NVP-BAW2881), Nintedanib (BIBF 1120), SU5402, SU1498, BFH772, Sorafenib, Sunitinib, Dovitinib (TK1258), Semaxanib (SU5416), hypericin, vatalanib, ZM306416, AAL993, SU4312, DMXAA or Foretinib.

In certain embodiments, the VEGF Receptor inhibitor is a multi-tyrosine kinase inhibitor, such as afatinib, imatinib, dacomitinib, dasatinib, ponatinib, KD-019, bosutinib, lapatinib ditosylate, AZD9291, neratinib, poziotinib, S-222611, suramin hexasodium, AL-6802, BGB-102, PB357, Pyrotinib, sunitinib, sorafenib tosylate, pazopanib, regorafenib, apatinib, axitinib, carbozantinib, lenvatinib, nintedanib, vandetanib, tivozanib, anlotinib, midostaurin, muparfostat, BMS-690514, ENMD-2076, golvatinib, lucitanib, motesanib, necuparinib, RAF265, famitinib, telatinib, X82, ALNVSP, altiratinib, ABT348, MGCD516, OB318, ODM203, HHGV678, LY-3012207, CS2164, ilorasertib, radotinib, bafetinib, NRCAN-019, ABL001, metatinib tromethamine, rebastinib tosylate or VX-15.

Tyrosine Kinase Inhibitors. In certain embodiments, the Medium includes a tyrosine kinase inhibitor, such as nilotinib, ponatinib, dasatinib, gefitinib, erlotinib, sunitinib, or cabozantinib. In certain preferred embodiments, the tyrosine kinase inhibitor is a Pan-ABL1 Kinase Inhibitor such as Ponatinib or Dasatinib.

In certain embodiments, the Medium includes both a VEGF Receptor kinase inhibitor and a Tyrosine Kinase inhibitor, which may be the same or different compounds, such as a combination of Ponatinib and Tivozanib.

TGF-beta or TGF-beta Receptor Inhibitor. TGF-β signaling is involved in many cellular functions, including cell growth, cell fate and apoptosis. Signaling typically begins with binding of a TGF-β superfamily ligand to a Type II receptor, which recruits and phosphorylates a Type I receptor. The Type 1 receptor then phosphorylates SMADs, which act as transcription factors in the nucleus and regulate target gene expression. Alternatively, TGF-β signaling can activate MAP kinase signaling pathways, for example, via p38 MAP kinase.

The TGF-β superfamily ligands comprise bone morphogenetic proteins (BMPs), growth and differentiation factors (GDFs), anti-Mullerian hormone (AMH), activin, nodal and TGF-βs.

A TGF-β inhibitor as used herein include an agent that reduces the activity of the TGF-β signaling pathway. There are many different ways of disrupting the TGF-β signaling pathway known in the art, any of which may be used in conjunction with the subject invention. For example, TGF-β signaling may be disrupted by: inhibition of TGF-β expression by a small-interfering RNA strategy; inhibition of furin (a TGF-β activating protease); inhibition of the pathway by physiological inhibitors, such as inhibition of BMP by Noggin, DAN or DAN-like proteins; neutralization of TGF-β with a monoclonal antibody; inhibition with small-molecule inhibitors of TGF-β receptor kinase 1 (also known as activin receptor-like kinase, ALK5), ALK4, ALK6, ALK7 or other TG\hat{ }-related receptor kinases; inhibition of Smad 2 and Smad 3Signaling by overexpression of their physiological inhibitor, Smad 7, or by using thioredoxin as an Smad anchor disabling Smad from activation (Fuchs, Inhibition of TGF-β Signaling for the Treatment of Tumor Metastasis and Fibrotic Diseases. Current Signal Transduction Therapy 6(1):29-43(15), 2011).

For example, a TGF-β inhibitor may target a serine/threonine protein kinase selected from: TGF-β receptor kinase 1, ALK4, ALK5, ALK7, or p38. ALK4, ALK5 and ALK7 are all closely related receptors of the TGF-β superfamily. ALK4 has GI number 91; ALK5 (also known as TGF-β receptor kinase 1) has GI number 7046; and ALK7 has GI number 658. An inhibitor of any one of these kinases is one that effects a reduction in the enzymatic activity of any one (or more) of these kinases. Inhibition of ALK and p38 kinase has previously been shown to be linked in B-cell lymphoma (Bakkebo et ah, “TGF\hat{ }-induced growth inhibition in B-cell lymphoma correlates with Smad 1/5 signaling and constitutively active p38MAPK,” BMC Immunol. 11:57, 2010).

In certain embodiments, a TGF-β inhibitor may bind to and inhibit the activity of a Smad protein, such as R-SMAD or SMAD1-5 SMAD1, SMAD2, SMAD3, SMAD4 or SMAD5).

In certain embodiments, a TGF-β inhibitor may bind to and reduces the activity of Ser/Thr protein kinase selected from: TGF-β receptor kinase 1, ALK4, ALK5, ALK7, or p38.

In certain embodiments, the medium of the invention comprises an inhibitor of ALK5.

In certain embodiments, the TGF-β inhibitor or TGF-β receptor inhibitor does not include a BMP antagonist (i.e., is an agent other than BMP antagonist).

Various methods for determining if a substance is a TGF-β inhibitor are known. For example, a cellular assay may be used in which cells are stably transfected with a reporter construct comprising the human PAI-1 promoter or Smad binding sites, driving a luciferase reporter gene. Inhibition of luciferase activity relative to control groups can be used as a measure of compound activity (De Gouville et ah, Br. J. Pharmacol. 145(2): 166-177, 2005, incorporated herein by reference). Another example is the ALPHASCREEN® phosphosensor assay for measurement of kinase activity (Drew et ah, J. Biomol. Screen. 16(2): 164-173, 2011, incorporated herein by reference).

A TGF-β inhibitor useful for the present invention may be a protein, a peptide, a small-molecule, a small-interfering RNA, an antisense oligonucleotide, an aptamer, an antibody or an antigen-binding portion thereof. The inhibitor may be naturally occurring or synthetic. Examples of small-molecule TGF-β inhibitors that can be used in the context of this invention include, but are not limited to, the small molecule inhibitors listed in Table 1 below:

TABLE 1
Small-molecule TGF inhibitors targeting receptor kinases

| Inhibitor | Targets | IC50 (nM) | Mol Wt | Name | Formula |
|---|---|---|---|---|---|
| A83-01 | ALK5 | 12 | 421.52 | 3-(6-Methyl-2- | C25H19N5S |
| | (TGF-β RI) | | | pyridinyl)-N-phenyl-4- | |
| | ALK4 | 45 | | (4-quinolinyl)-1H- | |
| | ALK7 | 7.5 | | pyrazole-1- | |
| | | | | carbothioamide | |
| SB-431542 | ALK5 | 94 | 384.39 | 4-[4-(1,3-benzodioxol- | C22H16N4O3 |
| | ALK4 | | | 5-yl)-5-(2-pyridinyl)- | |
| | ALK7 | | | 1H-imidazol-2- | |
| | | | | yl]benzamide | |
| SB-505124 | ALK5 | 47 | 335.4 | 2-(5-benzo[1,3]dioxol- | C20H21N3O2 |
| | ALK4 | 129 | | 5-y1-2-tert-butyl- | |
| | | | | 3Himidazol- | |
| | | | | 4-yl)-6-methylpyridine | |
| | | | | hydrochloride hydrate | |
| SB-525334 | ALK5 | 14.3 | 343.42 | 6-[2-(1,1- | C21H21N5 |
| | | | | Dimethylethyl)-5-(6- | |
| | | | | methyl-2-pyridinyl)- | |
| | | | | 1H-imidazol-4- | |
| | | | | yl]quinoxaline | |
| SD-208 | ALK5 | 49 | 352.75 | 2-(5-Chloro-2- | C17H10ClFN6 |
| | | | | fluorophenyl)-4-[(4- | |
| | | | | pyridyl)amino]pteridine | |
| LY-36494 | TGR-β RI | 59 | 272.31 | 4-[3-(2-Pyridinyl)-1H- | C17H12N4 |
| | TGF-β RII | 400 | | pyrazol-4-yl]-quinoline | |
| | MLK-7K | 1400 | | | |
| SJN-2511 | ALK5 | 23 | 287.32 | 2-(3-(6- | C17H13N5 |
| | | | | Methylpyridine-2-yl)- | |
| | | | | 1H-pyrazol-4-yl)-1,5- | |
| | | | | naphthyridine | |

One or more of any of the inhibitors listed in Table 1 above, or a combination thereof, may be used as a TGF-β inhibitor in the subject invention. In certain embodiments, the combination may include: SB-525334 and SD-208 and A83-01; SD-208 and A83-01; or SD208 and A83-01.

One of skill in the art will appreciate that a number of other small-molecule inhibitors exist that are primarily designed to target other kinases, but at high concentrations may also inhibit TGF-β receptor kinases. For example, SB-203580 is a p38 MAP kinase inhibitor that, at high concentrations (for example, approximate 10 μM or more) may inhibit ALK5. Any such inhibitor that inhibits the TGF-β signaling pathway may also be used in this invention. In certain embodiments, A83-01 may be added to the culture medium at a concentration of between 10 nM and 10 μM, or between 20 nM and 5 μM, or between 50 nM and 1 μM. In certain embodiments, A83-01 may be added to the medium at about 500 nM. In certain embodiments, A83-01 may be added to the culture medium at a concentration of between 350-650 nM, 450-550 nM, or about 500 nM. In certain embodiments, A83-01 may be added to the culture medium at a concentration of between 25-75 nM, 40-60 nM, or about 50 nM.

SB-431542 may be added to the culture medium at a concentration of between 80 nM and 80 μM, or between 100 nM and 40 μM, or between 500 nM and 10 μM, or between 1-5 μM. For example, SB-431542 may be added to the culture medium at about 2 μM.

SB-505124 may be added to the culture medium at a concentration of between 40 nM and 40 μM, or between 80 nM and 20 μM, or between 200 nM and 1 μM. For example, SB505124 may be added to the culture medium at about 500 nM.

SB-525334 may be added to the culture medium at a concentration of between 10 nM and 10 μM, or between 20 nM and 5 μM, or between 50 nM and 1 μM. For example, SB525334 may be added to the culture medium at about 100 nM.

LY 364947 may be added to the culture medium at a concentration of between 40 nM and 40 μM, or between 80 nM and 20 μM, or between 200 nM and 1 μM. For example, LY 364947 may be added to the culture medium at about 500 nM.

SD-208 may be added to the culture medium at a concentration of between 40 nM and 40 μM, or between 80 nM and 20 μM, or between 200 nM and 1 μM. For example, SD-208 may be added to the culture medium at about 500 nM.

S JN 2511 may be added to the culture medium at a concentration of between 20 nM and 20 μM, or between 40 nM and 10 μM, or between 100 nM and 1 μM. For example, A83-01 may be added to the culture medium at approximately 200 nM.

p38 Inhibitor. A “p38 inhibitor” may include an inhibitor that, directly or indirectly, negatively regulates p38 signaling, such as an agent that binds to and reduces the activity of at least one p38 isoform. p38 protein kinases (see, GI number 1432) are part of the family of mitogen activated protein kinases (MAPKs). MAPKs are serine/threonine specific protein kinases that respond to extracellular stimuli, such as environmental stress and inflammatory cytokines, and regulate various cellular activities, such as gene expression, differentiation, mitosis, proliferation, and cell survival/apoptosis. The p38 MAPKs exist as α, β, β2, γ and δ isoforms.

Various methods for determining if a substance is a p38 inhibitor are known, such as: phospho-specific antibody detection of phosphorylation at Thr180/Tyr182, which provides a well-established measure of cellular p38 activation or inhibition; biochemical recombinant kinase assays; tumor necrosis factor alpha (TNFa) secretion assays; and DiscoverRx high throughput screening platform for p38 inhibitors. Several p38 activity assay kits also exist (e.g., Millipore, SigmaAldrich).

In certain embodiments, high concentrations (e.g., more than 100 nM, or more than 1 μM, more than 10 μM, or more than 100 μM) of a p38 inhibitor may have the effect of inhibiting TGF-β. In other embodiments, the p38 inhibitor does not inhibit TGF-β signaling.

Various p38 inhibitors are known in the art (for example, see Table 1). In some embodiments, the inhibitor that directly or indirectly negatively regulates p38 signaling is selected from the group consisting of SB-202190, SB-203580, VX-702, VX-745, PD169316, RO-4402257 and BIRB-796.

In certain embodiments, the medium comprises both: a) an inhibitor that binds to and reduces the activity of any one or more of the kinases from the group consisting of: ALK4, ALK5 and ALK7; and b) an inhibitor that binds to and reduces the activity of p38.

In certain embodiments, the medium comprises an inhibitor that binds to and reduces the activity of ALK5 and an inhibitor that binds to and reduces the activity of p38.

In one embodiment, the inhibitor binds to and reduces the activity of its target (for example, TGF-β and/or p38) by more than 10%; more than 30%; more than 60%; more than 80%; more than 90%; more than 95%; or more than 99% compared to a control, as assessed by a cellular assay. Examples of cellular assays for measuring target inhibition are well known in the art as described above.

An inhibitor of TGF-β and/or p38 may have an IC50 value equal to or less than 2000 nM; less than 1000 nM; less than 100 nM; less than 50 nM; less than 30 nM; less than 20 nM or less than 10 mM. The IC50 value refers to the effectiveness of an inhibitor in inhibiting its target's biological or biochemical function. The IC50 indicates how much of a particular inhibitor is required to inhibit a kinase by 50%. IC50 values can be calculated in accordance with the assay methods set out above. An inhibitor of TGF-β and/or p38 may exist in various forms, including natural or modified substrates, enzymes, receptors, small organic molecules, such as small natural or synthetic organic molecules of up to 2000 Da, preferably 800 Da or less, peptidomimetics, inorganic molecules, peptides, polypeptides, antisense oligonucleotides aptamers, and structural or functional mimetics of these including small molecules.

In certain embodiments, the inhibitor of TGF-β and/or p38 may also be an aptamer. As used herein, the term “aptamer” refers to strands of oligonucleotides (DNA or RNA) that can adopt highly specific three-dimensional conformations. Aptamers are designed to have high binding affinities and specificities towards certain target molecules, including extracellular and intracellular proteins. Aptamers may be produced using, for example, Systematic Evolution of Ligands by Exponential Enrichment (SELEX) process (see, for example, Tuerk and Gold, Systematic evolution of ligands by exponential enrichment: RNA ligands to bacteriophage T4 DNA Polymerase. Science 249:505-510, 1990, incorporated herein by reference).

In certain embodiments, the TGF-β and/or p38 inhibitor may be a small synthetic molecule with a molecular weight of between 50 and 800 Da, between 80 and 700 Da, between 100 and 600 Da, or between 150 and 500 Da.

In certain embodiments, the TGF-β and/or p38 inhibitor comprises a pyridinylimidazole or a 2,4-disubstituted teridine or a quinazoline, for example comprises:

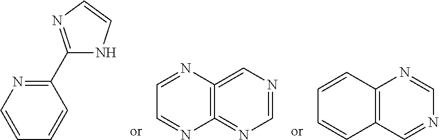

Particular examples of TGF-β and/or p38 inhibitors that may be used in accordance with the invention include, but are not limited to: SB-202190, SB-203580, SB-206718, SB227931, VX-702, VX-745, PD-169316, RO-4402257, BIRB-796, A83-01SB-431542, SB505124, SB-525334, LY 364947, SD-208, SJ 2511 (see Table 2).

For example, SB-202190 may be added to the culture medium at a concentration of between 50 nM and 100 μM, or between 100 nM and 50 μM, or between 1 μM and 50 μM.

For example, SB-202190 may be added to the culture medium at approximately 10 μM. SB-203580 may be added to the culture medium at a concentration of between 50 nM and 100 μM, or between 100 nM and 50 μM, or between 1 μM and 50 μM. For example, SB-203580 may be added to the culture medium at approximately 10 μM.

VX-702 may be added to the culture medium at a concentration of between 50 nM and 100 μM, or between 100 nM and 50 μM, or between 1 μM and 25 μM. For example, VX-702 may be added to the culture medium at approximately 5 μM.

VX-745 may be added to the culture medium at a concentration of between 10 nM and 50 μM, or between 50 nM and 50 μM, or between 250 nM and 10 μM. For example, VX-745 may be added to the culture medium at approximately 1 μM.

PD-169316 may be added to the culture medium at a concentration of between 100 nM and 200 μM, or between 200 nM and 100 μM, or between 1 μM and 50 μM. For example, PD169316 may be added to the culture medium at approximately 20 μM.

RO-4402257 may be added to the culture medium at a concentration of between 10 nM and 50 μM, or between 50 nM and 50 μM, or between 500 nM and 10 μM. For example, RO-4402257 may be added to the culture medium at approximately 1 μM.

BIRB-796 may be added to the culture medium at a concentration of between 10 nM and 50 μM, or between 50 nM and 50 μM, or between 500 nM and 10 μM. For example, BIRB-796 may be added to the culture medium at approximately 1 μM.

See Table 1 and associated text above for the applicable concentrations for the other factors in Table 2.

TABLE 2
Exemplary TGF-β and/or p38 Inhibitors
Exemplary TGF-β and/or p38 Inhibitors

| Inhibitor | Targets | IC50 (nM) | Mol Wt | Name | Formula |
|---|---|---|---|---|---|
| A83-01 | ALK5 | 12 | 421.52 | 3-(6-Methyl-2- | C25H19N5S |
| | (TGF-βRI) | | | pyridinyl)-N-phenyl-4- | |
| | ALK4 | 45 | | (4-quinolinyl)-1H- | |
| | ALK7 | 7.5 | | pyrazole-1- | |
| | | | | carbothioamide | |
| SB-431542 | ALK5 | 94 | 384.39 | 4-[4-(1,3-benzodioxol- | C22H16N4O3 |
| | ALK4 | | | 5-yl)-5-(2-pyridinyl)- | |
| | ALK7 | | | 1H-imidazol-2- | |
| | | | | yl]benzamide | |
| SB-505124 | ALK5 | 47 | 335.4 | 2-(5-benzo[1,3]dioxol- | C20H21N3O2 |
| | ALK4 | 129 | | 5-y1-2-tert-butyl- | |
| | | | | 3Himidazol- | |
| | | | | 4-yl)-6-methylpyridine | |
| | | | | hydrochloride hydrate | |
| SB-525334 | ALK5 | 14.3 | 343.42 | 6-[2-(1,1- | C21H21N5 |
| | | | | Dimethylethyl)-5-(6- | |
| | | | | methyl-2-pyridinyl)- | |
| | | | | 1H-imidazol-4- | |
| | | | | yl]quinoxaline | |
| SD-208 | ALK5 | 49 | 352.75 | 2-(5-Chloro-2- | C17H10ClFN6 |
| | | | | fluorophenyl)-4-[(4- | |
| | | | | pyridyl)amino]pteridine | |
| LY-36494 | TGR-βRI | 59 | 272.31 | 4-[3-(2-Pyridinyl)-1H- | C17H12N4 |
| | TGF-βRII | 400 | | pyrazol-4-yl]-quinoline | |
| | MLK-7K | 1400 | | | |
| LY364947 | ALK5 | 59 | 272.30 | 4-[3-(2-pyridinyl)-1H- | C₁₇H₁₂N₄ |
| | | | | pyrazol-4-yl]-quinoline | |
| SJN-2511 | ALK5 | 23 | 287.32 | 2-(3-(6- | C17H13N5 |
| | | | | Methylpyridine-2-yl)- | |
| | | | | 1H-pyrazol-4-yl)-1,5- | |
| | | | | naphthyridine | |
| SB-202190 | p38 MAP | 38 | 331.35 | 4-[4-(4-Fluorophenyl)- | C20H14N3OF |
| | kinase | | | 5-(4-pyridinyl)-1H- | |
| | p38α | 50 | | imidazol-2-yl]phenol | |
| | p38β | 100 | | | |
| SB-203580 | p38 | 50 | 377.44 | 4-[5-(4-Fluorophenyl)- | C21H16FN3OS |
| | p38β2 | 500 | | 2-[4- | |
| | | | | (methylsulfonyl)phenyl]- | |
| | | | | 1H-imidazol-4- | |
| | | | | yl]pyridine | |
| VX-702 | p38α | 4-20; | 404.32 | 6- | C19H12F4N4O2 |
| | | (Kd = 3.7) | | [(Aminocarbonyl)(2,6- | |
| | p38β | Kd = 17 | | difluorophenyl)amino]- | |
| | | | | 2-(2,4-difluorophenyl)- | |
| | | | | 3-pyridinecarboxamide | |
| VX-745 | p38α | 10 | 436.26 | 5-(2,6-Dichlorophenyl)- | C19H9Cl2F2N3OS |
| | | | | 2-[2,4- | |
| | | | | difluorophenyl)thio]- | |
| | | | | 6H-pyrimido [1,6- | |
| | | | | b]pyridazin-6-one | |
| PD-169316 | p38 | 89 | 360.3 | 4-[5-(4-fluorophenyl)- | C20H13FN4O |
| | | | | 2-(4-nitrophenyl)-1H- | |
| | | | | imidazol-4-yl]-pyridine | |
| RO- | p38α | 14 | | Pyrido[2,3-d]pyrimidin- | |
| 4402257 | p38β | 480 | | 7(8H)-one,6-(2,4- | |
| | | | | difluorophenoxy)-2-[[3- | |
| | | | | hydroxy-1-(2- | |
| | | | | hydroxyethyl)propyl]ami- | |
| | | | | no]-8-methyl- | |
| BIRB-796 | p38 | 4 | 527.67 | 1-[2-(4-methylphenyl)- | C31H37N5O3 |
| | | | | 5-tert-butyl-pyrazol-3- | |
| | | | | yl]-3-[4-(2-morpholin- | |
| | | | | 4-ylethoxy)naphthalen- | |
| | | | | 1-yl]urea ::3-[2-(4- | |
| | | | | methylphenyl)-5-tert- | |
| | | | | butyl-pyrazol-3-yl]-1- | |
| | | | | [4-(2-morpholin-4- | |
| | | | | ylethoxy)naphthalen-1- | |
| | | | | yl]urea ::3-[3-tert-butyl- | |
| | | | | 1-(4-methylphenyl)- | |
| | | | | 1H-pyrazol-5-yl]-1-{4- | |
| | | | | [2-(morpholin-4- | |
| | | | | yl)ethoxy]naphthalen- | |
| | | | | 1-yl}urea | |

Thus, in some embodiments, the inhibitor that directly or indirectly, negatively regulates TGF-β and/or p38 signaling is added to the culture medium at a concentration of between 1 nM and 100 μM, between 10 nM and 100 μM, between 100 nM and 10 μM, or about 1 μM. For example, wherein the total concentration of the one or more inhibitor is between 10 nM and 100 μM, between 100 nM and 10 μM, or about 1 μM.

Oct4-activating Agent. An Oct4-activating agent is an agent that can activate Oct4 promoter-driven reporter genes, such as a luciferase gene under the transcriptional control of an Oct4-promoter, and more preferably is an able to activate both Oct4 and Nanog promoter-driven reporter genes. Furthermore, when added to the reprogramming mixture along with the quartet reprogramming factors (Oct4, Sox2, c-Myc, and Klf4), an Oct4-activating agent enhances the iPSC reprogramming efficiency and accelerated the reprogramming process. Exemplary Oct4-activating Agents are taught in, for example, US Patent Application 20150191701 and Li et al. (2012) “Identification of Oct4-activating compounds that enhance reprogramming efficiency”. PNAS 109(51):20853-8.

In certain embodiments, the Oct4-activating agent is represented in formula:

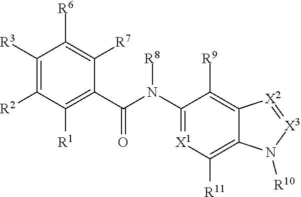

wherein,
X¹ is C(R¹²) or N;
X² is C(R⁴) or N;
X³ is C(R⁵) or N;
R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl, wherein R² and R³ are optionally joined to form a substituted or unsubstituted heterocycloalkyl or substituted or unsubstituted heteroaryl.

In certain preferred embodiments, R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, substituted or unsubstituted alkyl, substituted or unsubstituted heteroalkyl, or substituted or unsubstituted heterocycloalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, substituted or unsubstituted alkyl, or substituted or unsubstituted heteroalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, substituted or unsubstituted C₁ to C₁₀ alkyl, substituted or unsubstituted 2 to 10 membered heteroalkyl or substituted or unsubstituted 3 to 8 membered heterocycloalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, substituted or unsubstituted C₁ to C₁₀ alkyl or substituted or unsubstituted 2 to 10 membered heteroalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, unsubstituted alkyl, unsubstituted heteroalkyl, or substituted heterocycloalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, unsubstituted alkyl or unsubstituted heteroalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, unsubstituted C₁ to C₁₀ alkyl, unsubstituted 2 to 10 membered heteroalkyl, or substituted 3 to 8 membered heterocycloalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, —CN, —NO₂, —NH₂, —CF₃, —CCl₃, —OH, —SH, —SO₃H, —C(O)OH, —C(O)NH₂, unsubstituted C₁ to C₁₀ alkyl or unsubstituted 2 to 10 membered heteroalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, unsubstituted C₁ to C₁₀ alkyl or unsubstituted 2 to 10 membered heteroalkyl.

In certain preferred embodiments, R¹, R², R³, R⁴, R⁵, R⁶, R⁷, R⁸, R⁹, R¹⁰, R¹¹ and R¹² are independently selected from hydrogen, halogen, —N(CH₃)₂, unsubstituted C₁ to C₅ alkyl or unsubstituted C₁ to C₅ alkoxy.

In certain preferred embodiments, R¹, R², R³, R⁴, R⁵, R⁶, R⁷, R⁸, R⁹, R¹⁰, R¹¹ and R¹² are independently selected from hydrogen, halogen, —N(CH₃)₂, unsubstituted C₁ to C₅ alkyl, methoxy, ethoxy or propoxy.

In certain embodiments, the Oct4-activating agent is selected from the group consisting of:

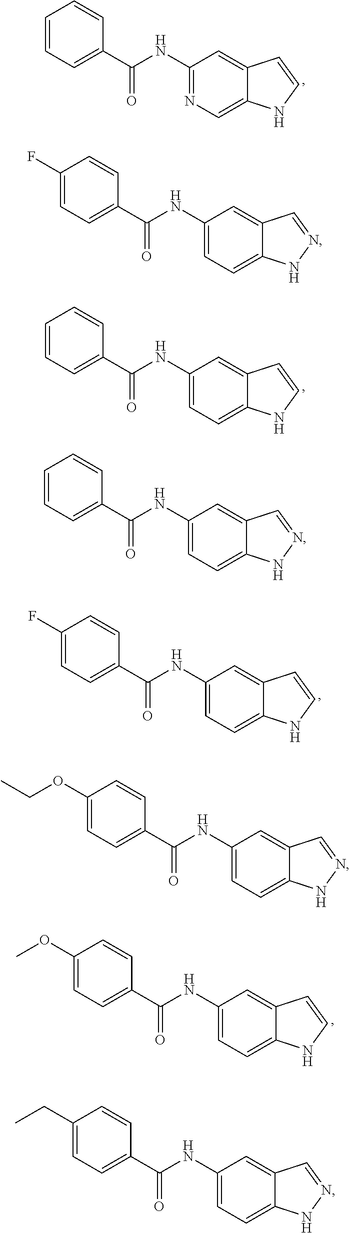

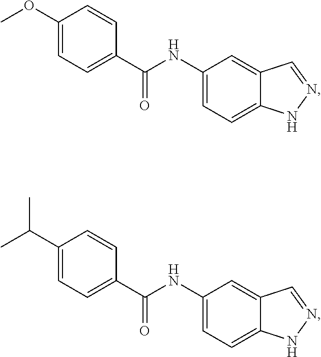 [SMILES: COc1ccc(C(=O)Nc2ccc3[nH]ncc3c2)cc1 ; CC(C)c1ccc(C(=O)Nc2ccc3[nH]ncc3c2)cc1]

In certain embodiments, the Oct4-activating agent is OAC1, having the structure:

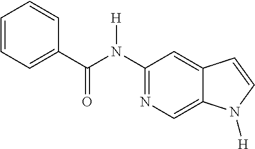 [SMILES: O=C(Nc1cc2cc[nH]c2cn1)c1ccccc1]

TrkA Inhibitors. Representative examples of TrkA inhibitors include BMS-754807, GW441756, PF-06273340, Sitravatinib (MGCD516), ANA-12, GNF-5837, Belizatinib (TSR-011), Larotrectinib (LOXO-101) sulfate, Lestaurtinib, Entrectinib (RXDX-101), GNF 5837 and AG-879.

Preferably the TrkA inhibitor is selective for TrkA relative to TrkB or TrkC, such as GW441756 and Sitravatinib (MGCD516).

Preferably the TrkA inhibitor is a potent, selective inhibitor of TrkA with IC50 of 10 nM or less, with an IC50 for inhibiting c-Raf1 and CDK2 at least 100-fold greater than the IC50 for inhibiting TrkA. such as GW441756.

Other representative Trk inhibitor can be one found in U.S. Pat. No. 9,187,489 and International Publication No. WO 2013/183578, both of which are incorporated by reference in their entireties herein. Exemplary Trk inhibitors include PLX7486 and DS-6051.

Non-limiting examples of Trk inhibitors can be found in U.S. Publication No. 2015/0306086 and International Publication No. WO 2013/074518, both of which are incorporated by reference in their entireties herein. Exemplary Trk inhibitors include TSR-011.

Further examples of Trk inhibitors can be found in U.S. Pat. No. 8,637,516, International Publication No. WO 2012/034091, U.S. Pat. No. 9,102,671, International Publication No. WO 2012/116217, U.S. Publication No. 2010/0297115, International Publication No. WO 2009/053442, U.S. Pat. No. 8,642,035, International Publication No. WO 2009092049, U.S. Pat. No. 8,691,221, International Publication No. WO2006131952, all of which are incorporated by reference in their entireties herein. Exemplary Trk inhibitors include GNF-4256, described in Cancer Chemother. Pharmacol. 75(1):131-141, 2015; and GNF-5837 (N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]urea), described in ACS Med. Chem. Lett. 3(2):140-145, 2012, each of which is incorporated by reference in its entirety herein.

Additional examples of Trk inhibitors include those disclosed in U.S. Publication No. 2010/0152219, U.S. Pat. No. 8,114,989, and International Publication No. WO 2006/123113, all of which are incorporated by reference in their entireties herein. Exemplary Trk inhibitors include AZ623, described in Cancer 117(6):1321-1391, 2011; AZD6918, described in Cancer Biol. Ther. 16(3):477-483, 2015; AZ64, described in Cancer Chemother. Pharmacol. 70:477-486, 2012; AZ-23 ((S)-5-Chloro-N2-(1-(5-fluoropyridin-2-yl)ethyl)-N4-(5-isopropoxy-1H-pyrazol-311)pyrimidine-2,4-diamine), described in Mol. Cancer Ther. 8:1818-1827, 2009; and AZD7451; each of which is incorporated by reference in its entirety.

A Trk inhibitor can include those described in U.S. Pat. Nos. 7,615,383; 7,384,632; 6,153,189; 6,027,927; 6,025,166; 5,910,574; 5,877,016; and 5,844,092, each of which is incorporated by reference in its entirety.

Further examples of Trk inhibitors include CEP-751, described in Int. J. Cancer 72:672-679, 1997; CT327, described in Acta Derm. Venereol. 95:542-548, 2015; compounds described in International Publication No. WO 2012/034095; compounds described in U.S. Pat. No. 8,673,347 and International Publication No. WO 2007/022999; compounds described in U.S. Pat. No. 8,338,417; compounds described in International Publication No. WO 2016/027754; compounds described in U.S. Pat. No. 9,242,977; compounds described in U.S. Publication No. 2016/0000783; sunitinib (N-(2-diethylaminoethyl)-5-[(Z)-(5-fluoro-2-oxo-1H-indol-3-ylidene)methyl]-2,4-dimethyl-1H-pyrrole-3-carboxamide), as described in PLoS One 9:e95628, 2014; compounds described in International Publication No. WO 2011/133637; compounds described in U.S. Pat. No. 8,637,256; compounds described in Expert. Opin. Ther. Pat. 24(7):731-744, 2014; compounds described in Expert Opin. Ther. Pat. 19(3):305-319, 2009; (R)-2-phenylpyrrolidine substituted imadizopyridazines, e.g., (4-((5-chloro-4-(methylamino)-7H-pyrrolo[2,3-d]pyrimidin-2-yl)amino)-3-methoxyphenyl)(morpholino)methanone as described in ACS Med. Chem. Lett. 6(5):562-567, 2015; GTx-186 and others, as described in PLoS One 8(12):e83380, 2013; K252a ((9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one), as described in Mol. Cell Biochem. 339(1-2):201-213, 2010; 4-aminopyrazolylpyrimidines, e.g., AZ-23 (((S)-5-chloro-N2-(1-(5-fluoropyridin-2-yl)ethyl)-N4-(5-isopropoxy-1H-pyrazol-3-yl)pyrimidine-2,4-diamine)), as described in J. Med. Chem. 51(15):4672-4684, 2008; PHA-739358 (danusertib), as described in Mol. Cancer Ther. 6:3158, 2007; Go 6976 (5,6,7,13-tetrahydro-13-methyl-5-oxo-12H-indolo[2,3-a]pyrrolo[3,4-c]carbazole-12-propanenitrile), as described in J. Neurochem, 72:919-924, 1999; GW441756 ((3Z)-3-[(1-methylindol-3-yl)methylidene]-1H-pyrrolo[3,2-b]pyridin-2-one), as described in IJAE 115:117, 2010; milciclib (PHA-848125AC), described in J. Carcinog. 12:22, 2013; AG-879 ((2E)-3-[3,5-Bis(1,1-dimethylethyl)-4-hydroxyphenyl]-2-cyano-2-propenethioamide); altiratinib (N-(4-((2-(cyclopropanecarboxamido)pyridin-4-yl)oxy)-2,5-difluorophenyl)-N-(4-fluorophenyl)cyclopropane-1,1-dicarboxamide); cabozantinib (N-(4-((6,7-Dimethoxyquinolin-4-yl)oxy)phenyl)-N′-(4-fluorophenyl)cyclopropane-1,1-dicarboxamide); lestaurtinib ((5S,6S,8R)-6-Hydroxy-6-(hydroxymethyl)-5-methyl-7,8,14,15-tetrahydro-5H-16-oxa-4b,8a,14-triaza-5,8-methanodibenzo[b, h]cycloocta[jkl]cyclopenta[e]-as-indacen-13(6H)-one); dovatinib (4-amino-5-fluoro-3-[6-(4-methylpiperazin-1-yl)-1H-benzimidazol-2-yl]quinolin-2(1H)-one mono 2-hydroxypropanoate hydrate); sitravatinib (N-(3-fluoro-4-((2-(5-(((2-methoxyethyl)amino)methyl)pyridin-2-yl)thieno[3,2-b]pyridin-7-yl)oxy)phenyl)-N-(4-fluorophenyl)cyclopropane-1,1-dicarboxamide); ONO-5390556; regorafenib (4-[4-({[4-Chloro-3-(trifluoromethyl)phenyl]carbamoyl}amino)-3-fluorophenoxy]-N-methylpyridine-2-carboxamide hydrate); VSR-902A; all of the references above are incorporated by reference in their entireties herein.

In some embodiments, a Trk inhibitor is selected from the group consisting of: (6R)-9-fluoro-2,11,15,19,20,23-hexaazapentacyclo[15.5.2.1^{7,11}.0^{2,6}.0^{20,24}]pentacosa-1 (23),7,9,17(24),18,21-hexaene-16,25-dione; (6R)-12-oxa-2,16,20,21,24,26-hexaazapentacyclo[16.5.2.1^{7,11}.0^{2,6}.0^{21,25}]hexacosa-1(24),7(26),8,10,18(25),19,22-heptaen-17-one; (6R)-9-fluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one; (6R)-9-fluoro-15-hydroxy-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one; (6R,13S)-9-fluoro-13-hydroxy-2,11,15,19,20,23-hexaazapentacyclo-[15.5.2.1^{7,11}.0^{2,6}.0^{20,24}]pentacosa-1(23),7,9,17(24),18,21-hexaene-16,25-dione; (6R,15R)-9-fluoro-15-hydroxy-13-oxa-2,11,17,21,22,25-hexaazapentacyclo-[17.5.2.0^{2,6}.0^{7,12}.0^{22,26}]hexacosa-1 (25),7,9,11,19(26),20,23-heptaen-18-one; (6R,13R)-9-fluoro-13-hydroxy-2,11,15,19,20,23-hexaazapentacyclo-[15.5.2.1^{7,11}.0^{2,6}.0^{20,24}]pentacosa-1(23),7,9,17(24),18,21-hexaene-16,25-dione; (6R)-9-fluoro-13-oxa-2,11,16,20,21,24-hexaazapentacyclo[16.5.2.0^{2,6}.0^{7,12}.0^{21,25}]pentacosa-1(24),7,9,11,18(25),19,22-heptaen-17-one; (6R)-9-fluoro-13-oxa-2,11,18,22,23,26-hexaazapentacyclo[18.5.2.0^{2,6}.0^{7,12}.0^{23,27}]heptacosa-1(26),7,9,11,20(27),21,24-heptaen-19-one; (6R)-9-fluoro-2,11,16,20,21,24-hexaazapentacyclo[16.5.2.1^{7,11}.0^{2,6}.0^{21,25}]hexacosa-1(24),7,9,18(25),19,22-hexaene-17,26-dione; (6R)-9-fluoro-2,11,13,16,20,21,24-heptaazapentacyclo[16.5.2.0^{2,6}.0^{7,12}.0^{21,25}]pentacosa-1(24),7,9,11,18(25),19,22-heptaen-17-one; (6R)-9-fluoro-2,11,13,17,21,22,25-heptaazapentacyclo[17.5.2.0^{2,6}.0^{7,12}.0^{22,26}]hexacosa-1(25),7,9,11,19(26),20,23-heptaen-18-one; (6R)-9-fluoro-13,16-dioxa-2,11,20,21,24-pentaazapentacyclo[16.5.2.0^{2,6}.0^{7,12}.0^{21,25}]-pentacosa-1 (24),7,9,11,18(25),19,22-heptaen-17-one; (6R)-9-fluoro-14-oxa-2,11,18,19,22-pentaazapentacyclo[14.5.2.1^{7,11}.0^{2,6}.0^{19,23}]tetracosa-1(22),7,9,16(23), 17,20-hexaene-15,24-dione; (6R)-9-fluoro-13,16-dioxa-2,11,17,21,22,25-hexaazapentacyclo[17.5.2.0^{2,6}.0^{7,12}.0^{22,26}]hexacosa-1(25),7,9,11,19(26),20,23-heptaen-18-one; (6R,13R)-9,13-difluoro-2,11,15,19,20,23-hexaazapentacyclo[15.5.2.1^{7,11}.0^{2,6}.0^{20,24}]pentacosa-1(23),7,9,17(24),18,21-hexaene-16,25-dione; (6R)-9-fluoro-17-methyl-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one; (6R)-9,15,15-trifluoro-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one; (6R)-9-fluoro-13-oxa-2,17,21,22,25-pentaazapentacyclo[17.5.2.0^{2,6}.0^{7,12}.0^{22,26}]hexacosa-1(25),7,9,11,19(26),20,23-heptaen-18-one; (6R)-9-fluoro-13-oxa-2,16,20,21,24-pentaazapentacyclo[16.5.2.0^{2,6}.0^{7,12}.0^{21,25}]pentacosa-1(24),7,9,11,18(25),19,22-heptaene; 1-[(6R)-9-fluoro-13-oxa-2,16,20,21,24-pentaazapentacyclo[16.5.2.0^{2,6}.0^{7,12}.0^{21,25}]pentacosa-1(24),7,9,11,18(25),19,22-heptaen-16-yl]ethan-1-one; 1-[(6R)-9-fluoro-13-oxa-2,16,20,21,24-pentaazapentacyclo[16.5.2.0^{2,6}.0^{7,12}.0^{21,25}]pentacosa-1(24),7,9,11,18(25),19,22-heptaen-16-yl]-2-hydroxyethan-1-one; (6R)-9-fluoro-13-oxa-2,17,21,22,25-pentaazapentacyclo[17.5.2.0^{2,6}.0^{7,12}.0^{22,26}]hexacosa-1(25),7,9,11,19(26),20,23-heptaene; (6R)-9-fluoro-16-methanesulfonyl-13-oxa-2,16,20,21,24-pentaazapentacyclo[16.5.2.0^{2,6}.0^{7,12}.0^{21,25}] pentacosa-1(24),7,9,11,18(25),19,22-heptaene; 2-[(6R)-9-fluoro-13-oxa-2,16,20,21,24-pentaazapentacyclo[16.5.2.0^{2,6}.0^{7,12}.0^{21,25}]pentacosa-1(24),7,9,11,18(25),19,22-heptaen-16-yl]acetic acid; (6R)-9-fluoro-17-methanesulfonyl-13-oxa-2,17,21,22,25-pentaazapentacyclo[17.5.2.0^{2,6}.0^{7,12}.0^{22,26}] hexacosa-1(25),7,9,11,19(26),20,23-heptaene; (6R)-N-ethyl-9-fluoro-13-oxa-2,17,21,22,25-pentaazapentacyclo[17.5.2.0^{2,6}.0^{7,12}.0^{22,26}]hexacosa-1(25),7,9,11,19(26),20,23-heptaene-17-carboxamide; (6R)-N-ethyl-9-fluoro-13-oxa-2,16,20,21,24-pentaazapentacyclo-[16.5.2.0^{2,6}.0^{7,12}0^{21,25}]pentacosa-1(24),7,9,11,18(25),19,22-heptaene-16-carboxamide; (6S)-9-fluoro-4,13-dioxa-2,11,17,21,22,25-hexaazapentacyclo[17.5.2.0^{2,6}.0^{7,12}.0^{22,26}]hexacosa-1(25),7(12),8,10,19(26),20,23-heptaene-3,18-dione; (6S)-9-fluoro-4,13-dioxa-2,11,16,20,21,24-hexaazapentacyclo [16.5.2.0^{2,6}.0^{7,12}.0^{21,25}]pentacosa-1(24),7(12),8,10,18(25),19,22-heptaene-3,17-dione; (6R)-9-fluoro-2,11,16,20,21,24-hexaazapentacyclo[16.5.2.0^{2,6}.0^{7,12}.0^{21,25}]pentacosa-1(24),7,9,11,18(25),19,22-heptaen-17-one; (6R)-9-fluoro-15-methyl-2,11,16,20,21,24-hexaazapentacyclo[16.5.2.0^{2,6}.0^{7,12}.0^{21,25}]pentacosa-1(24),7,9,11,18(25),19,22-heptaen-17-one; (6R,13R)-9-fluoro-13-methyl-2,11,15,19,20,23-hexaazapentacyclo[15.5.2.1^{7,11}.0^{2,6}.0^{20,24}]pentacosa-1(23),7,9,17(24),18,21-hexaene-16,25-dione; (6R,13S)-9-fluoro-13-methyl-2,11,15,19,20,23-hexaazapentacyclo [15.5.2.1^{7,11}.0^{2,6}.0^{20,24}]pentacosa-1(23),7,9,17(24),18,21-hexaene-16,25-dione; (6R)-9-fluoro-15,15-dimethyl-13-oxa-2,11,17,21,22,25-hexaazapentacyclo[17.5.2.0^{2,6}.0^{7,12}.0^{22,26}]hexacosa-1(25),7,9,11,19(26),20,23-heptaen-18-one; (6R)-9-fluoro-15,15-dimethyl-2,11,16,20,21,24-hexaazapentacyclo[16.5.2.0^{2,6}.0^{7,12}.0^{21,25}]pentacosa-1(24),7,9,11,18(25),19,22-heptaen-17-one; (6R)-9-fluoro-13-oxa-2,11,16,17,21,25,26,29-octaazahexacyclo[21.5.2.0^{2,6}.0^{7,12}.0^{16,20}.0^{26,30}]triaconta-1(29),7,9,11,17,19,23(30),24,27-nonaen-22-one; (6R)-9-fluoro-13-oxa-2,11,19,21,25,26,29-heptaazahexacyclo[21.5.2.0^{2,6}.0^{7,12}.0^{15,20}.0^{26,30}]triaconta-1(29),7,9,11,15(20),16,18,23(30),24,27-decaen-22-one; (6R)-9-fluoro-13,13-dimethyl-2,11,15,19,20,23-hexaazapentacyclo [15.5.2.1^{7,11}.0^{2,6}.0^{20,24}]pentacosa-1(23),7,9,17(24),18,21-hexaene-16,25-dione; (4R,6R,15S)-9-fluoro-4,15-dihydroxy-13-oxa-2,17,21,22,25-pentaazapentacyclo[17.5.2.0^{2,6}.0^{7,12}.0^{22,26}]hexacosa-1(25),7(12),8,10,19(26),20,23-heptaen-18-one; (4R,6S,15S)-9-fluoro-4,15-dihydroxy-13-oxa-2,17,21,22,25-pentaazapentacyclo [17.5.2.0^{2,6}.0^{7,12}.0^{22,26}]hexacosa-1(25),7(12),8,10,19(26),20,23-heptaen-18-one; (4R,6R)-9-fluoro-4-hydroxy-13-oxa-2,17,21,22,25-pentaazapentacyclo[17.5.2.0^{2,6}.0^{7,12}.0^{22,26}]hexacosa-1(25),7(12),8,10,19(26),20,23-heptaen-18-one; (4R,6S)-9-fluoro-4-hydroxy-13-oxa-2,17,21,22,25-pentaazapentacyclo[17.5.2.0^{2,6}.0^{7,12}.0^{22,26}]hexacosa-1(25),7(12),8,10,19(26),20,23-heptaen-18-one; (4R,6R)-9-fluoro-4-hydroxy-13-oxa-2,16,20,21,24-pentaazapentacyclo[16.5.2.0^{2,6}.0^{7,12}.0^{21,25}]pentacosa-1(24),7,9,11,18(25),19,22-heptaen-17-one; (4R,6S)-9-fluoro-4-hydroxy-13-oxa-2,16,20,21,24-pentaazapentacyclo [16.5.2.0^{2,6}.0^{7,12}.0^{21,25}]pentacosa-1(24),7,9,11,18(25),19,22-heptaen-17-one; (4R,6R,15R)-9-fluoro-4,15-dihydroxy-13-oxa-2,17,21,22,25-pentaazapentacyclo[17.5.2.0^{2,6}.0^{7,12}.0^{22,26}]hexacosa-1(25),7(12),8,10,19(26),20,23-heptaen-18-one; (4R,6S,15R)-9-fluoro-4,15-dihydroxy-13-oxa-2,17,21,22,25-pentaazapentacyclo [17.5.2.0^{2,6}.0^{7,12}.0^{22,26}]hexacosa-1(25),7(12),8,10,19(26),20,23-heptaen-18-one; and (15S)-4,4,9-trifluoro-15-hydroxy-13-oxa-2,17,21,22,25-pentaazapentacyclo[17.5.2.0^{2,6}.0^{7,12}.0^{22,26}]hexacosa-1(25),7(12),8,10,19(26),20,23-heptaen-18-one, or a pharmaceutically acceptable salt thereof.

In some embodiments, a Trk inhibitor is selected from the group consisting of: (R)-N-tert-butyl-5-(2-(2,5-difluorophenyl)pyrrolidin-1-yl)pyrazolo[1,5-a]pyrimidine-3-carboxamide; (R)-5-(2-(2,5-difluorophenyl)pyrrolidin-1-yl)-N-(pyridin-2-yl)pyrazolo[1,5-a]pyrimidine-3-carboxamide; (R)-5-(2-(2,5-difluorophenyl)pyrrolidin-1-yl)-N-(3-methylpyridin-2-yl)pyrazolo[1,5-a]-pyrimidine-3-carboxamide; (R)-5-(2-(2,5-difluorophenyl)pyrrolidin-1-yl)-N-(2-morpholinoethyl)pyrazolo[1,5-a]pyrimidine-3-carboxamide; (R)-5-(2-(2,5-difluorophenyl)pyrrolidin-1-yl)-N-((5-methylfuran-2-yl)methyl)pyrazolo[1,5-a]pyrimidine-3-carboxamide; (R)-5-(2-(2,5-difluorophenyl)pyrrolidin-1-yl)-N-(1-methyl-1H-pyrazol-3-yl)pyrazolo[1,5-a]pyrimidine-3-carboxamide; 5-((R)-2-(2,5-difluorophenyl)pyrrolidin-1-yl)-N-((trans)-4-hydroxycyclohexyl)pyrazolo[1,5-a]pyrimidine-3-carboxamide; (R)-5-(2-(2,5-difluorophenyl)pyrrolidin-1-yl)-N-(1-hydroxy-2-methylpropan-2-yl)pyrazolo[1,5-a]pyrimidine-3-carboxamide; (R)-5-(2-(2,5-difluorophenyl)pyrrolidin-1-yl)-N-(2-methyl-1-morpholinopropan-2-yl)pyrazolo[1,5-a]pyrimidine-3-carboxamide; (R)-5-(2-(2,5-difluorophenyl)pyrrolidin-1-yl)-N-methylpyrazolo[1,5-a]pyrimidine-3-carboxamide; (R)-1-(5-(2-(2, 5-difluorophenyl)pyrrolidin-1-yl)pyrazolo[1,5-a]pyrimidine-3-carbonyl)piperidine-4-carboxylic acid; (R)-2-(1-(5-(2-(2,5-difluorophenyl)pyrrolidin-1-yl)pyrazolo[1,5-a]pyrimidine-3-carbonyl)piperidin-4-yl)acetic acid; (R)-N-cyclopropyl-5-(2-(2,5-difluorophenyl)pyrrolidin-1-yl)pyrazolo[1,5-a]pyrimidine-3-carboxamide; (R)-N-cyclobutyl-5-(2-(2,5-difluorophenyl)pyrrolidin-1-yl)pyrazolo[1,5-a]pyrimidine-3-carboxamide; N-((2S)-bicyclo[2.2.1]heptan-2-yl)-5-((R)-2-(2,5-difluorophenyl)pyrrolidin-1-yl)pyrazolo[1,5-a]pyrimidine-3-carboxamide; (R)-5-(2-(2,5-difluorophenyl)pyrrolidin-1-yl)-N-(1-(hydroxymethyl)cyclopropyl)pyrazolo[1,5-a]pyrimidine-3-carboxamide; (R)-5-(2-(2,5-difluorophenyl)pyrrolidin-1-yl)-N-(2-hydroxy-2-methylpropyl)pyrazolo[1,5-a]pyrimidine-3-carboxamide; (5-((R)-2-(2,5-difluorophenyl)pyrrolidin-1-yl)pyrazolo[1,5-a]pyrimidin-3-yl)((S)-3-hydroxypyrrolidin-1-yl)methanone; (5-((R)-2-(2,5-difluorophenyl)pyrrolidin-1-yl)pyrazolo[1,5-a]pyrimidin-3-yl)((R)-3-hydroxypyrrolidin-1-yl)methanone; (R)-5-(2-(2,5-difluorophenyl)pyrrolidin-1-yl)-N-(tetrahydro-2H-pyran-4-yl)pyrazolo[1,5-a]pyrimidine-3-carboxamide; (R)-5-(2-(2,5-difluorophenyl)pyrrolidin-1-yl)-N-((1-methyl-1H-imidazol-4-yl)methyl) pyrazole[1,5-a]pyrimidine-3-carboxamide; (R)-5-(2-(2,5-difluorophenyl)pyrrolidin-1-yl)-N-((1-methyl-1H-pyrazol-4-yl)methyl)pyrazolo[1,5-a]pyrimidine-3-carboxamide; (R)-5-(2-(2,5-difluorophenyl)pyrrolidin-1-yl)-N-(2-(1-methyl-1H-imidazol-5-yl)ethyl)pyrazolo[1,5-a]pyrimidine-3-carboxamide; (R)-5-(2-(2,5-difluorophenyl)pyrrolidin-1-yl)-N-(2-(2-oxoimidazolidin-1-yl)ethyl) pyrazole[1,5-a]pyrimidine-3-carboxamide; (R)-N-(2-(1H-imidazol-4-yl)ethyl)-5-(2-(2,5-difluorophenyl)pyrrolidin-1-yl)pyrazole [1,5-a]pyrimidine-3-carboxamide; 5-((R)-2-(2,5-difluorophenyl)pyrrolidin-1-yl)-N-((R)-2,3-dihydroxypropyl)pyrazolo[1,5-a]pyrimidine-3-carboxamide; (R)-5-(2-(2,5-difluorophenyl)pyrrolidin-1-yl)-N,N-dimethylpyrazolo[1,5-a]pyrimidine-3-carboxamide; (R)-N-(2-(1H-imidazol-1-yl)ethyl)-5-(2-(2,5-difluorophenyl)pyrrolidin-1-yl)pyrazolo[1,5-a]pyrimidine-3-carboxamide; 5-((R)-2-(2,5-difluorophenyl)pyrrolidin-1-yl)-N-((S)-2,3-dihydroxypropyl)pyrazolo[1,5-a]pyrimidine-3-carboxamide; (R)-5-(2-(2,5-difluorophenyl)pyrrolidin-1-yl)pyrazolo[1,5-a]pyrimidine-3-carboxamide; (R)-(5-(2-(2,5-difluorophenyl)pyrrolidin-1-yl)pyrazolo[1,5-a]pyrimidin-3-yl)(3-hydroxyazetidin-1-yl)methanone; (R)-(5-(2-(2,5-difluorophenyl)pyrrolidin-1-yl)pyrazolo[1,5-a]pyrimidin-3-yl)(3-hydroxy-3-methylazetidin-1-yl)methanone; Trans-4-(5-((R)-2-(2,5-difluorophenyl)pyrrolidin-1-yl)pyrazolo[1,5-a]pyrimidine-3-carboxamido)cyclohexanecarboxylic acid; 5-((R)-2-(5-fluoro-2-methoxyphenyl)pyrrolidin-1-yl)-N-((trans)-4-hydroxycyclohexyl)pyrazolo[1,5-a]pyrimidine-3-carboxamide; 5-((R)-2-(3-fluorophenyl)pyrrolidin-1-yl)-N-((trans)-4-hydroxycyclohexyl)pyrazolo[1,5-a]pyrimidine-3-carboxamide; (R)-N-tert-butyl-5-(2-(3-fluorophenyl)pyrrolidin-1-yl)pyrazolo[1,5-a]pyrimidine-3-carboxamide; (R)-N-cyclopropyl-5-(2-(3-fluorophenyl)pyrrolidin-1-yl)pyrazolo[1,5-a]pyrimidine-3-carboxamide; (R)-N-(2-cyanopropan-2-yl)-5-(2-(2,5-difluorophenyl)pyrrolidin-1-yl)pyrazolo[1,5-a]pyrimidine-3-carboxamide; (R)-N-(cyanomethyl)-5-(2-(2,5-difluorophenyl)pyrrolidin-1-yl)pyrazolo[1,5-a]pyrimidine-3-carboxamide; (R)-5-(2-(2,5-difluorophenyl)pyrrolidin-1-yl)-N-(1-fluoro-2-methylpropan-2-yl)pyrazolo[1,5-a]pyrimidine-3-carboxamide; N-cyclopropyl-5-((2R,4R)-2-(3-fluorophenyl)-4-hydroxypyrrolidin-1-yl)pyrazolo[1,5-a]pyrimidine-3-carboxamide; N-tert-butyl-5-((2R,4R)-2-(3-fluorophenyl)-4-hydroxypyrrolidin-1-yl)pyrazolo[1,5-a]pyrimidine-3-carboxamide; 5-((2R,4R)-2-(3-fluorophenyl)-4-hydroxypyrrolidin-1-yl)-N-methylpyrazolo[1,5-a]pyrimidine-3-carboxamide; (R)-5-(2-(2,5-difluorophenyl)pyrrolidin-1-yl)-N-(1-(methylsulfonyl)piperidin-4-yl)pyrazolo[1,5-a]pyrimidine-3-carboxamide; (R)-5-(2-(2,5-difluorophenyl)pyrrolidin-1-yl)-N-(1-sulfamoylpiperidin-4-yl)pyrazolo[1,5-a]pyrimidine-3-carboxamide; (R)-5-(2-(2,5-difluorophenyl)pyrrolidin-1-yl)-N-(2-(methylsulfonamido)ethyl)pyrazolo[1,5-a]pyrimidine-3-carboxamide; (R)-5-(2-(2,5-difluorophenyl)pyrrolidin-1-yl)-N-(2-sulfamoylethyl)pyrazolo[1,5-a]pyrimidine-3-carboxamide; (R)-N-cyclopropyl-5-(2-(5-fluoro-2-methoxyphenyl)pyrrolidin-1-yl)pyrazolo[1,5-a]pyrimidine-3-carboxamide; (R)-5-(2-(5-fluoro-2-methoxyphenyl)pyrrolidin-1-yl)-N-(2-hydroxy-2-methylpropyl)pyrazolo[1,5-a]pyrimidine-3-carboxamide; 5-((R)-2-(2, 5-difluorophenyl)pyrrolidin-1-yl)-N-(4-hydroxy-4-methyl cyclohexyl)pyrazolo[1,5-a]pyrimidine-3-carboxamide (Diastereomer 1); 5-((R)-2-(2,5-difluorophenyl)pyrrolidin-1-yl)-N-(4-hydroxy-4-methylcyclohexyl)pyrazolo[1,5-a]pyrimidine-3-carb oxamide (Diastereomer 2); (R)-N-cyclopropyl-5-(2-(5-fluoropyridin-3-yl)pyrrolidin-1-yl)pyrazolo[1,5-a]pyrimidine-3-carboxamide; (R)-N-tert-butyl-5-(2-(5-fluoropyridin-3-yl)pyrrolidin-1-yl)pyrazolo[1,5-a]pyrimidine-3-carboxamide; (R)-5-(2-(5-fluoro-2-methoxyphenyl)pyrrolidin-1-yl)-N-(2-morpholinoethyl)-pyrazolo[1,5-a]pyrimidine-3-carboxamide; N-((S)-2,3-dihydroxypropyl)-5-((R)-2-(5-fluoro-2-methoxyphenyl)pyrrolidin-1-yl)pyrazolo[1,5-a]pyrimidine-3-carboxamide; N-((R)-2,3-dihydroxypropyl)-5-((R)-2-(5-fluoro-2-methoxyphenyl)pyrrolidin-1-yl)pyrazolo[1,5-a]pyrimidine-3-carboxamide; (R)-5-(2-(2,5-difluorophenyl)pyrrolidin-1-yl)-N-(2-methyl-1-(methylsulfonamido)propan-2-yl)pyrazolo[1,5-a]pyrimidine-3-carboxamide; (R)-N-(2-amino-2-methylpropyl)-5-(2-(2,5-difluorophenyl)pyrrolidin-1-yl)pyrazolo[1,5-a]pyrimidine-3-carboxamide; (R)-N-tert-butyl-5-(4,4-difluoro-2-(3-fluorophenyl)pyrrolidin-1-yl)pyrazolo[1,5-a]pyrimidine-3-carboxamide; (R)-5-(2-(2, 5-difluorophenyl)pyrrolidin-1-yl)-N-(1,3-dihydroxy-2-methylpropan-2-yl)pyrazolo[1,5-a]pyrimidine-3-carboxamide; 5-((R)-2-(2,5-difluorophenyl)pyrrolidin-1-yl)-N-((3S,4R)-3-fluoropiperidin-4-yl)pyrazolo[1,5-a]pyrimidine-3-carboxamide; N-((S)-2,3-dihydroxypropyl)-5-((R)-2-(5-fluoro-2-(trifluoromethyl)phenyl)pyrrolidin-1-yl)pyrazolo[1,5-a]pyrimidine-3-carboxamide; N-((R)-2,3-dihydroxypropyl)-5-((R)-2-(5-fluoro-2-(trifluoromethyl)phenyl)pyrrolidin-1-yl)pyrazolo[1,5-a]pyrimidine-3-carboxamide; (R)-5-(2-(5-fluoro-2-(trifluoromethyl)phenyl)pyrrolidin-1-yl)pyrazolo[1,5-a]pyrimidine-3-carboxamide; (R)-5-(2-(2,5-difluorophenyl)pyrrolidin-1-yl)-N-methoxypyrazolo[1,5-a]pyrimidine-3-carboxamide; (R)-5-(5-(2, 5-difluorophenyl)-2,2-dimethylpyrrolidin-1-yl)pyrazolo[1,5-a]pyrimidine-3-carboxamide; (R)-N-cyclopropyl-5-(5-(2,5-difluorophenyl)-2,2-dimethylpyrrolidin-1-yl)-pyrazolo[1,5-a]pyrimidine-3-carboxamide; (R)-N-(2-cyanopropan-2-yl)-5-(2-(5-fluoropyridin-3-yl)pyrrolidin-1-yl)pyrazolo[1,5-a]pyrimidine-3-carboxamide; (R)-5-(2-(5-fluoropyridin-3-yl)pyrrolidin-1-yl)-N-(1-(methylsulfonyl)piperidin-4-yl)pyrazolo[1,5-a]pyrimidine-3-carboxamide; (R)-N-(1-fluoro-2-methylpropan-2-yl)-5-(2-(5-fluoropyridin-3-yl)pyrrolidin-1-yl)pyrazolo[1,5-a]pyrimidine-3-carboxamide; (R)-5-(2-(5-fluoropyridin-3-yl)pyrrolidin-1-yl)-N-(tetrahydro-2H-pyran-4yl)pyrazolo[1,5-a]pyrimidine-3-carboxamide; (R)-5-(2-(5-fluoropyridin-3-yl)pyrrolidin-1-yl)-N-methoxypyrazolo[1,5-a]pyrimidine-3-carboxamide; (R)-5-(2-(3-fluorophenyl)pyrrolidin-1-yl)pyrazolo[1,5-a]pyrimidine-3-carboxamide; ((R)-5-(2-(3-fluorophenyl)pyrrolidin-1-yl)-N-methoxypyrazolo[1,5-a]pyrimidine-3-carboxamide; (R)-5-(2-(3-fluoro-5-(2-morpholinoethoxy)phenyl)pyrrolidin-1-yl)pyrazolo[1,5-a]pyrimidine-3-carboxamide; (R)-N-cyclopropyl-5-(2-(3-fluoro-5-(2-methoxyethoxy)phenyl)pyrrolidin-1-yl)pyrazole[1,5-a]pyrimidine-3-carboxamide; (R)-5-(2-(3-fluoro-5-(2-methoxyethoxy)phenyl)pyrrolidin-1-yl)pyrazolo[1,5-a]pyrimidine-3-carboxamide; (R)-N-cyclopropyl-5-(2-(5-fluoro-2-methoxypyridin-3-yl)pyrrolidin-1-yl)pyrazolo[1,5-a]pyrimidine-3-carboxamide; (R)-N-tert-butyl-5-(2-(5-fluoro-2-methoxypyridin-3-yl)pyrrolidin-1-yl)pyrazolo[1,5-a]pyrimidine-3-carboxamide; (R)-5-(2-(5-fluoro-2-methoxypyridin-3-yl)pyrrolidin-1-yl)-N-(1-fluoro-2-methylpropan-2-yl)pyrazolo[1,5-a]pyrimidine-3-carboxamide; (R)-5-(2-(5-fluoro-2-methoxypyridin-3-yl)pyrrolidin-1-yl)pyrazolo[1,5-a]pyrimidine-3-carboxamide; (R)-5-(2-(5-fluoro-2-methoxypyridin-3-yl)pyrrolidin-1-yl)-N-methoxypyrazolo[1,5-a]pyrimidine-3-carboxamide; (R)-1-(5-(2-(2,5-difluorophenyl)pyrrolidin-1-yl)pyrazolo[1,5-a]pyrimidine-3-carboxamido)-cyclopropanecarboxylic acid; (R)-N-cyclopropyl-5-(2-(3-fluoro-5-(2-morpholinoethoxy)phenyl)pyrrolidin-1-yl)pyrazolo[1,5-a]pyrimidine-3-carboxamide; (R)-5-(2-(5-fluoro-2-(2-morpholinoethoxy)phenyl)pyrrolidin-1-yl)pyrazolo[1,5-a]pyrimidine-3-carboxamide; (R)-N-cyclopropyl-5-(2-(5-fluoro-2-(2-morpholinoethoxy)phenyl) pyrrolidin-1-yl)pyrazolo[1,5-a]pyrimidine-3-carboxamide; 5-((R)-2-(2, 5-difluorophenyl)pyrrolidin-1-yl)-N-((S)-2,3-dihydroxypropoxy)pyrazolo[1-,5-a]pyrimidine-3-carboxamide; (R)-5-(2-(5-fluoro-2-(2-methoxyethoxy)phenyl)pyrrolidin-1-yl)pyrazolo[1,5-a]pyrimidine-3-carboxamide; (R)-N-cyclopropyl-5-(2-(5-fluoro-2-(2-methoxyethoxy)phenyl) pyrrolidin-1-yl)pyrazolo[1,5-a]pyrimidine-3-carboxamide; (R)-5-(2-(5-fluoropyridin-3-yl)pyrrolidin-1-yl)-N-(1-methylcyclopropyl)pyrazolo[1,5-a]pyrimidine-3-carboxamide; (R)-(5-(2-(5-fluoropyridin-3-yl)pyrrolidin-1-yl)pyrazolo[1,5-a]pyrimidin-3-yl)(3-hydroxy-3-methylazetidin-1-yl)methanone; (R)-5-(2-(5-fluoropyridin-3-yl)pyrrolidin-1-yl)-N-isopropylpyrazolo[1,5-a]pyrimidine-3-carboxamide; (R)-(5-(2-(5-fluoropyridin-3-yl)pyrrolidin-1-yl)pyrazolo[1,5-a]pyrimidin-3-yl)(pyrrolidin-1-yl)methanone; (R)-N-(5-fluoropyridin-2-yl)-5-(2-(5-fluoropyridin-3-yl)pyrrolidin-1-yl)pyrazolo[1,5-a]pyrimidine-3-carboxamide; (R)-(5-(2-(5-fluoropyridin-3-yl)pyrrolidin-1-yl)pyrazolo[1,5-a]pyrimidin-3-yl)(3-methoxyazetidin-1-yl)methanone; N-(3-chloro-2-fluoropropyl)-5-((R)-2-(5-fluoropyridin-3-yl)pyrrolidin-1-yl)pyrazolo[1,5-a]pyrimidine-3-carboxamide; (R)-5-(2-(5-fluoropyridin-3-yl)pyrrolidin-1-yl)-N-(1-(trifluoromethyl)cyclopropyl)pyrazolo[1,5-a]pyrimidine-3-carboxamide; 5-((R)-2-(5-fluoropyridin-3-yl)pyrrolidin-1-yl)-N-((trans)-4-hydroxycyclohexyl)pyrazolo[1,5-a]pyrimidine-3-carboxamide; 5-((R)-2-(5-fluoropyridin-3-yl)pyrrolidin-1-yl)-N-((cis)-4-hydroxycyclohexyl)pyrazolo[1,5-a]pyrimidine-3-carboxamide; (R)-N-cyclobutyl-5-(2-(5-fluoropyridin-3-yl)pyrrolidin-1-yl)pyrazolo[1,5-a]pyrimidine-3-carboxamide; (R)-5-(2-(5-fluoropyridin-3-yl)pyrrolidin-1-yl)-N-(1-methylcyclobutyl)pyrazolo[1,5-a]pyrimidine-3-carboxamide; 5-((R)-2-(5-fluoropyridin-3-yl)pyrrolidin-1-yl)-N-((1S,2S)-2-hydroxycyclopentyl)pyrazolo[1,5-a]pyrimidine-3-carboxamide; 5-((R)-2-(5-fluoropyridin-3-yl)pyrrolidin-1-yl)-N-((1S,2R)-2-hydroxycyclopentyl)pyrazolo[1,5-a]pyrimidine-3-carboxamide; 5-((R)-2-(5-fluoropyridin-3-yl)pyrrolidin-1-yl)-N-((1S,3S)-3-hydroxycyclopentyl)pyrazolo[1,5-a]pyrimidine-3-carboxamide; (R)-N-(cyclopropylmethyl)-5-(2-(5-fluoropyridin-3-yl)pyrrolidin-1-yl)pyrazolo[1,5-a]pyrimidine-3-carboxamide; (R)-5-(2-(5-fluoropyridin-3-yl)pyrrolidin-1-yl)-N-(1-(hydroxymethyl)cyclopropyl)pyrazolo[1,5-a]pyrimidine-3-carboxamide; (R)-(5-(2-(5-fluoropyridin-3-yl)pyrrolidin-1-yl)pyrazolo[1,5-a]pyrimidin-3-yl)(3-hydroxyazetidin-1-yl)methanone; 5-((R)-2-(5-fluoropyridin-3-yl)pyrrolidin-1-yl)-N-((S)-2-hydroxypropyl)pyrazolo[1,5-a]pyrimidine-3-carboxamide; 5-((R)-2-(5-fluoropyridin-3-yl)pyrrolidin-1-yl)-N-((R)-2-hydroxypropyl)pyrazolo[1,5-a]pyrimidine-3-carboxamide; (R)-5-(2-(5-fluoropyridin-3-yl)pyrrolidin-1-yl)-N-(2-hydroxy-2-methylpropyl)pyrazolo[1,5-a]pyrimidine-3-carboxamide; (R)-5-(2-(5-fluoropyridin-3-yl)pyrrolidin-1-yl)-N-(2-hydroxyethyl)pyrazolo[1,5-a]pyrimidine-3-carboxamide; N-(1-cyclopropylethyl)-5-((R)-2-(5-fluoropyridin-3-yl)pyrrolidin-1-yl)pyrazolo[1,5-a]pyrimidine-3-carboxamide; (R)-5-(2-(5-fluoropyridin-3-yl)pyrrolidin-1-yl)-N-methylpyrazolo[1,5-a]pyrimidine-3-carboxamide; 5-((R)-2-(5-fluoropyridin-3-yl)pyrrolidin-1-yl)-N-((R)-1-hydroxypropan-2-yl)pyrazolo[1,5-a]pyrimidine-3-carboxamide; 5-((R)-2-(5-fluoropyridin-3-yl)pyrrolidin-1-yl)-N-((S)-1-hydroxypropan-2-yl)pyrazolo[1,5-a]pyrimidine-3-carboxamide; (R)-5-(2-(5-fluoropyridin-3-yl)pyrrolidin-1-yl)pyrazolo[1,5-a]pyrimidine-3-carboxamide; 5-((R)-2-(5-fluoropyridin-3-yl)pyrrolidin-1-yl)-N-(1-methoxypropan-2-yl)pyrazolo[1,5-a]pyrimidine-3-carboxamide; 5-((R)-2-(5-fluoropyridin-3-yl)pyrrolidin-1-yl)-N-(2-hydroxy-3-methoxypropyl)pyrazolo[1,5-a]pyrimidine-3-carboxamide; 5-((R)-2-(5-fluoropyridin-3-yl)pyrrolidin-1-yl)-N-((trans)-2-hydroxycyclopentyl)pyrazolo[1,5-a]pyrimidine-3-carboxamide; 5-((R)-2-(5-fluoropyridin-3-yl)pyrrolidin-1-yl)-N-((S)-1-hydroxy-3-methylbutan-2-yl)pyrazolo[1,5-a]pyrimidine-3-carboxamide; 5-((R)-2-(5-fluoropyridin-3-yl)pyrrolidin-1-yl)-N-((R)-1-hydroxy-3-methylbutan-2-yl)pyrazolo[1,5-a]pyrimidine-3-carboxamide; N-((R)-1-cyclopropylethyl)-5-((R)-2-(5-fluoropyridin-3-yl)pyrrolidin-1-yl)pyrazolo[1,5-a]pyrimidine-3-carboxamide; N-((S)-1-cyclopropylethyl)-5-((R)-2-(5-fluoropyridin-3-yl)pyrrolidin-1-yl)pyrazolo[1,5-a]pyrimidine-3-carboxamide; (R)-5-(2-(5-fluoropyridin-3-yl)pyrrolidin-1-yl)-N-(3-hydroxy-2,2-dimethyl-propyl)pyrazolo[1,5-a]pyrimidine-3-carboxamide; (R)-azetidin-1-yl(5-(2-(5-fluoropyridin-3-yl)pyrrolidin-1-yl)pyrazolo[1,5-a]pyrimidin-3-yl)methanone; (R)-(5-(2-(5-fluoropyridin-3-yl)pyrrolidin-1-yl)pyrazolo[1,5-a]pyrimidin3-yl)(3-(hydroxymethyl)azetidin-1-yl)methanone; (5-((R)-2-(5-fluoropyridin-3-yl)pyrrolidin-1-yl)pyrazolo[1,5-a]pyrimidin-3-yl)((S)-3-hydroxypyrrolidin-1-yl)methanone; 5-((R)-2-(5-fluoropyridin-3-yl)pyrrolidin-1-yl)-N-((R)-1,1,1-trifluoropropan-2-yl)pyrazolo[1,5-a]pyrimidine-3-carboxamide; 5-((R)-2-(5-fluoropyridin-3-yl)pyrrolidin-1-yl)-N-((S)-1,1,1-trifluoropropan-2-yl)pyrazolo[1,5-a]pyrimidine-3-carboxamide; (R)-5-(2-(5-fluoropyridin-3-yl)pyrrolidin-1-yl)-N-(2,2,2-trifluoroethyl)pyrazolo[1,5-a]pyrimidine-3-carboxamide; (R)-5-(2-(5-fluoropyridin-3-yl)pyrrolidin-1-yl)-N-(1-hydroxy-2-methylpropan-2-yl)pyrazolo[1,5-a]pyrimidine-3-carboxamide; 5-((R)-2-(5-fluoropyridin-3-yl)pyrrolidin-1-yl)-N-((1R,2R)-2-hydroxycyclopentyl)pyrazolo[1,5-a]pyrimidine-3-carboxamide; (R)-N-(2,2-difluoroethyl)-5-(2-(5-fluoropyridin-3-yl)pyrrolidin-1-yl)pyrazolo[1,5-a]pyrimidine-3-carboxamide; 5-((R)-2-(5-fluoropyridin-3-yl)pyrrolidin-1-yl)-N-((1R,2S)-2-hydroxycyclopentyl)pyrazolo[1,5-a]pyrimidine-3-carboxamide; 5-((R)-2-(5-fluoropyridin-3-yl)pyrrolidin-1-yl)-N-((1R,2R)-2-hydroxycyclohexyl)pyrazolo[1,5-a]pyrimidine-3-carboxamide; (R)-(5-(2-(5-fluoropyridin-3-yl)pyrrolidin-1-yl)pyrazolo[1,5-a]pyrimidin-3-yl)(piperidin-1-yl)methanone; 5-((R)-2-(5-fluoropyridin-3-yl)pyrrolidin-1-yl)-N-((2R,3S,4S)-3-(hydroxymethyl)bicyclo[2.2.1]heptan-2-yl)pyrazolo[1,5-a]pyrimidine-3-carboxamide; (R)-(5-(2-(5-fluoro-2-methoxypyridin-3-yl)pyrrolidin-1-yl)pyrazolo[1,5-a]pyrimidin-3-yl)(3-hydroxyazetidin-1-yl)methanone; 5-((R)-2-(5-fluoro-2-methoxypyridin-3-yl)pyrrolidin-1-yl)-N-((trans)-4-hydroxycyclohexyl)pyrazolo[1,5-a]pyrimidine-3-carboxamide; (R)-tert-butyl 3-(5-(2-(5-fluoro-2-methoxypyridin-3-yl)pyrrolidin-1-yl)pyrazolo[1,5-a]pyrimidine-3-carboxamido)propylcarbamate; (R)-N-(3-aminopropyl)-5-(2-(5-fluoro-2-oxo-1,2-dihydropyridin-3-yl)pyrrolidin-1-yl)pyrazolo[1,5-a]pyrimidine-3-carboxamide; N-((S)-2,3-dihydroxypropyl)-5-((R)-2-(5-fluoro-2-methoxypyridin-3-yl)pyrrolidin-1-yl)pyrazolo[1,5-a]pyrimidine-3-carboxamide; N-((S)-3-chloro-2-hydroxypropyl)-5-((R)-2-(5-fluoro-2-methoxypyridin-3-yl)pyrrolidin-1-yl)pyrazolo[1,5-a]pyrimidine-3-carboxamide; N-((R)-3-chloro-2-hydroxypropyl)-5-((R)-2-(5-fluoro-2-methoxypyridin-3-yl)pyrrolidin-1-yl)pyrazolo[1,5-a]pyrimidine-3-carboxamide; (R)-N-(2-chloroethoxy)-5-(2-(5-fluoro-2-methoxypyridin-3-yl)pyrrolidin-1-yl)pyrazolo[1,5-a]pyrimidine-3-carboxamide; (R)-(5-(2-(5-fluoro-2-methoxypyridin-3-yl)pyrrolidin-1-yl)pyrazolo[1,5-a]pyrimidin-3-yl)(3-hydroxy-3-methylazetidin-1-yl)methanone; (R)-5-(2-(5-fluoro-2-methoxypyridin-3-yl)pyrrolidin-1-yl)-N-(3-hydroxypropyl)pyrazolo[1,5-a]pyrimidine-3-carboxamide; N-(2,3-dihydroxypropyl)-5-((R)-2-(5-fluoro-2-methoxypyridin-3-yl)pyrrolidin-1-yl)pyrazolo[1,5-a]pyrimidine-3-carboxamide; N-((R)-2,3-dihydroxypropyl)-5-((R)-2-(5-fluoro-2-methoxypyridin-3-yl)pyrrolidin-1-yl)pyrazolo[1,5-a]pyrimidine-3-carboxamide; (R)-5-(2-(5-fluoro-2-methoxypyridin-3-yl)pyrrolidin-1-yl)-N-(4-hydroxybutyl)pyrazolo[1,5-a]pyrimidine-3-carboxamide; (R)-N-(2-tert-butoxyethoxy)-5-(2-(5-fluoro-2-methoxypyridin-3-yl)pyrrolidin-1-yl)pyrazolo[1,5-a]pyrimidine-3-carboxamide; (R)-5-(2-(5-fluoro-2-methoxypyridin-3-yl)pyrrolidin-1-yl)-N-methylpyrazolo[1,5-a]pyrimidine-3-carboxamide; 5-((R)-2-(5-fluoro-2-methoxypyridin-3-yl)pyrrolidin-1-yl)-N-((1S,3S)-3-hydroxycyclopentyl)pyrazolo[1,5-a]pyrimidine-3-carboxamide; (R)-5-(2-(5-fluoro-2-methoxypyridin-3-yl)pyrrolidin-1-yl)-N-(2-hydroxyethyl)pyrazolo[1,5-a]pyrimidine-3-carboxamide; 5-((R)-2-(5-fluoro-2-methoxypyridin-3-yl)pyrrolidin-1-yl)-N-((S)-2-hydroxypropyl)pyrazolo[1,5-a]pyrimidine-3-carboxamide; 5-((R)-2-(5-fluoro-2-methoxypyridin-3-yl)pyrrolidin-1-yl)-N-((R)-2-hydroxypropyl)pyrazolo[1,5-a]pyrimidine-3-carboxamide; (R)-5-(2-(5-fluoro-2-methoxypyridin-3-yl)pyrrolidin-1-yl)-N-(2-hydroxy-2-methylpropyl)pyrazolo[1,5-a]pyrimidine-3-carboxamide; (R)-N-(1,3-dihydroxypropan-2-yl)-5-(2-(5-fluoro-2-methoxypyridin-3-yl)pyrrolidin-1-yl)pyrazolo[1,5-a]pyrimidine-3-carboxamide; (R)-5-(2-(5-fluoro-2-methoxypyridin-3-yl)pyrrolidin-1-yl)-N-(6-oxo-1,6-dihydropyridin-3-yl)pyrazolo[1,5-a]pyrimidine-3-carboxamide; R)-5-(2-(5-fluoro-2-methoxypyridin-3-yl)pyrrolidin-1-yl)-N-(1-(methylsulfonyl)piperidin-4-yl)pyrazolo[1,5-a]pyrimidine-3-carboxamide; (R)-N-(2-chloroethyl)-5-(2-(5-fluoro-2-methoxypyridin-3-yl)pyrrolidin-1yl)pyrazolo[1,5-a]pyrimidine-3-carboxamide; (R)-N-(2-bromoethoxy)-5-(2-(5-fluoro-2-methoxypyridin-3-yl)pyrrolidin-1-yl)pyrazolo[1,5-a]pyrimidine-3-carboxamide; 5-(2-(2,5-difluorophenyl)pyrrolidin-1-yl)-N-(2-hydroxyethyl)pyrazolo[1,5a]pyrimidine-3-carboxamide; 5-((R)-2-(2,5-difluorophenyl)pyrrolidin-1-yl)-N-(2-hydroxypropyl)pyrazolo[1,5-a]pyrimidine-3-carboxamide; 5-((R)-2-(2,5-difluorophenyl)pyrrolidin-1-yl)-N-(2-hydroxypropyl)pyrazolo[1,5-a]pyrimidine-3-carboxamide; 5-((R)-2-(2,5-difluorophenyl)pyrrolidin-1-yl)-N-(3-hydroxy-2,2-dimethylpropyl)pyrazolo[1,5-a]pyrimidine-3-carboxamide; 5-((R)-2-(2,5-difluorophenyl)pyrrolidin-1-yl)-N-((1S,3S)-3-hydroxycyclopentyl)pyrazolo[1,5-a]pyrimidine-3-carboxamide; 5-((R)-2-(2,5-difluorophenyl)pyrrolidin-1-yl)-N-(2-(4-hydroxypiperidin-1-yl)ethyl)pyrazolo[1,5-a]pyrimidine-3-carboxamide; 5-((R)-2-(2,5-difluorophenyl)pyrrolidin-1-yl)-N-(2-(4-methylpiperazin-1-yl)ethyl)pyrazolo[1,5-a]pyrimidine-3-carboxamide; 5-((R)-2-(2,5-difluorophenyl)pyrrolidin-1-yl)-N-(2-methoxyethyl)pyrazolo[1,5-a]pyrimidine-3-carboxamide; 5-((R)-2-(2,5-difluorophenyl)pyrrolidin-1-yl)-N-(1,3-dihydroxypropan-2-yl)pyrazolo[1,5-a]pyrimidine-3-carboxamide; 5-((R)-2-(2, 5-difluorophenyl)pyrrolidin-1-yl)-N-((2S,3R)-1,3-dihydroxybutan-2-yl)pyrazolo[1,5-a]pyrimidine-3-carboxamide; 5-((R)-2-(2,5-difluorophenyl)pyrrolidin-1-yl)-N-((2 S,3S)-1,3-dihydroxybutan-2-yl)pyrazolo[1,5-a]pyrimidine-3-carboxamide; 5-((R)-2-(2,5-difluorophenyl)pyrrolidin-1-yl)-N-((2R,3S)-1,3-dihydroxybutan-2-yl)pyrazolo[1,5-a]pyrimidine-3-carboxamide; 5-((R)-2-(2,5-difluorophenyl)pyrrolidin-1-yl)-N-((S)-1-hydroxypropan-2-yl)pyrazolo[1,5-a]pyrimidine-3-carboxamide; 5-((R)-2-(2, 5-difluorophenyl)pyrrolidin-1-yl)-N-((S)-1-hydroxybutan-2-yl)pyrazolo[1,5-a]pyrimidine-3-carboxamide; 5-((R)-2-(2,5-difluorophenyl)pyrrolidin-1-yl)-N-((S)-1-hydroxy-3-methylbutan-2-yl)pyrazolo[1,5-a]pyrimidine-3-carboxamide; 5-((R)-2-(2,5-difluorophenyl)pyrrolidin-1-yl)-N-((S)-1-hydroxy-3,3-dimethylbutan-2-yl)pyrazolo[1,5-a]pyrimidine-3-carboxamide; N-cyclopropyl-5-(2-(5-fluoro-2-methylpyridin-3-yl)pyrrolidin-1-yl)pyrazolo[1,5-a]pyrimidine-3-carboxamide; N-cyclopropyl-5-(2-(2-ethyl-5-fluoropyridin-3-yl)pyrrolidin-1-yl)pyrazolo-[1,5-a]pyrimidine-3-carboxamide; (R)-N-tert-butyl-5-(2-(5-fluoro-2-methylpyridin-3-yl)pyrrolidin-1-yl)pyrazolo[1,5-a]pyrimidine-3-carboxamide; (R)-5-(2-(5-fluoro-2-methylpyridin-3-yl)pyrrolidin-1-yl)-N-isopropylpyrazolo[1,5-a]pyrimidine-3-carboxamide; (R)-N-cyclobutyl-5-(2-(5-fluoro-2-methylpyridin-3-yl)pyrrolidin-1-yl)pyrazolo[1,5-a]pyrimidine-3-carboxamide; (R)-5-(2-(5-fluoro-2-methylpyridin-3-yl)pyrrolidin-1-yl)-N-methylpyrazolo-[1,5-a]pyrimidine-3-carboxamide; (R)-5-(2-(5-fluoro-2-methylpyridin-3-yl)pyrrolidin-1-yl)pyrazolo[1,5-a]pyrimidine-3-carboxamide; (R)-5-(2-(5-fluoro-2-methylpyridin-3-yl)pyrrolidin-1-yl)-N-(2-hydroxyethyl)pyrazolo[1,5-a]pyrimidine-3-carboxamide; (R)-5-(2-(5-fluoro-2-methylpyridin-3-yl)pyrrolidin-1-yl)-N-((R)-2-hydroxypropyl)pyrazolo[1,5-a]pyrimidine-3-carboxamide; (R)-5-(2-(5-fluoro-2-methylpyridin-3-yl)pyrrolidin-1-yl)-N-(1-methylcyclopropyl)pyrazolo[1,5-a]pyrimidine-3-carboxamide; (R)-5-(2-(5-fluoro-2-methylpyridin-3-yl)pyrrolidin-1-yl)-N-(2-methoxyethyl)pyrazolo[1,5-a]pyrimidine-3-carboxamide; (R)-(5-(2-(5-fluoro-2-methylpyridin-3-yl)pyrrolidin-1-yl)pyrazolo[1,5-a]pyrimidin-3-yl)(3-hydroxyazetidin-1-yl)methanone; (R)-5-(2-(5-fluoro-2-methylpyridin-3-yl)pyrrolidin-1-yl)-N-(1-(hydroxymethyl)cyclopropyl)pyrazolo[1,5-a]pyrimidine-3-carboxamide; 5-((R)-2-(5-fluoro-2-methylpyridin-3-yl)pyrrolidin-1-yl)-N-((trans)-4-hydroxycyclohexyl)pyrazolo[1,5-a]pyrimidine-3-carboxamide; 5-((R)-2-(5-fluoro-2-methylpyridin-3-yl)pyrrolidin-1-yl)-N-((cis)-4-hydroxycyclohexyl)pyrazolo[1,5-a]pyrimidine-3-carboxamide; 5-((R)-2-(5-fluoro-2-methylpyridin-3-yl)pyrrolidin-1-yl)-N-((1S,3S)-3-hydroxycyclopentyl)pyrazolo[1,5-a]pyrimidine-3-carboxamide; 5-((R)-2-(5-fluoro-2-methylpyridin-3-yl)pyrrolidin-1-yl)-N-((1R,2R)-2-hydroxycyclopentyl)pyrazolo[1,5-a]pyrimidine-3-carboxamide; 5-((R)-2-(5-fluoro-2-methylpyridin-3-yl)pyrrolidin-1-yl)-N-((R)-quinuclidin-3-yl)pyrazolo[1,5-a]pyrimidine-3-carboxamide; 5-((R)-2-(2-ethyl-5-fluoropyridin-3-yl)pyrrolidin-1-yl)-N-((trans)-4-hydroxycyclohexyl)pyrazolo[1,5-a]pyrimidine-3-carboxamide; 5-((R)-2-(2-ethyl-5-fluoropyridin-3-yl)pyrrolidin-1-yl)-N-((1S,3S)-3-hydroxycyclopentyl)pyrazolo[1,5-a]pyrimidine-3-carboxamide; (R)-5-(2-(2-ethyl-5-fluoropyridin-3-yl)pyrrolidin-1-yl)-N-(2-hydroxy-2-methylpropyl)pyrazolo[1,5-a]pyrimidine-3-carboxamide; (R)-N-tert-butyl-5-(2-(5-fluoro-2-oxo-1,2-dihydropyridin-3-yl)pyrrolidin-1-yl)pyrazolo[1,5-a]pyrimidine-3-carboxamide; (R)-N-(2-chloroethyl)-5-(2-(5-fluoro-2-oxo-1,2-dihydropyridin-3-yl)pyrrolidin-1-yl)pyrazolo[1,5-a]pyrimidine-3-carboxamide; N-cyclopropyl-5-((2R)-2-(2-((2,2-dimethyl-1,3-dioxolan-4-yl)methoxy)-5-fluorophenyl)pyrrolidin-1-yl)pyrazolo[1,5-a]pyrimidine-3-carboxamide; 5-((2R)-2-(2-((2,2-dimethyl-1,3-dioxolan-4-yl)methoxy)-5-fluorophenyl)pyrrolidin-1-yl)pyrazolo[1,5-a]pyrimidine-3-carboxamide; N-cyclopropyl-5-((2R)-2-(3-((2,2-dimethyl-1,3-dioxolan-4-yl)methoxy)-5-fluorophenyl)pyrrolidin-1-yl)pyrazolo[1,5-a]pyrimidine-3-carboxamide; 5-((2R)-2-(3-((2,2-dimethyl-1,3-dioxolan-4-yl)methoxy)-5-fluorophenyl)pyrrolidin-1-yl)pyrazolo[1,5-a]pyrimidine-3-carboxamide; N-cyclopropyl-5-((2R)-2-(3-(2,3-dihydroxypropoxy)-5-fluorophenyl)pyrrolidin-1-yl)pyrazolo[1,5-a]pyrimidine-3-carboxamide; 5-((2R)-2-(3-(2,3-dihydroxypropoxy)-5-fluorophenyl)pyrrolidin-1-yl)pyrazolo[1,5-a]pyrimidine-3-carboxamide; N-cyclopropyl-5-((2R)-2-(2-(2,3-dihydroxypropoxy)-5-fluorophenyl)pyrrolidin-1-yl)pyrazolo[1,5-a]pyrimidine-3-carboxamide; 5-((2R)-2-(2-(2,3-dihydroxypropoxy)-5-fluorophenyl)pyrrolidin-1-yl)pyrazolo[1,5-a]pyrimidine-3-carboxamide; 5-((2R,5 S)-2-(5-fluoropyridin-3-yl)-5-(hydroxymethyl)pyrrolidin-1-yl)-N-((R)-1,1,1-trifluoropropan-2-yl)pyrazolo[1,5-a]pyrimidine-3-carboxamide; 5-((2R,5S)-2-(5-fluoropyridin-3-yl)-5-(hydroxymethyl)pyrrolidin-1-yl)-N-((S)-1,1,1-trifluoropropan-2-yl)pyrazolo[1,5-a]pyrimidine-3-carboxamide; 5-((2R,5S)-2-(5-fluoropyridin-3-yl)-5-(hydroxymethyl)pyrrolidin-1-yl)-N-(1-methylcyclopropyl)pyrazolo[1,5-a]pyrimidine-3-carboxamide; 5-((2R,5S)-2-(5-fluoropyridin-3-yl)-5-(hydroxymethyl)pyrrolidin-1-yl)-N-isopropyl-pyrazolo[1,5-a]pyrimidine-3-carboxamide; 5-((2R,4S)-2-(3-fluorophenyl)-4-hydroxypyrrolidin-1-yl)-N-((S)-1,1,1-trifluoropropan-2-yl)pyrazolo[1,5-a]pyrimidine-3-carboxamide; 5-((2R,4S)-2-(3-fluorophenyl)-4-hydroxypyrrolidin-1-yl)-N-isopropylpyrazolo[1,5-a]pyrimidine-3-carboxamide; 5-((2R,4S)-2-(3-fluorophenyl)-4-hydroxypyrrolidin-1-yl)-N-methylpyrazolo[1,5-a]pyrimidine-3-carboxamide; 5-((2S,5R)-5-(5-fluoropyridin-3-yl)-2-(hydroxymethyl)-2-methylpyrrolidin-1-yl)-N-isopropylpyrazolo[1,5-a]pyrimidine-3-carboxamide; 5-((2S,5R)-5-(5-fluoropyridin-3-yl)-2-(hydroxymethyl)-2-methylpyrrolidin-1-yl)-N-((S)-1,1,1-trifluoropropan-2-yl)pyrazolo[1,5-a]pyrimidine-3-carboxamide; (R)-(5-(2-(2-amino-5-fluoropyridin-3-yl)pyrrolidin-1-yl)pyrazolo[1,5-a]pyrimidin-3-yl)(azetidin-1-yl)methanone; (R)-tert-butyl 3-(5-(2-(2-chloro-5-fluoropyridin-3-yl)pyrrolidin-1-yl)pyrazolo[1,5-a]pyrimidine-3-carboxamido)propylcarbamate; (R)-N-(3-aminopropyl)-5-(2-(2-chloro-5-fluoropyridin-3-yl)pyrrolidinyl)pyrazolo[1,5-a]pyrimidine-3-carboxamide; (R)-N-(2-tert-butoxyethoxy)-5-(2-(2-chloro-5-fluoropyridin-3-yl)pyrrolidin-1-yl)pyrazolo[1,5-a]pyrimidine-3-carboxamide; (R)-5-(2-(2-chloro-5-fluoropyridin-3-yl)pyrrolidin-1-yl)-N-(2-hydroxyethoxy)pyrazolo[1,5-a]pyrimidine-3-carboxamide; (R)-N-tert-butyl-5-(2-(5-fluoro-1-methyl-2-oxo-1,2-dihydropyridin-3-yl)pyrrolidin-1-yl)pyrazolo[1,5-a]pyrimidine-3-carboxamide; (R)-5-(2-(5-fluoro-1-methyl-2-oxo-1,2-dihydropyridin-3-yl)pyrrolidin-1-yl)-N-isopropylpyrazolo[1,5-a]pyrimidine-3-carboxamide; (R)-N-cyclopropyl-5-(2-(5-fluoro-1-methyl-2-oxo-1,2-dihydropyridin-3-yl)pyrrolidin-1-yl)pyrazolo[1,5-a]pyrimidine-3-carboxamide; (R)-5-(2-(5-fluoro-1-methyl-2-oxo-1,2-dihydropyridin-3-yl)pyrrolidin-1-yl)-N-(6-methylpyridin-3-yl)pyrazolo[1,5-a]pyrimidine-3-carboxamide; (R)-N-cyclobutyl-5-(2-(5-fluoro-1-methyl-2-oxo-1,2-dihydropyridin-3-yl)pyrrolidin-1-yl)pyrazolo[1,5-a]pyrimidine-3-carboxamide; (R)-5-(2-(5-fluoro-1-methyl-2-oxo-1,2-dihydropyridin-3-yl)pyrrolidin-1-yl)-N-(pyridin-3-yl)pyrazolo[1,5-a]pyrimidine-3-carboxamide; (R)-N-(cyclopropylmethyl)-5-(2-(5-fluoro-1-methyl-2-oxo-1,2-dihydropyridin-3-yl)pyrrolidin-1-yl)pyrazolo[1,5-a]pyrimidine-3-carboxamide; 5-((R)-2-(5-fluoro-1-methyl-2-oxo-1,2-dihydropyridin-3-yl)pyrrolidin-1-yl)-N-((S)-1-hydroxy-3,3-dimethylbutan-2-yl)pyrazolo[1,5-a]pyrimidine-3-carboxamide; 5-((R)-2-(5-fluoro-1-methyl-2-oxo-1,2-dihydropyridin-3-yl)pyrrolidin-1-yl)-N-((1R,2R)-2-hydroxycyclohexyl)pyrazolo[1,5-a]pyrimidine-3-carboxamide; N-((R)-1-cyclopropylethyl)-5-((R)-2-(5-fluoro-1-methyl-2-oxo-1,2-dihydropyridin-3-yl)pyrrolidin-1-yl)pyrazolo[1,5-a]pyrimidine-3-carboxamide; N-((S)-1-cyclopropylethyl)-5-((R)-2-(5-fluoro-1-methyl-2-oxo-1,2-dihydropyridin-3-yl)pyrrolidin-1-yl)pyrazolo[1,5-a]pyrimidine-3-carboxamide; (R)-5-(2-(5-fluoro-1-methyl-2-oxo-1,2-dihydropyridin-3-yl)pyrrolidin-1-yl)-N-(1-methylcyclopropyl)pyrazolo[1,5-a]pyrimidine-3-carboxamide; 5-((R)-2-(5-fluoro-1-methyl-2-oxo-1,2-dihydropyridin-3-yl)pyrrolidin-1-yl)-N-((trans)-4-hydroxycyclohexyl)pyrazolo[1,5-a]pyrimidine-3-carboxamide; (R)-5-(2-(5-fluoro-1-methyl-2-oxo-1,2-dihydropyridin-3-yl)pyrrolidin-1-yl)-N-(5-fluoropyridin-2-yl)pyrazolo[1,5-a]pyrimidine-3-carboxamide; (R)-5-(2-(5-fluoro-2-methoxypyridin-3-yl)pyrrolidin-1-yl)-N-(3-methyl-1H-pyrazol-5-yl)pyrazolo[1,5-a]pyrimidine-3-carboxamide; (R)-5-(2-(5-fluoro-2-methoxypyridin-3-yl)pyrrolidin-1-yl)-N-(1-methyl-1H-pyrazol-3-yl)pyrazolo[1,5-a]pyrimidine-3-carboxamide; (R)-N-(3-cyclopropyl-1H-pyrazol-5-yl)-5-(2-(5-fluoro-2-methoxypyridin-3-yl)pyrrolidin-1-yl)pyrazolo[1,5-a]pyrimidine-3-carboxamide; (R)-N-(3-ethyl-1H-pyrazol-5-yl)-5-(2-(5-fluoro-2-methoxypyridin-3-yl)pyrrolidin-1-yl)pyrazolo[1,5-a]pyrimidine-3-carboxamide; and (R)-5-(2-(5-fluoro-2-methoxypyridin-3-yl)pyrrolidin-1-yl)-N-(1-isopropyl-1H-pyrazol-3-yl)pyrazolo[1,5-a]pyrimidine-3-carboxamide, or a pharmaceutically acceptable salt thereof.

In some embodiments, a Trk inhibitor is selected from the group consisting of: 5-fluoro-2-[[(1S)-1-(5-fluoro-2-pyridyl)ethyl]amino]-6-[(5-isopropoxy-1H-pyrazol-3-yl)amino]pyridine-3-carbonitrile; ((2E)-3-[3,5-Bis(1,1-dimethylethyl)-4-hydroxyphenyl]-2-cyano-2-propenethioamide); 2,2-dichloro-N-[3-[(7-chloroquinolin-4-yl)amino]propyl]-N-methylacetamide; N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea; (S)-5-chloro-N2-(1-(5-fluoropyridin-2-yl)ethyl)-N4-(5-isopropoxy-1H-pyrazol-3-yl)pyrimidine-(S)-N-(1-(5-fluoropyrimidin-2-yl)ethyl)-3-(5-isopropoxy-1H-pyrazol-3-yl)-3H-imidazo[4,5-b]pyridin-5-amine, 4-diamine; 5,6,7,13-tetrahydro-13-methyl-5-oxo-12H-indolo[2,3-a]pyrrolo[3,4-c]carbazole-12-propanenitrile; 1,3-dihydro-3-[(1-methyl-1H-indol-3-yl)methylene]-2H-pyrrolo[3,2-b]pyridin-2-one; or a pharmaceutically acceptable salt thereof.

In some embodiments, a Trk inhibitor is selected from the group consisting of: (2R)-2-({4-[(5-cyclopropyl-1H-pyrazol-3-yl)amino]-5-fluoropyrimidin-2-yl}-amino)-2-(4-fluorophenyl)ethanol; 5-bromo-N⁴-(3-cyclopropyl-1H-pyrazol-5-yl)-N²-[(1S)-1-(4-fluorophenyl)ethyl]pyrimidine-2,4-diamine; (2R)-2-({5-chloro-4-[(3-cyclopropyl-1H-pyrazol-5-yl)amino]pyrimidin-2-yl}-amino)-2-(4-fluorophenyl)ethanol; (2R)-2-({5-chloro-4-[(3-isopropoxy-1H-pyrazol-5-yl)amino]pyrimidin-2-yl}amino)-2-(4-fluorophenyl)ethanol; (3S)-3-({5-chloro-4-[(5-cyclopropyl-1H-pyrazol-3-yl)amino]pyrimidin-2-yl}-amino)-3-(4-fluorophenyl)-N-methylpropanamide; 2-({5-chloro-2-{[(1S)-1-(4-fluorophenyl)ethyl]amino}-6-[(5-isopropoxy-1H-pyrazol-3-yl)amino]-pyrimidin-4-yl}amino)propane-1,3-diol; 2-[(5-chloro-6-[(3-cyclopropyl-1H-pyrazol-5-yl)amino]-2-{[(1S)-1-(4-fluorophenyl)ethyl]amino]pyrimidin-4-yl)amino}propane-1,3-diol; 5-chloro-N⁴-(5-cyclopropyl-1H-pyrazol-3-yl)-N²-[(1S)-(4-fluoro-phenyl)-ethyl]-6-(4-methyl-piperazin-1-yl)-pyrimidine-2,4-diamine; (2R)-2-({4-[(5-cyclopropyl-1H-pyrazol-3-yl)amino]-7-fluoroquinazolin-2-yl}amino)-2-(4-fluorophenyl)ethanol; and 2-[(5-chloro-6-[(5-cyclopropyl-1H-pyrazol-3-yl)amino]-2-{[(1R)-1-(4-fluorophenyl)-2-hydroxyethyl]amino}pyrimidin-4-yl)amino]propane-1,3-diol; or an acceptable salt thereof.

In some embodiments, a Trk inhibitor is selected from the group consisting of: 1-(3-tert-butyl-1-phenyl-1H-pyrazol-5-yl)-3-(trans-1-(2-methoxyethyl)-4-phenylpyrrolidin-3-yl)urea; 1-(3-tert-butyl-1-p-tolyl-1H-pyrazol-5-yl)-3-(trans-1-(2-methoxyethyl)-4-phenylpyrrolidin-3-yl)urea hydrochloride; trans-1-(4-(3,4-difluorophenyl)-1-(2-methoxyethyl)pyrrolidin-3-yl)-3-(2-phenyl-2,4,5,6-tetrahydrocyclopenta[c]pyra-zol-3-yl)urea; trans-1-(4-(3,4-difluorophenyl)-1-(2-methoxyethyl)pyrrolidin-3-yl)-3-(3-isopropyl-1-phenyl-1H-pyrazol-5-yl)urea; 1-(3-tert-butyl-1-methyl-1H-pyrazol-5-yl)-3-(trans-1-(2-methoxyethyl)-4-phenylpyrrolidin-3-yl)urea; 1-(1,3-dimethyl-1H-pyrazol-5-yl)-3-(trans-1-(2-methoxyethyl)-4-phenylpyrrolidin-3-yl)urea; 1-(3-tert-butyl-1-(pyridin-3-yl)-1H-pyrazol-5-yl)-3-(trans-1-(2-methoxyethyl)-4-phenylpyrrolidin-3-yl)urea; 1-(3-tert-butyl-1-(4-fluorophenyl)-1H-pyrazol-5-yl)-3-(trans-1-(2-methoxy-ethyl)-4-phenylpyrrolidin-3-yl)urea; 1-(3-cyclopropyl-1-phenyl-1H-pyrazol-5-yl)-3-(trans-1-(2-methoxyethyl)-4phenylpyrrolidin-3-yl)urea; 1-(1,3-diphenyl-1H-pyrazol-5-yl)-3-(trans-1-(2-methoxyethyl)-4-phenylpyrrolidin-3-yl)urea; 1-(trans-1-(2-methoxyethyl)-4-phenylpyrrolidin-3-yl)-3-(3-methyl-1-phenyl-1H-pyrazol-5-yl)urea; 1-(3-isopropyl-1-phenyl-1H-pyrazol-5-yl)-3-(trans-1-(2-methoxyethyl)-4-phenylpyrrolidin-3-yl)urea; 1-(trans-1-(2-methoxyethyl)-4-phenylpyrrolidin-3-yl)-3-(1-methyl-3-phenyl-1H-pyrazol-5-yl)urea; 1-(trans-1-(2-methoxyethyl)-4-phenylpyrrolidin-3-yl)-3-(2-phenyl-2,4,5,6-tetrahydrocyclopenta[c]pyra-zol-3-yl)urea; 1-(trans-1-(2-methoxyethyl)-4-phenylpyrrolidin-3-yl)-3-(1-phenyl-1H-pyrazol-5-yl)urea; 1-(3-tert-butyl-1-(2-fluorophenyl)-1H-pyrazol-5-yl)-3-(trans-1-(2-methoxyethyl)-4-phenylpyrrolidin-3-yl)urea; 1-(3-tert-butyl-1-(3-fluorophenyl)-1H-pyrazol-5-yl)-3-(trans-1-(2-methoxyethyl)-4-phenylpyrrolidin-3-yl)urea; 1-(3,4-dimethyl-1-phenyl-1H-pyrazol-5-yl)-3-(trans-1-(2-methoxyethyl)-4-phenylpyrrolidin-3-yl)urea; 1-(trans-1-(2-methoxyethyl)-4-phenylpyrrolidin-3-yl)-3-(2-(pyridin-3-yl)-2,4,5,6-tetrahydrocyclopenta[c]pyra-zol-3-yl)urea; 1-(trans-1-(2-methoxyethyl)-4-phenylpyrrolidin-3-yl)-3-(1-methyl-1H-pyrazol-5-yl)urea; 1-(trans-1-(2-methoxyethyl)-4-phenylpyrrolidin-3-yl)-3-(2-phenyl-2,4,5,6-tetrahydrocyclopenta[c]pyrazol-3-yl)thiourea; 1-(2-(3-fluorophenyl)-2,4,5,6-tetrahydrocyclopenta[c]pyrazol-3-yl)-3-(trans-1-(2-methoxyethyl)-4-phenylpyrrolidin-3-yl)urea; 1-(2-(4-fluorophenyl)-2,4,5,6-tetrahydrocyclopenta[c]pyrazol-3-yl)-3-(trans-1-(2-methoxyethyl)-4-phenylpyrrolidin-3-yl)urea; 1-(3-cyclopentyl-1-phenyl-1H-pyrazol-5-yl)-3-(trans-1-(2-methoxyethyl)-4-phenylpyrrolidin-3-yl)urea; 1-(1-ethyl-3-phenyl-1H-pyrazol-5-yl)-3-(trans-1-(2-methoxyethyl)-4-phenylpyrrolidin-3-yl)urea; 1-(trans-1-(2-methoxyethyl)-4-phenylpyrrolidin-3-yl)-3-(2-phenyl-4,5,6,7-tetrahydro-2H-indazol-3-yl)urea; 1-(trans-1-(2-methoxyethyl)-4-phenylpyrrolidin-3-yl)-3-(2-methyl-2,4,5,6-tetrahydrocyclopenta[c]pyrazol-3-yl)urea; 1-(1,3-dimethyl-4-phenyl-1H-pyrazol-5-yl)-3-(trans-1-(2-methoxyethyl)-4-phenylpyrrolidin-3-yl)urea; 1-(3-tert-butyl-1-o-tolyl-1H-pyrazol-5-yl)-3-(trans-1-(2-methoxyethyl)-4-phenylpyrrolidin-3-yl)urea; 1-(3-tert-butyl-1-m-tolyl-1H-pyrazol-5-yl)-3-(trans-1-(2-methoxyethyl)-4-phenylpyrrolidin-3-yl)urea; 1-(trans-1-(2-methoxyethyl)-4-phenylpyrrolidin-3-yl)-3-(1-methyl-4-phenyl-1H-pyrazol-5-yl)urea; 1-(4-cyano-3-methyl-1-phenyl-1H-pyrazol-5-yl)-3-(trans-1-(2-methoxyethyl)-4-phenylpyrrolidin-3-yl)urea; 1-(trans-1-(2-methoxyethyl)-4-phenylpyrrolidin-3-yl)-3-(2-(1-methyl-1H-pyrazol-4-yl)-2,4,5,6-tetrahydrocyclopenta[c]pyrazol-3-yl)urea; 1-(3-tert-butyl-1-(tetrahyro-2H-pyran-4-yl)-1H-pyrazol-5-yl)-3-(trans-1-(2-methoxyethyl)-4-phenylpyrrolidin-3-yl)urea; 1-(trans-1-(2-methoxyethyl)-4-phenylpyrrolidin-3-yl)-3-(2-(pyridin-2-yl)-2,4,5,6-tetrahydrocyclopenta[c]pyrazol-3-yl)urea; 1-(6,6-dimethyl-2-phenyl-2,4,5,6-tetrahydroxyclopenta [c]pyrazol-3-yl)-3-(trans-1-(2-methoxyethyl)-4-phenyl-pyrrolidin-3-yl)urea; 1-(7,7-dimethyl-2-phenyl-4,5,6,7-tetrahydro-2H-indazol-3-yl)-3-(trans-1-(2-methoxyethyl)-4-phenylpyrrolidin-3-yl)urea; 1-(trans-1-(2-methoxyethyl)-4-(pyridin-4-yl)pyrrolidin-3-yl)-3-(2-phenyl-2,4,5,6-tetrahydrocyclopenta[c]pyrazol-3-yl)urea; trans-1-(4-(3-fluorophenyl)-1-(2-methoxyethyl)pyrrolidin-3-yl)-3-(2-phenyl-2,4,5,6-tetrahydrocyclopenta[c]pyrazol-3-yl)urea; trans-1-(-4-(3-fluorophenyl)-1-(2-methoxyethyl)pyrrolidin-3-yl)-3-(3-isopropyl-1-phenyl-1H-pyrazol-5-yl)urea; trans-1-(4-(4-fluorophenyl)-1-(2-methoxyethyl)pyrrolidin-3-yl)-3-(3-isopropyl-1-phenyl-1H-pyrazol-5-yl)urea; trans-1-(4-(3-chlorophenyl)-1-(2-methoxyethyl)pyrrolidin-3-yl)-3-(3-isopropyl-1-phenyl-1H-pyrazol-5-yl)urea; trans-1-(4-(2-fluorophenyl)-1-(2-methoxyethyl)pyrrolidin-3-yl)-3-(3-isopropyl-1-phenyl-1H-pyrazol-5-yl)urea; trans-1-(3-isopropyl-1-phenyl-1H-pyrazol-5-yl)-3-(1-(2-methoxyethyl)-4-(thiophen-2-yl)pyrrolidin-3-yl)urea; 1-((3,4-trans)-4-(2,4-dimethylthiazol-5-yl)-1-(2-methoxyethyl)pyrrolidin-3-yl)-3-(2-phenyl-2,4,5,6-tetrahydrocyclopenta[c]pyrazol-3-yl)urea; 1-(trans-1-(2-methoxyethyl)-4-(oxazol-5-yl)pyrrolidin-3-yl)-3-(2-phenyl-2,4,5,6-tetrahydrocyclopenta[c]pyrazol-3-yl)urea; 1-(trans-4-(isoxazol-5-yl)-1-(2-methoxyethyl)pyrrolidin-3-yl)-3-(2-phenyl-2,4,5,6-tetrahydrocyclopenta[c]pyrazol-3-yl)urea; 1-((3,4-trans)-1-(2-methoxyethyl)-4-(3-methoxyphenyl)pyrrolidin-3-yl)-3-(2-phenyl-2,4,5,6-tetrahydrocyclopenta[c]pyrazol-3-yl)urea; 1-(1-(2-methoxyethyl)-4-(thiazol-2-yl)pyrrolidin-3-yl)-3-(2-phenyl-2,4,5,6-tetrahydrocyclopenta[c]pyrazol-3-yl)urea; 1-((3S,4R)-1-(2-methoxyethyl)-4-phenylpyrrolidin-3-yl)-3-(2-phenyl-2,4,5,6-tetrahydrocyclopenta[c]pyrazol-3-yl)urea; 1-(1,3-diphenyl-1H-pyrazol-5-yl)-3-((3S,4R)-1-(2-methoxyethyl)-4-phenylpyrrolidin-3-yl)urea; 1-((3S,4R)-1-(2-methoxyethyl)-4-phenylpyrrolidin-3-yl)-3-(1-methyl-3-phenyl-1H-pyrazol-5-yl)urea; 1-((3S,4R)-1-(2-methoxyethyl)-4-phenylpyrrolidin-3-yl)-3-(1-phenyl-3-(trifluoromethyl)-1H-pyrazol-5-yl)urea; 1-(1,4-dimethyl-3-phenyl-1H-pyrazol-5-yl)-3-((3S,4R)-1-(2-methoxyethyl)-4-phenylpyrrolidin-3-yl)urea; 1-(3-cyclopropyl-1-methyl-1H-pyrazol-5-yl)-3-((3S,4R)-4-(3,5-difluorophenyl)-1-(2-methoxyethyl)pyrrolidin-3-yl)urea; 1-((3S,4R)-4-(3,5-difluorophenyl)-1-(2-methoxyethyl)pyrrolidin-3-yl)-3-(1-methyl-3-(pyridin-2-yl)-1H-pyrazol-5-yl)urea; 1-((3S,4R)-4-(3,5-difluorophenyl)-1-(2-methoxyethyl)pyrrolidin-3-yl)-3-(1-methyl-3-(pyridin-3-yl)-1H-pyrazol-5-yl)urea; 1-((3S,4R)-4-(3,4-difluorophenyl)-1-(2-methoxyethyl)pyrrolidin-3-yl)-3-(1,1′-dimethyl-1H,1′H-3,4′-bipyrazol-5-yl)urea; 1-(3-(3-cyanophenyl)-1-methyl-1H-pyrazol-5-yl)-3-((3S,4R)-4-(3,4-difluorophenyl)-1-(2-methoxyethyl)pyrrolidin-3-yl)urea; 1-(3-(4-cyanophenyl)-1-methyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-(imidazo[1,2-a]pyridin-5-yl)-1-methyl-1H-pyrazol-5-yl)urea; 1-(4-chloro-1,3-diphenyl-1H-pyrazol-5-yl)-3-((3S,4R)-4-(3,4-difluorophenyl)-1-(2-methoxyethyl)pyrrolidin-3-yl)urea; 1-(4-bromo-1,3-diphenyl-1H-pyrazol-5-yl)-3-((3S,4R)-4-(3,4-difluorophenyl)-1-(2-methoxyethyl)pyrrolidin-3-yl)urea; 1-(4-chloro-3-methyl-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(1,3-dimethyl-4-phenyl-1H-pyrazol-5-yl)urea; 1-(4-cyano-3-methyl-1-phenyl-1H-pyrazol-5-yl)-3-((3S,4R)-4-(3,4-difluorophenyl)-1-(2-methoxyethyl)pyrrolidin-3-yl)urea; 1-(4-chloro-1-methyl-3-phenyl-1H-pyrazol-5-yl)-3-((3S,4R)-4-(3,4-difluorophenyl)-1-(2-methoxyethyl)pyrrolidin-3-yl)urea; 1-(4-bromo-1-methyl-3-phenyl-1H-pyrazol-5-yl)-3-((3S,4R)-4-(3,4-difluorophenyl)-1-(2-methoxyethyl)pyrrolidin-3-yl)urea; 1-(4-cyano-3-(cyanomethyl)-1-phenyl-1H-pyrazol-5-yl)-3-((3S,4R)-4(3,4-difluorophenyl)-1-(2-methoxyethyl)pyrrolidin-3-yl)urea; 1-(3-(2-cyanopropan-2-yl)-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-ethyl-4-methyl-1-phenyl-1H-pyrazol-5-yl)urea; 1-((3S,4R)-4-(3,4-difluorophenyl)-1-(2-methoxyethyl)pyrrolidin-3-yl)-3-(1′-methyl-1-phenyl-1H,1′H-3,4′-bipyrazol-5-yl)urea; 1-((3S,4R)-4-(3,4-difluorophenyl)-1-(2-methoxyethyl)pyrrolidin-3-yl)-3-(4-methyl-3-(oxetan-3-ylmethoxy)-1-phenyl-1H-pyrazol-5-yl)urea; 1-((3S,4R)-4-(3,4-difluorophenyl)-1-(2-methoxyethyl)pyrrolidin-3-yl)-3-(4-methyl-3-((3-methyloxetan-3-yl)methoxy)-1-phenyl-1H-pyrazol-5-yl)urea; 1-((3S,4R)-4-(3,4-difluorophenyl)-1-(2-methoxyethyl)pyrrolidin-3-yl)-3-(3-(((S)-2,2-dimethyl-1,3-dioxolan-4-yl)methoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(((R)-2,2-dimethyl-1,3-dioxolan-4-yl)methoxy)-4-methyl-1-phenyl-1H-pyrazol-5-yl)urea; 1-(3,4-dimethyl-1-phenyl-1H-pyrazol-5-yl)-3-((3S,4R)-1-(2-methoxyethyl)-4-phenylpyrrolidin-3-yl)urea; tert-butyl 3-(3-((3S,4R)-1-(2-methoxyethyl)-4-phenylpyrrolidin-3-yl)ureido)-2-phenyl-4,6-dihydropyrrolo[3,4-c]pyrazole-5 (2H)-carboxylate; 1-(3-isopropyl-4-methyl-1-phenyl-1H-pyrazol-5-yl)-3-((3S,4R)-1-(2-methoxyethyl)-4-phenylpyrrolidin-3-yl)urea; 1-((3S,4R)-1-(2-methoxyethyl)-4-phenylpyrrolidin-3-yl)-3-(2-phenyl-4,6-dihydro-2H-furo[3,4-c]pyrazol-3-yl)urea; 1-((3S,4R)-1-(2-methoxyethyl)-4-phenylpyrrolidin-3-yl)-3-(2-phenyl-4,6-dihydro-2H-thieno[3,4-c]pyrazol-3-yl)urea; 1-((3S,4R)-4-(3,5-difluorophenyl)-1-(2-methoxyethyl)pyrrolidin-3-yl)-3-(3,4-dimethyl-1-phenyl-1H-pyrazol-5-yl)urea; 1-((3S,4R)-4-(3,5-difluorophenyl)-1-(2-methoxyethyl)pyrrolidin-3-yl)-3-(2-phenyl-2,4,5,6-tetrahydrocyclopenta[c]pyrazol-3-yl)urea; 1-((3S,4R)-4-(3,5-difluorophenyl)-1-(2-methoxyethyl)pyrrolidin-3-yl)-3-(3-isopropyl-1-phenyl-1H-pyrazol-5-yl)urea; 1-((3S,4R)-4-(3,5-difluorophenyl)-1-(2-methoxyethyl)pyrrolidin-3-yl)-3-(3-methyl-1-phenyl-1H-pyrazol-5-yl)urea; 1-((3S,4R)-4-(3,4-difluorophenyl)-1-(2-methoxyethyl)pyrrolidin-3-yl)-3-(2-phenyl-2,4,5,6-tetrahydrocyclopenta[c]pyrazol-3-yl)urea; 1-((3S,4R)-4-(3,5-difluorophenyl)-1-(2-methoxyethyl)pyrrolidin-3-yl)-3-(2-phenyl-4,6-dihydro-2H-furo[3,4-c]pyrazol-3-yl)urea; 1-((3S,4R)-4-(3,5-difluorophenyl)-1-(2-methoxyethyl)pyrrolidin-3-yl)-3-(2-phenyl-4,6-dihydro-2H-thieno[3,4-c]pyrazol-3-yl)urea; 1-((3S,4R)-4-(3,4-difluorophenyl)-1-(2-methoxyethyl)pyrrolidin-3-yl)-3-(2-phenyl-4,6-dihydro-2H-furo[3,4-c]pyrazol-3-yl)urea; 1-((3S,4R)-4-(3,4-difluorophenyl)-1-(2-methoxyethyl)pyrrolidin-3-yl)-3-(2-phenyl-4,6-dihydro-2H-thieno[3,4-c]pyrazol-3-yl)urea; 1-((3S,4R)-4-(3,4-difluorophenyl)-1-(2-methoxyethyl)pyrrolidin-3-yl)-3-(3,4-dimethyl-1-phenyl-1H-pyrazol-5-yl)urea; 1-((3S,4R)-4-(3,4-difluorophenyl)-1-(2-methoxyethyl)pyrrolidin-3-yl)-3-(1-methyl-3-phenyl-1H-pyrazol-5-yl)urea; 1-(3-(1-hydroxy-2-methylpropan-2-yl)-1-phenyl-1H-pyrazol-5-yl)-3-((3S,4R)-1-(2-methoxyethyl)-4-phenylpyrrolidin-3-yl)urea; 1-((3S,4R)-1-(2-methoxyethyl)-4-phenylpyrrolidin-3-yl)-3-(5-oxido-2-phenyl-4,6-dihydro-2H-thieno[3,4-c]pyrazol-3-yl)urea; 1-((3S,4R)-1-(2-methoxyethyl)-4-phenylpyrrolidin-3-yl)-3-(1-methyl-3-(pyridin-4-yl)-1H-pyrazol-5-yl)urea; 1-((3S,4R)-4-(3,4-difluorophenyl)-1-(2-methoxyethyl)pyrrolidin-3-yl)-3-(1-methyl-3-(pyridin-4-yl)-1H-pyrazol-5-yl)urea; 1-((3S,4R)-4-(3,5-difluorophenyl)-1-(2-methoxyethyl)pyrrolidin-3-yl)-3-(1-methyl-3-phenyl-1H-pyrazol-5-yl)urea; 1-((3S,4R)-4-(3,5-difluorophenyl)-1-(2-methoxyethyl)pyrrolidin-3-yl)-3-(1-methyl-3-(thiophen-2-yl)-1H-pyrazol-5-yl)urea; 1-((3S,4R)-1-(2-methoxyethyl)-4-phenylpyrrolidin-3-yl)-3-(3-(methoxymethyl)-1-phenyl-1H-pyrazol-5-yl)urea; 1-((3S,4R)-1-(2-methoxyethyl)-4-phenylpyrrolidin-3-yl)-3-(3-(methoxymethyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(1-methyl-3-p-tolyl-1H-pyrazol-5-yl)urea; 1-((3S,4R)-4-(3,4-difluorophenyl)-1-(2-methoxyethyl)pyrrolidin-3-yl)-3-(1-methyl-3-m-tolyl-1H-pyrazol-5-yl)urea; 1-((3S,4R)-4-(3,4-difluorophenyl)-1-(2-methoxyethyl)pyrrolidin-3-yl)-3-(1-methyl-3-o-tolyl-1H-pyrazol-5-yl)urea; 1-((3S,4R)-4-(3,4-difluorophenyl)-1-(2-methoxyethyl)pyrrolidin-3-yl)-3-(3-(3-methoxyphenyl)-1-methyl-1H-pyrazol-5-yl)urea; 1-((3S,4R)-4-(3,4-difluorophenyl)-1-(2-methoxyethyl)pyrrolidin-3-yl)-3-(3-(2-methoxyphenyl)-1-methyl-1H-pyrazol-5-yl)urea; 1-(3-(4-fluorophenyl)-1-methyl-1H-pyrazol-5-yl)-3-((3S,4R)-1-(2-methoxyethyl)-4-phenylpyrrolidin-3-yl)urea; 1-((3S,4R)-1-(2-methoxyethyl)-4-phenylpyrrolidin-3-yl)-3-(3-(4-methoxyphenyl)-1-methyl-1H-pyrazol-5-yl)urea; 1-((3S,4R)-1-(2-methoxyethyl)-4-(3-(trifluoromethyl)phenyl)pyrrolidin-3-yl)-3-(1-methyl-3-phenyl-1H-pyrazol-5-yl)urea; 1-((3S,4R)-4-(3-fluorophenyl)-1-(2-methoxyethyl)pyrrolidin-3-yl)-3-(1-methyl-3-phenyl-1H-pyrazol-5-yl)urea; 1-((3S,4R)-4-(2,4-difluorophenyl)-1-(2-methoxyethyl)pyrrolidin-3-yl)-3-(1-methyl-3-phenyl-1H-pyrazol-5-yl)urea; 1-((3S,4R)-4-(3-fluorophenyl)-1-(2-methoxyethyl)pyrrolidin-3-yl)-3-(3-(4fluorophenyl)-1-methyl-1H-pyrazol-5-yl)urea; 1-((3S,4R)-4-(3,4-difluorophenyl)-1-(2-methoxyethyl)pyrrolidin-3-yl)-3-(3-(4-fluorophenyl)-1-methyl-1H-pyrazol-5-yl)urea; 1-((3S,4R)-4-(3,4-difluorophenyl)-1-(2-methoxyethyl)pyrrolidin-3-yl)-3-(3-(3-fluorophenyl)-1-methyl-1H-pyrazol-5-yl)urea; 1-((3S,4R)-4-(3,4-difluorophenyl)-1-(2-methoxyethyl)pyrrolidin-3-yl)-3-(3-(2-fluorophenyl)-1-methyl-1H-pyrazol-5-yl)urea; 1-((3S,4R)-4-(3-fluorophenyl)-1-(2-methoxyethyl)pyrrolidin-3-yl)-3-(2-phenyl-2,4,5,6-tetrahydrocyclopenta[c]pyrazol-3-yl)urea; 1-(3-(1-hydroxy-2-methylpropan-2-yl)-4-methyl-1-phenyl-1H-pyrazol-5-yl)-3-((3S,4R)-1-(2-methoxyethyl)-4-phenylpyrrolidin-3-yl)urea; 1-((3S,4R)-4-(3,4-difluorophenyl)-1-(2-methoxyethyl)pyrrolidin-3-yl)-3-(3-(1-hydroxy-2-methylpropan-2-yl)-4-methyl-1-phenyl-1H-pyrazol-5-yl)urea; 1-(3-(4-chlorophenyl)-1-methyl-1H-pyrazol-5-yl)-3-((3S,4R)-4-(3,4difluorophenyl)-1-(2-methoxyethyl)pyrrolidin-3-yl)urea; 1-((3S,4R)-4-(2, 5-difluorophenyl)-1-(2-methoxyethyl)pyrrolidin-3-yl)-3-(3-(4-fluorophenyl)-1-methyl-1H-pyrazol-5-yl)urea; methyl 4-(5-(3-((3S,4R)-4-(3,4-difluorophenyl)-1-(2-methoxyethyl)pyrrolidin-3-yl)ureido)-1-methyl-1H-pyrazol-3-yl)benzoate; 1-((3S,4R)-4-(3,4-difluorophenyl)-1-(2-methoxyethyl)pyrrolidin-3-yl)-3-(1-(2-hydroxyethyl)-3-phenyl-1H-pyrazol-5-yl)urea; 1-((3S,4R)-4-(3,4-difluorophenyl)-1-(2-methoxyethyl)pyrrolidin-3-yl)-3-(3-(methoxymethyl)-4-methyl-1-phenyl-1H-pyrazol-5-yl)urea; 1-((3S,4R)-4-(3,5-difluorophenyl)-1-(2-methoxyethyl)pyrrolidin-3-yl)-3-(3-(methoxymethyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(1-methyl-3-(4-(methylthio)phenyl)-1H-pyrazol-5-yl)urea; 1-((3S,4R)-4-(3,4-difluorophenyl)-1-(2-methoxyethyl)pyrrolidin-3-yl)-3-(1,3-diphenyl-1H-pyrazol-5-yl)urea; 1-((3S,4R)-4-(3,4-difluorophenyl)-1-(2-methoxyethyl)pyrrolidin-3-yl)-3-(3-(3-methoxypropyl)-4-methyl-1-phenyl-1H-pyrazol-5-yl)urea; 1-((3S,4R)-4-(4-fluorophenyl)-1-(2-methoxyethyl)pyrrolidin-3-yl)-3-(2-phenyl-2,4,5,6-tetrahydrocyclopenta[c]pyrazol-3-yl)urea; 1-(3,4-dimethyl-1-phenyl-1H-pyrazol-5-yl)-3-((3S,4R)-4-(4-fluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-(4-(2-methoxyethoxy)phenyl)-1-methyl-1H-pyrazol-5-yl)urea; 1-((3S,4R)-4-(3,4-difluorophenyl)-1-(2-methoxyethyl)pyrrolidin-3-yl)-3-(4-methoxy-3-methyl-1-phenyl-1H-pyrazol-5-yl)urea; 1-((3S,4R)-4-(3,4-difluorophenyl)-1-(2-methoxyethyl)pyrrolidin-3-yl)-3-(3-(hydroxymethyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2-hydroxyethyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2-methoxyethyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-ethoxy-4-methyl-1-phenyl-1H-pyrazol-5-yl)urea; 1-(3-(benzyloxy)-1-methyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-(2-methoxyethoxy)-4-methyl-1-phenyl-1H-pyrazol-5-yl)urea; 1-((3S,4R)-4-(3,5-difluorophenyl)-1-(2-methoxyethyl)pyrrolidin-3-yl)-3-(3-ethoxy-4-methyl-1-phenyl-1H-pyrazol-5-yl)urea; trans-1-(3-ethoxy-4-methyl-1-phenyl-1H-pyrazol-5-yl)-3-1-(2-methoxyethyl)-4-phenylpyrrolidin-3-yl)urea; 1-((3S,4R)-4-(3,4-difluorophenyl)-1-(2-methoxyethyl)pyrrolidin-3-yl)-3-(3-methoxy-4-methyl-1-phenyl-1H-pyrazol-5-yl)urea; 1-(3-(cyanomethoxy)-4-methyl-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluoro-phenyl)-1-(2-methoxyethyl) pyrrolidin-3-yl)-3-(3-(4-methoxybenzyloxy)-4-methyl-1-phenyl-1H-pyrazol-5-yl)urea; 1-((3S,4R)-4-(4-fluorophenyl)-1-(2-methoxyethyl)pyrrolidin-3-yl)-3-(3-methoxy-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2-fluoroethoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2-hydroxy-2-methylpropoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-ethoxy-1-phenyl-1H-pyrazol-5-yl)urea; 1-(3-ethoxy-4-methyl-1-phenyl-1H-pyrazol-5-yl)-3-((3S,4R)-4-(4-fluorophenyl)-1-(2-methoxyethyl)pyrrolidin-3-yl)urea; 1-(3-ethoxy-4-methyl-1-phenyl-1H-pyrazol-5-yl)-3-((3S,4R)-4-(3-fluorophenyl)-1-(2-methoxyethyl)pyrrolidin-3-yl)urea; 1-((3S,4R)-4-(3,5-difluorophenyl)-1-(2-methoxyethyl)pyrrolidin-3-yl)-3-(3-(2-hydroxyethoxy)-4-methyl-1-phenyl-1H-pyrazol-5-yl)urea; 1-(2-cyclohexyl-2,4,5,6-tetrahydrocyclopenta[c]pyrazol-3-yl)-3-((3S,4R)-4-(3,4-difluorophenyl)-1-(2-methoxyethyl)pyrrolidin-3-yl)urea; 1-((3S,4R)-1-(2-methoxyethyl)-4-phenylpyrrolidin-3-yl)-3-(2-(pyridin-4-yl)-2,4,5,6-tetrahydrocyclopenta[c]pyrazol-3-yl)urea; 1-((3S,4R)-4-(3,4-difluorophenyl)-1-(2-methoxyethyl)pyrrolidin-3-yl)-3-(1-methyl-3-(5-methylpyrazin-2-yl)-1H-pyrazol-5-yl)urea; 1-((3S,4R)-4-(3,4-difluorophenyl)-1-(2-methoxyethyl)pyrrolidin-3-yl)-3-(1,4-dimethyl-3-(5-methylpyrazin-2-yl)-1H-pyrazol-5-yl)urea; ethyl 5-(3-((3S,4R)-4-(3,4-difluorophenyl)-1-(2-methoxyethyl)pyrrolidin-3-yl)ureido)-1-phenyl-1H-pyrazole-4-carboxylate; 1-((3S,4R)-4-(3,4-difluorophenyl)-1-(2-methoxyethyl)pyrrolidin-3-yl)-3-(1-methyl-3-(pyrazin-2-yl)-1H-pyrazol-5-yl)urea; 1-((3S,4R)-4-(3,4-difluorophenyl)-1-(2-methoxyethyl)pyrrolidin-3-yl)-3-(3-methoxy-1-methyl-4-phenyl-1H-pyrazol-5-yl)urea; 1-((3S,4R)-4-(3,4-difluorophenyl)-1-(2-methoxyethyl)pyrrolidin-3-yl)-3-(3-ethoxy-1-methyl-4-phenyl-1H-pyrazol-5-yl)urea; 1-((3S,4R)-1-(2-methoxyethyl)-4-phenylpyrrolidin-3-yl)-3-(2-phenyl-2,4,5,6-tetrahydropyrrolo[3,4-c]pyrazol-3-yl)urea dihydrochloride; 1-(5-acetyl-2-phenyl-2,4,5,6-tetrahydropyrrolo[3,4-c]pyrazol-3-yl)-3-((3S,4R)-1-(2-methoxyethyl)-4-phenylpyrrolidin-3-yl)urea; 1-((3S,4R)-4-(3,4-difluorophenyl)-1-(2-methoxyethyl)pyrrolidin-3-yl)-3-(4-(hydroxymethyl)-3-(methoxymethyl)-1-phenyl-1H-pyrazol-5-yl)urea; 4-(5-(3-((3S,4R)-4-(3,4-difluorophenyl)-1-(2-methoxyethyl)pyrrolidin-3-yl)ureido)-1-methyl-1H-pyrazol-3-yl)benzoic acid; 4-(5-(3-((3S,4R)-4-(3,4-difluorophenyl)-1-(2-methoxyethyl)pyrrolidin-3-yl)ureido)-1-methyl-1H-pyrazol-3-yl)benzamide; 4-(5-(3-((3S,4R)-4-(3,4-difluorophenyl)-1-(2-methoxyethyl)pyrrolidin-3-yl)ureido)-1-methyl-1H-pyrazol-3-yl)-N-methylbenzamide; 4-(5-(3-((3S,4R)-4-(3,4-difluorophenyl)-1-(2-methoxyethyl)pyrrolidin-3-yl)ureido)-1-methyl-1H-pyrazol-3-yl)-N,N-dimethylbenzamide; 1-((3S,4R)-4-(3,4-difluorophenyl)-1-(2-methoxyethyl)pyrrolidin-3-yl)-3-(3-(4-(hydroxymethyl)phenyl)-1-methyl-1H-pyrazol-5-yl)urea; 1-((3S,4R)-4-(3,4-difluorophenyl)-1-(2-methoxyethyl)pyrrolidin-3-yl)-3-(1-methyl-3-(4-(methyl sulfonyl)phenyl)-1H-pyrazol-5-yl)urea; 1-((3S,4R)-4-(3,4-difluorophenyl)-1-(2-methoxyethyl)pyrrolidin-3-yl)-3-(4-fluoro-3-methyl-1-phenyl-1H-pyrazol-5-yl)urea; 1-((3S,4R)-4-(3,4-difluorophenyl)-1-(2-methoxyethyl)pyrrolidin-3-yl)-3-(4-fluoro-1-methyl-3-phenyl-1H-pyrazol-5-yl)urea; 1-((3S,4R)-4-(3,4-difluorophenyl)-1-(2-methoxyethyl)pyrrolidin-3-yl)-3-(4-fluoro-1,3-diphenyl-1H-pyrazol-5-yl)urea; 2-methoxyethyl 4-(5-(3-((3S,4R)-4-(3,4-difluorophenyl)-1-(2-methoxyethyl)pyrrolidin-3-yl)ureido)-1-methyl-1H-pyrazol-3-yl)benzoate; 1-((3S,4R)-4-(3,4-difluorophenyl)-1-(2-methoxyethyl)pyrrolidin-3-yl)-3-(5,5-dioxido-2-phenyl-4,6-dihydro-2H-thieno[3,4-c]pyrazol-3-yl)urea; 1-(5,5-dioxido-2-phenyl-4,6-dihydro-2H-thieno[3,4-c]pyrazol-3-yl)-3-((3S,4R)-1-(2-methoxyethyl)-4-phenylpyrrolidin-3-yl)urea; 1-((3S,4R)-4-(3,5-difluorophenyl)-1-(2-methoxyethyl)pyrrolidin-3-yl)-3-(5,5-dioxido-2-phenyl-4,6-dihydro-2H-thieno[3,4-c]pyrazol-3-yl)urea; 1-((3S,4R)-4-(3,4-difluorophenyl)-1-(2-methoxyethyl)pyrrolidin-3-yl)-3-(4-methyl-3-(2-(methylsulfonyl)ethoxy)-1-phenyl-1H-pyrazol-5-yl)urea; 1-((3S,4R)-4-(3,4-difluorophenyl)-1-(2-methoxyethyl)pyrrolidin-3-yl)-3-(4-(hydroxymethyl)-1-phenyl-1H-pyrazol-5-yl)urea; 1-((3S,4R)-4-(3,4-difluorophenyl)-1-(2-methoxyethyl)pyrrolidin-3-yl)-3-(3-((R)-2,3-dihydroxypropoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-((S)-2,3-dihydroxypropoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2-hydroxyethoxy)-4-methyl-1-phenyl-1H-pyrazol-5-yl)urea hydrochloride; 1-((3S,4R)-4-(3,4-difluorophenyl)-1-(2-methoxyethyl)pyrrolidin-3-yl)-3-(3-((S)-2-hydroxypropoxy)-4-methyl-1-phenyl-1H-pyrazol-5-yl)urea hydrochloride; 1-((3R,4S)-4-hydroxy-1-(2-methoxyethyl)-4-phenylpyrrolidin-3-yl)-3-(2-phenyl-2,4,5,6-tetrahydrocyclopenta[c]pyrazol-3-yl)urea; 1-((3R,4S)-4-fluoro-1-(2-methoxyethyl)-4-phenylpyrrolidin-3-yl)-3-(2-phenyl-2,4,5,6-tetrahydrocyclopenta[c]pyrazol-3-yl)urea; 1-(trans-4-phenyl-1-(2-(trifluoromethoxy)ethyl)pyrrolidin-3-yl)-3-(2-phenyl-2,4,5,6-tetrahydrocyclopenta[c]pyrazol-3-yl)urea; 1-(trans-1-(2-(methylthio)ethyl)-4-phenylpyrrolidin-3-yl)-3-(2-phenyl-2,4,5,6-tetrahydrocyclopenta[c]pyrazol-3-yl)urea; 1-((3S,4R)-1-((S)-2-methoxypropyl)-4-phenylpyrrolidin-3-yl)-3-(2-phenyl-2,4,5,6-tetrahydrocyclopenta[c]pyrazol-3-yl)urea; 1-((3,4-trans)-4-phenyl-1-(4,4,4-trifluorobutyl)pyrrolidin-3-yl)-3-(2-phenyl-2,4,5,6-tetrahydrocyclopenta[c]pyrazol-3-yl)urea; 1-((3S,4R)-1-(cyanomethyl)-4-(3,4-difluorophenyl)pyrrolidin-3-yl)-3-(2-phenyl-2,4,5,6-tetrahydrocyclopenta[c]pyrazol-3-yl)urea; 1-((3S,4R)-1-(cyanomethyl)-4-(3,4-difluorophenyl)pyrrolidin-3-yl)-3-(3-(2-methoxyethoxy)-4-methyl-1-phenyl-1H-pyrazol-5-yl)urea; 1-((3,4-trans)-1-(cyanomethyl)-4-phenylpyrrolidin-3-yl)-3-(2-phenyl-2,4,5,6-tetrahydrocyclopenta[c]pyrazol-3-yl)urea; 1-((3S,4R)-1-(cyanomethyl)-4-phenylpyrrolidin-3-yl)-3-(2-phenyl-2,4,5,6-tetrahydrocyclopenta[c]pyrazol-3-yl)urea; 2-((3R,4S)-3-phenyl-4-(3-(2-phenyl-2,4,5,6-tetrahydrocyclopenta[c]pyrazol-3-yl)ureido)pyrrolidin-1-yl)acetamide; 1-((3S,4R)-4-(3,4-difluorophenyl)-1-(2-hydroxyethyl)pyrrolidin-3-yl)-3-(3,4-dimethyl-1-phenyl-1H-pyrazol-5-yl)urea; 1-((trans)-1-(3,3,4,4,4-pentafluorobutyl)-4-phenylpyrrolidin-3-yl)-3-(2-phenyl-2,4,5,6-tetrahydrocyclopenta[c]pyrazol-3-yl)urea; 1-((trans)-1-ethyl-4-phenylpyrrolidin-3-yl)-3-(2-phenyl-2,4,5,6-tetrahydrocyclopenta[c]pyrazol-3-yl)urea; 1-((trans)-4-phenyl-1-(2,2,2-trifluoroethyl)pyrrolidin-3-yl)-3-(2-phenyl2,4,5,6-tetrahydrocyclopenta[c]pyrazol-3-yl)urea; 1-(3,4-dimethyl-1-phenyl-1H-pyrazol-5-yl)-3-((trans)-4-phenyl-1-(2,2,2-trifluoroethyl)pyrrolidin-3-yl)urea; 1-(3-(2-methoxyethoxy)-4-methyl-1-phenyl-1H-pyrazol-5-yl)-3-((trans)-4-phenyl-1-(2,2,2-trifluoroethyl)pyrrolidin-3-yl)urea; 1-((trans)-4-phenyl-1-(2,2,2-trifluoroethyl)pyrrolidin-3-yl)-3-(2-phenyl2,4,5,6-tetrahydrocyclopenta[c]pyrazol-3-yl)urea; 1-(3,4-dimethyl-1-phenyl-1H-pyrazol-5-yl)-3-((3S,4R)-4-phenyl-1-(2,2,2-trifluoroethyl)pyrrolidin-3-yl)urea; 1-(3-(2-methoxyethoxy)-4-methyl-1-phenyl-1H-pyrazol-5-yl)-3-((3S,4R)-4-phenyl-1-(2,2,2-trifluoroethyl)pyrrolidin-3-yl)urea; 1-((3S,4R)-4-(3-fluorophenyl)-1-(2,2,2-trifluoroethyl)pyrrolidin-3-yl)-3(2-phenyl-2,4,5,6-tetrahydrocyclopenta[c]pyrazol-3-yl)urea; 1-(3,4-dimethyl-1-phenyl-1H-pyrazol-5-yl)-3-((3S,4R)-4-(3-fluorophenyl)-1-(2,2,2-trifluoroethyl)pyrrolidin-3-yl)urea; 1-((3S,4R)-4-(3-fluorophenyl)-1-(2,2,2-trifluoroethyl)pyrrolidin-3-yl)-3(3-(2-methoxyethoxy)-4-methyl-1-phenyl-1H-pyrazol-5-yl)urea; 1-((3S,4R)-4-(3-fluorophenyl)-1-(2,2,2-trifluoroethyl)pyrrolidin-3-yl)-3(1-methyl-3-phenyl-1H-pyrazol-5-yl)urea; 1-((3R,4S)-4-(3-fluorophenyl)-1-(2,2,2-trifluoroethyl)pyrrolidin-3-yl)-3-(2-phenyl-2,4,5,6-tetrahydrocyclopenta[c]pyrazol-3-yl)urea; 1-(3-ethoxy-4-methyl-1-phenyl-1H-pyrazol-5-yl)-3-((3R,3S)-4-(3-fluorophenyl)-1-(2,2,2-trifluoroethyl)pyrrolidin-3-yl)urea; 1-((trans)-1-(1,3-difluoropropan-2-yl)-4-phenylpyrrolidin-3-yl)-3-(2-phenyl-2,4,5,6-tetrahydrocyclopenta[c]pyrazol-3-yl)urea; (trans)-tert-butyl 3-(3-methoxyphenyl)-4-(3-(2-phenyl-2,4,5,6-tetrahydrocyclopenta[c]pyrazol-3-yl)ureido)pyrrolidine-1-carboxylate; 1-((trans)-4-(3-chlorophenyl)-1-(2,2,2-trifluoroethyl)pyrrolidin-3-yl)-3(2-phenyl-2,4,5,6-tetrahydrocyclopenta[c]pyrazol-3-yl)urea; 1-(2-phenyl-2,4,5,6-tetrahydrocyclopenta[c]pyrazol-3-yl)-3-((trans)-4-(pyridin-2-yl)-1-(2,2,2-trifluoroethyl)pyrrolidin-3-yl)urea; 1-((trans)-4-(4-fluorophenyl)-1-(2,2,2-trifluoroethyl)pyrrolidin-3-yl)-3(2-phenyl-2,4,5,6-tetrahydrocyclopenta[c]pyrazol-3-yl)urea; 1-((trans)-4-(4-chlorophenyl)-1-(2,2,2-trifluoroethyl)pyrrolidin-3-yl)-3(2-phenyl-2,4,5,6-tetrahydrocyclopenta[c]pyrazol-3-yl)urea; 1-((trans)-4-(2-chlorophenyl)-1-(2,2,2-trifluoroethyl)pyrrolidin-3-yl)-3(2-phenyl-2,4,5,6-tetrahydrocyclopenta[c]pyrazol-3-yl)urea; 1-(2-phenyl-2,4,5,6-tetrahydrocyclopenta[c]pyrazol-3-yl)-3-((trans)-4-(pyridin-3-yl)-1-(2,2,2-trifluoroethyl)pyrrolidin-3-yl)urea; 1-((trans)-4-(2-fluorophenyl)-1-(2,2,2-trifluoroethyl)pyrrolidin-3-yl)-3(2-phenyl-2,4,5,6-tetrahydrocyclopenta[c]pyrazol-3-yl)urea; 1-((trans)-4-(4-fluorophenyl)-1-(2,2-difluoroethyl)pyrrolidin-3-yl)-3-(2phenyl-2,4,5,6-tetrahydrocyclopenta[c]pyrazol-3-yl)urea; 1-((3S,4R)-4-(3,4-difluorophenyl)-1-(1H-pyrazol-4-yl)pyrrolidin-3-yl)-3-(3-ethoxy-4-methyl-1-phenyl-1H-pyrazol-5-yl)urea; 1-((3S,4R)-4-(3,5-difluorophenyl)-1-(1H-pyrazol-4-yl)pyrrolidin-3-yl)-3-(3-ethoxy-4-methyl-1-phenyl-1H-pyrazol-5-yl)urea; 1-((3S,4R)-4-(3,5-difluorophenyl)-1-(1H-pyrazol-3-yl)pyrrolidin-3-yl)-3-(3-ethoxy-4-methyl-1-phenyl-1H-pyrazol-5-yl)urea; 1-((3S,4R)-4-(3,4-difluorophenyl)-1-(3-methyl-1H-pyrazol-4-yl)pyrrolidin3-yl)-3-(3-ethoxy-4-methyl-1-phenyl-1H-pyrazol-5-yl)urea; 1-((3S,4R)-4-(3,4-difluorophenyl)-1-(3-(trifluoromethyl)-1H-pyrazol-4-yl)pyrrolidin-3-yl)-3-(3-ethoxy-4-methyl-1-phenyl-1H-pyrazol-5-yl)urea; 1-((3S,4R)-4-(3,4-difluorophenyl)-1-(1H-pyrazol-4-yl)pyrrolidin-3yl)-3-(3-((S)-2-hydroxypropoxy)-4-methyl-1-phenyl-1H-pyrazol-5-yl)urea; 1-((3S,4R)-4-(3,4-difluorophenyl)-1-(1H-pyrazol-4-yl)pyrrolidin-3yl)-3-(3-((R)-2,3-dihydroxypropoxy)-4-methyl-1-phenyl-1H-pyrazol-5-yl)urea; 1-((3S,4R)-4-(3,5-difluoro-phenyl)-1-(1H-pyrazol-4-yl)pyrrolidin3-yl)-3-(3,4-dimethyl-1-phenyl-1H-pyrazol-5-yl)urea; 1-((3S,4R)-4-(3,4-difluorophenyl)-1-(1H-pyrazol-4-yl)pyrrolidin-3-yl)-3-(3,4-dimethyl-1-phenyl-1H-pyrazol-5-yl)urea; 1-((3S,4R)-4-(3,4-difluorophenyl)-1-(1H-pyrazol-4-yl)pyrrolidin-3-yl)-3-(3-(3-methoxypropyl)-4-methyl-1-phenyl-1H-pyrazol-5-yl)urea; 1-((3S,4R)-4-(3,4-difluorophenyl)-1-(1H-pyrazol-4-yl)pyrrolidin-3-yl)-3-(3-(2-methoxyethoxy)-4-methyl-1-phenyl-1H-pyrazol-5-yl)urea; 1-((3S,4R)-4-(3,4-difluorophenyl)-1-(1H-pyrazol-4-yl)pyrrolidin-3-yl)-3-(3-(2-hydroxy-2-methylpropoxy)-4-methyl-1-phenyl-1H-pyrazol-5-yl)urea; 1-(3-ethoxy-4-methyl-1-phenyl-1H-pyrazol-5-yl)-3-((3S,4R)-1-(1-methyl-1H-pyrazol-5-yl)-4-phenylpyrrolidin-3-yl)urea; 1-(3-ethoxy-4-methyl-1-phenyl-1H-pyrazol-5-yl)-3-((3R,4S)-1-(1-methyl-1Hpyrazol-5-yl)-4-phenylpyrrolidin-3-yl)urea; 1-((3S,4R)-4-(3,5-difluorophenyl)-1-(1-methyl-1H-pyrazol-5-yl)pyrrolidin3-yl)-3-(3-ethoxy-4-methyl-1-phenyl-1H-pyrazol-5-yl)urea; 1-((3R,4S)-4-(3,5-difluorophenyl)-1-(1-methyl-1H-pyrazol-5-yl)pyrrolidin3-yl)-3-(3-ethoxy-4-methyl-1-phenyl-1H-pyrazol-5-yl)urea; 1-((3S,4R)-4-(3,4-difluorophenyl)-1-phenylpyrrolidin-3-yl)-3-(3-ethoxy-4methyl-1-phenyl-1H-pyrazol-5-yl)urea; 1-((3S,4R)-4-(3,4-difluorophenyl)-1-(2-methoxyphenyl)pyrrolidin-3-yl)-3-(3-ethoxy-4-methyl-1-phenyl-1H-pyrazol-5-yl)urea; 1-(3-ethoxy-4-methyl-1-phenyl-1H-pyrazol-5-yl)-3-((3S,4R)-1-(2-fluorophenyl)-4-phenylpyrrolidin-3-yl)urea; 1-(3-ethoxy-4-methyl-1-phenyl-1H-pyrazol-5-yl)-3-((3S,4R)-1-(4-fluorophenyl)-4-phenylpyrrolidin-3-yl)urea; 1-(3-ethoxy-4-methyl-1-phenyl-1H-pyrazol-5-yl)-3-((3S,4R)-1-(2-methylphenyl)-4-phenylpyrrolidin-3-yl)urea; 1-(3-ethoxy-4-methyl-1-phenyl-1H-pyrazol-5-yl)-3-((3S,4R)-1-(2-methoxyphenyl)-4-phenylpyrrolidin-3-yl)urea; 1-(3-ethoxy-4-methyl-1-phenyl-1H-pyrazol-5-yl)-3-((3S,4R)-1-(2-chlorophenyl)-4-phenylpyrrolidin-3-yl)urea; 1-(3-ethoxy-4-methyl-1-phenyl-1H-pyrazol-5-yl)-3-((3S,4R)-4-phenyl-1-(2-(trifluoromethoxy)phenyl)pyrroli-din-3-yl)urea; 1-((3S,4R)-1-(2,6-difluorophenyl)-4-phenylpyrrolidin-3-yl)-3-(3-ethoxy-4methyl-1-phenyl-1H-pyrazol-5-yl)urea; 1-(3-ethoxy-4-methyl-1-phenyl-1H-pyrazol-5-yl)-3-((3S,4R)-1-(2-methoxypyridin-4-yl)-4-phenylpyrrolidin-3-yl)urea; 1-(3-ethoxy-4-methyl-1-phenyl-1H-pyrazol-5-yl)-3-((3S,4R)-1-(2-methoxypyridin-3-yl)-4-phenylpyrrolidin-3-yl)urea; 1-(3-ethoxy-4-methyl-1-phenyl-1H-pyrazol-5-yl)-3-((3S,4R)-1-(2-ethoxypyridin-3-yl)-4-phenylpyrrolidin-3-yl)urea; 1-(1′,4-dimethyl-1-phenyl-1H,1′H-[3,4′-bipyrazol]-5-yl)-3-((3S,4R)-1-(2-methoxypyridin-3-yl)-4-phenylpyrrolidin-3-yl)urea; 1-((3S,4R)-1-(2-methoxypyridin-3-yl)-4-phenylpyrrolidin-3-yl)-3-(4-methyl-1,3-diphenyl-1H-pyrazol-5-yl)urea; 1-(4-bromo-1′-methyl-1-phenyl-1H,1′H-[3,4′-bipyrazol]-5-yl)-3-((3S,4R)-1-(2-methoxypyridin-3-yl)-4-phenylpyrrolidin-3-yl)urea; 1-(4-bromo-1,3-diphenyl-1H-pyrazol-5-yl)-3-((3S,4R)-1-(2-methoxypyridin-3-yl)-4-phenylpyrrolidin-3-yl)urea; 1-((3S,4R)-1-((1,2,3-thiadiazol-4-yl)methyl)-4-(3,4-difluorophenyl)pyrrolidin-3-yl)-3-(2-phenyl-2,4,5,6-tetrahydro cyclopenta[c]pyrazol-3-yl)urea; 1-((3S,4R)-1-((1,2,3-thiadiazol-4-yl)methyl)-4-(3,4-difluorophenyl)pyrrolidin-3-yl)-3-(3-ethoxy-4-methyl-1-phenyl-1H-pyrazol-5-yl)urea; 1-((3S,4R)-1-((1,2,3-thiadiazol-4-yl)methyl)-4-(3,4-difluorophenyl)pyrrolidin-3-yl)-3-(3-(cyanomethoxy)-4-methyl-1-phenyl-1H-pyrazol-5-yl)urea; 1-((3S,4R)-1-((1,2,3-thiadiazol-4-yl)methyl)-4-(3,4-difluorophenyl)pyrrolidin-3-yl)-3-(1′,4-dimethyl-1-phenyl-1H, 11′H-3,4′-bipyrazol-5-yl)urea; 1-((3S,4R)-1-((1,2,3-thiadiazol-4-yl)methyl)-4-(3,4-difluorophenyl)pyrrolidin-3-yl)-3-(4-methyl-3-(1-methyl-1H-imidazol-4-yl)-1-phenyl-1H-pyrazol-5-yl)urea; 1-((3S,4R)-4-(3,4-difluorophenyl)-1-((1-methyl-1H-1,2,3-triazol-4-yl)methyl)pyrrolidin-3-yl)-3-(2-phenyl-2,4,5,6-tetrahydrocyclopenta[c]pyrazol-3-yl)urea; 1-((3S,4R)-4-(3,4-difluorophenyl)-1-(1,3-dimethoxypropan-2-yl)pyrrolidin3-yl)-3-(3-ethoxy-4-methyl-1-phenyl-1H-pyrazol-5-yl)urea; 1-((3S,4R)-4-(3,4-difluorophenyl)-1-(1-methoxypropan-2-yl)pyrrolidin-3-yl)-3-(3-ethoxy-4-methyl-1-phenyl-1H-pyrazol-5-yl)urea; 1-((trans)-4-(4-fluorophenyl)-1-(2-(methylamino)ethyl)pyrrolidin-3-yl)-3(2-phenyl-2,4,5,6-tetrahydrocyclopenta[c]pyrazol-3-yl)urea; 1-((trans)-1-((1H-imidazol-2-yl)methyl)-4-phenylpyrrolidin-3-yl)-3-(2-phenyl-2,4,5,6-tetrahydrocyclopenta[c]pyrazol-3-yl)urea; methyl 3-methoxy-2-((trans)-3-phenyl-4-(3-(2-phenyl-2,4,5,6-tetrahydrocyclopenta[c]pyrazol-3-yl)ureido)pyrrolidin-1-yl)propanoate; 1-((3S,4R)-4-(3,4-difluorophenyl)-1-(1-methoxypropan-2-yl)pyrrolidin-3-yl)-3-(3-ethoxy-4-methyl-1-phenyl-1H-pyrazol-5-yl)urea; 1-((3S,4R)-4-(3,4-difluorophenyl)-1-(1-hydroxy-3-methoxypropan-2-yl)pyrrolidin-3-yl)-3-(2-phenyl-2,4,5,6-tetrahydrocyclopenta[c]pyrazol-3-yl)urea; 1-((3S,4R)-4-(3,4-difluorophenyl)-1-(3-hydroxy-1-methoxy-3-methylbutan-2-yl)pyrrolidin-3-yl)-3-(2-phenyl-2,4,5,6-tetrahydrocyclopenta[c]pyrazol-3-yl)urea; 2-((3R,4S)-3-(3,4-difluorophenyl)-4-(3-(2-phenyl-2,4,5,6-tetrahydrocyclopenta[c]pyrazol-3-yl)ureido)pyrrolidin-1-yl)-3-methoxypropanoic acid hydrochloride; 1-((3S,4R)-4-(3,4-difluorophenyl)-1-(1-hydroxy-3-methoxypropan-2-yl)pyrrolidin-3-yl)-3-(3-ethoxy-4-methyl-1-phenyl-1H-pyrazol-5-yl)urea; 1-(4-chloro-1′-methyl-1-phenyl-1H,1′H-3,4′-bipyrazol-5-yl)-3-((3S,4R)-4-(3,4-difluorophenyl)-1-(1-hydroxy-3-methoxypropan-2-yl)pyrrolidin-3-yl)urea; 1-((3S,4R)-4-(3,4-difluorophenyl)-1-(2-methoxyethyl)pyrrolidin-3yl)-3-(3-methoxy-1-phenyl-4-(trifluoromethyl)-1H-pyrazol-5-yl)urea; 1-(3-(2-fluoroethoxy)-4-methyl-1-phenyl-1H-pyrazol-5-yl)-3-((3R,4S)-4-phenyl-1-(2,2,2-trifluoroethyl)pyrrolidin-3-yl)urea; 1-(3-ethoxy-4-methyl-1-phenyl-1H-pyrazol-5-yl)-3-((3R,4S)-4-phenyl-1-(2,2,2-trifluoroethyl)pyrrolidin-3-yl)urea; 1-(3-(cyanomethoxy)-4-methyl-1-phenyl-1H-pyrazol-5-yl)-3-((3R,4S)-4-phenyl-1-(2,2,2-trifluoroethyl)pyrrolidin-3-yl)urea; 1-(1′,4-dimethyl-1-phenyl-1H,1′H-3,4′-bipyrazol-5-yl)-3-((3R,4S)-4-phenyl-1-(2,2,2-trifluoroethyl)pyrrolidin-3-yl)urea; 1-(4-chloro-1′-methyl-1-phenyl-1H,1′H-3,4′-bipyrazol-5-yl)-3-((3R,4S)-4-phenyl-1-(2,2,2-trifluoroethyl)pyrrolidin-3-yl)urea; 1-(3-(((S)-2,2-dimethyl-1,3-dioxolan-4-yl)methoxy)-4-methyl-1-phenyl-1H-pyrazol-5-yl)-3-((3R,4S)-4-phenyl-1-(2,2,2-trifluoroethyl)pyrrolidin-3-yl)urea; 1-(3-((R)-2,3-dihydroxypropoxy)-4-methyl-1-phenyl-1H-pyrazol-5-yl)-3-((3R,4S)-4-phenyl-1-(2,2,2-trifluoroethyl)pyrrolidin-3-yl)urea; (R,S)1-((2.alpha.,3.beta.,4.alpha.)-2-methyl-4-phenyl-1-(2,2,2-trifluoroethyl)pyrrolidin-3-yl)-3-(2-phenyl-2,4,5,6-tetrahydrocyclopenta[c]pyrazol-3-yl)urea; (R,S)-1-((3 (3,4.alpha.,5.alpha.)-5-methyl-4-phenyl-1-(2,2,2-trifluoroethyl)pyrrolidin-3-yl)-3-(2-phenyl-2,4,5,6-tetrahydrocyclopenta[c]pyrazol-3-yl)urea; 1-((3S,4R)-4-(3,4-difluorophenyl)-1-((S)-1,1,1-trifluoro-3-hydroxypropan-2-yl)pyrrolidin-3-yl)-3-(3-ethoxy-4-methyl-1-phenyl-1H-pyrazol-5-yl)urea; 1-((3S,4R)-4-(3,4-difluorophenyl)-1-((S)-1,1,1-trifluoro-3-methoxypropan2-yl)pyrrolidin-3-yl)-3-(3-ethoxy-4-methyl-1-phenyl-1H-pyrazol-5-yl)urea; 1-((3S,4R)-4-(3,4-difluorophenyl)-1-((S)-1,1,1-trifluoro-3-methoxypropan-2-yl)pyrrolidin-3-yl)-3-(3-((S)-2-hydroxypropoxy)-4-methyl-1-phenyl-1H-pyrazol-5-yl)urea; 1-(4-chloro-1′-methyl-1-phenyl-1H,1′H-3,4′-bipyrazol-5-yl)-3-((3S,4R)-4-(3,4-difluorophenyl)-1-((R)-1,1,1-trifluoro-3-methoxypropan-2-yl)pyrrolidin-3-yl)urea; 1-((3S,4R)-4-(3,4-difluorophenyl)-1-((R)-1,1,1-trifluoro-3-methoxypropan2-yl)pyrrolidin-3-yl)-3-(3-((S)-2-hydroxypropoxy)-4-methyl-1-phenyl-1H-pyrazol-5-yl)urea; 1-((3S,4R)-4-(3,4-difluorophenyl)-1-(2-methoxyethyl) pyrrolidin-3-yl)-3-(3-methyl-4-(methylthio)-1-phenyl-1H-pyrazol-5-yl)urea; 1-((3S,4R)-4-(3,5-difluorophenyl)-1-(2-methoxyethyl)pyrrolidin-3yl)-3-(3-(3-methoxypropyl)-4-methyl-1-phenyl-1H-pyrazol-5-yl)urea; 1-(3-(1,1-difluoro-2-hydroxyethyl)-4-methyl-1-phenyl-1H-pyrazol-5-yl)-3-((3S,4R)-4-(3,4-difluorophenyl)-1-(2-methoxyethyl)pyrrolidin-3-yl)urea; 1-(3-(1,1-difluoro-2-hydroxyethyl)-4-methyl-1-phenyl-1H-pyrazol-5yl)-3-((3S,4R)-4-(3,5-difluorophenyl)-1-(2-methoxyethyl)pyrrolidin-3-yl)urea; 1-(3-(1,1-difluoro-2-hydroxyethyl)-4-methyl-1-phenyl-1H-pyrazol-5-yl)-3-((3S,4R)-4-(4-fluorophenyl)-1-(2-methoxyethyl)pyrrolidin-3-yl)urea; 1-(3-(1,1-difluoro-2-hydroxyethyl)-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,5-difluorophenyl)-1-(2-methoxyethyl)pyrrolidin-3-yl)-3-(3-(2-hydroxyethyl)-4-methyl-1-phenyl-1H-pyrazol-5-yl)urea; 1-((3S,4R)-4-(4-fluorophenyl)-1-(2-methoxyethyl)pyrrolidin-3-yl)-3-(3-(2hydroxyethyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2-hydroxy-2-methylpropyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-((S)-2-hydroxypropyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-((R)-2-hydroxypropyl)-4-methyl-1-phenyl-1H-pyrazol-5-yl)urea; ethyl 5-(3-((3S,4R)-4-(3,4-difluorophenyl)-1-(2-methoxyethyl)pyrrolidin-3-yl)ureido)-4-methyl-1-phenyl-1H-pyrazole-3-carboxylate; 5-(3-((3S,4R)-4-(4-fluorophenyl)-1-(2-methoxyethyl)pyrrolidin-3-yl)ureido)-N,4-dimethyl-1-phenyl-1H-pyrazole-3-carboxamide; 1-(trans-4-(3-chloro-4-fluorophenyl)-1-(2-methoxyethyl)pyrrolidin-3-yl)-3-(1′,4-dimethyl-1-phenyl-1H,1′H-[3,4′-bipyrazol]-5-yl)urea; 1-(trans-4-(4-chloro-3-fluorophenyl)-1-(2-methoxyethyl)pyrrolidin-3-yl)-3-(1′,4-dimethyl-1-phenyl-1H,1′H-[3,4′-bipyrazol]-5-yl)urea; 1-(trans-4-(3-chloro-5-fluorophenyl)-1-(2-methoxyethyl)pyrrolidin-3-yl)-3-(1′,4-dimethyl-1-phenyl-1H,1′H-[3,4′-bipyrazol]-5-yl)urea; 1-(trans-4-(3-chlorophenyl)-1-(2-methoxyethyl)pyrrolidin-3-yl)-3-(1′,4-dimethyl-1-phenyl-1H,1′H-[3,4′-bipyrazol]-5-yl)urea; 1-((3S,4R)-4-(3,4-difluorophenyl)-1-(2-methoxyethyl)pyrrolidin-3-yl)-3-(4-methyl-3-(5-methyl-1,3,4-oxadiazol-2-yl)-1-phenyl-1H-pyrazol-5-yl)urea; 1-((3S,4R)-4-(3,4-difluorophenyl)-1-(2-methoxyethyl)pyrrolidin-3-yl)-3-(4-methyl-3-(3-methyl-1,2,4-oxadiazol-5-yl)-1-phenyl-1H-pyrazol-5-yl)urea; 1-((3S,4R)-4-(3,4-difluorophenyl)-1-(2-methoxyethyl)pyrrolidin-3-yl)-3-(4-methyl-1-phenyl-3-(3-(trifluoromethyl)-1,2,4-oxadiazol-5-yl)-1H-pyrazol-5-yl)urea; 1-((3S,4R)-4-(4-fluorophenyl)-1-(2-methoxyethyl)pyrrolidin-3-yl)-3-(4-methyl-1-phenyl-3-(3-(trifluoromethyl)-1,2,4-oxadiazol-5-yl)-1H-pyrazol-5-yl)urea; 5-(3-(trans-4-(3-chloro-4-fluorophenyl)-1-(2-methoxyethyl)pyrrolidin-3-yl)ureido)-N,4-dimethyl-1-phenyl-1H-pyrazole-3-carboxamide; 5-(3-(trans-4-(4-chloro-3-fluorophenyl)-1-(2-methoxyethyl)pyrrolidin-3-yl)ureido)-N,4-dimethyl-1-phenyl-1H-pyrazole-3-carboxamide; 1-(trans-4-(4-chloro-3-fluorophenyl)-1-(2-methoxyethyl)pyrrolidin-3-yl)-3-(4-methyl-3-(1-methyl-2-oxo-1,2-dihydropyridin-4-yl)-1-phenyl-1H-pyrazol5-yl)urea; 1-(trans-4-(3-chloro-4-fluorophenyl)-1-(2-methoxyethyl)pyrrolidin-3-yl)-3-(4-methyl-3-(1-methyl-2-oxo-1,2-dihydropyridin-4-yl)-1-phenyl-1H-pyrazol5-yl)urea; 1-((3S,4R)-4-(3,4-difluorophenyl)-1-(2-methoxyethyl)pyrrolidin-3-yl)-3-(1′,4,5′-trimethyl-1-phenyl-1H,1′H-[3,3′-bipyrazol]-5-yl)urea; 1-((3S,4R)-4-(3,5-difluorophenyl)-1-(2-methoxyethyl)pyrrolidin-3-yl)-3-(1′,4,5′-trimethyl-1-phenyl-1H,1′H-[3,3′-bipyrazol]-5-yl)urea; 1-((3S,4R)-4-(4-fluorophenyl)-1-(2-methoxyethyl)pyrrolidin-3-yl)-3-(1′,4,5′-trimethyl-1-phenyl-1H,1′H-[3,3′-bipyrazol]-5-yl)urea; 1-((3S,4R)-4-(3,4-difluorophenyl)-1-(2-methoxyethyl)pyrrolidin-3-yl)-3-(2′,4,5′-trimethyl-1-phenyl-1H,2′H-[3,3′-bipyrazol]-5-yl)urea; 1-(4-cyclopropyl-1′-methyl-1-phenyl-1H,1′H-[3,4′-bipyrazol]-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-isopropyl-1′-methyl-1-phenyl-1H,1′H-[3,4′-bipyrazol]-5-yl)urea; 1-((3S,4R)-4-(3,4-difluorophenyl)-1-(2-methoxyethyl)pyrrolidin-3-yl)-3-(4-ethyl-1′-methyl-1-phenyl-1H,1′H-[3,4′-bipyrazol]-5-yl)urea; 1-((3S,4R)-4-(3,4-difluorophenyl)-1-(2-methoxyethyl)pyrrolidin-3-yl)-3-(1-(4-fluorophenyl)-1′,4-dimethyl-1H,1′H-[3,4′-bipyrazol]-5-yl)urea; 1-((3S,4R)-4-(3,4-difluorophenyl)-1-(2-methoxyethyl)pyrrolidin-3-yl)-3-(1-(3-fluorophenyl)-1′,4-dimethyl-1H,1′H-[3,4′-bipyrazol]-5-yl)urea; 1-((3S,4R)-4-(3,4-difluorophenyl)-1-(2-methoxyethyl)pyrrolidin-3-yl)-3-(1-(2-fluorophenyl)-1′,4-dimethyl-1H,1′H-[3,4′-bipyrazol]-5-yl)urea; 1-((3S,4R)-4-(3,4-difluorophenyl)-1-(2-methoxyethyl)pyrrolidin-3-yl)-3-(1-(3-chlorophenyl)-1′,4-dimethyl-1H,1′H-[3,4′-bipyrazol]-5-yl)urea; 1-(1-(3-chloro-4-fluorophenyl)-1′,4-dimethyl-1H,1′H-[3,4′-bipyrazol]-5-yl)-3-((3S,4R)-4-(3,4-difluorophenyl)-1-(2-methoxyethyl)pyrrolidin-3-yl)urea; 1-(1-(3-chloro-2-fluorophenyl)-1′,4-dimethyl-1H,1′H-[3,4′-bipyrazol]-5-yl)-3-((3S,4R)-4-(3,4-difluorophenyl)-1-(2-methoxyethyl)pyrrolidin-3-yl)urea; 1-((3S,4R)-4-(4-fluorophenyl)-1-(2-methoxyethyl)pyrrolidin-3-yl)-3-(1-(4-fluorophenyl)-1′,4-dimethyl-1H,1′H-[3,4′-bipyrazol]-5-yl)urea; 1-((3S,4R)-4-(4-fluorophenyl)-1-(2-methoxyethyl)pyrrolidin-3-yl)-3-(1-(3-fluorophenyl)-1′,4-dimethyl-1H,1′H-[3,4′-bipyrazol]-5-yl)urea; 1-((3S,4R)-4-(4-fluorophenyl)-1-(2-methoxyethyl)pyrrolidin-3-yl)-3-(1-(2-fluorophenyl)-1′,4-dimethyl-1H,1′H-[3,4′-bipyrazol]-5-yl)urea; 1-((3S,4R)-4-(3,4-fluorophenyl)-1-(2-methoxyethyl)pyrrolidin-3-yl)-3-(1-(3-chlorophenyl)-1′,4-dimethyl-1H,1′H-[3,4′-bipyrazol]-5-yl)urea; 1-(1-(3-chloro-4-fluorophenyl)-1′,4-dimethyl-1H,1′H-[3,4′-bipyrazol]-5-yl)-3-((3S,4R)-4-(4-difluorophenyl)-1-(2-methoxyethyl)pyrrolidin-3-yl)urea; 1-(1-(3-chloro-2-fluorophenyl)-1′,4-dimethyl-1H,1′H-[3,4′-bipyrazol]-5-yl)-3-((3S,4R)-4-(4-difluorophenyl)-1-(2-methoxyethyl)pyrrolidin-3-yl)urea; 1-((3S,4R)-4-(2,4-difluorophenyl)-1-(2-methoxyethyl)pyrrolidin-3-yl)-3-(1′,4-dimethyl-1-phenyl-1H,1′H-[3,4′-bipyrazol]-5-yl)urea; 1-((3S,4R)-4-(3-cyanophenyl)-1-(2-methoxyethyl)pyrrolidin-3-yl)-3-(1′,4-dimethyl-1-phenyl-1H,1′H-[3,4′-bipyrazol]-5-yl)urea; 1-((3S,4R)-4-(4-cyanophenyl)-1-(2-methoxyethyl)pyrrolidin-3-yl)-3-(1′,4-dimethyl-1-phenyl-1H,1′H-[3,4′-bipyrazol]-5-yl)urea; 1-(1′,4-dimethyl-1-phenyl-1H,1′H-[3,4′-bipyrazol]-5-yl)-3-((3S,4R)-1-(2-methoxyethyl)-4-(p-tolyl)pyrrolidin-3-yl)urea; 1-((3S,4R)-4-(3,4-difluorophenyl)-1-(2-methoxyethyl)pyrrolidin-3-yl)-3-(4-methyl-1,3-diphenyl-1H-pyrazol-5-yl)urea; 1-((3S,4R)-4-(3,5-difluorophenyl)-1-(2-methoxyethyl)pyrrolidin-3-yl)-3-(4-methyl-1,3-diphenyl-1H-pyrazol-5-yl)urea; 1-((3S,4R)-4-(3,4,5-trifluorophenyl)-1-(2-methoxyethyl)pyrrolidin-3-yl)-3-(4-methyl-1,3-diphenyl-1H-pyrazol-5-yl)urea; 1-((3S,4R)-4-(4-fluorophenyl)-1-(2-methoxyethyl)pyrrolidin-3-yl)-3-(4-methyl-1,3-diphenyl-1H-pyrazol-5-yl)urea; 1-(4-bromo-3-methyl-1-phenyl-1H-pyrazol-5-yl)-3-trans-1-(2-methoxyethyl)4-(1-methyl-1H-pyrazol-4-yl)pyrrolidin-3-yl)urea; 1-(1′,4-dimethyl-1-phenyl-1H,1′H-[3,4′-bipyrazol]-5-yl)-3-(trans-1-(2-methoxyethyl)-4-(1-methyl-1H-pyrazol-4-yl)pyrrolidin-3-yl)urea; 1-(1′,4-dimethyl-1-phenyl-1H,1′H-[3,4′-bipyrazol]-5-yl)-3-((trans-1-(2-methoxyethyl)-4-(1,2,3-thiadiazol-4-yl)pyrrolidin-3-yl)urea; 1-(3-ethoxy-4-methyl-1-phenyl-1H-pyrazol-5-yl)-3-(trans-1-(2-methoxyethyl)-4-(3-(trifluoromethyl)phenyl)pyrroli-din-3-yl)urea; 1-(3,4-dimethyl-1-phenyl-1H-pyrazol-5-yl)-3-((3S,4R)-1-(2-methoxyethyl)-4-(3-(trifluoromethyl)phenyl)pyrrol-idin-3-yl)urea; 1-((3S,4R)-4-(5-fluoropyridin-3-yl)-1-(2-methoxyethyl)pyrrolidin-3-yl)-3(2-phenyl-2,4,5,6-tetrahydrocyclopenta[c]pyrazol-3-yl)urea; 1-((3R,4S)-4-(5-fluoropyridin-3-yl)-1-(2-methoxyethyl)pyrrolidin-3-yl)-3(2-phenyl-2,4,5,6-tetrahydrocyclopenta[c]pyrazol-3-yl)urea; 1-(3-(2-fluoroethoxy)-4-methyl-1-phenyl-1H-pyrazol-5-yl)-3-((3S,4R)-4-(5fluoropyridin-3-yl)-1-(2-methoxyethyl)pyrrolidin-3-yl)urea; 1-(3-(2-fluoroethoxy)-4-methyl-1-phenyl-1H-pyrazol-5-yl)-3-((3R,4S)-4-(5fluoropyridin-3-yl)-1-(2-methoxyethyl)pyrrolidin-3-yl)urea; 1-(trans-4-(5-fluoropyridin-3-yl)-1-(2-methoxyethyl)pyrrolidin-3-yl)-3-(2-phenyl-2,4,5,6-tetrahydrocyclopenta[c]pyrazol-3-yl)urea; 1-(trans-4-(5-chloropyridin-3-yl)-1-(2-methoxyethyl) pyrrolidin-3-yl)-3-(3-ethoxy-4-methyl-1-phenyl-1H-pyrazol-5-yl)urea; 1-(trans-4-(5-chloropyridin-3-yl)-1-(2-methoxyethyl)pyrrolidin-3-yl)-3-(3,4-dimethyl-1-phenyl-1H-pyrazol-5-yl)urea; 1-(trans-4-(5-fluoropyridin-3-yl)-1-(2-methoxyethyl)pyrrolidin-3-yl)-3-(4-methyl-1,3-diphenyl-1H-pyrazol-5-yl)urea; 1-(1′,4-dimethyl-1-phenyl-1H,1′H-[3,4′-bipyrazol]-5-yl)-3-(trans-4-(5-fluoropyridin-2-yl)-1-(2-methoxyethyl)pyrrolidin-3-yl)urea; 1-(1′,4-dimethyl-1-phenyl-1H,1′H-[3,4′-bipyrazol]-5-yl)-3-((3S,4R)-4-(3-fluoropyridin-4-yl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-methyl-3-(1-methyl-1H-1,2,4-triazol-3-yl)-1-phenyl-1H-pyrazol-5-yl)urea; 1-((3S,4R)-4-(3,4-difluorophenyl)-1-(2-methoxyethyl)pyrrolidin-3-yl)-3-(1′-(2-methoxyethyl)-4-methyl-1-phenyl-1H,1′H-[3,4′-bipyrazol]-5-yl)urea; 1-(3-cyano-4-methyl-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(1′-(2-hydroxyethyl)-4-methyl-1-phenyl-1H,1′H-[3,4′-bipyrazol]-5-yl)urea; 1-((3S,4R)-4-(3,4-difluorophenyl)-1-(2-methoxyethyl)pyrrolidin-3-yl)-3-(4-methyl-3-(2-methyl-2H-1,2,3-triazol-4-yl)-1-phenyl-1H-pyrazol-5-yl)urea; 1-(3-bromo-4-methyl-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(5-methyl-6-oxo-2-phenyl-2,4,5,6-tetrahydropyrrolo[3,4-c]pyrazol-3-yl)urea; 1-((3S,4R)-4-(3,5-difluorophenyl)-1-(2-methoxyethyl)pyrrolidin-3-yl)-3-(5-methyl-6-oxo-2-phenyl-2,4,5,6-tetrahydropyrrolo[3,4-c]pyrazol-3-yl)urea; 1-((3S,4R)-4-(3,4-difluorophenyl)-1-(2-methoxyethyl)pyrrolidin-3-yl)-3-(4-methyl-3-((5-methyl-1,3,4-oxadiazol-2-yl)methoxy)-1-phenyl-1H-pyrazol-5-yl)urea; 1-(4-chloro-3-ethoxy-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-ethoxy-4-fluoro-1-phenyl-1H-pyrazol-5-yl)urea; 1-(4-bromo-3-(2-hydroxy-2-methylpropoxy)-1-phenyl-1H-pyrazol-5-yl)-3-((3S,4R)-4-(3,4-difluorophenyl)-1-(2-methoxyethyl)pyrrolidin-3-yl)urea; 1-(4-chloro-3-(2-hydroxy-2-methylpropoxy)-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-((S)-2-hydroxybutoxy)-4-methyl-1-phenyl-1H-pyrazol-5-yl)urea; ethyl 2-((5-(3-((3S,4R)-4-(3,4-difluorophenyl)-1-(2-methoxyethyl)pyrrolidin-3-yl)ureido)-4-methyl-1-phenyl-1H-pyrazol-3-yl)oxy)acetate; 1-((3S,4R)-4-(4-fluorophenyl)-1-(2-methoxyethyl)pyrrolidin-3-yl)-3-(3-(2hydroxy-2-methylpropoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2-hydroxy-2-methylpropoxy)-4-methyl-1-phenyl-1H-pyrazol-5-yl)urea; 1-((3S,4R)-4-(4-fluorophenyl)-1-(2-methoxyethyl)pyrrolidin-3-yl)-3-(3-((R)-2-hydroxypropoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-((R)-2-hydroxypropoxy)-4-methyl-1-phenyl-1H-pyrazol-5-yl)urea; 1-((3S,4R)-4-(3,5-difluorophenyl)-1-(2-methoxyethyl)pyrrolidin-3-yl)-3-(3-((R)-2-hydroxypropoxy)-4-methyl-1-phenyl-1H-pyrazol-5-yl)urea; 1-((3S,4R)-4-(3,5-difluorophenyl)-1-(2-methoxyethyl)pyrrolidin-3-yl)-3-(4-methyl-1-phenyl-3-((R)-3,3,3-trifluoro-2-hydroxypropoxy)-1H-pyrazol-5-yl)urea; 1-((3S,4R)-4-(4-fluorophenyl)-1-(2-methoxyethyl)pyrrolidin-3-yl)-3-(3-((S)-2-hydroxypropoxy)-4-methyl-1-phenyl-1H-pyrazol-5-yl)urea; 1-((3S,4R)-4-(3,5-difluorophenyl)-1-(2-methoxyethyl)pyrrolidin-3-yl)-3-(4-methyl-1-phenyl-3-((S)-3,3,3-trifluoro-2-hydroxypropoxy)-1H-pyrazol-5-yl)urea; 1-(4-chloro-3-(2-hydroxy-2-methylpropoxy)-1-phenyl-1H-pyrazol-5-yl)-3-((3S,4R)-4-(3,5-difluorophenyl)-1-(2-methoxyethyl)pyrrolidin-3-yl)urea; 1-(4-chloro-3-(2-hydroxy-2-methylpropoxy)-1-phenyl-1H-pyrazol-5-yl)-3-((3S,4R)-4-(4-fluorophenyl)-1-(2-methoxyethyl)pyrrolidin-3-yl)urea; 1-(4-chloro-3-((R)-2-hydroxypropoxy)-1-phenyl-1H-pyrazol-5-yl)-3-((3S,4R)-4-(3,4-difluorophenyl)-1-(2-methoxyethyl)pyrrolidin-3-yl)urea; 1-(4-chloro-3-((R)-2-hydroxypropoxy)-1-phenyl-1H-pyrazol-5-yl)-3-((3S,4R)-4-(3,5-difluorophenyl)-1-(2-methoxyethyl)pyrrolidin-3-yl)urea; 1-(4-chloro-3-((R)-2-hydroxypropoxy)-1-phenyl-1H-pyrazol-5-yl)-3-((3S,4R)-4-(4-fluorophenyl)-1-(2-methoxyethyl)pyrrolidin-3-yl)urea; 1-(4-bromo-3-((R)-2-hydroxypropoxy)-1-phenyl-1H-pyrazol-5-yl)-3-((3S,4R)4-(3,4-difluorophenyl)-1-(2-methoxyethyl)pyrrolidin-3-yl)urea; 1-(4-bromo-3-((R)-2-hydroxypropoxy)-1-phenyl-1H-pyrazol-5-yl)-3-((3S,4R)4-(3,5-difluorophenyl)-1-(2-methoxyethyl)pyrrolidin-3-yl)urea; 1-(4-bromo-3-((R)-2-hydroxypropoxy)-1-phenyl-1H-pyrazol-5-yl)-3-((3S,4R)4-(4-fluorophenyl)-1-(2-methoxyethyl)pyrrolidin-3-yl)urea; 1-((3S,4R)-4-(3,5-difluorophenyl)-1-(2-methoxyethyl)pyrrolidin-3-yl)-3-(3-((R)-2-hydroxybutoxy)-4-methyl-1-phenyl-1H-pyrazol-5-yl)urea; 1-((3S,4R)-4-(4-fluorophenyl)-1-(2-methoxyethyl)pyrrolidin-3-yl)-3-(3-((R)-2-hydroxybutoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-((R)-2-hydroxybutoxy)-4-methyl-1-phenyl-1H-pyrazol-5-yl)urea; ethyl 4-bromo-5-(3-((3S,4R)-4-(3,4-difluorophenyl)-1-(2-methoxyethyl)pyrrolidin-3-yl)ureido)-1-phenyl-1H-pyrazole-3-carboxylate; 1-((3S,4R)-4-(4-fluorophenyl)-1-(2-methoxyethyl)pyrrolidin-3-yl)-3-(1′-(2-methoxyethyl)-4-methyl-1-phenyl-1H,1′H-[3,4′-bipyrazol]-5-yl)urea; 1-((3S,4R)-4-(3-fluorophenyl)-1-(2-methoxyethyl)pyrrolidin-3-yl)-3-(1′-(2-methoxyethyl)-4-methyl-1-phenyl-1H,1′H-[3,4′-bipyrazol]-5-yl)urea; 1-((3S,4R)-4-(3-fluorophenyl)-1-(2-methoxyethyl)pyrrolidin-3-yl)-3-(4-methyl-3-(2-methyl-2H-1,2,3-triazol-4-yl)-1-phenyl-1H-pyrazol-5-yl)urea; 1-((3S,4R)-4-(4-fluorophenyl)-1-(2-methoxyethyl)pyrrolidin-3-yl)-3-(4-methyl-3-(2-methyl-2H-1,2,3-triazol-4-yl)-1-phenyl-1H-pyrazol-5-yl)urea; 1-((3S,4R)-4-(3,4-difluorophenyl)-1-(2-methoxyethyl)pyrrolidin-3-yl)-3-(4-methyl-3-(2-morpholinoethoxy)-1-phenyl-1H-pyrazol-5-yl)urea; 1-((3S,4R)-4-(3,4-difluorophenyl)-1-(2-methoxyethyl)pyrrolidin-3-yl)-3-(3-(2-(1,3-dioxoisoindolin-2-yl)ethoxy)-4-methyl-1-phenyl-1H-pyrazol5-yl)urea; tert-butyl 4-(2-((5-(3-((3S,4R)-4-(3,4-difluorophenyl)-1-(2-methoxyethyl)pyrrolidin3-yl)ureido)-4-methyl-1-phenyl-1H-pyrazol-3-yl)oxy)ethyl)piperazine-1-carboxylate; Trans-1-(2-methoxyethyl)-4-phenylpyrrolidin-3-yl)-3-(2-phenyl-2H-indazol3-yl)urea; 1-((3S,4R)-4-(3,4-difluorophenyl)-1-(2-methoxyethyl)pyrrolidin-3-yl)-3-(2-phenyl-2H-indazol-3-yl)urea; 1-((3S,4R)-4-(3,4-difluorophenyl)-1-(2-methoxyethyl)pyrrolidin-3-yl)-3-(4-methyl-3-((1-methyl-1H-1,2,4-triazol-3-yl)methoxy)-1-phenyl-1H-pyrazol-5yl)urea; 1-((3S,4R)-4-(3,5-difluorophenyl)-1-(2-methoxyethyl)pyrrolidin-3-yl)-3-(3-((R)-2,2-dimethyl-1,3-dioxolan-4-yl)methoxy)-4-methyl-1-phenyl-1H-pyrazol-5-yl)urea; 1-((3S,4R)-4-(3,5-difluorophenyl)-1-(2-methoxyethyl)pyrrolidin-3-yl)-3-(3-(((S)-2,2-dimethyl-1,3-dioxolan-4-yl)methoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-ethoxy-4-methyl-1-(pyrazin-2-yl)-1H-pyrazol-5-yl)urea; 1-((3S,4R)-4-(3,4-difluorophenyl)-1-(2-methoxyethyl)pyrrolidin-3-yl)-3-(2-(pyridazin-4-yl)-2,4,5,6-tetrahydrocyclopenta[c]pyrazol-3-yl)urea; 5-(3-((3S,4R)-4-(3,4-difluorophenyl)-1-(2-methoxyethyl)pyrrolidin-3-yl)ureido)-4-methyl-1-phenyl-1H-pyrazol-3-yl dimethylcarbamate; 5-(3-((3S,4R)-4-(3,4-difluorophenyl)-1-(2-methoxyethyl)pyrrolidin-3-yl)ureido)-4-methyl-1-phenyl-1H-pyrazol-3-yl morpholine-4-carboxylate; 1-(3-(((S)-2,2-dimethyl-1,3-dioxolan-4-yl)methoxy)-4-methyl-1-phenyl-1H-pyrazol-5-yl)-3-((3S,4R)-1-(2-methoxyethyl)-4-(3,4,5-trifluorophenyl)pyrrolidin-3-yl)urea; 1-(3-(((R)-2,2-dimethyl-1,3-dioxolan-4-yl)methoxy)-4-methyl-1-phenyl-1H-pyrazol-5-yl)-3-((3S,4R)-1-(2-methoxyethyl)-4-(3,4,5-trifluorophenyl)pyrrolidin-3-yl)urea; 1-(3-((S)-2-(tert-butyldimethyl silyloxy)propoxy)-4-methyl-1-phenyl-1-pyrazol-5-yl)-3-((3S,4R)-1-(2-methoxyethyl)-4-(3,4,5-trifluorophenyl)pyrrolidin-3-yl)urea; 1-(3-(2-hydroxy-2-methylpropoxy)-4-methyl-1-phenyl-1H-pyrazol-5-yl)-3-((3S,4R)-1-(2-methoxyethyl)-4-(3,4,5-trifluorophenyl)pyrrolidin-3-yl)urea; 1-(3-((S)-2-(tert-butyldimethylsilyloxy)-3-methoxypropoxy)-4-methyl-1-phenyl-1H-pyrazol-5-yl)-3-((3S,4R)-4-(3,4-difluorophenyl)-1-(2-methoxyethyl)pyrrolidin-3-yl)urea; 1-(4-chloro-3-(methoxy-methyl)-1-phenyl-1H-pyrazol-5-yl)-3-((3S,4R)-4-(3,4-difluorophenyl)-1-(2-methoxyethyl)pyrrolidin-3-yl)urea; 1-(4-bromo-3-(methoxy-methyl)-1-phenyl-1H-pyrazol-5-yl)-3-((3S,4R)-4-(3,4-difluorophenyl)-1-(2-methoxyethyl)pyrrolidin-3-yl)urea; 1-(4-chloro-3-(methoxy-methyl)-1-phenyl-1H-pyrazol-5-yl)-3-((3S,4R)-4-(3,5-difluorophenyl)-1-(2-methoxyethyl)pyrrolidin-3-yl)urea; 1-(4-bromo-3-(methoxy-methyl)-1-phenyl-1H-pyrazol-5-yl)-3-((3S,4R)-4-(3,5-difluorophenyl)-1-(2-methoxyethyl)pyrrolidin-3-yl)urea; 1-(4-chloro-3-(methoxy-methyl)-1-phenyl-1H-pyrazol-5-yl)-3-((3S,4R)-4-(4fluorophenyl)-1-(2-methoxyethyl)pyrrolidin-3-yl)urea; 1-(4-bromo-3-(methoxy-methyl)-1-phenyl-1H-pyrazol-5-yl)-3-((3S,4R)-4-(4-fluorophenyl)-1-(2-methoxyethyl)pyrrolidin-3-yl)urea; 1-(4-chloro-3-(1,1-difluoro-2-hydroxyethyl)-1-phenyl-1H-pyrazol-5-yl)-3-((3S,4R)-4-(3,4-difluorophenyl)-1-(2-methoxyethyl)pyrrolidin-3-yl)urea; 1-(4-chloro-3-(1,1-difluoro-2-hydroxyethyl)-1-phenyl-1H-pyrazol-5yl)-3-((3S,4R)-4-(3,5-difluorophenyl)-1-(2-methoxyethyl)pyrrolidin-3-yl)urea; 1-(4-chloro-3-(1,1-difluoro-2-hydroxyethyl)-1-phenyl-1H-pyrazol-5-yl)-3-((3S,4R)-4-(4-fluorophenyl)-1-(2-methoxyethyl)pyrrolidin-3-yl)urea; 1-(4-chloro-3-((S)-2-hydroxypropyl)-1-phenyl-1H-pyrazol-5-yl)-3((3S,4R)-4-(3,4-difluorophenyl)-1-(2-methoxyethyl)pyrrolidin-3-yl)urea; 1-(4-chloro-3-((R)-2-hydroxypropyl)-1-phenyl-1H-pyrazol-5-yl)-3-((3S,4R)-4-(3,4-difluorophenyl)-1-(2-methoxyethyl)pyrrolidin-3-yl)urea; 1-(4-bromo-3-((R)-2-hydroxypropyl)-1-phenyl-1H-pyrazol-5-yl)-3-((3S,4R)-4-(3,4-difluorophenyl)-1-(2-methoxyethyl)pyrrolidin-3-yl)urea; 1-(4-chloro-3-(2-hydroxy-2-methylpropyl)-1-phenyl-1H-pyrazol-5-yl)-3-((3S,4R)-4-(3,4-difluorophenyl)-1-(2-methoxyethyl)pyrrolidin-3-yl)urea; 1-(4-chloro-3-(3-methyl-1,2,4-oxadiazol-5-yl)-1-phenyl-1H-pyrazol5-yl)-3-((3S,4R)-4-(3,4-difluorophenyl)-1-(2-methoxyethyl)pyrrolidin-3-yl)urea; 1-(4-bromo-3-(2-cyanopropan-2-yl)-1-phenyl-1H-pyrazol-5-yl)-3-((3S,4R)-4(3,4-difluorophenyl)-1-(2-methoxyethyl)pyrrolidin-3-yl)urea; 1-(4-chloro-3-(2-cyanopropan-2-yl)-1-phenyl-1H-pyrazol-5-yl)-3-((3S,4R)-4-(3,4-difluorophenyl)-1-(2-methoxyethyl)pyrrolidin-3-yl)urea; 1-(4-bromo-1′-methyl-1-phenyl-1H,1′H-[3,4′-bipyrazol]-5-yl)-3-((3S,4R)-4(3,5-difluorophenyl)-1-(2-methoxyethyl)pyrrolidin-3-yl)urea; 1-(4-bromo-1′-methyl-1-phenyl-1H,1′H-[3,4′-bipyrazol]-5-yl)-3-((3S,4R)-4(3,4-difluorophenyl)-1-(2-methoxyethyl)pyrrolidin-3-yl)urea; 1-(4-bromo-1′-methyl-1-phenyl-1H,1′H-[3,4′-bipyrazol]-5-yl)-3-((3-4-(4-difluorophenyl)-1-(2-methoxyethyl)pyrrolidin-3-yl)urea; 1-(4-chloro-1′-methyl-1-phenyl-1H,1′H-[3,4′-bipyrazol]-5-yl)-3-((3S,4R)-4-(3,5-difluorophenyl)-1-(2-methoxyethyl)pyrrolidin-3-yl)urea; 1-(4-chloro-1′-methyl-1-phenyl-1H,1′H-[3,4′-bipyrazol]-5-yl)-3-((3S,4R)-4-(3,4-difluorophenyl)-1-(2-methoxyethyl)pyrrolidin-3-yl)urea; 1-(4-chloro-1′-methyl-1-phenyl-1H,1′H-[3,4′-bipyrazol]-5-yl)-3-((3S,4R)-4-(4-fluorophenyl)-1-(2-methoxyethyl)pyrrolidin-3-yl)urea; 1-(4-chloro-1′-methyl-1-phenyl-1H,1′H-[3,4′-bipyrazol]-5-yl)-3-((3S,4R)-4-phenyl-1-(2-methoxyethyl)pyrrolidin-3-yl)urea; 1-(4-chloro-1,3-diphenyl-1H-pyrazol-5-yl)-3-((3S,4R)-4-(3,5-difluorophenyl)-1-(2-methoxyethyl)pyrrolidin-3-yl)urea; 1-(4-bromo-1,3-diphenyl-1H-pyrazol-5-yl)-3-((3S,4R)-4-(3,5-difluorophenyl)-1-(2-methoxyethyl)pyrrolidin-3-yl)urea; 1-(4-bromo-1,3-diphenyl-1H-pyrazol-5-yl)-3-((3S,4R)-4-(4-fluorophenyl)-1(2-methoxyethyl)pyrrolidin-3-yl)urea; 1-(4-chloro-3-methyl-1-phenyl-1H-pyrazol-5-yl)-3-((3S,4R)-4-(3,5-difluorophenyl)-1-(2-methoxyethyl)pyrrolidin-3-yl)urea; 1-(4-bromo-3-methyl-1-phenyl-1H-pyrazol-5-yl)-3-((3S,4R)-4-(3,5-difluorophenyl)-1-(2-methoxyethyl)pyrrolidin-3-yl)urea; 1-(4-chloro-1′-methyl-1-phenyl-1H,1′H-[3,4′-bipyrazol]-5-yl)-3-((3S,4R)-4-(5-fluoropyridin-3-yl)-1-(2-methoxyethyl)pyrrolidin-3-yl)urea; 1-(4-fluoro-1′-methyl-1-phenyl-1H,1′H-[3,4′-bipyrazol]-5-yl)-3-((3S,4R)-4-(5-fluoropyridin-3-yl)-1-(2-methoxyethyl)pyrrolidin-3-yl)urea; 1-(4-bromo-3-methyl-1-phenyl-1H-pyrazol-5-yl)-3-trans-4-(5-fluoropyridin-3-yl)-1-(2-methoxyethyl)pyrrolidin-3-yl)urea; 1-(4-bromo-3-methyl-1-phenyl-1H-pyrazol-5-yl)-3-trans-4-(5-fluoropyridin3-yl)-1-(2-methoxyethyl)pyrrolidin-3-yl)urea; 1-(4-bromo-1,3-diphenyl-1H-pyrazol-5-yl)-3-(trans-4-(5-fluoropyridin-3-yl)-1-(2-methoxyethyl)pyrrolidin-3-yl)urea; 1-(4-chloro-1,3-diphenyl-1H-pyrazol-5-yl)-3-(trans-4-(5-fluoropyridin-3-yl)-1-(2-methoxyethyl)pyrrolidin-3-yl)urea; 1-(4-bromo-3-methyl-1-phenyl-1H-pyrazol-5-yl)-3-(trans-4-(5-fluoropyridin-2-yl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-fluoro-1′-methyl-1-phenyl-1H,1′H-[3,4′-bipyrazol]-5-yl)urea; 1-((3S,4R)-4-(3,5-difluorophenyl)-1-(2-methoxyethyl)pyrrolidin-3-yl)-3-(4-fluoro-1′-methyl-1-phenyl-1H,1′H-[3,4′-bipyrazol]-5-yl)urea; 1-(4-bromo-1′-methyl-1-phenyl-1H,1′H-[3,4′-bipyrazol]-5-yl)-3-((3S,4R)-4(5-fluoropyridin-3-yl)-1-(2-methoxyethyl)pyrrolidin-3-yl)urea; 1-(1′,4-dimethyl-1-phenyl-1H,1′H-[3,4′-bipyrazol]-5-yl)-3-((3S,4R)-4-(4-fluorophenyl)-1-(2-methoxyethyl)pyrrolidin-3-yl)urea; 1-(1′,4-dimethyl-1-phenyl-1H,1′H-[3,4′-bipyrazol]-5-yl)-3-((3S,4R)-4-(3,5-difluorophenyl)-1-(2-methoxyethyl)pyrrolidin-3-yl)urea; 1-(1′,4-dimethyl-1-phenyl-1H,1′H-[3,4′-bipyrazol]-5-yl)-3-((3S,4R)-4-(3,4,5-trifluorophenyl)-1-(2-methoxyethyl)pyrrolidin-3-yl)urea; 1-(1′,4-dimethyl-1-phenyl-1H,1′H-[3,4′-bipyrazol]-5-yl)-3-((3S,4R)-4-(3,4-difluorophenyl)-1-(2-methoxyethyl)pyrrolidin-3-yl)urea; 1-(1′,4-dimethyl-1-phenyl-1H,1′H-[3,4′-bipyrazol]-5-yl)-3-((3S,4R)-4-(fluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(1′-(4-methoxybenzyl)-4-methyl-1-phenyl-1H,1′H-[3,4′-bipyrazol]-5-yl)urea; 1-((3S,4R)-4-(3,4-difluorophenyl)-1-(2-methoxyethyl)pyrrolidin-3-yl)-3-(1′-(4-methoxybenzyl)-4-methyl-1-phenyl-1H,1′H-[3,4′-bipyrazol]-5-yl)urea trifluoroacetate; 2-(4-chloro-5-(3-((3S,4R)-4-(3,4-difluorophenyl)-1-(2-methoxyethyl)pyrrolidin-3-yl)ureido)-1-phenyl-1H-pyrazol-3-yl)ethyl acetate; 1-(4-chloro-3-(2-hydroxyethyl)-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-(cis-3-hydroxycyclobutyl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(trans-3-hydroxycyclobutyl)-4-methyl-1-phenyl-1H-pyrazol-5-yl)urea; 1-(4-chloro-3-(cis-3-hydroxycyclobutyl)-1-phenyl-1H-pyrazol-5-yl)3-((3S,4R)-4-(3,4-difluorophenyl)-1-(2-methoxyethyl)pyrrolidin-3-yl)urea; 1-(4-chloro-3-((1r,3S)-3-hydroxycyclobutyl)-1-phenyl-1H-pyrazol-5yl)-3-(trans-4-(3,4-difluorophenyl)-1-(2-methoxyethyl)pyrrolidin-3-yl)urea; 1-((3S,4R)-4-(4-fluorophenyl)-1-(2-methoxyethyl)pyrrolidin-3-yl)3-(3-(cis-3-hydroxycyclobutyl)-4-methyl-1-phenyl-1H-pyrazol-5-yl)urea; 1-((3S,4R)-4-(4-fluorophenyl)-1-(2-methoxyethyl)pyrrolidin-3-yl)-3-(3-(trans-3-hydroxycyclobutyl)-4-methyl-1-phenyl-1H-pyrazol-5-yl)urea; 1-((3S,4R)-4-(3,5-difluorophenyl)-1-(2-methoxyethyl)pyrrolidin-3-yl)-3-(3-(cis-3-hydroxycyclobutyl)-4-methyl-1-phenyl-1H-pyrazol-5-yl)urea; 1-((3S,4R)-4-(3,5-difluorophenyl)-1-(2-methoxyethyl)pyrrolidin-3-yl)-3-(3-(trans-3-hydroxycyclobutyl)-4-methyl-1-phenyl-1H-pyrazol-5-yl)urea; 5-(3-((3S,4R)-4-(3,4-difluorophenyl)-1-(2-methoxyethyl)pyrrolidin-3-yl)ureido)-4-methyl-1-phenyl-1H-pyrazole-3-carboxylic acid; 5-(3-((3S,4R)-4-(3,4-difluorophenyl)-1-(2-methoxyethyl)pyrrolidin-3-yl)ureido)-N,4-dimethyl-1-phenyl-1H-pyrazole-3-carboxamide; 5-(3-((3S,4R)-4-(3,4-difluorophenyl)-1-(2-methoxyethyl)pyrrolidin-3-yl)ureido)-N,N,4-trimethyl-1-phenyl-1H-pyrazole-3-carboxamide; 5-(3-((3S,4R)-4-(3,4-difluorophenyl)-1-(2-methoxyethyl)pyrrolidin-3-yl)ureido)-N-ethyl-4-methyl-1-phenyl-1H-pyrazole-3-carboxamide; 5-(3-((3S,4R)-4-(3,4-difluorophenyl)-1-(2-methoxyethyl)pyrrolidin-3-yl)ureido)-N-isopropyl-4-methyl-1-phenyl-1H-pyrazole-3-carboxamide; 5-(3-((3S,4R)-4-(3,4-difluorophenyl)-1-(2-methoxyethyl)pyrrolidin-3-yl)ureido)-4-methyl-1-phenyl-1H-pyrazole-3-carboxamide; 1-((3S,4R)-4-(3,5-difluorophenyl)-1-(2-methoxyethyl)pyrrolidin-3-yl)-3-(3-ethoxy-4-(hydroxymethyl)-1-phenyl-1H-pyrazol-5-yl)urea; 1-((3S,4R)-4-(3-chloro-4-fluorophenyl)-1-(2-methoxyethyl)pyrrolidin-3-yl)-3-(1′,4-dimethyl-1-phenyl-1H,1′H-3,4′-bipyrazol-5-yl)urea; 1-((3S,4R)-4-(4-chloro-3-fluorophenyl)-1-(2-methoxyethyl)pyrrolidin-3-yl)-3-(1′,4-dimethyl-1-phenyl-1H,1′H-3,4′-bipyrazol-5-yl)urea; 1-((3S,4R)-4-(3-chloro-5-fluorophenyl)-1-(2-methoxyethyl)pyrrolidin-3-yl)-3-(1′,4-dimethyl-1-phenyl-1H,1′H-3,4′-bipyrazol-5-yl)urea; 2-((3R,4S)-3-(3,4-difluorophenyl)-4-(3-(3-ethoxy-4-methyl-1-phenyl-1H-pyrazol-5-yl)ureido)pyrrolidin-1-yl)acetate; 1-((3S,4R)-4-(3,4-difluorophenyl)-1-(3,3,3-trifluoro-2-hydroxypropyl)pyrrolidin-3-yl)-3-(3-ethoxy-4-methyl-1-phenyl-1H-pyrazol-5-yl)urea; 1-((3S,4R)-4-(3,4-difluorophenyl)-1-(2-hydroxypropyl)pyrrolidin-3-yl)-3-(3-ethoxy-4-methyl-1-phenyl-1H-pyrazol-5-yl)urea; 1-((3S,4R)-1-(2-cyanoethyl)-4-(3,4-difluorophenyl)pyrrolidin-3-yl)-3-(3-ethoxy-4-methyl-1-phenyl-1H-pyrazol-5-yl)urea; 2-((3R,4S)-3-(3,4-difluorophenyl)-4-(3-(3-ethoxy-4-methy-1-phenyl-1H-pyrazol-5-yl)ureido)pyrrolidin-1-yl)-N-methylacetamide; 1-(1-cyclohexyl-3,4-dimethyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-(3-hydroxy-2-(hydroxymethyl)propoxy)-4-methyl-1-phenyl-1H-pyrazol-5-yl)urea; 1-((3S,4R)-4-(3,4-difluorophenyl)-1-(2-methoxyethyl)pyrrolidin3-yl)-3-(4-methyl-1-phenyl-3-(2,2,2-trifluoroethoxy)-1H-pyrazol-5-yl)urea; 1-((3S,4R)-4-(4-fluorophenyl)-1-(2-methoxyethyl)pyrrolidin-3-yl)-3-(4-methyl-1-phenyl-3-(2,2,2-trifluoroethoxy)-1H-pyrazol-5-yl)urea; 1-((3S,4R)-4-(3,5-difluorophenyl)-1-(2-methoxyethyl)pyrrolidin-3-yl)-3-(4-methyl-1-phenyl-3-(2,2,2-trifluoroethoxy)-1H-pyrazol-5-yl)urea; 1-(3-(2,2-difluoroethoxy)-4-methyl-1-phenyl-1H-pyrazol-5-yl)-3-((3S,4R)-4-(3,4-difluorophenyl)-1-(2-methoxyethyl)pyrrolidin-3-yl)urea; 1-(4-chloro-1-phenyl-3-(2,2,2-trifluoroethoxy)-1H-pyrazol-5-yl)-3-((3S,4R)-4-(3,4-difluorophenyl)-1-(2-methoxyethyl)pyrrolidin-3-yl)urea; 1-(4-chloro-1-phenyl-3-(pyridin-2-yl)-1H-pyrazol-5-yl)-3-((3S,4R)-4-(3,4difluorophenyl)-1-(2-methoxyethyl)pyrrolidin-3-yl)urea; 1-(4-chloro-1-phenyl-3-(pyridin-4-yl)-1H-pyrazol-5-yl)-3-((3S,4R)-4-(3,4difluorophenyl)-1-(2-methoxyethyl)pyrrolidin-3-yl)urea; 1-(4-chloro-1-phenyl-3-(pyridin-3-yl)-1H-pyrazol-5-yl)-3-((3S,4R)-4-(3,4difluorophenyl)-1-(2-methoxyethyl)pyrrolidin-3-yl)urea; 1-(4-bromo-1-phenyl-3-(pyridin-3-yl)-1H-pyrazol-5-yl)-3-((3S,4R)-4-(3,4-difluorophenyl)-1-(2-methoxyethyl)pyrrolidin-3-yl)urea; 1-(4-bromo-1-phenyl-3-(pyridin-2-yl)-1H-pyrazol-5-yl)-3-((3S,4R)-4-(3,4-difluorophenyl)-1-(2-methoxyethyl)pyrrolidin-3-yl)urea; 1-(4-chloro-3-phenyl-1-(pyridin-3-yl)-1H-pyrazol-5-yl)-3-((3S,4R)-4-(3,4difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-methyl-1-phenyl-3-(pyridin-3-yl)-1H-pyrazol-5-yl)urea; 1-((3S,4R)-4-(3,4-difluorophenyl)-1-(2-methoxyethyl)pyrrolidin-3-yl)-3-(4-methyl-1-phenyl-3-(pyridin-4-yl)-1H-pyrazol-5-yl)urea; 1-((3S,4R)-4-(3,4-difluorophenyl)-1-(2-methoxyethyl)pyrrolidin-3-yl)-3-(4-methyl-1-phenyl-3-(pyridin-2-yl)-1H-pyrazol-5-yl)urea; 1-((3S,4R)-4-(3,4-difluorophenyl)-1-(2-methoxyethyl)pyrrolidin-3-yl)-3-(3-(5-fluoropyridin-3-yl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(5-fluoropyridin-3-yl)-4-methyl-1-(pyridin-3-yl)-1H-pyrazol-5-yl)urea; 1-((3S,4R)-4-(3,4-difluorophenyl)-1-(2-methoxyethyl)pyrrolidin-3-yl)-3-(1′,4-dimethyl-1-phenyl-1H,1′H-[3,3′-bipyrazol]-5-yl)urea; 1-(1′,4-dimethyl-1-phenyl-1H,1′H-[3,3′-bipyrazol]-5-yl)-3-((3S,4R)-4-(4-fluorophenyl)-1-(2-methoxyethyl)pyrrolidin-3-yl)urea; 1-((3S,4R)-4-(3,5-difluorophenyl)-1-(2-methoxyethyl)pyrrolidin-3-yl)-3-(1′,4-dimethyl-1-phenyl-1H,1′H-[3,3′-bipyrazol]-5-yl)urea; 1-((3S,4R)-4-(3,4-difluorophenyl)-1-(2-methoxyethyl)pyrrolidin-3-yl)-3-(2′,4-dimethyl-1-phenyl-1H,2′H-[3,3′-bipyrazol]-5-yl)urea; 1-((3S,4R)-4-(3,4-difluorophenyl)-1-(2-methoxyethyl)pyrrolidin-3-yl)-3-(1-(5-fluoropyridin-3-yl)-1′,4-dimethyl-1H,1′H-[3,4′-bipyrazol]-5-yl)urea; 1-((3S,4R)-4-(3,4-difluorophenyl)-1-(2-methoxyethyl)pyrrolidin-3-yl)-3-(1′,4-dimethyl-1-(5-methylpyridin-3-yl)-1H,1′H-[3,4′-bipyrazol]-5-yl)urea; 1-(1-(5-chloropyridin-3-yl)-1′,4-dimethyl-1H,1′H-[3,4′-bipyrazol]-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-methyl-1-phenyl-1′-(2,2,2-trifluoro-1-(2,2,2-trifluoroethoxy)ethyl)-1H,1′H-[3,4′-bipyrazol]-5-yl)urea; 1-((3S,4R)-4-(3,4-difluorophenyl)-1-(2-methoxyethyl)pyrrolidin-3-yl)-3-(4-methyl-1-phenyl-1′-(2,2,2-trifluoroethyl)-1H,1′H-[3,4′-bipyrazol]-5-yl)urea; 1-(1′-(cyclopropylmethyl)-4-methyl-1-phenyl-1H,1′H-[3,4′-bipyrazol]-5-yl)-3-((3S,4R)-4-(3,4-difluorophenyl)-1-(2-methoxyethyl)pyrrolidin3-yl)urea; 1-(1′-(cyclopropanecarbonyl)-4-methyl-1-phenyl-1H,1′H-[3,4′-bipyrazol]-5yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-methyl-1′-(methylsulfonyl)-1-phenyl-1H,1′H-[3,4′-bipyrazol]-5-yl)urea; 1-((3S,4R)-4-(3,4-difluorophenyl)-1-(2-methoxyethyl)pyrrolidin-3-yl)-3-(1′-isopropyl-4-methyl-1-phenyl-1H,1′H-[3,4′-bipyrazol]-5-yl)urea; 1-((3S,4R)-4-(3,4-difluorophenyl)-1-(2-methoxyethyl)pyrrolidin-3-yl)-3-(4-methyl-1-phenyl-3-(pyrimidin-5-yl)-1H-pyrazol-5-yl)urea; 1-((3S,4R)-4-(3,4-difluorophenyl)-1-(2-methoxyethyl)pyrrolidin-3-yl)-3-(4-methyl-3-(1-methyl-2-oxo-1,2-dihydropyridin-4-yl)-1-phenyl-1H-pyrazol-5-yl)urea; 1-((3S,4R)-4-(3,4-difluorophenyl)-1-(2-methoxyethyl)pyrrolidin-3-yl)-3-(4-methyl-3-(1-methyl-6-oxo-1,6-dihydropyridin-3-yl)-1-phenyl-1H-pyrazol-5-yl)urea; 1-((3S,4R)-4-(3,4-difluorophenyl)-1-(2-methoxyethyl)pyrrolidin-3-yl)-3-(1′,4,5′-trimethyl-1-phenyl-1H,1′H-[3,4′-bipyrazol]-5-yl)urea; 1-((3S,4R)-4-(3,4-difluorophenyl)-1-(2-methoxyethyl)pyrrolidin-3-yl)-3-(1′,3′,4-trimethyl-1-phenyl-1H,1′H-[3,4′-bipyrazol]-5-yl)urea; 1-(1′-cyclopropyl-4-methyl-1-phenyl-1H,1′H-[3,4′-bipyrazol]-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-methyl-3-(2-methylthiazol-5-yl)-1-phenyl-1H-pyrazol-5-yl)urea; 1-((3S,4R)-4-(3,4-difluorophenyl)-1-(2-methoxyethyl)pyrrolidin-3-yl)-3-(4-methyl-3-(2-methylpyrimidin-5-yl)-1-phenyl-1H-pyrazol-5-yl)urea; 1-(3-(2-aminopyrimidin-5-yl)-4-methyl-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-(2,4-dimethylthiazol-5-yl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2,6-dimethylpyridin-4-yl)-4-methyl-1-phenyl-1H-pyrazol-5-yl)urea; 1-(3-(6-aminopyridin-3-yl)-4-methyl-1-phenyl-1H-pyrazol-5-yl)-3-((3S,4R)4-(3,4-difluorophenyl)-1-(2-methoxyethyl)pyrrolidin-3-yl)urea; 1-(3-bromo-4-methyl-1-phenyl-1H-pyrazol-5-yl)-3-((3S,4R)-4-(4-fluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-methyl-3-(6-oxo-1-(2,2,2-trifluoroethyl)-1,6-dihydropyridin-3-yl)-1-phenyl-1H-pyrazol-5-yl)urea; 1-((3S,4R)-4-(4-fluorophenyl)-1-(2-methoxyethyl)pyrrolidin-3-yl)-3-(1′-isopropyl-4-methyl-1-phenyl-1H,1′H-[3,4′-bipyrazol]-5-yl)urea; 1-((3S,4R)-4-(4-fluorophenyl)-1-(2-methoxyethyl)pyrrolidin-3-yl)-3-(4-methyl-3-(1-methyl-6-oxo-1,6-dihydropyridin-3-yl)-1-phenyl-1H-pyrazol-5-yl)urea; 1-(3-bromo-4-methyl-1-phenyl-1H-pyrazol-5-yl)-3-((3R,4S)-4-phenyl-1-(2,2,2-trifluoroethyl)pyrrolidin-3-yl)urea; 1-(4-methyl-3-(1-methyl-6-oxo-1,6-dihydropyridin-3-yl)-1-phenyl-1H-pyrazol-5-yl)-3-((3R,4S)-4-phenyl-1-(2,2,2-trifluoroethyl)pyrrolidin-3-yl)urea; 1-(3-(2-aminopyrimidin-5-yl)-4-methyl-1-phenyl-1H-pyrazol-5-yl)-3((3R,4S)-4-phenyl-1-(2,2,2-trifluoroethyl)pyrrolidin-3-yl)urea bis(2,2,2-trifluoroacetate; 1-((3S,4R)-4-(3,4-difluorophenyl)-1-(2-methoxyethyl)pyrrolidin-3-yl)-3-(1′-ethyl-4-methyl-1-phenyl-1H,1′H-[3,4′-bipyrazol]-5-yl)urea; 1-(1′-ethyl-4-methyl-1-phenyl-1H,1′H-[3,4′-bipyrazol]-5-yl)-3-((3S,4R)-4(4-fluorophenyl)-1-(2-methoxyethyl)pyrrolidin-3-yl)urea; 5-(3-((3S,4R)-4-(3,4-difluorophenyl)-1-(2-methoxyethyl)pyrrolidin-3-yl)ureido)-4-methyl-1-phenyl-1H-pyrazol-3-yl trifluoromethanesulfonate; 1-((3S,4R)-4-(3,4-difluorophenyl)-1-(2-methoxyethyl)pyrrolidin-3-yl)-3-(3-(2-methoxypyrimidin-5-yl)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-(2-(dimethylamino)pyrimidin-5-yl)-4-methyl-1-phenyl-1H-pyrazol-5-yl)urea; 1-((3S,4R)-4-(4-fluorophenyl)-1-(2-methoxyethyl)pyrrolidin-3-yl)-3-(3-(2-methoxypyrimidin-5-yl)-4-methyl-1-phenyl-1H-pyrazol-5-yl)urea; 1-(3-(2-(dimethylamino)pyrimidin-5-yl)-4-methyl-1-phenyl-1H-pyrazol-5-yl)-3-((3S,4R)-4-(4-fluorophenyl)-1-(2-methoxyethyl)pyrrolidin-3-yl)urea; 1-(1′-ethyl-4-methyl-1-phenyl-1H,1′H-[3,4′-bipyrazol]-5-yl)-3-((3S,4R)-4(3-fluorophenyl)-1-(2-methoxyethyl)pyrrolidin-3-yl)urea; 1-((3S,4R)-4-(3-fluorophenyl)-1-(2-methoxyethyl)pyrrolidin-3-yl)-3-(4-methyl-3-(1-methyl-2-oxo-1,2-dihydropyridin-4-yl)-1-phenyl-1H-pyrazol-5-yl)urea; 1-((3S,4R)-4-(4-fluorophenyl)-1-(2-methoxyethyl)pyrrolidin-3-yl)-3-(4-methyl-3-(1-methyl-2-oxo-1,2-dihydropyridin-4-yl)-1-phenyl-1H-pyrazol-5-yl)urea; 1-(3-cyclopropyl-4-methyl-1-phenyl-1H-pyrazol-5-yl)-3-((3S,4R)-4-(3,4-difluorophenyl)-1-(2-methoxyethyl)pyrrolidin-3-yl)urea; 1-(3-cyclopropyl-4-methyl-1-phenyl-1H-pyrazol-5-yl)-3-((3S,4R)-4-(4-fluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-(1-isopropyl-6-oxo-1,6-dihydropyridin-3-yl)-4-methyl-1-phenyl-1H-pyrazol5-yl)urea; 1-((3S,4R)-4-(4-fluorophenyl)-1-(2-methoxyethyl)pyrrolidin-3-yl)-3-(3-(1 isopropyl-6-oxo-1,6-dihydropyridin-3-yl)-4-methyl-1-phenyl-1H-pyrazol-5-yl)urea; 1-((3S,4R)-4-(3,5-difluorophenyl)-1-(2-methoxyethyl)pyrrolidin-3-yl)-3-(3-((S)-2-hydroxypropoxy)-4-methyl-1-phenyl-1H-pyrazol-5-yl)urea dihydrochloride; 1-((3S,4R)-4-(3,5-difluorophenyl)-1-(2-methoxyethyl)pyrrolidin-3-yl)-3-(4-methyl-1-phenyl-3-(2-(piperazin-1-yl)ethoxy)-1H-pyrazol-5-yl)urea trihydrochloride; 1-(3-(benzyloxy)-4-chloro-1-methyl-1H-pyrazol-5-yl)-3-((3S,4R)-4-(3,4-difluorophenyl)-1-(2-methoxyethyl)pyrrolidin-3-yl)urea; 2-((5-(3-((3S,4R)-4-(3,4-difluorophenyl)-1-(2-methoxyethyl)pyrrolidin-3-yl)ureido)-4-methyl-1-phenyl-1H-pyrazol-3-yl)oxy)acetic acid; 2-((5-(3-((3S,4R)-4-(3,4-difluorophenyl)-1-(2-methoxyethyl)pyrrolidin-3-yl)ureido)-4-methyl-1-phenyl-1H-pyrazol-3-yl)oxy)-N-ethylacetamide; 1-((3S,4R)-4-(3,4-difluorophenyl)-1-(2-methoxyethyl)pyrrolidin-3-yl)-3-(4-ethyl-3-(2-hydroxy-2-methylpropoxy)-1-phenyl-1H-pyrazol-5-yl)urea; 1-(3-(2-aminoethoxy)-4-methyl-1-phenyl-1H-pyrazol-5-yl)-3-((3S,4R)-4-(3,4-difluorophenyl)-1-(2-methoxyethyl)pyrrolidin-3-yl)urea; N-(2-((5-(3-((3S,4R)-4-(3,4-difluorophenyl)-1-(2-methoxyethyl)pyrrolidin3-yl)ureido)-4-methyl-1-phenyl-1H-pyrazol-3-yl)oxy)ethyl)methanesulfonamide; N-(2-((5-(3-((3S,4R)-4-(3,4-difluorophenyl)-1-(2-methoxyethyl)pyrrolidin-3-yl)ureido)-4-methyl-1-phenyl-1H-pyrazol-3-yl)oxy)ethyl)acetamide; 1-(3-(2-(4-acetylpiperazin-1-yl)ethoxy)-4-methyl-1-phenyl-1H-pyrazol-5-yl)-3-((3S,4R)-4-(3,4-difluorophenyl)-1-(2-methoxyethyl)pyrrolidin3-yl)urea; 2-((5-(3-((3S,4R)-4-(3,4-difluorophenyl)-1-(2-methoxyethyl)pyrrolidin-3-yl)ureido)-4-methyl-1-phenyl-1H-pyrazol-3-yl)oxy)acetamide; N-(5-(3-((3S,4R)-4-(3,4-difluorophenyl)-1-(2-methoxyethyl)pyrrolidin-3-yl)ureido)-3-ethoxy-1-phenyl-1H-pyrazol-4-yl)-2,2,2-trifluoroacetamide; 1-(4-amino-3-ethoxy-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-ethoxy-4-(2-hydroxyethyl)-1-phenyl-1H-pyrazol-5-yl)urea; 1-((3S,4R)-4-(3,4-difluorophenyl)-1-(2-methoxyethyl)pyrrolidin-3-yl)-3-(4-methyl-3-(2-(4-methylpiperazin-1-yl)ethoxy)-1-phenyl-1H-pyrazol-5-yl)urea trihydrochloride; 1-((3S,4R)-4-(3,4-difluorophenyl)-1-(2-methoxyethyl)pyrrolidin-3-yl)-3-(4-methyl-3-(2-morpholino-2-oxoethoxy)-1-phenyl-1H-pyrazol-5-yl)urea; 4-bromo-5-(3-((3S,4R)-4-(3,4-difluorophenyl)-1-(2-methoxyethyl)pyrrolidin-3-yl)ureido)-1-phenyl-1H-pyrazole-3-carboxylic acid; 4-bromo-5-(3-((3S,4R)-4-(3,4-difluorophenyl)-1-(2-methoxyethyl)pyrrolidin-3-yl)ureido)-N-methyl-1-phenyl-1H-pyrazole-3-carboxamide; 4-bromo-5-(3-((3S,4R)-4-(3,4-difluorophenyl)-1-(2-methoxyethyl)pyrrolidin-3-yl)ureido)-N-methoxy-1-phenyl-1H-pyrazole-3-carboxamide; 1-(4-chloro-1′-(2-methoxyethyl)-1-phenyl-1H,1′H-[3,4′-bipyrazol]-5-yl)-3((3S,4R)-4-(3,4-difluorophenyl)-1-(2-methoxyethyl)pyrrolidin-3-yl)urea; 1-((3S,4R)-4-(3,5-difluorophenyl)-1-(2-methoxyethyl)pyrrolidin-3-yl)-3-(3-((S)-2,3-dihydroxypropoxy)-4-methyl-1-phenyl-1H-pyrazol-5-yl)urea; 1-((3S,4R)-4-(3,5-difluorophenyl)-1-(2-methoxyethyl)pyrrolidin-3-yl)-3-(3-((R)-2,3-dihydroxypropoxy)-4-methyl-1-phenyl-1H-pyrazol-5-yl)urea; 1-(3-((R)-2,3-dihydroxypropoxy)-4-methyl-1-phenyl-1H-pyrazol-5-yl)-3-((3S,4R)-1-(2-methoxyethyl)-4-(3,4,5-trifluorophenyl)pyrrolidin-3-yl)urea; 1-(3-((S)-2,3-dihydroxypropoxy)-4-methyl-1-phenyl-1H-pyrazol-5yl)-3-((3S,4R)-1-(2-methoxyethyl)-4-(3,4,5-trifluorophenyl)pyrrolidin-3-yl)urea; 1-(3-((S)-2-hydroxypropoxy)-4-methyl-1-phenyl-1H-pyrazol-5-yl)-3-((3S,4R)-1-(2-methoxyethyl)-4-(3,4,5-trifluorophenyl)pyrrolidin-3-yl)urea; 1-((3S,4R)-4-(3,4-difluorophenyl)-1-(2-methoxyethyl)pyrrolidin-3-yl)-3-(3-((S)-2-hydroxy-3-methoxypropoxy)-4-methyl-1-phenyl-1H-pyrazol-5-yl)urea; 1-((3S,4R)-4-(3,4-difluorophenyl)-1-(2-methoxyethyl)pyrrolidin-3-yl)-3-(3-((R)-2-hydroxy-3-methoxypropoxy)-4-methyl-1-phenyl-1H-pyrazol-5-yl)urea; 1-(3-((S)-2-hydroxy-3-methoxypropoxy)-4-methyl-1-phenyl-1H-pyrazol-5-yl)3-((3S,4R)-1-(2-methoxyethyl)-4-(3,4,5-trifluorophenyl)pyrrolidin-3-yl)urea; 1-(3-((R)-2-hydroxy-3-methoxypropoxy)-4-methyl-1-phenyl-1H-pyrazol-5-yl)-3-((3S,4R)-1-(2-methoxyethyl)-4-(3,4,5-trifluorophenyl)pyrrolidin-3-yl)urea; 1-(4-bromo-1,1′-dimethyl-1H,1′H-[3,4′-bipyrazol]-5-yl)-3-((3S,4R)4-(3,4-difluorophenyl)-1-(2-methoxyethyl)pyrrolidin-3-yl)urea; 1-(4-chloro-1,1′-dimethyl-1H,1′H-[3,4′-bipyrazol]-5-yl)-3-((3S,4R)-4-(3,4-difluorophenyl)-1-(2-methoxyethyl)pyrrolidin-3-yl)urea; 1-(4-chloro-1-phenyl-3-(tetrahydro-2H-pyran-4-yl)-1H-pyrazol-5-yl)-3-((3S,4R)-4-(3,4-difluorophenyl)-1-(2-methoxyethyl)pyrrolidin-3-yl)urea; Tert-butyl 4-(4-chloro-5-(3-((3S,4R)-4-(3,4-difluorophenyl)-1-(2-methoxyethyl)pyrrolidin-3-yl)ureido)-1phenyl-1H-pyrazol-3-yl)piperidine-1-carboxylate; 1-(4-chloro-1-phenyl-3-(tetrahydro-2H-pyran-4-yl)-1H-pyrazol-5-yl)-3-((3S,4R)-4-(4-fluorophenyl)-1-(2-methoxyethyl)pyrrolidin-3-yl)urea; 1-(4-chloro-3-(3,5-dimethylisoxazol-4-yl)-1-phenyl-1H-pyrazol-5-yl)-3-((3S,4R)-4-(3,4-difluorophenyl)-1-(2-methoxyethyl)pyrrolidin-3-yl)urea; (R)-tert-butyl 2-(4-chloro-5-(3-((3S,4R)-4-(3,4-difluorophenyl)-1-(2-methoxyethyl)pyrrolidin-3-yl)ureido)-1phenyl-1H-pyrazol-3-yl)pyrrolidine-1-carboxylate; (S)-tert-butyl 2-(4-chloro-5-(3-((3S,4R)-4-(3,4-difluorophenyl)-1-(2-methoxyethyl)pyrrolidin-3-yl)ureido)-1phenyl-1H-pyrazol-3-yl)pyrrolidine-1-carboxylate; 1-(4-bromo-1-phenyl-3-(tetrahydro-2H-pyran-4-yl)-1H-pyrazol-5-yl)-3-((3S,4R)-4-(3,4-difluorophenyl)-1-(2-methoxyethyl)pyrrolidin-3-yl)urea; Tert-butyl 4-(4-bromo-5-(3-((3S,4R)-4-(3,4-difluorophenyl)-1-(2-methoxyethyl)pyrrolidin-3-yl)ureido)-1phenyl-1H-pyrazol-3-yl)piperidine-1-carboxylate; 1-(4-bromo-1-phenyl-3-(tetrahydro-2H-pyran-4-yl)-1H-pyrazol-5-yl)-3-((3S,4R)-4-(4-fluorophenyl)-1-(2-methoxyethyl)pyrrolidin-3-yl)urea; 1-(4-bromo-3-(3,5-dimethylisoxazol-4-yl)-1-phenyl-1H-pyrazol-5-yl)-3-difluorophenyl)-1-(2-methoxyethyl)pyrrolidin-3-yl)urea; (R)-tert-butyl 2-(4-bromo-5-(3-((3S,4R)-4-(3,4-difluorophenyl)-1-(2-methoxyethyl)pyrrolidin-3-yl)ureido)-1-phenyl-1H-pyrazol-3-yl)pyrrolidine-1-carboxylate; tert-butyl 4-((4-bromo-5-(3-((3S,4R)-4-(3,4-difluorophenyl)-1-(2-methoxyethyl)pyrrolidin-3-yl)ureido)-1phenyl-1H-pyrazol-3-yl)methoxy)piperidine-1-carboxylate; 1-((3S,4R)-4-(3,4-difluorophenyl)-1-(2-methoxyethyl)pyrrolidin-3-yl)-3-(I-phenyl-3-(piperidin-4-yl)-1H-pyrazol-5-yl)urea dihydrochloride; 1-(4-chloro-1-phenyl-3-(piperidin-4-yl)-1H-pyrazol-5-yl)-3-((3S,4R)-4-(3,4-difluorophenyl)-1-(2-methoxyethyl)pyrrolidin-3-yl)urea dihydrochloride; 1-(4-bromo-1-phenyl-3-(piperidin-4-yl)-1H-pyrazol-5-yl)-3-((3S,4R)-4-(3,4-difluorophenyl)-1-(2-methoxyethyl)pyrrolidin-3-yl)urea dihydrochloride; 1-((3S,4R)-4-(3,4-difluorophenyl)-1-(2-methoxyethyl)pyrrolidin-3-yl)-3-(4-methyl-1-phenyl-3-((R)-pyrrolidin-2-yl)-1H-pyrazol-5-yl)urea dihydrochloride; 1-((3S,4R)-4-(3,4-difluorophenyl)-1-(2-methoxyethyl)pyrrolidin-3-yl)-3-(4-methyl-1-phenyl-3-((S)-pyrrolidin-2-yl)-1H-pyrazol-5-yl)urea dihydrochloride; 1-((3S,4R)-4-(3,4-difluorophenyl)-1-(2-methoxyethyl)pyrrolidin-3-yl)-3-(4-chloro-1-phenyl-3-((R)-pyrrolidin-2-yl)-1H-pyrazol-5-yl)urea dihydrochloride; 1-((3S,4R)-4-(3,5-difluorophenyl)-1-(2-methoxyethyl)pyrrolidin-3-yl)-3-(4-methyl-1-phenyl-3-((S)-pyrrolidin-2-yl)-1H-pyrazol-5-yl)urea dihydrochloride; 1-((3S,4R)-4-(3,4-difluorophenyl)-1-(2-methoxyethyl)pyrrolidin-3-yl)-3-(4-chloro-1-phenyl-3-((S)-pyrrolidin-2-yl)-1H-pyrazol-5-yl)urea dihydrochloride; 1-(4-bromo-1-phenyl-3-((R)-pyrrolidin-2-yl)-1H-pyrazol-5-yl)-3-((3S,4R)-4-(3,4-difluorophenyl)-1-(2-methoxyethyl)pyrrolidin-3-yl)urea dihydrochloride; 1-((3S,4R)-4-(3,4-difluorophenyl)-1-(2-methoxyethyl)pyrrolidin-3-yl)-3-(1-phenyl-3-((piperidin-4-yloxy)methyl)-1H-pyrazol-5-yl)urea dihydrochloride; 1-(4-chloro-1-phenyl-3-((piperidin-4-yloxy)methyl)-1H-pyrazol-5-yl)-3-((3S,4R)-4-(3,4-difluorophenyl)-1-(2-methoxyethyl)pyrrolidin-3-yl)urea dihydrochloride; 1-(4-bromo-1-phenyl-3-((piperidin-4-yloxy)methyl)-1H-pyrazol-5-yl)-3-((3S,4R)-4-(3,4-difluorophenyl)-1-(2-methoxyethyl)pyrrolidin-3-yl)urea dihydrochloride; 1-(4-bromo-3-(1-(methylsulfonyl)piperidin-4-yl)-1-phenyl-1H-pyrazol-5-yl)-3-((3S,4R)-4-(3,4-difluorophenyl)-1-(2-methoxyethyl)pyrrolidin-3-yl)urea; 1-(3-(1-acetylpiperidin-4-yl)-4-bromo-1-phenyl-1H-pyrazol-5-yl)-3((3S,4R)-4-(3,4-difluorophenyl)-1-(2-methoxyethyl)pyrrolidin-3-yl)urea; 1-(4-chloro-1-phenyl-3-(1-(trifluoromethylsulfonyl)piperidin-4-yl)-1H-pyrazol-5-yl)-3-((3S,4R)-4-(3,4-difluorophenyl)-1-(2-methoxyethyl)pyrrolidin-3-yl)urea hydrochloride; 1-((3S,4R)-4-(3,4-difluorophenyl)-1-(2-methoxyethyl)pyrrolidin-3-yl)-3-(4-methyl-3-((R)-1-(methylsulfonyl)pyrrolidin-2-yl)-1-phenyl-1H-pyrazol-5-yl)urea; 1-(3-((R)-1-acetylpyrrolidin-2-yl)-4-methyl-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(4-methyl-3-((R)-1-methylpyrrolidin-2-yl)-1-phenyl-1H-pyrazol-5-yl)urea dihydrochloride; 1-((3S,4R)-4-(3,5-difluorophenyl)-1-(2-methoxyethyl)pyrrolidin-3-yl)-3-(4-methyl-3-((S)-1-methylpyrrolidin-2-yl)-1-phenyl-1H-pyrazol-5-yl)urea dihydrochloride; 1-(4-bromo-3-((1-(methyl sulfonyl)piperidin-4-yloxy)methyl)-1-phenyl-1H-pyrazol-5-yl)-3-((3S,4R)-4-(3,4-difluorophenyl)-1-(2-methoxyethyl)pyrrolidin-3-yl)urea; 1-(3-((1-acetylpiperidin-4-yloxy)methyl)-4-bromo-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3,4-difluorophenyl)-1-(2-methoxyethyl)pyrrolidin-3-yl)-3-(3-(4-isopropyl-5-oxo-4,5-dihydro-1,3,4-oxadiazol-2-yl)-4-methyl-1phenyl-1H-pyrazol-5-yl)urea; 1-((3S,4R)-4-(3,4-difluorophenyl)-1-(2-methoxyethyl)pyrrolidin-3-yl)-3-(4-methyl-3-(4-methyl-5-oxo-4,5-dihydro-1,3,4-oxadiazol-2-yl)-1-phenyl-1H-pyrazol-5-yl)urea; 1-((3S,4R)-4-(4-fluorophenyl)-1-(2-methoxyethyl)pyrrolidin-3-yl)-3-(4-methyl-1-phenyl-3-(pyrazin-2-yloxy)-1H-pyrazol-5-yl)urea; 1-((3S,4R)-1-(2-methoxyethyl)-4-(3,4,5-trifluorophenyl)pyrrolidin-3-yl)-3-(4-methyl-3-(1-methyl-6-oxo-1, 6-dihydropyridin-3-yl)-1-phenyl-1H-pyrazol-5-yl)urea; 1-((3S,4R)-4-(3,4-difluorophenyl)-1-(2-methoxyethyl)pyrrolidin-3-yl)-3-(3-methoxy-1-phenyl-4-(trifluoromethyl)-1H-pyrazol-5-yl)urea; 1-((3S,4R)-4-(3,5-difluorophenyl)-1-(2-methoxyethyl)pyrrolidin-3-yl)-3-(3-methoxy-1-phenyl-4-(trifluoromethyl)-1H-pyrazol-5-yl)urea; 1-((3S,4R)-4-(4-fluorophenyl)-1-(2-methoxyethyl)pyrrolidin-3-yl)-3-(3-methoxy-1-phenyl-4-(trifluoromethyl)-1H-pyrazol-5-yl)urea; 1-((3S,4R)-4-(3,4-difluorophenyl)-1-(2-methoxyethyl)pyrrolidin-3-yl)-3-(3-methoxy-1-phenyl-4-(trifluoromethyl)-1H-pyrazol-5-yl)urea; 1-((trans)-4-(4-chloro-3-fluorophenyl)-1-(2-methoxyethyl)pyrrolidin-3-yl)-3-(4-methyl-5-oxo-2-phenyl-2, 5-dihydro-1H-pyrazol-3-yl)urea; 1-((trans)-4-(3-chloro-4-fluorophenyl)-1-(2-methoxyethyl) pyrrolidin-3-yl)-3-(4-methyl-5-oxo-2-phenyl-2,5-dihydro-1H-pyrazol-3-yl)urea; 1-((3S,4R)-4-(3,5-difluorophenyl)-1-(2-methoxyethyl)pyrrolidin-3-yl)-3-(4-methyl-5-oxo-2-phenyl-2, 5-dihydro-1H-pyrazol-3-yl)urea; 1-((3S,4R)-1-(2-methoxyethyl)-4-(3,4,5-trifluorophenyl)pyrrolidin-3-yl)-3-(4-methyl-5-oxo-2-phenyl-2,5-dihydro-1H-pyrazol-3-yl)urea; 1-((3S,4R)-4-(3-fluorophenyl)-1-(2-methoxyethyl)pyrrolidin-3-yl)-3-(4-methyl-5-oxo-2-phenyl-2,5-dihydro-1H-pyrazol-3-yl)urea; 1-((trans)-4-(3-chloro-5-fluorophenyl)-1-(2-methoxyethyl)pyrrolidin-3-yl)-3-(4-methyl-5-oxo-2-phenyl-2, 5-dihydro-1H-pyrazol-3-yl)urea; 1-(4-cyano-3-methoxy-1-phenyl-1H-pyrazol-5-yl)-3-((3S,4R)-4-(3,4-difluorophenyl)-1-(2-methoxyethyl)pyrrolidin-3-yl)urea; 1-((3S,4R)-4-(3-chloro-5-fluorophenyl)-1-(2-methoxyethyl)pyrrolidin-3-yl)-3-(4-methyl-5-oxo-2-phenyl-2, 5-dihydro-1H-pyrazol-3-yl)urea; 1-(4-cyano-1-phenyl-3-(trifluoromethyl)-1H-pyrazol-5-yl)-3-((3S,4R)-4-(3,4-difluorophenyl)-1-(2-methoxyethyl)pyrrolidin-3-yl)urea; 1-(4-cyano-5-oxo-2-phenyl-2, 5-dihydro-1H-pyrazol-3-yl)-3-((3S,4R)-4-(3,4-difluorophenyl)-1-(2-methoxyethyl)pyrrolidin-3-yl)urea; 5-(3-((3S,4R)-4-(3,4-difluorophenyl)-1-(2-methoxyethyl)pyrrolidin-3-yl)ureido)-3-methoxy-1-phenyl-1H-pyrazole-4-carboxamide; 5-(3-((3S,4R)-4-(3,4-difluoro-phenyl)-1-(2-methoxyethyl)pyrrolidin-3-yl)ureido)-3-methyl-1-phenyl-1H-pyrazole-4-carboxamide; 5-(3-((3S,4R)-4-(3,4-difluorophenyl)-1-(2-methoxyethyl)pyrrolidin-3-yl)ureido)-3-ethyl-1-phenyl-1H-pyrazole-4-carboxamide; 5-(3-((3S,4R)-4-(3,4-difluorophenyl)-1-(2-methoxyethyl)pyrrolidin-3-yl)ureido)-1-phenyl-3-(trifluoromethyl)-1H-pyrazole-4-carboxamide; 5-(3-((trans)-4-(3-chloro-4-fluorophenyl)-1-(2-methoxyethyl)pyrrolidin-3yl)ureido)-3-methyl-1-phenyl-1H-pyrazole-4-carboxamide; 5-(3-((trans)-4-(4-chloro-3-fluorophenyl)-1-(2-methoxyethyl)pyrrolidin-3yl)ureido)-3-methyl-1-phenyl-1H-pyrazole-4-carboxamide; 5-(3-((trans)-4-(3-chloro-5-fluorophenyl)-1-(2-methoxyethyl)pyrrolidin-3yl)ureido)-3-methyl-1-phenyl-1H-pyrazole-4-carboxamide; 5-(3-((3S,4R)-4-(3,5-difluorophenyl)-1-(2-methoxyethyl)pyrrolidin-3-yl)ureido)-3-methyl-1-phenyl-1H-pyrazole-4-carboxamide; 5-(3-((3S,4R)-4-(3,4-difluorophenyl)-1-(2-methoxyethyl)pyrrolidin-3-yl)ureido)-1-phenyl-1H-pyrazole-4-carboxamide; 5-(3-((3S,4R)-4-(3,4-difluorophenyl)-1-(2-methoxyethyl)pyrrolidin-3-yl)ureido)-1-phenyl-1H-pyrazole-4-carboxamide; 1-(4-bromo-3-methyl-1-phenyl-1H-pyrazol-5-yl)-3-((3S,4R)-4-(3,4-difluorophenyl)-1-(2-methoxyethyl)pyrrolidin-3-yl)guanidine dihydrochloride; 1-(4-bromo-3-methyl-1-phenyl-1H-pyrazol-5-yl)-3-((3S,4R)-4-(3,4-difluorophenyl)-1-(2-methoxyethyl)pyrrolidin-3-yl)thiourea; 1-((3S,4R)-4-(3,4-difluorophenyl)-1-(2-methoxyethyl)pyrrolidin-3-yl)-3-(4-methyl-3-(1-methyl-6-oxo-1, 6-dihydropyridin-3-yl)-1-phenyl-1H-pyrazol-5-yl)thiourea; 1-((3S,4R)-4-(3,4-difluorophenyl)-1-(2-methoxyethyl)pyrrolidin-3-yl)-3-(1′,4-dimethyl-1-phenyl-1H,1′H-3,4′-bipyrazol-5-yl)thiourea; 1-((3S,4R)-4-(4-fluorophenyl)-1-(2-methoxyethyl)pyrrolidin-3-yl)-3-(1′,4dimethyl-1-phenyl-1H,1′H-3,4′-bipyrazol-5-yl)thiourea; Trans-1-(2-methoxyethyl)-4-phenylpyrrolidin-3-yl)-3-(2-phenylpyrazolo[1,5-a]pyridin-3-yl)urea; 1-((3S,4R)-4-(3,4-difluorophenyl)-1-(2-methoxyethyl)pyrrolidin-3-yl)-3-(2-phenylpyrazolo[1,5-a]pyridin-3-yl)urea; Trans-1-(2-methoxyethyl)-4-phenylpyrrolidin-3-yl)-3-(pyrazolo[1,5-a]pyridin-3-yl)urea; 1-((3S,4R)-4-(3,4-difluorophenyl)-1-(2-methoxyethyl)pyrrolidin-3-yl)-3-(pyrazolo[1,5-a]pyridin-3-yl)urea; 1-((3S,4R)-4-(3,4-difluorophenyl)-1-(2-methoxyethyl)pyrrolidin-3-yl)-3-(5-methyl-3-phenyl-1-(pyrazin-2-yl)-1H-pyrazol-4-yl)urea; 1-((3S,4R)-4-(3,4-difluorophenyl)-1-(2-methoxyethyl)pyrrolidin-3-yl)-3-(1,5-dimethyl-3-phenyl-1H-pyrazol-4-yl)urea; 1-((3S,4R)-4-(3,5-difluorophenyl)-1-(2-methoxyethyl)pyrrolidin-3-yl)-3-(1,5-dimethyl-3-phenyl-1H-pyrazol-4-yl)urea; 1-((3S,4R)-4-(3,4-difluorophenyl)-1-(2-methoxyethyl)pyrrolidin-3-yl)-3-(1-isopropyl-5-methyl-3-phenyl-1H-pyrazol-4-yl)urea; 1-((3S,4R)-4-(3,5-difluorophenyl)-1-(2-methoxyethyl)pyrrolidin-3-yl)-3-(1-isopropyl-5-methyl-3-phenyl-1H-pyrazol-4-yl)urea; 1-((3S,4R)-4-(3,4-difluorophenyl)-1-(2-methoxyethyl)pyrrolidin-3-yl)-3-(5-methyl-3-phenyl-1-(2,2,2-trifluoroethyl)-1H-pyrazol-4-yl)urea; 1-((3S,4R)-4-(3,4-difluorophenyl)-1-(2-methoxyethyl)pyrrolidin-3-yl)-3-(1-ethyl-5-methyl-3-phenyl-1H-pyrazol-4-yl)urea; 1-((3S,4R)-4-(3,4-difluorophenyl)-1-(2-methoxyethyl)pyrrolidin-3-yl)-3-(1-ethyl-3-methyl-5-phenyl-1H-pyrazol-4-yl)urea; 1-((3S,4R)-4-(3,4-difluorophenyl)-1-(2-methoxyethyl)pyrrolidin-3-yl)-3-(1-methyl-5-phenyl-3-(trifluoromethyl)-1H-pyrazol-4-yl)urea; 1-((3S,4R)-4-(3,4-difluorophenyl)-1-(2-methoxyethyl)pyrrolidin-3-yl)-3-(1-methyl-3-phenyl-5-(trifluoromethyl)-1H-pyrazol-4-yl)urea; 1-((3S,4R)-4-(3,4-difluorophenyl)-1-(2-methoxyethyl)pyrrolidin-3-yl)-3-(3-methyl-1-phenyl-1H-pyrazol-4-yl)urea; 1-((3S,4R)-4-(3,4-difluorophenyl)-1-(2-methoxyethyl)pyrrolidin-3-yl)-3-(I-phenyl-3-(trifluoromethyl)-1H-pyrazol-4-yl)urea; or an acceptable salt thereof.

In some embodiments, a Trk inhibitor is selected from the group consisting of: 5-Chloro-N⁴-(5-cyclopropyl-1H-pyrazol-3-yl)-N²-(1-phenylethyl)pyrimidine-2,4-diamine; 5-Bromo-N⁴-(3-ethyl-1H-pyrazol-5-yl)-N²-(1-phenylethyl) pyrimidine-2,4-diamine; N⁴-(3-tert-Butyl-1H-pyrazol-5-yl)-5-chloro-N²-(1-phenylethyl)pyrimidine-2,4-diamine; N⁴-(3-Cyclopropyl-1H-pyrazol-5-yl)-N²-(1-phenylethyl)-5-(trifluoromethyl)pyrimidine-2,4-diamine; 5-Bromo-N⁴-(3-cyclopropyl-1H-pyrazol-5-yl)-N²-[(1S)-1-(4-fluorophenyl)ethyl]pyrimidine-2,4-diamine; 5-Bromo-N⁴-(3-cyclopropyl-1H-pyrazol-5-yl)-N²-[(1S)-1-phenylpropyl]pyrimidine-2,4-diamine; 5-Bromo-N⁴-(3-cyclopropyl-1H-pyrazol-5-yl)-N²-[(1S)-1-(4-nitrophenyl)ethyl]pyrimidine-2,4-diamine; (2R)-2-({5-Bromo-4-[(3-cyclopropyl-1H-pyrazol-5-yl)amino]pyrimidin-2yl}amino)-2-phenylethanol; 5-Bromo-N⁴-(5-cyclopropyl-1H-pyrazol-3-yl)-N²-(1-phenylethyl)pyrimidine-2,4-diamine; 5-Chloro-N⁴-(5-cyclopropyl-1H-pyrazol-3-yl)-N²-(1-phenylpropyl)pyrimidine-2,4-diamine.

Syk Inhibitors. Particularly in those embodiments which are feeder-free, the medium may also include a SYK (Spleen Tyrosine Kinase) inhibitor. Representative SYK inhibitor can be is selected from the group consisting of Entospletinib (GS-9973), Fostamatinib (R788), R406, cerdulatinib (PRT062070) and TAK-659.

In some embodiments, the Syk inhibitor is entospletinib, which has the following structure:

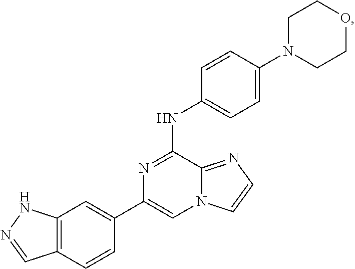 [SMILES: c1cn2cc(-c3ccc4cn[nH]c4c3)nc(Nc3ccc(N4CCOCC4)cc3)c2n1]

The chemical name of entospletinib is 6-(1H-indazol-6-yl)-N-(4-morpholinophenyl)imidazo[1,2-a]pyrazin-8-amine. Entospletinib or a pharmaceutically acceptable salt, solvate, or polymorph thereof may be prepared by according to procedures described in U.S. Pat. Nos. 8,748,607 and 8,450,321, and U.S. Patent Application Publication No. 2015/0038505.

In some embodiments, the Syk inhibitor is a compound of Formula:

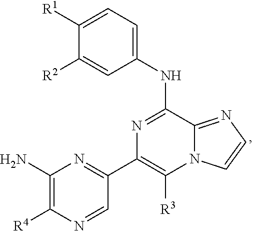

or an ester, stereoisomer, or tautomer thereof, wherein:
R1 is:

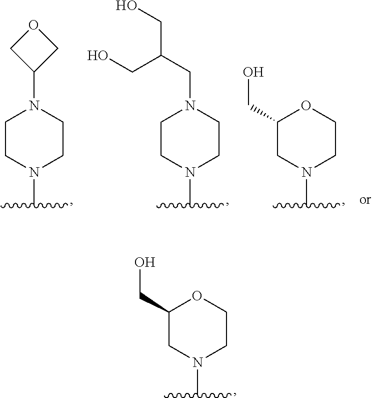

wherein [symbol] indicates the point of attachment to the remainder of the compound of formula,
R2 is H or 2-hydroxyethoxy, R3 is H or methyl, and R4 is H or methyl.

In one embodiment each of R2, R3, and R4 is H, and R1 is as defined above. In one embodiment, R2 is H, R3 is methyl, and R4 is H, and R1 is as defined above. In one embodiment, R2 is H, R3 is H, and R4 is methyl, and R1 is as defined above. In one embodiment, R2 is 2-hydroxyethoxy, R3 is methyl, and R4 is H, and R1 is as defined above. In one embodiment, R2 is 2-hydroxyethoxy, R3 is methyl, and R4 is H, and R1 is as defined above. In one embodiment, R2 is 2-hydroxyethoxy, R3 is H, and R4 is methyl, and R1 is as defined above.

In one embodiment, the SYK inhibitor is selected from:

| | |
|---|---|
| 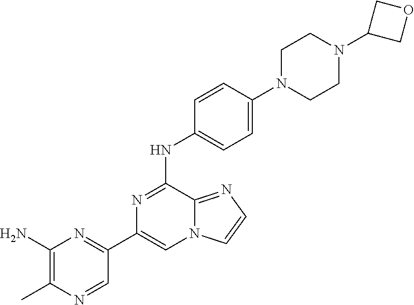 [SMILES: Cc1ncc(-c2cn3ccnc3c(Nc3ccc(N4CCN(C5COC5)CC4)cc3)n2)nc1N] | 6-(6-amino-5-methylpyrazin2-yl)-N-(4- (4-(oxetan-3yl)piperazin-1- yl)phenyl)imidazo[1,2a]pyrazin-8-amine |
| 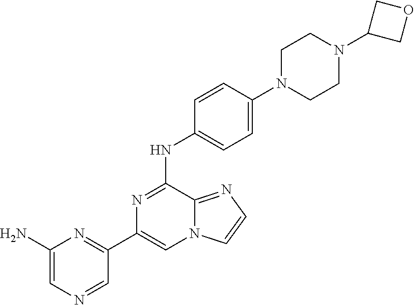 [SMILES: Nc1cncc(-c2cn3ccnc3c(Nc3ccc(N4CCN(C5COC5)CC4)cc3)n2)n1] | 6-(6-aminopyrazin-2-yl)-N(4-(4-(oxetan- 3-yl)piperazin1- yl)phenyl)imidazo[1,2a]pyrazin-8-amine |
| 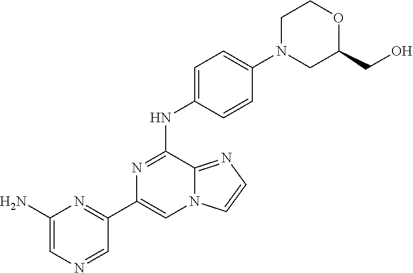 [SMILES: Nc1cncc(-c2cn3ccnc3c(Nc3ccc(N4CCO[C@@H](CO)C4)cc3)n2)n1] | (R)-(4-(4-((6-(6aminopyrazin- 2yl)imidazo[1,2-a]pyrazin- 8yl)amino)phenyl)morpholin2- yl)methanol |
| 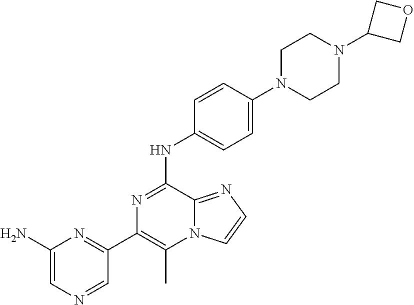 [SMILES: Cc1c(-c2cncc(N)n2)nc(Nc2ccc(N3CCN(C4COC4)CC3)cc2)c2nccn12] | 6-(6-aminopyrazin-2-yl)-5methyl-N-(4- (4-(oxetan-3yl)piperazin- 1yl)phenyl)imidazo[1,2a]pyrazin-8- amine |
| 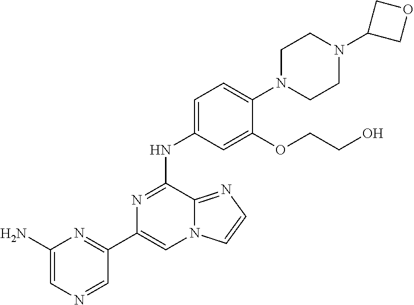 [SMILES: Nc1cncc(-c2cn3ccnc3c(Nc3ccc(N4CCN(C5COC5)CC4)c(OCCO)c3)n2)n1] | 2-(5-((6-(6-aminopyrazin- 2yl)imidazo[1,2-a]pyrazin-8yl)amino)-2- (4-(oxetan-3yl)piperazin- 1yl)phenoxy)ethan-1-ol |
| 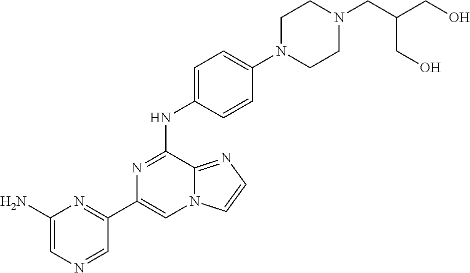 [SMILES: Nc1cncc(-c2cn3ccnc3c(Nc3ccc(N4CCN(CC(CO)CO)CC4)cc3)n2)n1] | 2-((4-(4-((6-(6-aminopyrazin2- yl)imidazo[1,2-a]pyrazin- 8yl)amino)phenyl)piperazin- 1yl)methyl)propane-1,3-diol |
| 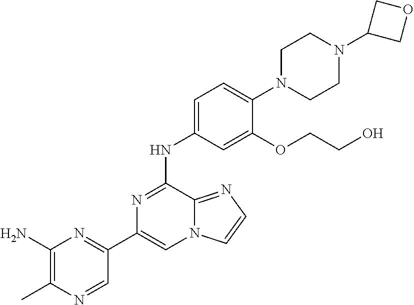 [SMILES: Cc1ncc(-c2cn3ccnc3c(Nc3ccc(N4CCN(C5COC5)CC4)c(OCCO)c3)n2)nc1N] | 2-(5-((6-(6-amino-5methylpyrazin- 2yl)imidazo[1,2-a]pyrazin-8yl)amino)-2- (4-(oxetan-3yl)piperazin- 1yl)phenoxy)ethan-1-ol |

Suitable Syk Inhibitors are described in U.S. Pat. No. 9,290,050.

Syk inhibitors, as employed in the present invention, include those compounds disclosed in U.S. Pat. Nos. 9,290,050 and 6,432,963, and U.S. Patent Application Publication No. US2004/0029902 A1 each of which are hereby incorporated by reference in their entirety. Exemplary Syk Inhibitors from these reference include, but are not limited to
2-(2-aminoethylamino)-4-(3-methylanilino)pyrimidine-5-carboxamide,
2-(2-aminoethylamino)-4-(3-trifluoromethylanilino)pyrimidine-5-carboxamide,
2-(4-aminobutylamino)-4-(3-trifluoromethylanilino)pyrimidine-5-carboxamide,
2-(2-aminoethylamino)-4-(3-bromoanilino)pyrimidine-5-carboxamide,
2-(2-aminoethylamino)-4-(3-nitroanilino)pyrimidine-5-carboxamide,
2-(2-aminoethylamino)-4-(3,5-dimethylanilino)pyrimidine-5-carboxamide,
2-(2-aminoethylamino)-4-(2-naphthylamino)pyrimidine-5-carboxamide,
2-(cis-2-aminocyclohexylamino)-4-(3-methylanilino)pyrimidine-5-carboxamide,
2-(cis-2-aminocyclohexylamino)-4-(3-bromo-anilino)pyrimidine-5-carboxamide,
2-(cis-2-aminocyclohexylamino)-4-(3,5-dichloroanilino)pyrimidine-5-carboxamide and 2-(cis-2-aminocyclohexylamino)-4-(3,4,5-trimethoxyanilino)pyrimidine-5-carboxamide,
N2,N4-[(2,2-Dimethyl-4H-benzo[1,4]oxazin-3-one)-6-yl]-5-fluoro-2,4-pyrimidinediamine,
N4-(3,4-Dichlorophenyl)-5-fluoro-N2-(indazoline-6-yl)-2,4-pyrimidinediamine,
N4-(3,4-Ethylenedioxyphenyl)-5-fluoro-N2-(1-methyl-indazoline-5-yl)-2,4-pyrimidinediamine,
N2,N4-Bis(3-hydroxyphenyl)-5-fluoro-2,4-pyrimidinediamine,
N2,N4-Bis(3,4-ethylenedioxyphenyl)-5-fluoro-2,4-pyrimidinediamine,
N4-(1,4-Benzoxazin-6-yl)-5-fluoro-N2-[3-(N-methylamino)carbonylmethyleneoxyphenyl]-2,4-pyrimidinediamine,
N2,N4-Bis(3-aminophenyl)-5-fluoro-2,4-pyrimidinediamine,
N4-(3,4-Ethylenedioxyphenyl)-5-fluoro-N2-[3-(N-methylamino)-carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
N4-(3-Hydroxyphenyl)-5-trifluoromethyl-N2-[3-(N-methylamino)carbonylmethyleneoxyphenyl]-2,4-pyrimidinediamine,
5-Fluoro-N4-[(1H)-indol-6-yl]-N2-[3-(N-methylamino)carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
5-Fluoro-N2-(3-methylaminocarbonylmethyleneoxyphenyl)-N4-[2-H-pyrido[3,2-b]-1,4-oxazin-3(4H)-one-6-yl]-2,4-pyrimidinediamine,
N4-(3,4-Ethylenedioxyphenyl)-5-fluoro-N2-[3-(2-hydroxyethylamino)carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidine-diamine,
N2,N4-Bis(indol-6-yl)-5-fluoro-2,4-pyrimidinediamine,
5-Fluoro-N2-[2-(2-hydroxy-1,1-dimethylethylamino)carbonylbenzofuran-5-yl]-N4-(3-hydroxyphenyl)-2,4-pyrimidinediamine,
N2-[3-(N2,3-Dihydroxypropylamino)carbonylmethyleneoxyphenyl]-N4-(3,4-ethylenedioxyphenyl)-5-fluoro-2,4-pyrimidinediamine,
N2-(3,5-Dimethoxyphenyl)-N4-(3,4-ethylenedioxyphenyl)-5-fluoro-2,4-pyrimidinediamine,
N4-(3,4-Ethylenedioxyphenyl)-5-fluoro-N2-[3-(1,3-oxazol-5-yl)phenyl]-2,4-pyrimidinediamine,
N4-(3,4-Ethylenedioxyphenyl)-5-fluoro-N2-[3-(N-methylamino)-carbonylmethyleneoxyphenyl]-2,4-pyrimidinediamine,
5-Fluoro-N2-(3-hydroxyphenyl)-N4-[4-(3-phenyl-1,2-4-oxadiazol-5-yl)methyleneoxyphenyl]-2,4-pyrimidinediamine,
N4-(3,4-Ethylenedioxyphenyl)-5-fluoro-N2-(indazolin-6-yl)-2,4-pyrimidinediamine,
5-Fluoro-N4-(3-hydroxyphenyl)-N2-(indazolin-6-yl)-2,4-pyrimidinediamine,
N4-(3,4-Ethylenedioxyphenyl)-5-fluoro-N2-(1-methyl-indazoline-5-yl)-2,4-pyrimidinediamine,
5-Fluoro-N4-(3-hydroxyphenyl)-N2-(1-methy-indazoline-5-yl)-2,4-pyrimidinediamine,
N4-(3,4-Ethylenedioxyphenyl)-5-fluoro-N2-[4-(3-phenyl-1,2,4-oxadiazol-5-yl)methyleneoxyphenyl]-2,4-pyrimidinediamine,
N4-(3,5-Dimethyl-4-hydroxyphenyl)-5-fluoro-N2-[3-[2-(N-morpholino)ethyleneoxy]phenyl]-2,4-pyrimidinediamine,
N4-(3,5-Dimethyl-4-hydroxyphenyl)-5-fluoro-N2-[3-[2-(N-morpholino)ethyloxy]phenyl]-2,4-pyrimidine-diamine,
N4-(3-Chloro-4-hydroxy-5-methylphenyl)-5-fluoro-N2-[3-[2-(N-morpholino)ethyloxy]phenyl]-2,4-pyrimidinediamine,
N2-(3-tert-Butylcarbonylaminophenyl)-N4-(3-hydroxyphenyl)-5-fluoro-2,4-pyrimidinediamine,
N4-(3-tert-Butylphenyl)-N2-[3-(N-methylamino)carbonylmethyleneoxyphenyl]-5-fluoro-2,4-pyrimidine-diamine,
N4-(3-tert-Butylphenyl)-N2-[3-(N2,3-dihydroxypropylamino)carbonylmethyleneoxyphenyl]-5-fluoro-2,4-pyrimidinediamine,
N2-[3-(N2,3-Dihydroxypropylamino)carbonylmethyleneoxyphenyl]-5-fluoro-N4-(3-isopropylphenyl)-2,4-pyrimidinediamine,
N4-[4-(Cyanomethyleneoxy)phenyl]-5-fluoro-N2-(3-hydroxyphenyl)-2,4-pyrimidinediamine,
N4-(3,5-Dimethyl-4-hydroxyphenyl)-5-fluoro-N2-[3-(N-piperazino)carbonylmethyleneoxyphenyl]-2,4-pyrimidinediamine,
N4-(3,5-Dimethyl-4-hydroxyphenyl)-5-fluoro-N2-[3-[2-(N-piperazino)ethoxy]phenyl]-2,4-pyrimidine-diamine bis hydrochloride salt,
N4-(3,4-Ethylenedioxyphenyl)-5-fluoro-N2-[4-(2-hydroxyethyloxy)phenyl]-2,4-pyrimidinediamine,
N4-(1,4-Benzoxazine-3-on-6-yl)-5-fluoro-N2-(3-hydroxyphenyl)-2,4-pyrimidinediamine,
(+/−)-5-Fluoro-N2-[(N-methylacetamido-2)-3-phenoxy]-N4-(2-methyl-1,4-benzoxazin-6-yl)-2,4-pyrimidi-nediamine,
N2-(1,4-Benzoxazin-3-on-6-yl)-5-fluoro-N4-(3-hydroxyphenyl)-2,4-pyrimidinediamine,
N4-(3-Chloro-4-trifluoromethoxyphenyl)-5-fluoro-N2-[3-(N-methylamino)carbonylmethyleneoxyphenyl]-2,4-pyrimidinediamine,
5-Fluoro-N4-(3-hydroxy-4-methylphenyl)-N2-[3-[(N-methylamino)carbonylmethyleneoxy]phenyl]-2,4-pyrimidinediamine,
5-Fluoro-N4-(3-hydroxyphenyl)-N2-[4-methyl-3-[(N-methylamino)carbonylmethyleneoxy]phenyl]-2,4-pyrimidinediamine,
5-Fluoro-N4-(3-hydroxy-4-methoxyphenyl)-N2-[3-(N-methylamino)carbonylmethyleneoxyphenyl]-2,4-pyrimidinediamine,
N4-(3-Chloro-4-methylphenyl)-5-fluoro-N2-[3-(N-methylamino)-carbonylmethyleneoxyphenyl]-2,4-pyri-midinediamine,
N4-(3-Chloro-4-methoxyphenyl)-5-fluoro-N2-[3-[(N-methylamino)carbonylmethyleneoxy]phenyl]-2,4-pyrimidinediamine,
5-Fluoro-N4-1(1H)-indol-5-yl]-N2-[3-[(N-methylamino)carbonylmethyleneoxy]phenyl]-2,4-pyrimidine-diamine,
5-Fluoro-N4-(3-hydroxyphenyl)-N2-[1-(methoxycarbonyl)methyl-indazoline-5-yl]-2,4-pyrimidinediamine,
5-Fluoro-N4-(3-hydroxyphenyl)-N2-[1-(3-hydroxypropyl)indazoline-6-yl]-2,4-pyrimidinediamine,
N4-(3,4-Ethylenedioxyphenyl)-5-fluoro-N2-[1-(3-hydroxypropyl)indazoline-5-yl]-2,4-pyrimidinediamine,
5-Fluoro-N4-(3-hydroxyphenyl)-N2-[1-(3-hydroxypropyl)indazoline-5-yl]-2,4-pyrimidinediamine,
5-Fluoro-N2-[1-(3-hydroxypropyl)indazoline-5-yl]-N4-(4-isopropoxyphenyl)-2,4-pyrimidinediamine,
N4-(3,4-Ethylenedioxyphenyl)-5-fluoro-N2-[1-[2(N-methylaminocarbonyl)ethyl]-indazoline-5-yl]-2,4-pyrimidinediamine,
5-Fluoro-N4-(4-isopropoxyphenyl)-N2-[1-[2(N-methylaminocarbonyl)ethyl]-indazoline-5-yl]-2,4-pyrimi-dinediamine,
N4-[(2,2-dimethyl-4H-benzo[1,4]oxazin-3-one)-6-yl]-5-fluoro-N2-[3-(methylaminocarbonylmethylene-oxy)phenyl]-2,4-pyrimidinediamine,
N4-[(2,2-Dimethyl-4H-benzo[1,4]oxazin-3-one)-6-yl]-5-fluoro-N2-(1-methylindazolin-5-yl)-2,4-pyrimidinediamine,
N4-[(2,2-Difluoro-4H-benzo[1,4]oxazin-3-one)-6-yl]-5-fluoro-N2-[3-(methylaminocarbonylmethyleneoxy)phenyl]-2,4-pyrimidinediamine,
N4-1(2,2-Dimethyl-4H-5-pyridol-1,4]oxazin-3-one)-6-yl]-5-fluoro-N2-[3-(methylaminocarbonylmethyleneoxy)phenyl]-2,4-pyrimidinediamine,
5-Fluoro-N2-(3-methylaminocarbonylmethyleneoxyphenyl)-N4-[2H-pyrido[3,2-b]-1,4-oxazin-3(4H)-one-6-yl]-2,4-pyrimidinediamine,
N4-(4-Amino-3,4-dihydro-2H-1-benzopyran-6-yl)-5-fluoro-N2-[3-(N-methylamino)carbonylmethyleneoxyphenyl]-2,4-pyrimidinediamine,
N4-(3-Chloro-4-hydroxy-5-methylphenyl)-5-fluoro-N2-[3-[2-(N-piperazino)ethoxy]phenyl]-2,4-pyrimidinediamine, and
N4-(3-Methylcarbonyloximephenyl)-5-fluoro-N2-[3-(N-methylamino)carbonylmethyleneoxyphenyl]-2,4-pyrimidinediamine,
   or salts thereof.

LPA receptor antagonist. Particularly in those embodiments which are feeder-free, the medium may also include an LPA receptor antagonist, such as an antagonist that inhibits LPA1 and LPA3-induced inositol phosphate production with Ki's for each of 1000 μM or less, and is a substantially weaker inhibition for LPA2, LPA4, LPA5, LPA6, i.e., with Ki's for each of 5000 μM or less. Ki16198 is a preferred LPA receptor antagonist, and is the methyl ester of Ki16425.

Other LPA receptor antagonists include H2L5765834, H2L5186303, Ki 16425, Ro 6842262 and C LPA5 4.

In one aspect, provided herein are LPA receptor inhibitors, or salts, solvates, polymorphs, prodrugs, metabolites, N-oxides, stereoisomers, or isomers thereof, having the structure shown in US20170042915A1, such as one of the following structures:

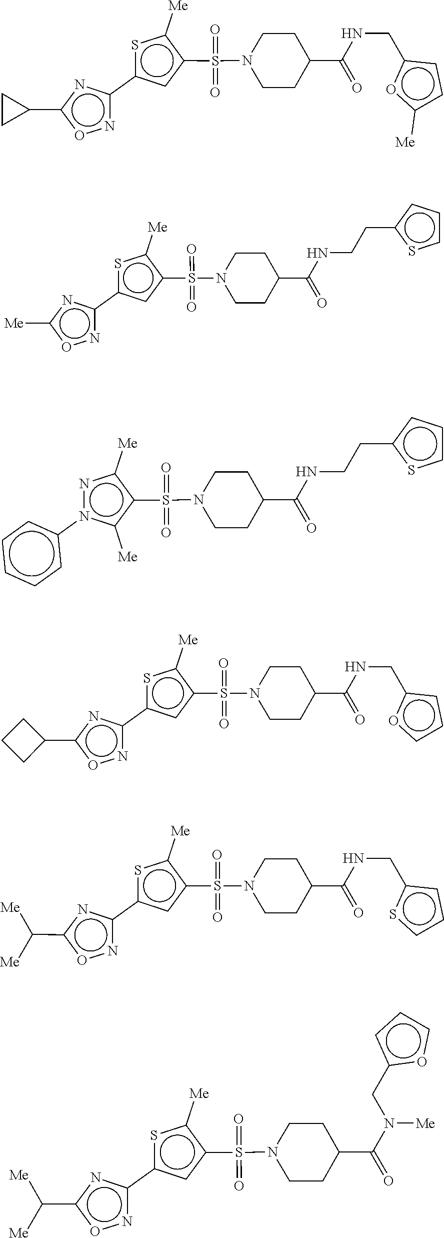

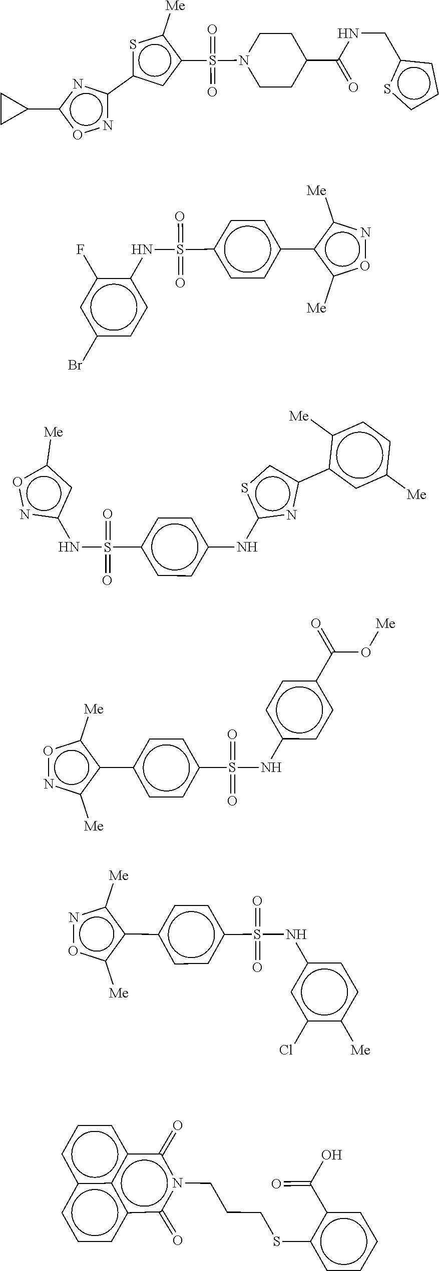

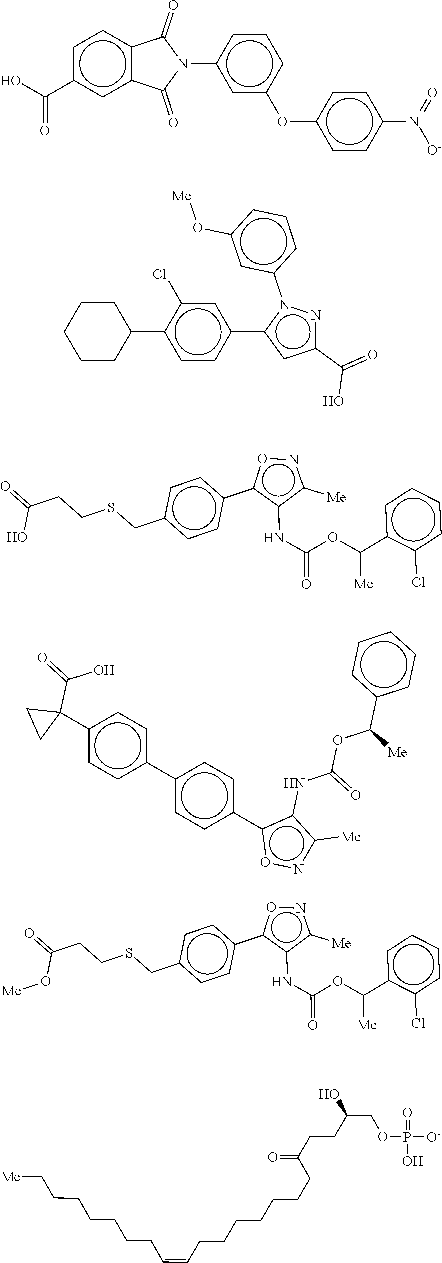

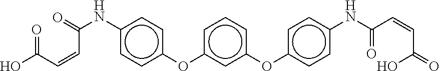

GSK3 inhibitor. Particularly in those embodiments which are feeder-free, the medium may also include a GSK3 Inhibitor. Exemplary GSK3 inhibitors include CHIR-99021 (CT99021) HCl, SB216763, CHIR-98014, TWS119, Tideglusib, SB415286, CHIR-99021 (CT99021), AZD2858, AZD1080, AR-A014418, TDZD-8, LY2090314, BIO-acetoxime, IM-12, 1-Azakenpaullone, Indirubin and 6-BIO.

Suitable GSK-3 inhibitors, methods for their synthesis and assays for GSK inhibition are also described in, for example, WO 03/004472, WO 03/055492, WO 03/082853, WO 2004/018455, WO 2004/037791, 06/001754, WO 07/040436, WO 07/040438, WO 07/040439, WO 07/040440, WO08/002244, WO08/002245 and Coghlan et al. Chemistry & Biology 2000, 7(10):793-803. GSK-3 inhibitors are also reviewed in, for example, Cohen et al. Nature Reviews Drug Discovery 2004, 3:479-487; Kramer et al. International Journal of Alzheimer's Disease Volume 2012, Article ID 381029, 32 pages; and Eldar-Finkelman et al., Front Mol Neurosci. 2011; 4:32. In one embodiment, the GSK-3 inhibitor is lithium, e.g., a lithium salt such as lithium carbonate, citrate, chloride, orotate, bromide or chloride. In another embodiment, the GSK-3 inhibitor is 3-(2,4-dichlorophenyl)-4-(I-methyl-IH-indol-3yl)-IH-pyrrole-2,5-dione (SB216763) or 3-(3-chloro-4-hydroxyphenylamino)-4-(2nitrophenyl)-IH-pyrrole-2,5-dione (SB-415286), the structures of which are shown as formulae below:

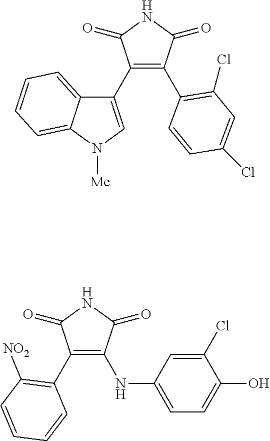 [SMILES: Cn1cc(C2=C(c3ccc(Cl)cc3Cl)C(=O)NC2=O)c2ccccc21 ; O=C1NC(=O)C(c2ccccc2[N+](=O)[O-])=C1Nc1ccc(O)c(Cl)c1]

(designated SB216763 and SB-415286 in the literature).

Further GSK3 inhibitors include 6-BIO, hymenialdisine, dibromocantharelline, CT98014, CT98023, CT99021, TWS119, AR-A014418, AZD-1080, kenpaullone, alsterpaullone, cazpaullone, aloisine A, manzamine A, palinurine, tricantine, TDZD-8, NP00111, P031115, P031112 (tideglusib), HMK-32 and L803-mts, the chemical structures and synthesis of which are described or referenced in Eldar-Finkelman et al., Front Mol Neurosci. 2011; 4:32.

CK2 inhibitor. Particularly in those embodiments which are feeder-free, the medium may also include a CK2 inhibitor, such as CX-4945 (Silmitasertib), CX-8184, DMAT, ellagic acid or TTP22.

Further exemplary CK2 inhibitors are one taught in PCT Publication WO 2017/070137 A1, such as a compound of formula:

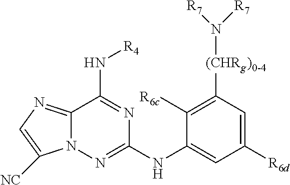

including enantiomers, diastereomers, tautomers, acceptable salts, prodrugs, hydrates, or solvates thereof, wherein
R₄ is selected from the group consisting of C₁₋₄ alkyl substituted with 1-3 Rₑ, C₃₋₆ cycloalkyl and heterocyclyl substituted with 1-3 Rₑ;
Two R₇ groups, together with the nitrogen atom to which they are both attached, form a 4- to 7-membered monocyclic or 7- to 12-membered bicyclic heterocycle containing carbon atoms and additional 1-3 heteroatoms selected from the group consisting of NR₈ₐ, O, and S(O)₂ and substituted with 1-4 Rs;
R₈, at each occurrence, is independently selected from the group consisting of H, F, Cl, Br, C₁₋₄ alkyl substituted with 1-4 Rₑ, ═O (ketone), C₂₋₄ alkenyl substituted with 1-5 Rₑ, —(CHR_{g})ᵣOR_{b}, —CHR_{g})ᵣS(O)ₚR_{c}, —(CHR_{g})ᵣC(═O)(CHR_{g})ᵣR_{d}, —(CHR_{g})ᵣNRₐRₐ, —(CHR_{g})ᵣC(═O)N RₐRₐ, —(CHR_{g})ᵣC(═O)NRₐS(O)ₚR_{c},—(CHR_{g})ᵣNRₐ(CR_{g}R_{g})ᵣC(═O)R_{d}, —(CHR_{g})ᵣNRₐC(═O)OR_{b}, —(C HR_{g})ᵣOC(═O)(CHR_{g})ᵣR_{d}, —(CHR_{g})ᵣOC(═O)(CHR_{g})ᵣC(═O)OR_{d}, —(CHR_{g})ᵣOC(═O)(CHR_{g})ᵣC(═O)N RₐRₐ, —(CHR_{g})ᵣOC(═O)(CHR_{g})ᵣNRₐC(═O)R_{b}, —(CHR_{g})ᵣOC(═O)(CHR_{g})ᵣNRₐRₐ, —(CHR_{g})ᵣNRₐC(═O)NRₐRₐ, —(CHR_{g})ᵣC(═O)(CH₂)ᵣOR_{b}, —(CHR_{g})ᵣC(═O)(CHR_{g})ᵣOC(═O)R_{b}, —(CHR_{g})ᵣS(0)₂NRₐRₐ, —(CHR_{g})ᵣNRₐS(0)ₚNRₐRₐ, —(CHR_{g})ᵣNRₐS(0)ₚRₑ, —OPO₃H, —(CHR_{g})ᵣ—C₃₋₆ cycloalkyl substituted with 1-5 Rₑ, —(CHR_{g})ᵣ-aryl substituted with 1-4 Rₑ and —(CHR_{g})ᵣ-heterocyclyl substituted with 1-4 Rₑ;
R₈ₐ is selected from the group consisting of H, C₁₋₄ alkyl substituted with 1-5 Rₑ, C₂₋₄ alkenyl substituted with 1-5 Rₑ, —(CHR_{g})ᵣOR_{b}, —(CHR_{g})ᵣS(O)ₚR_{c}, —(CHRg)rC(═O)(CHRg)rRd, —CHRg)rNRaRa, (CHRg)rC(═O)NRaRa, —(CHR_{g})ᵣC(═O)NRₐS(O)ₚRₑ, —(CHR_{g})ᵣNRₐ(CR_{g}R_{g})ᵣC(═O)R_{d}, —(CHR_{g})ᵣNHC(═O)OR_{b}, —(CHR_{g})ᵣOC(═O)(CHR_{g})ᵣR_{d}, —(CHR_{g})ᵣOC(═O)(CHR_{g})ᵣC(═O)OR_{d}, —(CHR_{g})ᵣOC(═O)(CHR_{g})ᵣC(═O)NRₐRₐ, —(CHR_{g})ᵣOC(═O)(CHR_{g})ᵣ NRₐC(═O)R_{b}, —(CHR_{g})ᵣOC(═O)(CHR_{g})ᵣNRₐRₐ, —(CHR_{g})ᵣNRₐ(CHR_{g})ᵣC(═O)NRₐRₐ, —(CHR_{g})ᵣC(═O)OR_{b}, —(CHR_{g})ᵣC(═O)(CHR_{g})ᵣOC(═O)R_{b}, —(CHR_{g})ᵣS(O)₂NRₐRₐ, —(CHR_{g})ᵣNRₐS(O)ₚNRₐRₐ, —(CHR_{g}rNRₐS(O)ₚR_{c}, —OPO₃H, —(CHR_{g})ᵣ-C₃₋₆ cycloalkyl substituted with 1-5 Rₑ, —(CHR_{g}),-aryl substituted with 1-4 Rₑ and —(CHR_{g})ᵣ-heterocyclyl substituted with 1-4 Rₑ;
Rₐ, at each occurrence, is independently selected from the group consisting of H, CN, C₁₋₆ alkyl substituted with 1-5 Rₑ, C₂₋₆ alkenyl substituted with 1-5 Rₑ, C₂₋₆ alkynyl substituted with 1-5 Rₑ, —(CH₂)ᵣ-C₃-iocarbocyclyl substituted with 1-5 Rₑ, and —(CH₂)ᵣ-heterocyclyl substituted with 1-5 Rₑ; or Rₐ and Rₐ together with the nitrogen atom to which they are both attached form a heterocyclic ring substituted with 1-5 Rₑ;
R_{b}, at each occurrence, is independently selected from the group consisting of H, C₁₋₆ alkyl substituted with 1-5 Rₑ, C₂₋₆ alkenyl substituted with 1-5 Rₑ, C₂₋₆ alkynyl substituted with 1-5 Rₑ, —(CH₂)ᵣ-C3-iocarbocyclyl substituted with 1-5 Rₑ, and —(CH2)ᵣ-heterocyclyl substituted with 1-5 Rₑ;
R_{c}, at each occurrence, is independently selected from the group consisting of Ci-6 alkyl substituted with 1-5 Rₑ, C₂₋₆ alkenyl substituted with 1-5 Rₑ, C₂₋₆ alkynyl substituted with 1-5 Rₑ, C₃₋₆ carbocyclyl, and heterocyclyl;
R_{d}, at each occurrence, is independently selected from the group consisting of H, OH, C₁₋₆ alkyl substituted with 1-5 Rₑ, C₂₋₆ alkenyl substituted with 1-5 Rₑ, C₂₋₆ alkynyl substituted with 1-5 Rₑ, —(CH₂)ᵣ-C3-iocarbocyclyl substituted with 1-5 Rₑ, and —(CH₂)ᵣ-heterocyclyl substituted with 1-5 Rₑ;
Rₑ, at each occurrence, is independently selected from the group consisting of H, N₃, C₁₋₆ alkyl substituted with 1-5 R_{f}, C₂₋₆ alkenyl, C₂₋₆ alkynyl, —(CH₂)ᵣ-C₃₋₆ cycloalkyl, (CH₂)ᵣ-heterocyclyl, F, C, Br, —(CH₂)ᵣCN, NO₂, ═O, —OPO₃H, —OSi(C₁₋₄ alkyl)₃, (CH₂)ᵣOC₁₋₅ alkyl, —(CH₂)ᵣO(CH₂)ᵣOC₁₋₅ alkyl, —(CH₂)ᵣOH, —(CH₂)ᵣS(O)₂C₁₋₅ alkyl, —(CH₂)ᵣS(O)₂R_{f}, —(CH₂)ᵣNHS(O)₂C₁₋₅ alkyl, —S(O)₂NH₂, —SH, —(CH₂)ᵣNR_{f}R_{f}, —(CH₂)ᵣNHC(═O)OR_{f}, —(CH₂)ᵣNHC(═O)R_{f}, —(CH₂)ᵣNHC(═NH)NR_{f}R_{f}, —(CH₂)ᵣC(═O)(CH₂)ᵣR, and —(CH₂)ᵣC(═O)OR_{f};
R_{f}, at each occurrence, is independently selected from the group consisting of H, —CH₂OH, —(CH₂)ᵣOC₁₋₅ alkyl, C₁₋₅ alkyl (optionally substituted with F, Cl, OH, NH₂), C₃₋₆ cycloalkyl optionally substituted with NH₂, —(CH₂)ᵣS(O)PC₁₋₄ alkyl, —NHC(═O)C₁₋₄ alkyl, —C(═O)NH₂, —C(═O)OC₁₋₄ alkyl, —C(═O)C₁₋₄ alkyl, —(CH₂)ᵣ-phenyl, —(CH₂)ᵣ-heterocyclyl optionally substituted with alkyl and CN, or R_{f} and R_{f} together with the nitrogen atom to which they are both attached form a heterocyclic ring optionally substituted with C₁₋₄ alkyl;
R_{g}, at each occurrence, is independently selected from the group consisting of H, F, OH, and C₁₋₅ alkyl;
p, at each occurrence, is independently selected from the group consisting of zero, 1, and 2; and
r, at each occurrence, is independently selected from the group consisting of zero, 1, 2, 3, 4, and 5.

Notch Agonist. The culture medium of the invention may additionally include a Notch agonist. Notch signaling has been shown to play an important role in cell-fate determination, as well as in cell survival and proliferation. Notch receptor proteins can interact with a number of surface-bound or secreted ligands, including but not limited to Jagged-1, Jagged-2, Delta-1 or Delta-like 1, Delta-like 3, Delta-like 4, etc. Upon ligand binding, Notch receptors are activated by serial cleavage events involving members of the ADAM protease family, as well as an intramembranous cleavage regulated by the gamma secretase presinilin. The result is a translocation of the intracellular domain of Notch to the nucleus, where it transcriptionally activates downstream genes.

A “Notch agonist” as used herein includes a molecule that stimulates a Notch activity in a cell by at least about 10%, at least about 20%, at least about 30%, at least about 50%, at least about 70%, at least about 90%, at least about 100%, at least about 3-fold, 5-fold, 10-fold, 20-fold, 50-fold, 100-fold, 200-fold, 500-fold, 1000-fold or more, relative to a level of a Notch activity in the absence of the Notch agonist. As is known in the art, Notch activity can be determined by, for example, measuring the transcriptional activity of Notch, by a 4×wtCBF1-luciferase reporter construct described by Hsieh et al. (Mol. Cell. Biol. 16:952-959, 1996, incorporated herein by reference).

In certain embodiments, the Notch agonist is selected from: Jagged-1, Delta-1 and Delta-like 4, or an active fragment or derivative thereof. In certain embodiments, the Notch agonist is DSL peptide (Dontu et al., Breast Cancer Res., 6:R605-R615, 2004), having the amino acid sequence CDDYYYGFGCNKFCRPR (SEQ ID NO: 36). The DSL peptide (ANA spec) may be used at a concentration between 10.mu·M and 100 nM, or at least 10 μM and not higher than 100 nM. In certain embodiments, the final concentration of Jagged-1 is about 0.1-10 μM; or about 0.2-5 μM; or about 0.5-2 μM; or about 1 μM.

In certain embodiments, any of the specific Notch agonist referenced herein, such as Jagged-1, Jagged-2, Delta-1 and Delta-like 4 may be replaced by a natural, synthetic, or recombinantly produced homologs or fragments thereof that retain at least about 80%, 85%, 90%, 95%, 99% of the respective Notch agonist activity, and/or homologs or fragments thereof that share at least about 60%, 70%, 80%, 90%, 95%, 97%, 99% amino acid sequence identity as measured by any art recognized sequence alignment software based on either a global alignment technique (e.g., the Needleman-Wunsch algorithm) or a local alignment technique (e.g., the Smith-Waterman algorithm).

The sequences of the representative Notch agonists referenced herein are represented in SEQ ID NOs. 28-35.

The Notch agonist may be added to the culture medium every 1, 2, 3, or 4 days during the first 1-2 weeks of culturing the stem cells.

Nicotinamide. The culture medium of the invention may additionally be supplemented with nicotinamide or its analogs, precursors, or mimics, such as methyl-nicotinamid, benazamid, pyrazinamide, thymine, or niacin. Nicotinamide may be added to the culture medium to a final concentration of between 1 and 100 mM, between 5 and 50 mM, or preferably between 5 and 20 mM. For example, nicotinamide may be added to the culture medium to a final concentration of approximately 10 mM. The similar concentrations of nicotinamide analogs, precursors, or mimics can also be used alone or in combination.

Extracellular Matrix (ECM). Extracellular matrix (ECM), used interchangeably herein with “basement membrane matrix,” is secreted by connective tissue cells, and comprises a variety of polysaccharides, water, elastin, and proteins that may comprise proteoglycans, collagen, entactin (nidogen), fibronectin, fibrinogen, fibrillin, laminin, and hyaluronic acid. ECM may provide the suitable substrate and microenvironment conductive for selecting and culturing the subject stem cells.

In certain embodiments, the subject stem cells are attached to or in contact with an ECM. Different types of ECM are known in the art, and may comprise different compositions including different types of proteoglycans and/or different combination of proteoglycans. The ECM may be provided by culturing ECM-producing cells, such as certain fibroblast cells. Examples of extracellular matrix-producing cells include chondrocytes that mainly produce collagen and proteoglycans; fibroblast cells that mainly produce type IV collagen, laminin, interstitial procollagens, and fibronectin; and colonic myofibroblasts that mainly produce collagens (type I, III, and V), chondroitin sulfate proteoglycan, hyaluronic acid, fibronectin, and tenascin-C.

In certain embodiments, at least some ECM is produced by the murine 3T3-J2 clone, which may be grown on top of the MATRIGEL™ basement membrane matrix (BD Biosciences) as feeder cell layer.

Alternatively, the ECM may be commercially provided. Examples of commercially available extracellular matrices are extracellular matrix proteins (Invitrogen) and MATRIGEL™ basement membrane matrix (BD Biosciences). The use of an ECM for culturing stem cells may enhance long-term survival of the stem cells and/or the continued presence of undifferentiated stem cells. An alternative may be a fibrin substrate or fibrin gel or a scaffold, such as glycerolized allografts that are depleted from the original cells.

In certain embodiments, the ECM for use in a method of the invention comprises at least two distinct glycoproteins, such as two different types of collagen or a collagen and laminin. The ECM may be a synthetic hydrogel extracellular matrix, or a naturally occurring ECM. In certain embodiments, the ECM is provided by MATRIGEL™ basement membrane matrix (BD Biosciences), which comprises laminin, entactin, and collagen IV.

Medium. A cell culture medium that is used in a method of the invention may comprise any cell culture medium, such as culture medium buffered at about pH 7.4 (e.g., between about pH 7.2-7.6) with a carbonate-based buffer. Many commercially available tissue culture media are potentially suitable for the methods of the invention, including, but are not limited to, Dulbecco's Modified Eagle Media (DMEM, e.g., DMEM without L-glutamine but with high glucose), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F10, Ham's F-12, Medium 199, and RPMI 1640 Media.

The cells may be cultured in an atmosphere comprising between 5-10% CO₂ (e.g., at least about 5% but no more than 10% C02, or about 5% C02). In certain embodiments, the cell culture medium is DMEM/F12 (e.g., 3:1 mixture) or RPMI 1640, supplemented with L-glutamine, insulin, Penicillin/streptomycin, and/or transferrin. In certain embodiments, Advanced DMEM/F12 or Advanced RPMI is used, which is optimized for serum free culture and already includes insulin. The Advanced DMEM/F12 or Advanced RPMI medium may be further supplemented with L-glutamine and Penicillin/streptomycin. In certain embodiments, the cell culture medium is supplemented with one or more a purified, natural, semisynthetic and/or synthetic factors described herein. In certain embodiments, the cell culture medium is supplemented by about 10% fetal bovine serum (FBS) that is not heat inactivated prior to use. Additional supplements, such as, for example, B-27® Serum Free Supplement (Invitrogen), N-Acetylcysteine (Sigma) and/or N2 serum free supplement (Invitrogen), or Neurobasal (Gibco), TeSR (StemGent) may also be added to the medium.

In certain embodiments, the medium may contain one or more antibiotics to prevent contamination (such as Penicillin/streptomycin). In certain embodiments, the medium may have an endotoxin content of less than 0.1 endotoxin units per mL, or may have an endotoxin content less than 0.05 endotoxin units per mL. Methods for determining the endotoxin content of culture media are known in the art.

A cell culture medium according to the invention allows the survival and/or proliferation and/or differentiation of epithelial stem cells on an extracellular matrix. The term “cell culture medium” as used herein is synonymous with “medium,” “culture medium,” or “cell medium.”

The modified (growth) medium of the invention comprises, in a base medium, (a) a ROCK (Rho Kinase) inhibitor; (b) a Wnt agonist; (c) a mitogenic growth factor; (d) a TGF-beta signaling pathway inhibitor, such as TGF-beta inhibitor, or a TGF-beta receptor inhibitor); and (e) insulin or IGF; and the medium optionally further comprising a Bone Morphogenetic Protein (BMP) antagonist.

Thus in one aspect, the invention provides a base medium (Base Medium) comprising: insulin or an insulin-like growth factor; T3 (3,3′,5-Triiodo-LThyronine); hydrocortisone; adenine; EGF; and 10% fetal bovine serum (without heat inactivation), in DMEM:F12 3:1 medium supplemented with L-glutamine.

In certain embodiments, the Base Medium comprises about: 5 μg/mL insulin; 2×10″9 M T3 (3,3′,5-Triiodo-LThyronine); 400 ng/mL hydrocortisone; 24.3 μg/mL adenine; 10 ng/mL EGF; and 10% fetal bovine serum (without heat inactivation), in DMEM:F12 3:1 medium supplemented with 1.35 mM L-glutamine.

In certain embodiments, the concentration for each of the medium components referenced in the immediate preceding paragraph is independently 2%, 5%, 10%, 15%, 20%, 25%, 30%, 35%, 40%, 45%, 50%, 60%, 70%, 80%, 90%, 95% higher or lower than the respective recited value, or 2-fold, 3-fold, 5-fold, 10-fold, 20-fold higher than the respective recited value. For example, in an illustrative medium, insulin concentration may be 6 μg/mL (20% higher than the recited 5 μg/mL), EGF concentration may be 5 ng/mL (50% lower than the recited 10 ng/mL), while the remaining components each has the same concentration recited above.

In a related aspect, the invention provides a base medium containing cholera enterotoxin. In other embodiments, the base medium does not contain cholera enterotoxin.

The Base Medium may further comprise one or more antibiotics, such as Pen/Strep, and/or gentamicin.

The base media may be used to produce Modified Growth Medium (or simply Modified Medium) by adding one or more of the factors above.

### 4. Protein Sequences of the Representative Medium Factors

Several representative (non-limiting) protein factors used in the media and methods of the invention are provided below. For each listed factor, numerous homologs or functional equivalents are known in the art and can be readily retrieved from public databases such as GenBank, EMBL, and/or NCBI RefSeq, just to name a few. Additional proteins or peptide fragments thereof, or polynucleotides encoding the same, including functional homologs from human or non-human mammals, can be readily retrieved from public sources through, for example, sequence-based searches such as NCBI BLASTp or BLASTn or both.

BMP inhibitors
Noggin: (GenBank: AAA83259.1), Homo sapiens:
(SEQ ID NO: 1)
MERCPSLGVT LYALVVVLGL RATPAGGQHY LHIRPAPSDN LPLVDLIEHP DPIFDPKEKD LNETLLRSLL
GGHYDPGFMA TSPPEDRPGG GGGAAGGAED LAELDQLLRQ RPSGAMPSEI KGLEFSEGLA QGKKQRLSKK
LRRKLQMWLW SQTFCPVLYA WNDLGSRFWP RYVKVGSCFS KRSCSVPEGM VCKPSKSVHL TVLRWRCQRR
GGQRCGWIPI QYPIISECKC SC
Chordin (GenBank: AAG35767.1), Homo sapiens:
(SEQ ID NO 2)
MPSLPAPPAP LLLLGLLLLG SRPARGAGPE PPVLPIRSEK EPLPVRGAAG CTFGGKVYAL DETWHPDLGE
PFGVMRCVLC ACEAPQWGRR TRGPGRVSCK NIKPECPTPA CGQPRQLPGH CCQTCPQERS SSERQPSGLS
FEYPRDPEHR SYSDRGEPGA EERARGDGHT DFVALLTGPR SQAVARARVS LLRSSLRFSI SYRRLDRPTR
IRFSDSNGSV LFEHPAAPTQ DGLVCGVWRA VPRLSLRLLR AEQLHVALVT LTHPSGEVWG PLIRHRALAA
ETFSAILTLE GPPQQGVGGI TLLTLSDTED SLHFLLLERG LLEPRSGGLT QVPLRLQILH QGQLLRELQA
NVSAQEPGFA EVLPNLTVQE MDWLVLGELQ MALEWAGRPG LRI SGHIAAR KSCDVLQSVL
CGADALIPVQ TGAAGSASLT LLGNGSLIYQ VQVVGTSSEV VAMTLETKPQ RRDQRTVLCH MAGLQPGGHT
AVGICPGLGA RGAHMLLQNE LFLNVGTKDF PDGELRGHVA ALPYCGHSAR HDTLPVPLAG ALVLPPVKSQ
AAGHAWLSLD THCHLHYEVL LAGLGGSEQG TVTAHLLGPP GTPGPRRLLK GFYGSEAQGV VKDLEPELLR
HLAKGMASLL ITTKGSPRGE LRGQVHIANQ CEVGGLRLEA AGAEGVRALG APDTASAAPP VVPGLPALAP
AKPGGPGRPR DPNTCFFEGQ QRPHGARWAP NYDPLCSLCT CQRRTVICDP VVCPPPSCPH PVQAPDQCCP
VCPEKQDVRD LPGLPRSRDP GEGCYFDGDR SWRAAGTRWH PVVPPFGLIK CAVCTCKGGT GEVHCEKVQC
PRLACAQPVR VNPTDCCKQC PVGSGAHPQL GDPMQADGPR GCRFAGQWFP ESQSWHPSVP PFGEMSCITC
RCGAGVPHCE RDDCSLPLSC GSGKESRCCS RCTAHRRPAP ETRTDPELEK EAEGS
Follistatin (GenBank: AAH04107.1) Homo sapiens:
(SEQ ID NO: 3)
MVRARHQPGG LCLLLLLLCQ FMEDRSAQAG NCWLRQAKNG RCQVLYKTEL SKEECCSTGR LSTSWTEEDV
NDNTLFKWMI FNGGAPNCIP CK-ETCENVDC GPGKKCRMNK KNKPRCVCAP
DCSNITWKGP VCGLDGKTYR NECALLKARC KEQPELEVQY QGRCKKTCRD VFCPGSSTCV VDQTNNAYCV
TCNRICPEPA SSEQYLCGND GVTYSSACHL RKASCLLGRS IGLAYEGKCI KAKSCEDIQC TGGKKCLWDF
KVGRGRCSLC DELCPDSKSD EPVCASDNAT YASECAMKEA ACSSGVLLEV KHSGSCNSIS EDTEEEEEDE
DQDYSFPISS ILEW
DAN (GenBank: BAA92265.1) Homo sapiens:
(SEQ ID NO: 4)
MLRVLVGAVL PAMLLAAPPP INKLALFPDK SAWCEAKNIT QIVGHSGCEA KSIQNRACLG QCFSYSVPNT
FPQSTESLVH CDSCMPAQSM WEIVTLECPG HEEVPRVDKL VEKILHCSCQ ACGKEPSHEG LSVYVQGEDG
PGSQPGTHPH PHPHPHPGGQ TPEPEDPPGA PHTEEEGAED
Cerberus (NCBI Reference Sequence: NP_005445.1) Homo sapiens:
(SEQ ID NO: 5)
MHLLLFQLLV LLPLGKTTRH QDGRQNQSSL SPVLLPRNQR ELPTGNHEEA EEKPDLFVAV PHLVATSPAG
EGQRQREKML SRFGREWKKP EREMHPSRDS DSEPFPPGTQ SLIQPIDGMK MEKSPLREEA KKFWHHFMFR
KTPASQGVIL PIKSHEVHWE TCRTVPFSQT ITHEGCEKVV VQNNLCFGKC GSVHFPGAAQ HSHTSCSHCL
PAKFTTMHLP LNCTELSSVI KVVMLVEECQ CKVKTEHEDG HILHAGSQDS FIPGVSA
Gremlin (GenBank: AAF06677.1) Homo sapiens:
(SEQ ID NO: 6)
MSRTAYTVGA LLLLLGTLLP AAEGKKKGSQ GAIPPPDKAQ HNDSEQTQSP QQPGSRNRGR GQGRGTAMPG
EEVLESSQEA LHVTERKYLK RDWCKTQPLK QTIHEEGCNS RTI INRFCYG QCNSFYIPRH
IRKEEGSFQS CSFCKPKKFT IMMVTLNCPE LQPPTKKKRV TRVKQCRCIS IDLD
Sclerostin/SOST (GenBank: AAK13451.1) Homo sapiens:
(SEQ ID NO: 7)
MQLPLALCLV CLLVHTAFRV VEGQGWQAFK NDATEI IPEL GEYPEPPPEL ENNKTMNRAE
NGGRPPHHPF ETKDVSEYSC RELHFTRYVT DGPCRSAKPV TELVCSGQCG PARLLPNAIG RGKWWRP SGP
DERCIPDRYR AQRVQLLCPG GEAPRARKVR LVASCKCKRL TRFHNQSELK DFGTEAARPQ KGRKPRPRAR
SAKANQAELE NAY
Decorin (GenBank: AAB60901.1) Homo sapiens:
(SEQ ID NO: 8)
MKATI ILLLL AQVSWAGPFQ QRGLFDFMLE DEASGIGPEV PDDRDFEPSL GPVCPFRCQC HLRVVQCSDL
alpha-2 macroglobulin (GenBank: EAW88590.1) Homo sapiens:
(SEQ ID NO 9)
MGKNKLLHPS LVLLLLVLLP TDASVSGKPQ YMVLVPSLLH TETTEKGCVL LSYLNETVTV SASLESVRGN
RSLFTDLEAE NDVLHCVAFA VPKSSSNEEV MFLEVQVKGP TQEFKKRTTV MVKNEDSLVF VQTDKSIYKP
GQTVKFRVVS MDENFHPLNE LIPLVYIQDP KGNRIAQWQS FQLEGGLKQF SFPLSSEPFQ GSYKVVVQKK
SGGRTEHPFT VEEFVLPKFE VQVTVPKIIT ILEEEMNVSV CGLYTYGKPV PGHVTVSICR KYSDASDCHG
EDSQAFCEKF SGQLNSHGCF YQQVKTKVFQ LKRKEYEMKL HTEAQIQEEG TVVELTGRQS SEITRTITKL
SFVKVDSHER QGIPFFGQVR LVDGKGVPIP NKVIFIRGNE ANYYSNATTD EHGLVQFSIN TTNVMGTSLT
VRVNYKDRSP CYGYQWVSEE HEEAHHTAYL VESPSKSFVH LEPMSHELPC GHTQTVQAHY ILNGGILLGL
KKLSFYYLIM AKGGIVRIGT HGLLVKQEDM KGHFSISIPV KSDIAPVARL LIYAVLPTGD VIGDSAKYDV
ENCLANKVDL SFSPSQSLPA SHAHLRVTAA PQSVCALRAV DQSVLLMKPD AELSASSVYN LLPEKDLIGE
PGPLNDQDDE DCINRHNVYI NGITYTPVSS TNEKDMYSEL EDMGLKAFTN SKIRKPKMCP QLQQYEMHGP
EGLRVGFYES DVMGRGHARL VHVEEPHTET VRKYFPETWI WDLVVVNSAG VAEVGVTVPD TITEWKAGAF
CLSEDAGLGI SSTASLRAFQ PFFVELTMPY SVIRGEAFTL KATVLNYLPK CIRVSVQLEA SPAFLAVPVE
KEQAPHCICA NGRQTVSWAV TPKSLGNVNF TVSAEALESQ ELCGTEVPSV PEHGRKDTVI KPLLVEPEGL
EKETTENSLL CPSGGEVSEE LSLKLPPNVV EESARASVSV LGDILGSAMQ NTQNLLQMPY GCGEQNMVLE
APNIYVLDYL NETQQLTPEI KSKAIGYLNT GYQRQLNYKH YDGSYSTEGE RYGRNQGNTW LTAFVLKTFA
QARAYIFIDE AHITQALIWL SQRQKDNGCF RSSGSLLNNA IKGGVEDEVT LSAYITIALL EIPLTVTHPV
VRNALFCLES AWKTAQEGDH GSHVYTKALL AYAFALAGNQ DKRKEVLKSL NEEAVKKDNS VHWERPQKPK
APVGHFYEPQ APSAEVEMTS YVLLAYLTAQ PAPESEDLTS ATNIVKWITK QQNAQGGFSS TQDTVVALHA
LSKYGAATFT RTGKAAQVTI QSSGTFSSKF QVDNNNRLLL QQVSLPELPG EYSMKVTGEG CVYLQTSLKY
NILPEKEEFP FALGVQTLPQ TCDEPKAHIS FQISLSVSYT GSRSASNMAI VDVKMVSGFI PLKPTVKMLE
RSNHVSRTEV SSNHVLIYLD KVSNQTLSLF FTVLQDVPVR DLKPAIVKVY DYYETDEFAI AEYNAPCSKD
LGNA
Wnt Agonists
R-spondin 1 (GenBank: ABC54570.1) Homo sapiens:
(SEQ ID NO: 10)
MRLGLCVVAL VLSWTHLTIS SRGIKGKRQR RISAEGSQAC AKGCELCSEV NGCLKCSPKL FILLERNDIR
QVGVCLPSCP PGYFDARNPD MNKCIKCKIE HCEACFSHNF CTKCKEGLYL HKGRCYPACP EGSSAANGTM
ECSSPAQCEM SEWSPWGPCS KKQQLCGERR GSEERTRRVL HAPVGDHAAC SDTKETRRCT VRRVPCPEGQ
KRRKGGQGRR ENANRNLARK ESKEAGAGSR RRKGQQQQQQ QGTVGPLTSA GPA
R-spondin 2 (NCBI Reference Sequence: NP_848660.3) Homo sapiens:
(SEQ ID NO: 11)
MQFRLESFAL I ILNCMDYSH CQGNRWRRSK RASYVSNPIC KGCLSCSKDN GCSRCQQKLF
FFLRREGMRQ YGECLHSCPS GYYGHRAPDM NRCARCRIEN CDSCFSKDFC TKCKVGFYLH RGRCFDECPD
GFAPLEETME CVEGCEVGHW SEWGTCSRNN RTCGFKWGLE TRTRQIVKKP VKDTILCPTI AESRRCKMTM
RHCPGGKRIP KAKEKRNKKK KRKLIERAQE QHSVFLATDR ANQ
R-spondin 3 (NCBI Reference Sequence: NP_116173.2) Homo sapiens:
(SEQ ID NO: 12)
MHLRLISWLF I ILNFMEYIG SQNASRGRRQ RRMHPNVSQG CQGGCATCSD YNGCLSCKPR
LFFALERIGM KQIGVCLSSC PSGYYGTRYP DINKCTKCKA DCDTCENKNF CTKCKSGFYL
HLGKCLDNCP EGLEANNHTM ECVSIVHCEV SEWNPWSPCT KKGKICGEKR GTETRVREI I
QHPSAKGNLC PPTNETRKCT VQRKKCQKGE RGKKGRERKR KKPNKGESKE AIPDSKSLES
SKEIPEQREN KQQQKKRKVQ DKQKSVSVST VH
R-spondin 4 (NCBI Reference Sequence: NP_001025042.2) Homo sapiens: isoform 1
(SEQ ID NO: 13)
MRAPLCLLLL VAHAVDMLAL NRRKKQVGTG LGGNCTGCI I CSEENGCSIC QQRLFLFIRR
EGIRQYGKCL HDCPPGYFGI RGQEVNRCKK CGATCESCFS QDFCIRCKRQ FYLYKGKCLP
TCPPGTLAHQ NTRECQGECE LGPWGGWSPC THINGKTCGSA WGLESRVREA GRAGHEEAAT
CQVLSESRKC PIQRPCPGER SPGQKKGRKD RRPRKDRKLD RRLDVRPRQP GLQP
R-spondin 4 (NCBI Reference Sequence: NP_001035096.1) Homo sapiens: isoform 2
(SEQ ID NO: 14)
MRAPLCLLLL VAHAVDMLAL NRRKKQVGTG LGGNCTGCI I CSEENGCSIC QQRLELFIRR
EGIRQYGKCL HDCPPGYFGI RGQEVNRCKK CGATCESCFS QDFCERCKRQ FYLYKGKCLP
TCPPGTLAHQ NTRECQERSP GQKKGRKDRR PRKDRKLDRR LDVRPRQPGL Q
Norrin
norrin precursor [Homo sapiens]
NCBI Reference Sequence: NP_000257.1
(SEQ ID NO: 15)
MRKHVLAASF SMLSLLVIMG DTDSKTDSSF IMDSDPRRCM RHHYVDS I SH PLYKCSSKMV
LLARCEGHCS QASRSEPLVS FSTVLKQPER SSCHCCRPQT SKLKALRLRC SGGMRLTATY RYILSCHCEE
CNS
WNT3A [Homo sapiens]
GenBank: BAB61052.1
(SEQ ID NO: 16)
MAPLGYFLLL CSLKQALGSY PIWWSLAVGP QYSSLGSQPI LCASIPGLVP KQLRFCRNYV EIMPSVAEGI
KIGIQECQHQ FRGRRWNCTT VHDSLAIFGP VLDKATRESA FVHAIASAGV AFAVTRSCAE GTAAICGCSS
RHQGSPGKGW KWGGCSEDIE FGGMVSREFA DARENRPDAR SAMNRHNNEA GRQAIASHMH LKCKCHGLSG
SCEVKTCWWS QPDFRAIGDF LKDKYDSASE MVVEKHRESR GWVETLRPRY TYFKVPTERD LVYYEASPNE
CEPNPETGSF GTRDRTCNVS SHGIDGCDLL CCGRGHNARA ERRREKCRCV FHWCCYVSCQ ECTRVYDVHT
CK
WNT6 [Homo sapiens]
GenBank: AAG45154.1
(SEQ ID NO: 17)
AVGSPLVMDP TSICRKARRL AGRQAELCQA EPEVVAELAR GARLGVRECQ FQFRFRRWNC SSHSKAFGRI
LQQDIRETAF VFAITAAGAS HAVTQACSMG ELLQCGCQAP RGRAPPRPSG LPGTPGPPGP AGSPEGSAAW
EWGGCGDDVD FGDEKSRLEM DARHKRGRGD IRALVQLHNN EAGRLAVRSH TRTECKCHGL SGSCALRTCW
QKLPPFREVG ARLLERFHGA SRVMGTNDGK ALLPAVRILK PPGRADLLYA ADSPDFCAPN RRTGSPGTRG
RACNSSAPDL SGCDLLCCGR GHRQESVQLE ENCLCRFHWC CVVQCHRCRV RKELSLCL
Mitogenic Factors
FGF-2 = bFGF (niProtKB/Swiss-Prot: P09038.3) Homo sapiens:
(SEQ ID NO: 18)
MVGVGGGDVE DVTPRPGGCQ I SGRGARGCN GIPGAAAWEA ALPRRRPRRH PSVNPRSRAA
GSPRTRGRRT EERPSGSRLG DRGRGRALPG GRLGGRGRGR APERVGGRGR GRGTAAPRAA
PAARGSRPGP AGTMAAGSIT TLPALPEDGG SGAFPPGHFK DPKRLYCKNG GEFLRIHPDG
RVDGVREKSD PHIKLQLQAE ERGVVSIKGV CANRYLAMKE DGRLLASKCV TDECFFFERL
ESNNYNTYRS RKYTSWYVAL KRIGQYKLGS KTGPGQKAIL FLPMSAKS
FGF7 (GenBank: CAG46799.1) Homo sapiens:
(SEQ ID NO: 19)
MHKWILTWIL PTLLYRSCFH IICLVGTISL ACNDMTPEQM ATNVNCSSPE RHTRSYDYME GGDIRVRRLF
CRTQWYLRID KRGKVKGTQE MKNNYNIMEI RTVAVGIVAI KGVESEFYLA MNKEGKLYAK KECNEDCNEK
ELILENHYNT YASAKWTHNG GEMFVALNQK GIPVRGKKTK KEQKTAHELP MAIT
FGF10 (GenBank: CAG46489.1) Homo sapiens:
(SEQ ID NO: 20)
MWKWILTHCA SAFPHLPGCC CCCFLLLFLV SSVPVTCQAL GQVMVSPEAT NSSSSSFSSP SSAGRHVRSY
NHLQGDVRWR KLFSFTKYFL KIEKNGKVSG TKKENCPYS I LEITSVEIGV VAVKAINSNY
YLAMNKKGKL YGSKEENNDC KLKERIEENG YNTYASENWQ HNGRQMYVAL NGKGAPRRGQ KTRRKNTSAH
FLPMVVHS
EGF (GenBank: EAX06257.1) Homo sapiens:
(SEQ ID NO 21)
MLLTLI ILLP VVSKFSFVSL SAPQHWSCPE GTLAGNGNST CVGPAPFLIF SHGNSIFRID
TEGTNYEQLV VDAGVSVIMD FHYNEKRIYW VDLERQLLQR VFLNGSRQER VCNIEKNVSG MAINWINEEV
IWSNQQEGII TVTDMKGNNS HILLSALKYP ANVAVDPVER FIFWSSEVAG SLYRADLDGV GVKALLETSE
KITAVSLDVL DKRLFWIQYN REGSNSLICS CDYDGGSVHI SKHPTQHNLF AMSLFGDRIF YSTWKMKTIW
IANKHTGKDM VRINLHSSFV PLGELKVVHP LAQPKAEDDT WEPEQKLCKL RKGNCSSTVC GQDLQSHLCM
CAEGYALSRD RKYCEDVNEC AFWNHGCTLG CKNEPGSYYC TCPVGFVLLP DGKRCHQLVS CPRNVSECSH
DCVLTSEGPL CFCPEGSVLE RDGKTCSGCS SPDNGGCSQL CVPLSPVSWE CDCFPGYDLQ LDEKSCAASG
PQPFLLFANS QDIRHMHFDG TDYGTLLSQQ MGMVYALDHD PVENKIYFAH TALKWIERAN MDGSQRERLI
EEGVDVPEGL AVDWIGRRFY WTDRGKSLIG RSDLNGKRSK IITKENISQP RGIAVHPMAK RLFWTDTGIN
PRIESSSLQG LGRLVIASSD LIWPSGITID FLTDKLYWCD AKQSVIEMAN LDGSKRRRLT QNDVGHPFAV
AVFEDYVWFS DWAMPSVMRV NKRIGKDRVR LQGSMLKPSS LVVVHPLAKP GADPCLYQNG GCEHICKKRL
GTAWCSCREG FMKASDGKTC LALDGHQLLA GGEVDLKNQV TPLDILSKTR VSEDNITESQ HMLVAEIMVS
DQDDCAPVGC SMYARCISEG EDATCQCLKG FAGDGKLCSD IDECEMGVPV CPPASSKCIN TEGGYVCRCS
EGYQGDGIHC LDIDECQLGE HSCGENASCT NTEGGYTCMC AGRLSEPGLI CPDSTPPPHL REDDHHYSVR
NSDSECPLSH DGYCLHDGVC MYIEALDKYA CNCVVGYIGE RCQYRDLKWW ELRHAGHGQQ QKVIVVAVCV
VVLVMLLLLS LWGAHYYRTQ KLLSKNPKNP YEESSRDVRS RRPADTEDGM SSCPQPWFVV IKEHQDLKNG
GQPVAGEDGQ AADGSMQPTS WRQEPQLCGM GTEQGCWIPV SSDKGSCPQV MERSFHMPSY GTQTLEGGVE
KPHSLLSANP LWQQRALDPP HQMELTQ
TGFa Homo sapiens: protransforming growth factor alpha isoform 1
preproprotein [Homo sapiens] NCBI Reference Sequence: NP_003227.1
(SEQ ID NO: 22)
MVPSAGQLAL FALGIVLAAC QALENSTSPL SADPPVAAAV VSHENDCPDS HTQFCFHGTC RFLVQEDKPA
CVCHSGYVGA RCEHADLLAV VAASQKKQAI TALVVVSIVA LAVLIITCVL IHCCQVRKHC EWCRALICRH
EKPSALLKGR TACCHSETVV
protransforming growth factor alpha isoform 2 preproprotein [Homo sapiens]
NCBI Reference Sequence: NP_001093161.1
(SEQ ID NO: 23)
MVPSAGQLAL FALGIVLAAC QALENSTSPL SDPPVAAAVV SHENDCPDSH TQFCFHGTCR FLVQEDKPAC
VCHSGYVGAR CEHADLLAVV AASQKKQAIT ALVVVSIVAL AVLIITCVLI HCCQVRKHCE WCRALICRHE
KPSALLKGRT ACCHSETVV
Transforming growth factor alpha [synthetic construct]
GenBank: AAX43291.1
(SEQ ID NO: 24)
MVPLAGQLAL FALGIVLAAC QALENSTSPL SDPPVAAAVV SHENDCPDSH TQFCFHGTCR FLVQEDKPAC
VCHSGYVGAR CEHADLLAVV AASQKKQAIT ALVVVSIVAL AVLIITCVLI HCCQVRKHCE WCRALICRHE
KPSALLKGRT ACCHSETVVL
TGF alpha containing:
(SEQ ID NO: 25)
VVSHENDCPD SHTQFCFHGT CRFLVQEDKP ACVCHSGYVG ARCEHA DLLA
BDNF (UniProtKB/Swiss-Prot: P23560.1) Homo sapiens:
(SEQ ID NO 26)
MTILFLTMVI SYFGCMKAAP MKEA IRGQG GLAYPGVRTH GTLESVNGPK AGSRGLTSLA
DTFEHVIEEL LDEDQKVRPN EENNKDADLY TSRVMLSSQV PLEPPLLFLL EEYKNYLDAA
NMSMRVRRHS DPARRGELSV CDSISEWVTA ADKKTAVDMS GGTVTVLEKV PVSKGQLKQY
FYETKCNPMG YTKEGCRGID KRHWNSQCRT TQSYVRALTM DSKKRIGWRF IRIDTSCVCT
LTIKRGR
KGF (GenBank: AAB21431.1) Homo sapiens:
(SEQ ID NO: 27)
MHKWILTWIL PTLLYRSCFH IICLVGTISL ACNDMTPEQM ATNVNCSSPE RHTRSYDYME GGDIRVRRLF
CRTQWYLRID KRGKVKGTQE MKNNYNIMEI RTVAVGIVAI KGVESEFYLA MNKEGKLYAK KECNEDCNEK
ELILENHYNT YASAKWTHNG GEMFVALNQK GIPVRGKKTK KEQKTAHELP MAIT

### 5. Methods for Differentiating the Stem Cells

The isolated stem cells (e.g., epithelial stem cells) may be induced to differentiate into differentiated cells that normally reside in the tissue or organ from which the stem cells 40 originates or are isolated. Other tissues include fallopian tubes, endometrium (uterus), male efferent ducts, male epididymis, male vas deferens, male ejaculatory duct, male bulbourethral glands, and seminal vesicle glands. The differentiated cells may express markers characteristic of the differentiated cells, and can be readily distinguished from the stem cells which do not express such differentiated cell markers.

### 6. Markers

In general, gene expression may be measured at RNA level for all of the markers described below. In addition, the expression of certain markers can also be detected by protein expression using, for example, antibody specific for proteins encoded by the marker genes.

### 7. Methods of Use

In a further aspect, the invention provides the use of the subject stem cells isolated from the various cultures in a drug discovery screen, toxicity assay, animalbased disease model, or in medicine, such as regenerative medicine.

Genetic manipulation of cloned stem cells. For instance, stem cells isolated by the methods of the invention are suitable for numerous types of genetic manipulation, including introduction of exogenous genetic materials that may modulate the expression of one or more target genes of interest. Such kind of gene therapy can be used, for example, in a method directed at repairing damaged or diseased tissue. In brief, any suitable vectors, including an adenoviral, eIntiviral, or retroviral gene delivery vehicle (see below), may be used to deliver genetic information, like DNA and/or RNA to any of the subject stem cells. A skilled person can replace or repair particular genes targeted in gene therapy. For example, a normal gene may be inserted into a nonspecific location within the genome of a diseased cell to replace a nonfunctional gene. In another example, an abnormal gene sequence can be replaced for a normal gene sequence through homologous recombination. Alternatively, selective reverse mutation can return a gene to its normal function. A further example is altering the regulation (the degree to which a gene is turned on or off) of a particular gene. Preferably, the stem cells are ex vivo treated by a gene therapy approach and are subsequently transferred to the mammal, preferably a human being in need of treatment.

Any art recognized methods for genetic manipulation may be applied to the stem cells so isolated, including transfection and infection (e.g., by a viral vector) by various types of nucleic acid constructs.

For example, heterologous nucleic acids (e.g., DNA) can be introduced into the subject stem cells by way of physical treatment (e.g., electroporation, sonoporation, optical transfection, protoplast fusion, impalefection, hydrodynamic delivery, nanoparticles, magnetofection), using chemical materials or biological vectors (viruses). Chemical-based transfection can be based on calcium phosphate, cyclodextrin, polymers (e.g., cationic polymers such as DEAE-dextran or polyethylenimine), highly branched organic compounds such as dendrimers, liposomes (such as cationic liposomes, lipofection such as lipofection using Lipofectamine, etc.), or nanoparticles (with or without chemical or viral functionalization).

A nucleic acid construct comprises a nucleic acid molecule of interest, and is generally capable of directing the expression of the nucleic acid molecule of interest in the cells into which it has been introduced.

In certain embodiments, the nucleic acid construct is an expression vector wherein a nucleic acid molecule encoding a gene product, such as a polypeptide or a nucleic acid that antagonizes the expression of a polypeptide (e.g., an siRNA, miRNA, shRNA, antisense sequence, aptamer, ribozyme etc.) is operably linked to a promoter capable of directing expression of the nucleic acid molecule in the target cells (e.g., the isolated stem cell).

The term “expression vector” generally refers to a nucleic acid molecule that is capable of effecting expression of a gene/nucleic acid molecule it contains in a cell compatible with such sequences. These expression vectors typically include at least suitable promoter sequences and optionally, transcription termination signals. A nucleic acid or DNA or nucleotide sequence encoding a polypeptide is incorporated into a DNA/nucleic acid construct capable of introduction into and expression in an in vitro cell culture as identified in a method of the invention.

A DNA construct prepared for introduction into a particular cell typically include a replication system recognized by the cell, an intended DNA segment encoding a desired polypeptide, and transcriptional and translational initiation and termination regulatory sequences operably linked to the polypeptide-encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading phase. However, enhancers need not be contiguous with a coding sequence whose transcription they control. Linking is accomplished by ligation at convenient restriction sites or at adapters or linkers inserted in lieu thereof.

The selection of an appropriate promoter sequence generally depends upon the host cell selected for the expression of a DNA segment. Examples of suitable promoter sequences include eukaryotic promoters well known in the art (see, e.g., Sambrook and Russell, Molecular Cloning: A Laboratory Manual, Third Edition, 2001). A transcriptional regulatory sequence typically includes a heterologous enhancer or promoter that is recognized by the cell. Suitable promoters include the CMV promoter. An expression vector includes the replication system and transcriptional and translational regulatory sequences together with the insertion site for the polypeptide encoding segment can be employed. Examples of workable combinations of cell lines and expression vectors are described in Sambrook and Russell (2001, supra) and in Metzger et al. (1988) Nature 334: 31-36.

Some aspects of the invention concern the use of a nucleic acid construct or expression vector comprising a nucleotide sequence as defined above, wherein the vector is a vector that is suitable for gene therapy. Vectors that are suitable for gene therapy are known in the art, such as those described in Anderson (Nature 392: 25-30, 1998); Walther and Stein (Drugs 60: 249-71, 2000); Kay et al. (Nat. Med. 7: 33-40, 2001); Russell (J. Gen. Virol. 81 :2573-604, 2000); Amado and Chen (Science 285:674-6, 1999); Federico (Curr. Opin. Biotechnol. 10:448-53, 1999); Vigna and Naldini (J. Gene Med. 2:308-16, 2000); Marin et al. (Mol. Med. Today 3:396-403, 1997); Peng and Russell (Curr. Opin. Biotechnol. 10:454-7, 1999); Sommerfelt (J. Gen. Virol. 80:3049-64, 1999); Reiser (Gene Ther. 7: 910-3, 2000); and references cited therein (all incorporated by reference). Examples include integrative and non-integrative vectors such as those based on retroviruses, adenoviruses (AdV), adenoassociated viruses (AAV), lentiviruses, pox viruses, alphaviruses, and herpes viruses.

A particularly suitable gene therapy vector includes an Adenoviral (Ad) and Adenoassociated virus (AAV) vector. These vectors infect a wide number of dividing and nondividing cell types. In addition, adenoviral vectors are capable of high levels of transgene expression. However, because of the episomal nature of the adenoviral and AAV vectors after cell entry, these viral vectors are most suited for therapeutic applications requiring only transient expression of the transgene (Russell, J. Gen. Virol. 81:2573-2604, 2000; Goncalves, Virol J. 2(1):43, 2005) as indicated above. Preferred adenoviral vectors are modified to reduce the host response as reviewed by Russell (2000, supra). Safety and efficacy of AAV gene transfer has been extensively studied in humans with encouraging results in the liver, muscle, CNS, and retina (Manno et ah, Nat. Medicine 2006; Stroes et al., ATYB 2008; Kaplitt, Feigin, Lancet 2009; Maguire, Simonelli et al. NEJM 2008; Bainbridge et al., NEJM 2008).

AAV2 is the best characterized serotype for gene transfer studies both in humans and experimental models. AAV2 presents natural tropism towards skeletal muscles, neurons, vascular smooth muscle cells and hepatocytes. Other examples of adeno-associated virusbased non-integrative vectors include AAVI, AAV3, AAV4, AAV5, AAV 6, AAV7, AAV8, AAV9, AAV 10, AAVI 1 and pseudotyped AAV. The use of non-human serotypes, like AAV8 and AAV9, might be useful to overcome these immunological responses in subjects, and clinical trials have just commenced (ClinicalTrials dot gov Identifier: NCT00979238). For gene transfer into a liver cell, an adenovirus serotype 5 or an AAV serotype 2, 7 or 8 have been shown to be effective vectors and therefore a preferred Ad or AAV serotype (Gao, Molecular Therapy 13:77-87, 2006).

An exemplary retroviral vector for application in the present invention is a lentiviral based expression construct. Lentiviral vectors have the unique ability to infect non-dividing cells (Amado and Chen, Science 285:674-676, 1999). Methods for the construction and use of lentiviral based expression constructs are described in U.S. Pat. Nos. 6,165,782, 6,207,455, 6,218,181, 6,277,633, and 6,323,031, and in Federico (Curr. Opin. Biotechnol. 10:448-53, 1999) and Vigna et al. (J. Gene Med. 2:308-16, 2000). Generally, gene therapy vectors will be as the expression vectors described above in the sense that they comprise a nucleotide sequence encoding a gene product (e.g., a polypeptide) of the invention to be expressed, whereby a nucleotide sequence is operably linked to the appropriate regulatory sequences as indicated above. Such regulatory sequence will at least comprise a promoter sequence. Suitable promoters for expression of a nucleotide sequence encoding a polypeptide from gene therapy vectors include, e.g., cytomegalovirus (CMV) intermediate early promoter, viral long terminal repeat promoters (LTRs), such as those from murine Moloney leukaemia virus (MMLV) rous sarcoma virus, or HTLV-1, the simian virus 40 (SV 40) early promoter and the herpes simplex virus thymidine kinase promoter. Additional suitable promoters are described below.

Several inducible promoter systems have been described that may be induced by the administration of small organic or inorganic compounds. Such inducible promoters include those controlled by heavy metals, such as the metallothionine promoter (Brinster et al, Nature 296:39-42, 1982; Mayo et al, Cell 29:99-108, 1982), RU-486 (a progesterone antagonist) (Wang et al, Proc. Natl. Acad. Sci. USA 91:8180-8184, 1994), steroids (Mader and White, Proc. Natl. Acad. Sci. USA 90:5603-5607, 1993), tetracycline (Gossen and Bujard, Proc. Natl. Acad. Sci. USA 89:5547-5551, 1992; U.S. Pat. No. 5,464,758; Furth et al, Proc. Natl. Acad. Sci. USA 91:9302-9306, 1994; Howe et al, J. Biol. Chem. 270:1416814174, 1995; Resnitzky et al, Mol. Cell. Biol. 14:1669-1679, 1994; Shockett et al, Proc. Natl. Acad. Sci. USA 92:6522-6526, 1995) and the tTAER system that is based on the multichimeric transactivator composed of a tetR polypeptide, as activation domain of VP 16, and a ligand binding domain of an estrogen receptor (Yee et al, 2002, U.S. Pat. No. 6,432,705).

Suitable promoters for nucleotide sequences encoding small RNAs for knock down of specific genes by RNA interference (see below) include, in addition to the above-mentioned polymerase II promoters, polymerase III promoters. The RNA polymerase III (pol III) is responsible for the synthesis of a large variety of small nuclear and cytoplasmic non-coding RNAs including 5S, U6, adenovirus VA1, Vault, telomerase RNA, and tRNAs. The promoter structures of a large number of genes encoding these RNAs have been determined and it has been found that RNA pol III promoters fall into three types of structures (for a review see Geiduschek and TocchiniValentini, Annu. Rev. Biochem. 57: 873-914, 1988; Willis, Eur. J. Biochem. 212: 1-11, 1993; Hernandez, J. Biol. Chem. 276:26733-36, 2001). Particularly suitable for expression of siRNAs are the type 3 of the RNA pol III promoters, whereby transcription is driven by cis-acting elements found only in the 5′-flanking region, i.e., upstream of the transcription start site. Upstream sequence elements include a traditional TATA box (Mattaj et al., Cell 55:435-442, 1988), proximal sequence element and a distal sequence element (DSE; Gupta and Reddy, Nucleic Acids Res. 19:2073-2075, 1991).

Examples of genes under the control of the type 3 pol III promoter are U6 small nuclear RNA (U6 snRNA), 7SK, Y, MRP, HI and telomerase RNA genes (see, e.g., Myslinski et al, Nucl. Acids Res. 21:2502-09, 2001).

A gene therapy vector may optionally comprise a second or one or more further nucleotide sequence coding for a second or further polypeptide. A second or further polypeptide may be a (selectable) marker polypeptide that allows for the identification, selection and/or screening for cells containing the expression construct. Suitable marker proteins for this purpose are, e.g., the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russell, Molecular Cloning: A Laboratory Manual (3rd edition), Cold Spring Harbor Laboratory, Cold Spring Harbor Laboratory Press, New York, 2001.

Alternatively, a second or further nucleotide sequence may encode a polypeptide that provides for fail-safe mechanism that allows a subject from the transgenic cells to be cured, if deemed necessary. Such a nucleotide sequence, often referred to as a suicide gene, encodes a polypeptide that is capable of converting a prodrug into a toxic substance that is capable of killing the transgenic cells in which the polypeptide is expressed. Suitable examples of such suicide genes include, e.g., the E. coli cytosine deaminase gene or one of the thymidine kinase genes from Herpes Simplex Virus, Cytomegalovirus and Varicella-Zoster virus, in which case ganciclovir may be used as prodrug to kill the IL-10 transgenic cells in the subject (see, e.g., Clair et al., Antimicrob. Agents Chemother. 31:844-849, 1987).

For knock down of expression of a specific polypeptide, a gene therapy vector or other expression construct is used for the expression of a desired nucleotide sequence that preferably encodes an RNAi agent, i.e., an RNA molecule that is capable of RNA interference or that is part of an RNA molecule that is capable of RNA interference. Such RNA molecules are referred to as siRNA (short interfering RNA, including, e.g., a short hairpin RNA). A desired nucleotide sequence comprises an antisense code DNA coding for the antisense RNA directed against a region of the target gene mRNA, and/or a sense code DNA coding for the sense RNA directed against the same region of the target gene mRNA. In a DNA construct of the invention, an antisense and sense code DNAs are operably linked to one or more promoters as herein defined above that are capable of expressing an antisense and sense RNAs, respectively. “siRNA” includes a small interfering RNA that is a shortlength double-stranded RNA that is not toxic in mammalian cells (Elbashir et ah, Nature 411:494-98, 2001; Caplen et al, Proc. Natl. Acad. Sci. USA 98:9742-47, 2001). The length is not necessarily limited to 21 to 23 nucleotides. There is no particular limitation in the length of siRNA as long as it does not show toxicity. “siRNAs” can be, e.g., at least about 15, 18 or 21 nucleotides and up to 25, 30, 35 or 49 nucleotides long. Alternatively, the double-stranded RNA portion of a final transcription product of siRNA to be expressed can be, e.g., at least about 15, 18 or 21 nucleotides, and up to 25, 30, 35 or 49 nucleotides long.

“Antisense RNA” is preferably an RNA strand having a sequence complementary to a target gene mRNA, and thought to induce RNAi by binding to the target gene mRNA.

“Sense RNA” has a sequence complementary to the antisense RNA, and annealed to its complementary antisense RNA to form siRNA.

The term “target gene” in this context includes a gene whose expression is to be silenced due to siRNA to be expressed by the present system, and can be arbitrarily selected. As this target gene, for example, genes whose sequences are known but whose functions remain to be elucidated, and genes whose expressions are thought to be causative of diseases are preferably selected. A target gene may be one whose genome sequence has not been fully elucidated, as long as a partial sequence of mRNA of the gene having at least 15 nucleotides or more, which is a length capable of binding to one of the strands (antisense RNA strand) of siRNA, has been determined. Therefore, genes, expressed sequence tags (ESTs) and portions of mRNA, of which some sequence (preferably at least 15 nucleotides) has been elucidated, may be selected as the “target gene” even if their full-length sequences have not been determined.

The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. A non-pairing portions can be contained to the extent that they do not interfere with siRNA formation. The “bulge” used herein may comprise 1 to 2 non-pairing nucleotides, and the double-stranded RNA region of siRNAs in which two RNA strands pair up contains preferably 1 to 7, more preferably 1 to 5 bulges.

The term “mismatch” as used herein may be contained in the double-stranded RNA region of siRNAs in which two RNA strands pair up, preferably 1 to 7, more preferably 1 to 5, in number. In certain mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, a double-stranded RNA region of siRNAs in which two RNA strands pair up may contain both bulge and mismatched, which sum up to, preferably 1 to 7, more preferably 1 to 5 in number. Such non-pairing portions (mismatches or bulges, etc.) can suppress the below described recombination between antisense and sense code DNAs and make the siRNA expression system as described below stable. Furthermore, although it is difficult to sequence stem loop DNA containing no non-pairing portion in the double-stranded RNA region of siRNAs in which two RNA strands pair up, the sequencing is enabled by introducing mismatches or bulges as described above. Moreover, siRNAs containing mismatches or bulges in the pairing double-stranded RNA region have the advantage of being stable in E. coli or animal cells.

The terminal structure of siRNA may be either blunt or cohesive (overhanging) as long as siRNA enables to silence the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 bp double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the “siRNA” is necessarily the cut off structure at both ends as described above, and may have a stem-loop structure in which ends of one side of double-stranded RNA are connected by a linker RNA (a “shRNA”). The length of the double-stranded RNA region (stem-loop portion) can be, e.g., at least 15, 18 or 21 nucleotides and up to 25, 30, 35 or 49 nucleotides long. Alternatively, the length of the double-stranded RNA region that is a final transcription product of siRNAs to be expressed is, e.g., at least 15, 18 or 21 nucleotides and up to 25, 30, 35 or 49 nucleotides long.

Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA, snRNA or viral RNA, or an artificial RNA molecule.

To express antisense and sense RNAs from the antisense and sense code DNAs respectively, a DNA construct of the present invention comprise a promoter as defined above. The number and the location of the promoter in the construct can in principle be arbitrarily selected as long as it is capable of expressing antisense and sense code DNAs. As a simple example of a DNA construct of the invention, a tandem expression system can be formed, in which a promoter is located upstream of both antisense and sense code DNAs. This tandem expression system is capable of producing siRNAs having the aforementioned cut off structure on both ends. In the stem-loop siRNA expression system (stem expression system), antisense and sense code DNAs are arranged in the opposite direction, and these DNAs are connected via a linker DNA to construct a unit. A promoter is linked to one side of this unit to construct a stem-loop siRNA expression system. Herein, there is no particular limitation in the length and sequence of the linker DNA, which may have any length and sequence as long as its sequence is not the termination sequence, and its length and sequence do not hinder the stem portion pairing during the mature RNA production as described above. As an example, DNA coding for the above-mentioned tRNA and such can be used as a linker DNA.

In both cases of tandem and stem-loop expression systems, the 5′ end may be have a sequence capable of promoting the transcription from the promoter. More specifically, in the case of tandem siRNA, the efficiency of siRNA production may be improved by adding a sequence capable of promoting the transcription from the promoters at the 5′ ends of antisense and sense code DNAs. In the case of stem-loop siRNA, such a sequence can be added at the 5′ end of the above-described unit. A transcript from such a sequence may be used in a state of being attached to siRNA as long as the target gene silencing by siRNA is not hindered. If this state hinders the gene silencing, it is preferable to perform trimming of the transcript using a trimming means (for example, ribozyme as are known in the art). It will be clear to the skilled person that an antisense and sense RNAs may be expressed in the same vector or in different vectors. To avoid the addition of excess sequences downstream of the sense and antisense RNAs, it is preferred to place a terminator of transcription at the 3′ ends of the respective strands (strands coding for antisense and sense RNAs). The terminator may be a sequence of four or more consecutive adenine (A) nucleotides.

Genome Editing. Genome editing may be used to change the genomic sequence of the subject cloned stem cells, including cloned cancer (or other disease) stem cells, by introducing heterologous transgene or by inhibiting expression of a target endogenous gene. Such genetically engineered stem cells can be used, for regenerative medicine (see below) or wound healing. Thus in certain embodiments, the subject methods of regenerative medicine (see below) comprise using a subject stem cell the genome sequence of which has been modified by genomic editing.

Genome editing may be performed using any art-recognized technology, such as ZFN/TALEN or CRISPR technologies (see review by Gaj et ah, Trends in Biotech. 31(7): 397-405, 2013, the entire text and all cited references therein are incorporated herein by reference). Such technologies enable one to manipulate virtually any gene in a diverse range of cell types and organisms, thus enabling a broad range of genetic modifications by inducing DNA double-strand (DSB) breaks that stimulate error-prone nonhomologous end joining (NHEJ) or homology-directed repair (HDR) at specific genomic locations.

Zinc-finger nucleases (ZFNs) and Transcription activator-like effector nucleases (TALENs) are chimeric nucleases composed of programmable, sequence-specific DNAbinding modules linked to a nonspecific DNA cleavage domain. They are artificial restriction enzymes (REs) generated by fusing a zinc-finger or TAL effector DNA binding domain to a DNA cleavage domain. A zinc-finger (ZF) or transcription activator-like effector (TALE) can be engineered to bind any desired target DNA sequence, and be fused to a DNA cleavage domain of an RE, thus creating an engineer restriction enzyme (ZFN or TALEN) that is specific for the desired target DNA sequence. When ZFN/TALEN is introduced into cells, it can be used for genome editing in situ. Indeed, the versatility of the ZFNs and TALENs can be expanded to effector domains other than nucleases, such as transcription activators and repressors, recombinases, transposases, DNA and histone methyl transferases, and histone acetyltransferases, to affect genomic structure and function.

The Cys2-His2 zinc-finger domain is among the most common types of DNA-binding motifs found in eukaryotes and represents the second most frequently encoded protein domain in the human genome. An individual zinc-finger has about 30 amino acids in a conserved ββα configuration. Key to the application of zinc-finger proteins for specific DNA recognition was the development of unnatural arrays that contain more than three zinc-finger domains. This advance was facilitated by the structure-based discovery of a highly conserved linker sequence that enabled construction of synthetic zinc-finger proteins that recognized DNA sequences 9-18 bp in length. This design has proven to be the optimal strategy for constructing zinc-finger proteins that recognize contiguous DNA sequences that are specific in complex genomes. Suitable zinc-fingers may be obtained by modular assembly approach (e.g., using a preselected library of zinc-finger modules generated by selection of large combinatorial libraries or by rational design). Zinc-finger domains have been developed that recognize nearly all of the 64 possible nucleotide triplets, preselected zinc-finger modules can be linked together in tandem to target DNA sequences that contain a series of these DNA triplets. Alternatively, selection-based approaches, such as oligomerized pool engineering (OPEN) can be used to select for new zinc-finger arrays from randomized libraries that take into consideration context-dependent interactions between neighboring fingers. A combination of the two approaches is also used.

Engineered zinc fingers are commercially available. Sangamo Biosciences (Richmond, CA, USA) has developed a propriety platform (CompoZr) for zinc-finger construction in partnership with SigmaAldrich (St. Louis, MO, USA), which platform allows investigators to bypass zinc-finger construction and validation altogether, and many thousands of proteins are already available. Broadly, zinc-finger protein technology enables targeting of virtually any sequence.

TAL effectors are proteins secreted by the plant pathogenic Xanthomonas bacteria, with DNA binding domain containing a repeated highly conserved 33-34 amino acid sequence, with the exception of the 12th and 13th amino acids. These two locations are highly variable (Repeat Variable Diresidue, or RVD) and show a strong correlation with specific nucleotide recognition. This simple relationship between amino acid sequence and DNA recognition has allowed for the engineering of specific DNA binding domains by selecting a combination of repeat segments containing the appropriate RVDs. Like zinc fingers, modular TALE repeats are linked together to recognize contiguous DNA sequences. Numerous effector domains have been made available to fuse to TALE repeats for targeted genetic modifications, including nucleases, transcriptional activators, and site-specific recombinases. Rapid assembly of custom TALE arrays can be achieved by using strategies include “Golden Gate” molecular cloning, high-throughput solid-phase assembly, and ligation-independent cloning techniques, all can be used in the instant invention for genome editing of the cloned stem cells.

TALE repeats can be easily assembled using numerous tools available in the art, such as a library of TALENs targeting 18,740 human protein-coding genes (Kim et al., Nat. Biotechnol. 31, 251-258, 2013). Custom-designed TALE arrays are also commercially available through, for example, Cellectis Bioresearch (Paris, France), Transposagen Biopharmaceuticals (Lexington, KY, USA), and Life Technologies (Grand Island, NY, USA).

The non-specific DNA cleavage domain from the end of a RE, such as the FokI endonuclease (or FokI cleavage domain variants, such as Sharkey, with mutations designed to improve cleavage specificity and/or cleavage activity), can be used to construct hybrid nucleases that are active in a yeast assay (also active in plant cells and in animal cells). To improve ZFN activity, transient hypothermic culture conditions can be used to increase nuclease expression levels; co-delivery of site-specific nucleases with DNA end-processing enzymes, and the use of fluorescent surrogate reporter vectors that allow for the enrichment of ZFN and TALEN-modified cells, may also be used. The specificity of ZFN-mediated genome editing can also be refined by using zinc-finger nickases (ZFNickases), which take advantage of the finding that induction of nicked DNA stimulates HDR without activating the error-prone NHEJ repair pathway.

The simple relationship between amino acid sequence and DNA recognition of the TALE binding domain allows for designable proteins. A publicly available software program (DNAWorks) can be used to calculate oligonucleotides suitable for assembly in a two step PCR. A number of modular assembly schemes for generating engineered TALE constructs have also been reported and known in the art. Both methods offer a systematic approach to engineering DNA binding domains that is conceptually similar to the modular assembly method for generating zinc finger DNA recognition domains.

Once the TALEN genes have been assembled, they are introduced into the target cell on a vector using any art recognized methods (such as electroporation or transfection using cationic lipid-based reagents, using plasmid vectors, various viral vectors such as adenoviral, AAV, and Integrase-deficient lentiviral vectors (IDLVs)). Alternatively, TALENs can be delivered to the cell as mRNA, which removes the possibility of genomic integration of the TALEN-expressing protein. It can also dramatically increase the level of homology directed repair (HDR) and the success of introgression during gene editing. Finally, direct delivery of purified ZFN/TALEN proteins into cells may also be used. This approach does not carry the risk of insertional mutagenesis, and leads to fewer off-target effects than delivery systems that rely on expression from nucleic acids, and thus may be optimally used for studies that require precise genome engineering in cells, such as the instant stem cells.

TALENs can be used to edit genomes by inducing double-strand breaks (DSB), which cells respond to with repair mechanisms. Non-homologous end joining (NHEJ) reconnects DNA from either side of a double-strand break where there is very little or no sequence overlap for annealing. A simple heteroduplex cleavage assay can be run which detects any difference between two alleles amplified by PCR. Cleavage products can be visualized on simple agarose gels or slab gel systems. Alternatively, DNA can be introduced into a genome through NHEJ in the presence of exogenous double-stranded DNA fragments.

Homology directed repair can also introduce foreign DNA at the DSB as the transfected double-stranded sequences are used as templates for the repair enzymes. TALENs have been used to generate stably modified human embryonic stem cell and induced pluripotent stem cell (iPSCs) clones to generate knockout C. elegans, rats, and zebrafish.

For stem cell based therapy, ZFNs and TALENs are capable of correcting the underlying cause of the disease, therefore permanently eliminating the symptoms with precise genome modifications. For example, ZFN-induced HDR has been used to directly correct the disease-causing mutations associated with X-linked severe combined immune deficiency (SCJD), hemophilia B, sickle-cell disease, al-antitrypsin deficiency and numerous other genetic diseases, either by repair defective target genes, or by knocking out a target gene. In addition, these site-specific nucleases can also be used to safely insert a therapeutic transgenes into the subject stem cell, at a specific “safe harbor” locations in the human genome. Such techniques, in combination with the stem cells of the invention, can be used in gene therapy, including treatments based on autologous stem cell transplantation, where one or more genes of the cloned (diseased or normal) stem cells are manipulated to increase or decrease/eliminate a target gene expression.

Alternatively, CRISPR/Cas system can also be used to efficiently induce targeted genetic alterations into the subject stem cells. CRISPR/Cas (CRISPR associated) systems or “Clustered Regulatory Interspaced Short Palindromic Repeats” are loci that contain multiple short direct repeats, and provide acquired immunity to bacteria and archaea. CRISPR systems rely on crRNA and tracrRNA for sequence-specific silencing of invading foreign DNA. The term “tracrRNA” stands for trans-activating chimeric RNA, which is noncoding RNA that promotes crRNA processing, and is required for activating RNA-guided cleavage by Cas9. CRISPR RNA or crRNA base pairs with tracrRNA to form a two-RNA structure that guides the Cas9 endonuclease to complementary DNA sites for cleavage.

Three types of CRISPR/Cas systems exist: in type II systems, Cas9 serves as an RNA-guided DNA endonuclease that cleaves DNA upon crRNA-tracrRNA target recognition. In bacteria, the CRISPR system provides acquired immunity against invading foreign DNA via RNA-guided DNA cleavage. The CRISPR/Cas system can be retargeted to cleave virtually any DNA sequence by redesigning the crRNA. Indeed, the CRISPR/Cas system has been shown to be directly portable to human cells by co-delivery of plasmids expressing the Cas9 endonuclease and the necessary crRNA components. These programmable RNA-guided DNA endonucleases have demonstrated multiplexed gene disruption capabilities and targeted integration in iPS cells, and can thus be used similarly in the subject stem cells.

Cancer stem cells. The methods and reagents of the invention also enable culturing and isolating cancer derived cancer stem cells (CSCs) from epithelial tissue samples/biopsies or from other stratified regenerative tissues, which in turn may be used in numerous applications previously impossible or impractical to carry out, partly due to the inability to obtaining such CSCs in large quantity and as single cell clones.

For example, the libraries of CSCs established from a single patient using the methods of the invention enable comparison between patient-matched sensitive and resistant clones for directed drug discovery efforts. Certain genes may be up-regulated or down-regulated in the resistant clones compared to the sensitive clones. Inhibitors for the up-regulated genes may be further validated as a drug target gene, by testing, for example, the ability of downregulation of the target gene in the resistant clones, and determining its effect on drug resistance. Conversely, restoring or overexpressing the down-regulated genes in the resistant clones may also overcome drug resistance.

Thus in one aspect, the invention provides a drug discovery method using CSCs isolated using the subject methods and media, for identifying genes up or down-regulated in drug resistant CSC clones, the method comprising: (1) using the method of the invention, obtaining a plurality of cell clones from a cancerous tissue (such as one from a cancer patient); (2) contacting the plurality of cell clones with one or more chemical compound (e.g., cancer drug), under conditions in which a small percentage (e.g., no more than 1%, 0.5%, 0.2%, 0.1%, 0.05%, 0.01% or fewer) of drug-resistant clones survive; (3) comparing gene expression profiles of the drug-resistant clones with that of the sensitive clones (e.g., one or more randomly picked plurality of cell clones before step (2), which are presumably sensitive to drug treatment), thus identifying genes up or down-regulated in the surviving drug resistant clones.

In certain embodiments, the method further comprises inhibiting the expression of an up-regulated gene in the surviving drug-resistant clone. For example, the up-regulated gene may be commonly up-regulated in two or more surviving drug-resistant clones, either from the same type of tumors or different types of tumors, either from the same patient, or from different patients. In certain embodiments, the up-regulated gene may be specific for the patient from whom the CSCs are isolated. This can be helpful in designing personalized medicine or treatment regimens for the patient.

In certain embodiments, the method further comprises restoring or increasing the expression of a down-regulated gene in the surviving drug-resistant clone. For example, the down-regulated gene may be commonly down-regulated in two or more surviving drug resistant clones, either from the same type of tumors or different types of tumors, either from the same patient, or from different patients. In certain embodiments, the down-regulated gene may be specific for the patient from whom the CSCs are isolated. This can also be helpful in designing personalized medicine or treatment regimens for the patient.

In a related aspect, the invention provides a drug discovery method using CSCs isolated using the subject methods and media, for identifying a candidate compound that inhibit the growth or promote the killing of a drug-resistant CSC, the method comprising: (1) using the method of the invention, obtaining a plurality of cell clones from a cancerous tissue (such as one from a cancer patient); (2) contacting the plurality of cell clones with one or more chemical compound (e.g., cancer drug), under conditions in which a small percentage (e.g., no more than 1%, 0.5%, 0.2%, 0.1%, 0.05%, 0.01% or fewer) of drug-resistant clones survive; (3) contacting the surviving drug-resistant clones with a plurality of candidate compounds, and (4) identifying one or more candidate compounds that inhibit the growth or promote the killing of the surviving drug-resistant clones. In certain embodiments, the method is performed using high-throughput screens format, for candidate drugs that target resistant cells.

In certain embodiments, the method further comprises testing general toxicity of the identified candidate compounds on the matching sensitive clones (e.g., one or more randomly picked plurality of cell clones before step (2), which are presumably sensitive to drug treatment), and/or the matching healthy cells from the same patient from whom the CSCs are isolated. Preferably, any identified candidate compounds specifically or preferentially inhibit the growth or promote the killing of the drug-resistant CSC, compared to the matching sensitive clones and/or the matching healthy cells.

In certain embodiments, the healthy cells are patient-matched normal stem cells similarly isolated using the methods and reagents of the invention.

The above embodiment is partly based on the discovery that, in many cases, drug resistant CSCs grow more slowly compared to drug-sensitive clones. While not wishing to be bound by any particular theory, Applicant believes that the slow growth is likely a consequence of gene expression alterations in the drug-resistant CSCs for evading chemotherapy. Thus, it is expected that certain agents may inhibit the growth or kill drug resistant cells preferentially while being less toxic than standard chemotherapy drugs (such as cisplatin or paclitaxel) used to treat the cancer in the first place.

In another aspect, the invention provides a method for identifying a suitable or effective treatment for a patient in need of treating a disease, the method comprising: (1) using the method of the invention, obtaining a plurality of stem cell clones from a disease tissue (such as a cancerous tissue) from the patient; (2) subjecting the plurality of cell clones to one or more candidate treatments; (3) determining the effectiveness of each of said one or more candidate treatments; thereby identifying a suitable or effective treatment for the patient in need of treating the disease. This can be useful, for example, when the patient has several possible treatment options, each may or may not be suitable or effective for the patient.

In a related aspect, the invention provides a method for screening for the most suitable or effective treatment among a plurality of candidate treatments, for treating a patient in need of treating a disease, the method comprising: (1) using the method of the invention, obtaining a plurality of stem cell clones from a disease tissue (such as a cancerous tissue) from the patient; (2) subjecting the plurality of cell clones to said candidate treatments; (3) comparing the relative effectiveness of said one or more candidate treatments; thereby identifying the most suitable or effective treatment for the patient. This can be useful, for example, when the patient has several alternative treatment options that may each be effective against a specific patient population but not necessarily effective for others.

In certain embodiments, the disease is a cancer, such as any of the cancers from which a cancer stem cell can be isolated.

In certain embodiments, the treatment is a chemotherapy regimen, such as one utilizing one or more chemo therapeutic agents. In certain embodiments, the treatment is radiotherapy. In certain embodiments, the treatment is immunotherapy, such as one using a cell-binding agent (e.g., antibody) that specifically binds to a surface ligand (e.g., surface antigen) of a cancer cell. In certain embodiments, the treatment is a combination therapy of surgery, chemotherapy, radiotherapy, and/or immunotherapy.

In certain embodiments, the disease is an inflammatory disease, a disease from which a disease-associated stem cell can be isolated, or any disease referenced herein.

In certain embodiments, the method further comprises treating the patient using one or more identified suitable or effective treatment for the disease.

In certain embodiments, the method further comprises producing a report that provides the effectiveness of each of said candidate treatments, such as the effectiveness of each of the candidate chemotherapeutic agents tested, either individually or in combination (including sequentially or simultaneously).

In certain embodiments, the method further comprises providing a recommendation for the most effective treatment.

In a related aspect, the invention provides kits and reagents for carrying out the methods of the invention.

In certain embodiments, the general screening method of the invention (not necessarily limited to cancer stem cells) is carried out in high-throughput/automatic fashion. For high-throughput purposes, the expanded stem cell population can be cultured in multiwell plates such as, for example, 96-well plates or 384-well plates. Libraries of molecules are used to identify a molecule that affects the plated stem cells. Preferred libraries include (without limitation) antibody fragment libraries, peptide phage display libraries, peptide libraries (e.g., LOPAP™, Sigma Aldrich), lipid libraries (BioMol), synthetic compound libraries (e.g., LOP AC™, Sigma Aldrich) or natural compound libraries (Specs, TimTec). Furthermore, genetic libraries can be used that induce or repress the expression of one of more genes in the progeny of the stem cells. These genetic libraries comprise cDNA libraries, antisense libraries, and siRNA or other non-coding RNA libraries.

The stem cells are preferably exposed to multiple concentrations of a test/candidate agent for a certain period of time. At the end of the exposure period, the cultures are evaluated for a pre-determined effect, such as any changes in a cell, including, but not limited to, a reduction in, or loss of, proliferation, a morphological change, and cell death.

The expanded stem cell population can also be used to identify drugs that specifically target epithelial carcinoma cells or stem cells isolated therefrom, but not the expanded stem cell population itself.

The ready cloning of cancer stem cells also enables immunological approaches to tumor destruction. The technology described herein enables the high-efficiency cloning of CSCs and therefore potentially provides information that would aid approaches to eradicating these cells via immune activation.

For example, upon isolating the CSCs (either drug-sensitive or drug-resistant), one or more epitopes of such CSCs, preferably CSC-specific epitopes compared to healthy control (e.g., epitopes on the cell surface or secretome of CSCs), may be used to vaccinate antigen presenting cells (APCs) to direct lymphocytes to target these CSCs. The immunological approaches might include, as was done to melanoma, the identification and targeting of molecules on the cell surface or secretome of CSCs that suppress immune surveillance.

### Regenerative Medicine

The subject stem cells may also be useful in regenerative medicine, for example in post-trauma, post-radiation, and/or post-surgery repair of the various damaged reproductive tissues or organs.

In yet another embodiment, a small biopsy or tissue sample can be taken from adult donors, and stem cells therein can be isolated and expanded, and optionally differentiated, to generate transplantable epithelium for regenerative purposes. The fact that the subject stem cells can be frozen and thawed and put back into culture without losing the stem cell character and without significant cell death further adds to the applicability of the subject stem cells for transplantation purposes.

Thus, the invention provides a stem cell or expanded clone thereof or differentiation product thereof (or collectively “stem cell” in the context of regenerative medicinal use) for use in transplantation into a mammal, preferably into a human. Also provided is a method of treating a patient in need of a transplant comprising transplanting a population of the stem cell of the invention into the patient, wherein the patient is a mammal, preferably a human.

Thus, another aspect of the invention provides a method of treating a human or nonhuman animal patient through cellular therapy. Such cellular therapy encompasses the application or administration of the stem cells of the invention (such as tissue matched stem cells of the invention) to the patient through any appropriate means. Specifically, such methods of treatment involve the regeneration of damaged tissue or wound healing. In accordance with the invention, a patient can be treated with allogeneic or autologous stem cells or clonal expansion thereof. “Autologous” cells are cells which originated from the same organism into which they are being re-introduced for cellular therapy, for example in order to permit tissue regeneration. However, the cells have not necessarily been isolated from the same tissue as the tissue they are being introduced into. An autologous cell does not require matching to the patient in order to overcome the problems of rejection. “Allogeneic” cells are cells which originated from an individual which is different from the individual into which the cells are being introduced for cellular therapy, for example in order to permit tissue regeneration, although of the same species. Some degree of patient matching may still be required to prevent the problems of rejection.

Generally, the stem cells of the invention are introduced into the body of the patient by injection or implantation. Generally, the cells will be directly injected into the tissue in which they are intended to act. Alternatively, the cells will be injected through the portal vein. A syringe containing cells of the invention and a pharmaceutically acceptable carrier is included within the scope of the invention. A catheter attached to a syringe containing cells of the invention and a pharmaceutically acceptable carrier is also included within the scope of the invention.

Stem cells of the invention can also be used in the regeneration of tissue. In order to achieve this function, cells may be injected or implanted directly into the damaged tissue, where they may multiply and eventually differentiate into the required cell type, in accordance with their location in the body, and/or after homing to their tissue of origin.

Alternatively, the subject stem cells can be injected or implanted directly into the damaged tissue. Tissues that are susceptible to treatment include all damaged tissues, particularly including those which may have been damaged by disease, injury, trauma, an autoimmune reaction, or by a viral or bacterial infection. In some embodiments of the invention, the stem cells of the invention are used to regenerate the lung, esophagus, stomach, small intestine, colon, intestinal metaplasia, fallopian tube, kidney, pancreas, bladder, liver, or gastric system, or a portion/section thereof.

In certain embodiments, the patient is a human, but may alternatively be a non-human mammal, such as a cat, dog, horse, cow, pig, sheep, rabbit or mouse.

In certain embodiments, the stem cells of the invention are injected into a patient using a syringe, such as a Hamilton syringe. The skilled person will be aware what the appropriate dosage of stem cells of the invention will be for a particular condition to be treated.

In certain embodiments, the stem cells of the invention, either in solution, in microspheres, or in microparticles of a variety of compositions, are administered into the artery irrigating the tissue or the part of the damaged organ in need of regeneration.

Generally, such administration will be performed using a catheter. The catheter may be one of the large variety of balloon catheters used for angioplasty and/or cell delivery or a catheter designed for the specific purpose of delivering the cells to a particular local of the body.

For certain uses, the stem cells may be encapsulated into microspheres made of a number of different biodegradable compounds, and with a diameter of about 15 μiη. This method may allow intravascularly administered stem cells to remain at the site of damage, and not to go through the capillary network and into the systemic circulation in the first passage. The retention at the arterial side of the capillary network may also facilitate their translocation into the extravascular space.

In certain embodiments, the stem cells may be retrograde injected into the vascular tree, either through a vein to deliver them to the whole body or locally into the particular vein that drains into the tissue or body part to which the stem cells are directed.

In another embodiment, the stem cells of the invention may be implanted into the damaged tissue adhered to a biocompatible implant. Within this embodiment, the cells may be adhered to the biocompatible implant in vitro, prior to implantation into the patient. As will be clear to a person skilled in the art, any one of a number of adherents may be used to adhere the cells to the implant, prior to implantation. By way of example only, such adherents may include fibrin, one or more members of the integrin family, one or more members of the cadherin family, one or more members of the selectin family, one or more cell adhesion molecules (CAMs), one or more of the immunoglobulin family and one or more artificial adherents. This list is provided by way of illustration only, and is not intended to be limiting. It will be clear to a person skilled in the art, that any combination of one or more adherents may be used.

In another embodiment, the stem cells of the invention may be embedded in a matrix, prior to implantation of the matrix into the patient. Generally, the matrix will be implanted into the damaged tissue of the patient. Examples of matrices include collagen based matrices, fibrin based matrices, laminin based matrices, fibronectin based matrices and artificial matrices. This list is provided by way of illustration only, and is not intended to be limiting. In a further embodiment, the stem cells of the invention may be implanted or injected into the patient together with a matrix forming component. This may allow the cells to form a matrix following injection or implantation, ensuring that the stem cells remain at the appropriate location within the patient. Examples of matrix forming components include fibrin glue liquid alkyl, cyanoacrylate monomers, plasticizers, polysaccharides such as dextran, ethylene oxide-containing oligomers, block co-polymers such as poloxamer and Pluronics, non-ionic surfactants such as Tween and Triton 8, and artificial matrix forming components. This list is provided by way of illustration only, and is not intended to be limiting. It will be clear to a person skilled in the art, that any combination of one or more matrix forming components may be used.

In a further embodiment, the stem cells of the invention may be contained within a microsphere. Within this embodiment, the cells may be encapsulated within the center of the microsphere. Also within this embodiment, the cells may be embedded into the matrix material of the microsphere. The matrix material may include any suitable biodegradable polymer, including but not limited to alginates, Poly ethylene glycol (PLGA), and polyurethanes. This list is provided by way of example only, and is not intended to be limiting.

In a further embodiment, the stem cells of the invention may be adhered to a medical device intended for implantation. Examples of such medical devices include stents, pins, stitches, splits, pacemakers, prosthetic joints, artificial skin, and rods. This list is provided by way of illustration only, and is not intended to be limiting. It will be clear to a person skilled in the art, that the cells may be adhered to the medical device by a variety of methods. For example, the stem cells may be adhered to the medical device using fibrin, one or more members of the integrin family, one or more members of the cadherin family, one or more members of the selectin family, one or more cell adhesion molecules (CAMs), one or more of the immunoglobulin family and one or more artificial adherents. This list is provided by way of illustration only, and is not intended to be limiting. It will be clear to a person skilled in the art, that any combination of one or more adherents may be used.

Accordingly, included within the scope of the invention are methods of treatment of a human or animal patient through cellular therapy. The term “animal” here denotes all mammalian animals, preferably human patients. It also includes an individual animal in all stages of development, including embryonic and fetal stages. For example, the patient may be an adult, or the therapy may be for pediatric use (e.g., newborn, child or adolescent). Such cellular therapy encompasses the administration of stem cells generated according to the invention to a patient through any appropriate means. Specifically, such methods of treatment involve the regeneration of damaged tissue or wound healing. The term “administration” as used herein refers to well recognized forms of administration, such as intravenous or injection, as well as to administration by transplantation, for example transplantation by surgery, grafting or transplantation of tissue engineered liver derived from the stem cells according to the present invention. In the case of cells, systemic administration to an individual may be possible, for example, by infusion into the superior mesenteric artery, the celiac artery, the subclavian vein via the thoracic duct, infusion into the heart via the superior vena cava, or infusion into the peritoneal cavity with subsequent migration of cells via subdiaphragmatic lymphatics, or directly into liver sites via infusion into the hepatic arterial blood supply or into the portal vein.

Between 10⁴ and 10¹³ cells per 100 kg person may be administered per infusion. Preferably, between about 1-5×10⁴ and 1-5×10⁷ cells may be infused intravenously per 100 kg person. More preferably, between about 1×10⁴ and 1×10⁶ cells may be infused intravenously per 100 kg person. In some embodiments, a single administration of the subject stem cells is provided. In other embodiments, multiple administrations are used. Multiple administrations can be provided over an initial treatment regime, for example, of 3-7 consecutive days, and then repeated at other times.

It will be clear to a skilled person that gene therapy can additionally be used in a method directed at repairing damaged or diseased tissue. Use can, for example, be made of an adenoviral or retroviral gene delivery vehicle to deliver genetic information, like DNA and/or RNA to stem cells. A skilled person can replace or repair particular genes targeted in gene therapy. For example, a normal gene may be inserted into a nonspecific location within the genome to replace a non-functional gene. In another example, an abnormal gene sequence can be replaced for a normal gene sequence through homologous recombination. Alternatively, selective reverse mutation can return a gene to its normal function. A further example is altering the regulation (the degree to which a gene is turned on or off) of a particular gene. Preferably, the stem cells are ex vivo treated by a gene therapy approach and are subsequently transferred to the mammal, preferably a human being in need of treatment. For example, stem cell-derived cells may be genetically modified in culture before transplantation into patients.

Toxicity assay. The expanded stem cell population can further replace the use of cell lines such as Caco-2 cells in toxicity assays of potential novel drugs or of known or novel food supplements. Such toxicity assay may be conducted using patient matched or tissue/organ matched stem cells, which may be useful in personalized medicine. A cell-based toxicity test is used for determining organ specific cytotoxicity.

Compounds that may be tested comprise cancer chemopreventive agents, environmental chemicals, food supplements, and potential toxicants. The cells are exposed to multiple concentrations of a test agent for certain period of time. The concentration ranges for test agents in the assay are determined in a preliminary assay using an exposure of five days and log dilutions from the highest soluble concentration. At the end of the exposure period, the cultures are evaluated for inhibition of growth. Data are analyzed to determine the concentration that inhibited end point by 50 percent (TC50).

For high-throughput purposes, epithelial stem cells are cultured in multiwell plates such as, for example, 96-well plates or 384-well plates. Libraries of molecules are used to identify a molecule that affects the stem cells. Preferred libraries comprise antibody fragment libraries, peptide phage display libraries, peptide libraries (e.g., LOPAP™, Sigma Aldrich), lipid libraries (BioMol), synthetic compound libraries (e.g., LOP AC™, Sigma Aldrich) or natural compound libraries (Specs, TimTec). Furthermore, genetic libraries can be used that induce or repress the expression of one of more genes in the progeny of the adenoma cells. These genetic libraries comprise cDNA libraries, antisense libraries, and siRNA or other noncoding RNA libraries. The cells are preferably exposed to multiple concentrations of a test agent for certain period of time. At the end of the exposure period, the cultures are evaluated. The term “affecting” is used to cover any change in a cell, including, but not limited to, a reduction in, or loss of, proliferation, a morphological change, and cell death.

Animal model. Another aspect of the invention provides an animal model comprising a subject stem cell, such as a subject cancer stem cell.

In certain embodiments, the animal is an immunodeficient non-human animal (such as a rodent, e.g., a mouse or a rat), since such animal is less likely to cause rejection reaction. As an immunodeficient animal, it is preferred to use a non-human animal deficient in functional T cells, such as a nude mouse and rat, and a non-human animal deficient in functional T and B cells, such as a SCID mouse and a NOD-SCID mouse. Particularly, a mouse deficient in T, B, and NK cells (for example, a severely immunodeficient mouse obtained by crossing a SCID, RAG2KO, or RAG1KO mouse with an IL-2Rgnu11 mouse, which includes NOD/SCID/gammacnu11 mouse, NOD-scid, IL-2Rgnu11 mouse, and BALB/cRag2nu11, IL-2Rgnu11 mouse), which shows excellent transplantability, is preferably used.

Regarding the age of non-human animals, when athymic nude mice, SCID mice, NOD/SCID mice, or NOG mice are used, those of 4100 weeks old are preferably used.

NOG mice can be produced, for example, by the method described in WO 2002/043477 (incorporated by reference), or can be obtained from the Central Institute for Experimental Animals or the Jackson Laboratory (NSG mice).

Cells to be transplanted may be any types of cells, including a stem cell mass/clone, a tissue section differentiated from the subject stem cell, singly dispersed stem cells, stem cells cultured after isolation or freeze/thaw, and stem cells transplanted to another animal and again isolated from the animal. The number of cells to be transplanted may be 10⁶ or less, but a greater number of cells may be transplanted. In certain embodiments, subcutaneous transplantation is preferable because of its simple transplantation techniques. However, the site of transplantation is not particularly limited and preferably appropriately selected depending on the animal used. The procedure for transplanting NOG established cancer cell lines is not particularly limited, and any conventional transplantation procedures can be used.

Such animal models can be used to, for example, search for drug target molecules and to assess drugs. Assessment methods for drugs include screening for drugs and screening for anticancer agents. Methods of searching for target molecules include, but are not limited to, methods for identifying genes such as DNAs and RNAs highly expressed in cancer stem cells (e.g., cancer stem cell markers) using Gene-chip analysis, and methods for identifying proteins, peptides, or metabolites highly expressed in cancer stem cells using proteomics.

Screening methods for searching for target molecules include methods in which substances that inhibit the growth of cancer stem cells are screened from a small molecule library, antibody library, micro RNA library, or RNAi library, etc., using cell growth inhibition assay. After an inhibitor is obtained, its target can be revealed.

Thus the invention also provides a method of identifying a target molecule of a drug, the method comprising: (1) producing a non-human animal model by transplanting a cancer stem cell of the invention to a non-human animal (e.g., an immuno-compromised mouse or rat); (2) before and after administering the drug, collecting a tissue section showing a tissue structure characteristic of a cancer development process of said cancer stem cell population or showing a biological property thereof; (3) examining/comparing the tissue sections (before vs. after) collected in (2) for the expression of a DNA, RNA, protein, peptide, or metabolite; and (4) identifying a DNA, RNA, protein, peptide or metabolite that varies depending on a structure formed from the cancer stem cells, a cancer development process originating from the cancer stem cells, or a biological property of the cancer stem cells, in the tissue section.

The invention also provides a method of assessing a drug, the method comprising: (1) producing a non-human animal model by transplanting a cancer stem cell of the invention to a non-human animal (e.g., an immuno-compromised mouse or rat); (2) administering a test substance to the non-human animal model of (1); (3) collecting a tissue section showing a tissue structure characteristic of a cancer development process originating from cancer stem cells or showing a biological property thereof; (4) observing a change in the cancer stem cells over time, cancer development process, or a biological property thereof, in the tissue section; and (5) identifying formation of a structure formed from the cancer stem cells, a cancer development process originating from the cancer stem cells, or a biological property of the cancer stem cells, that is inhibited by the test substance.

The invention also provides a method of screening for a drug, the method comprising: (1) producing a non-human animal model by transplanting a cancer stem cell of the invention to a non-human animal (e.g., an immuno-compromised mouse or rat); (2) administering a test substance to the non-human animal model of (1); (3) collecting a tissue section that shows a tissue structure characteristic of a cancer development process originating from cancer stem cells, or shows a biological property thereof; (4) observing a change in the cancer stem cells over time, cancer development process, or a biological property thereof, in the tissue section; and (5) identifying a test substance that inhibits formation of a structure formed from specific cancer stem cells, a cancer development process originating from cancer stem cells, or a biological property of cancer stem cells.

### 8. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Illustrative Example

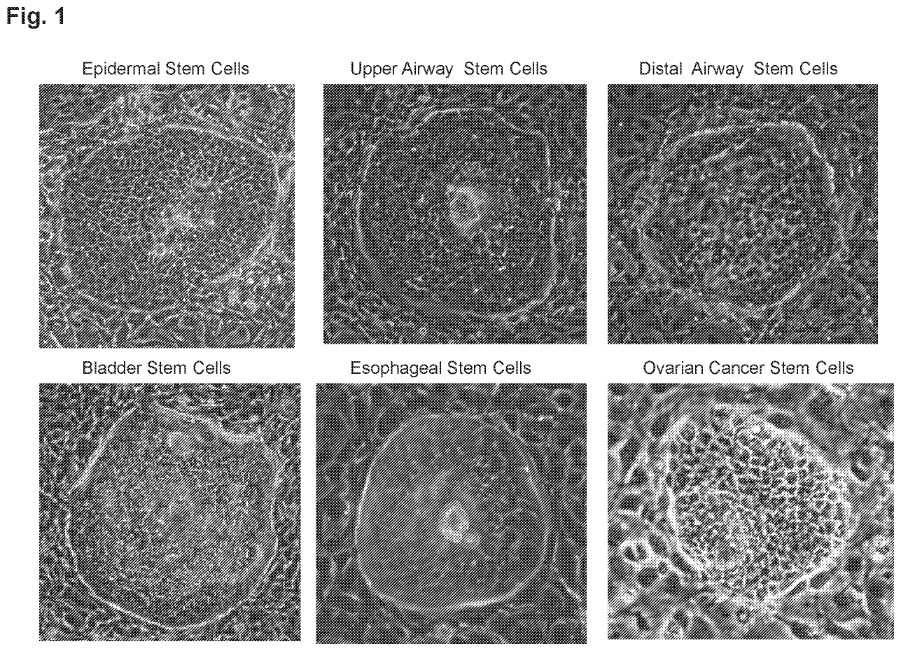

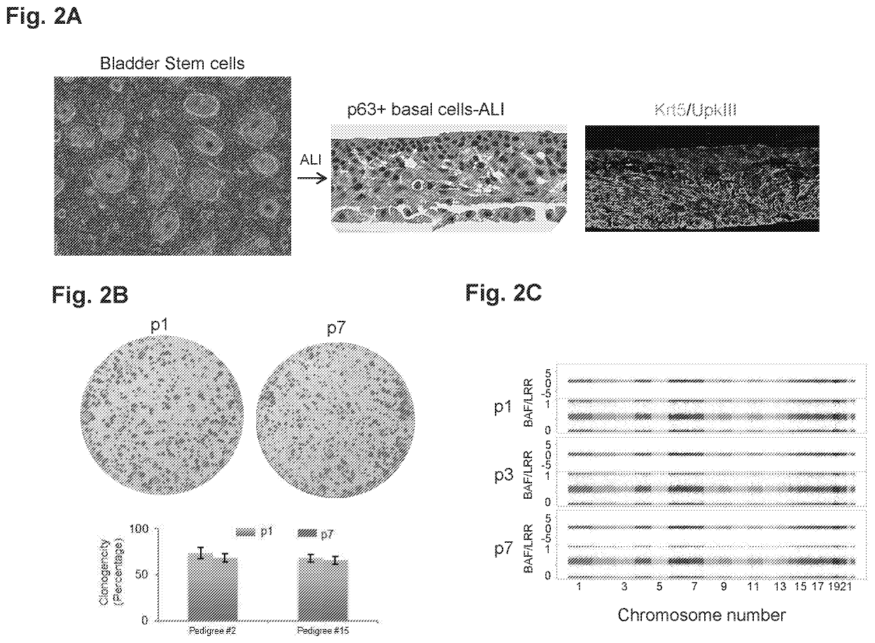

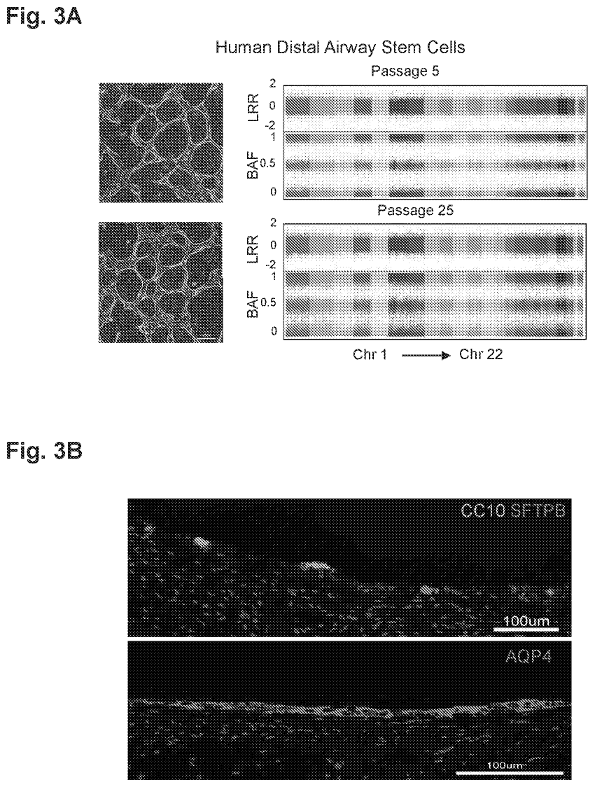

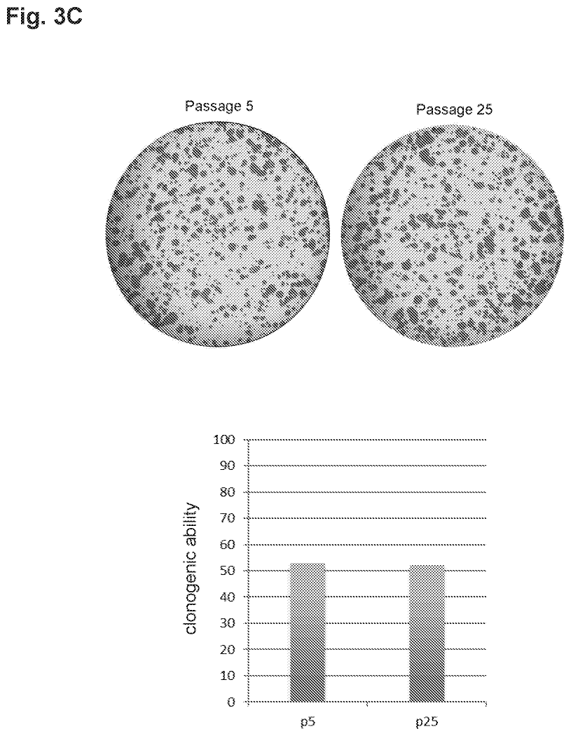

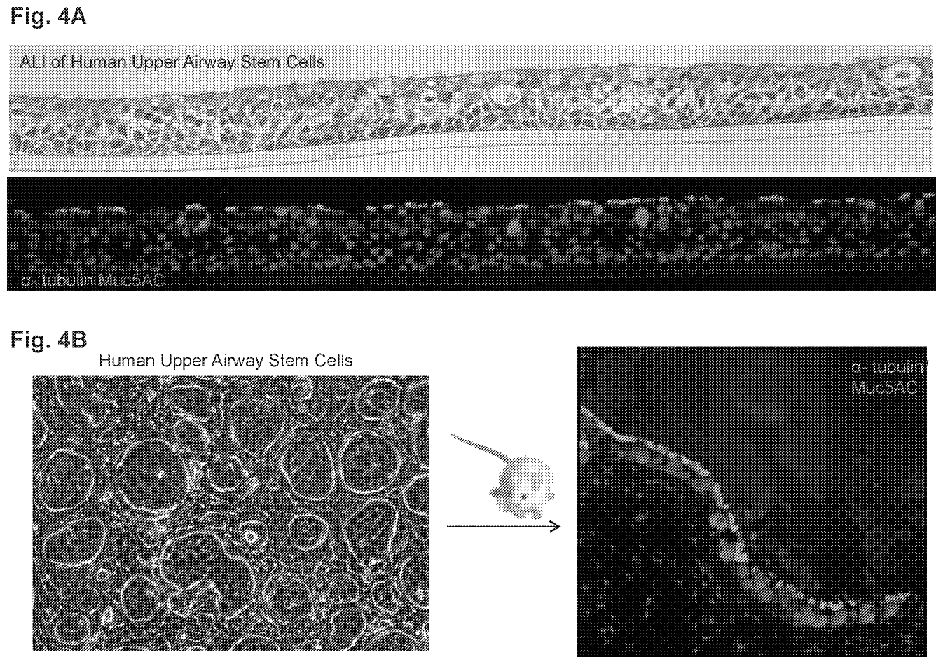

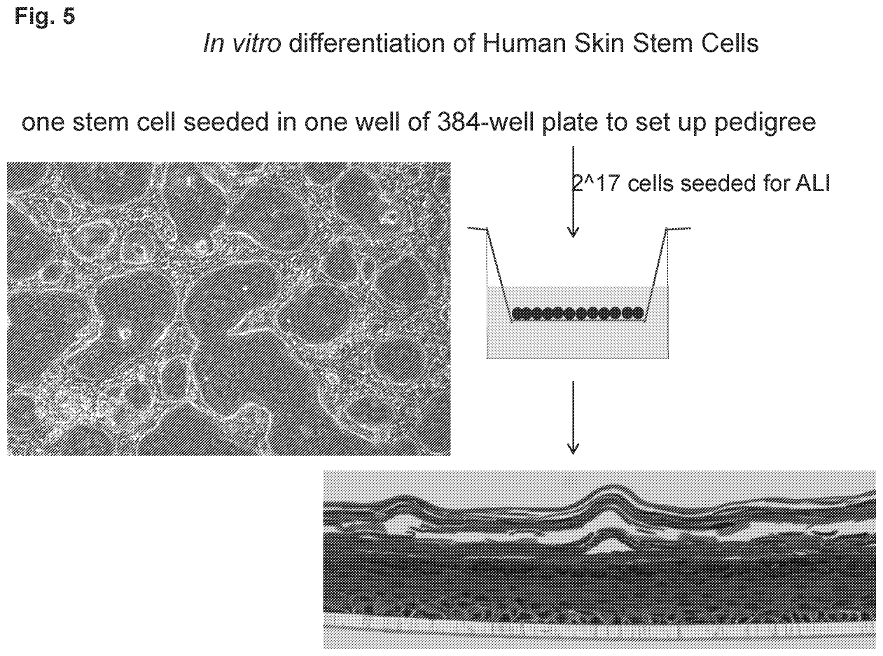

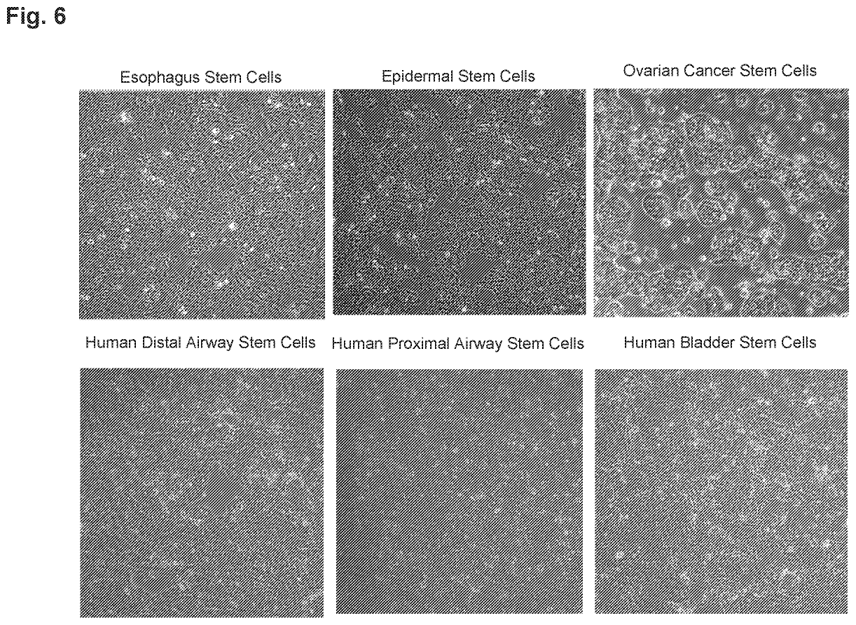

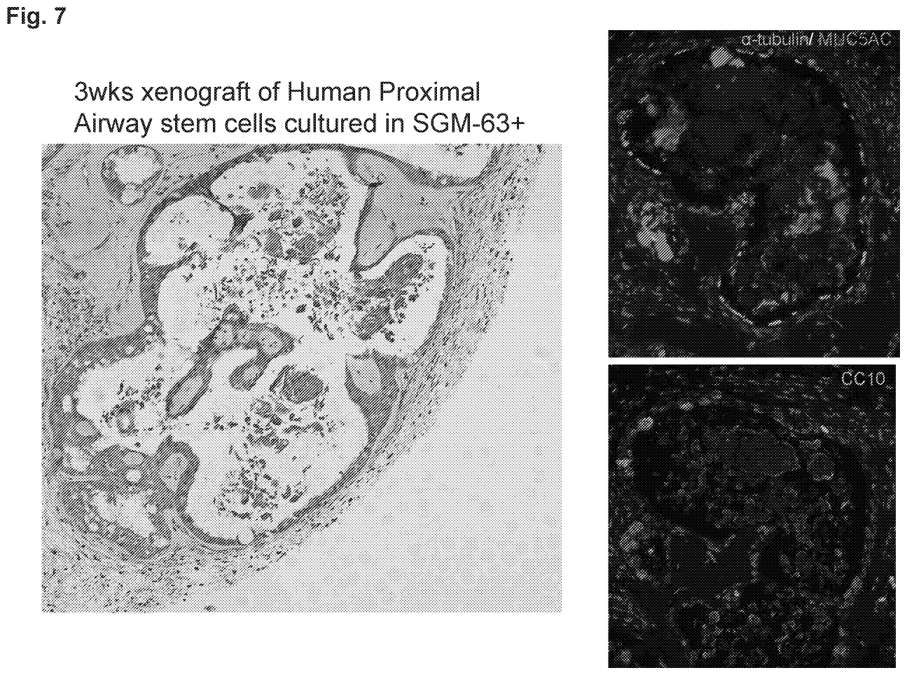

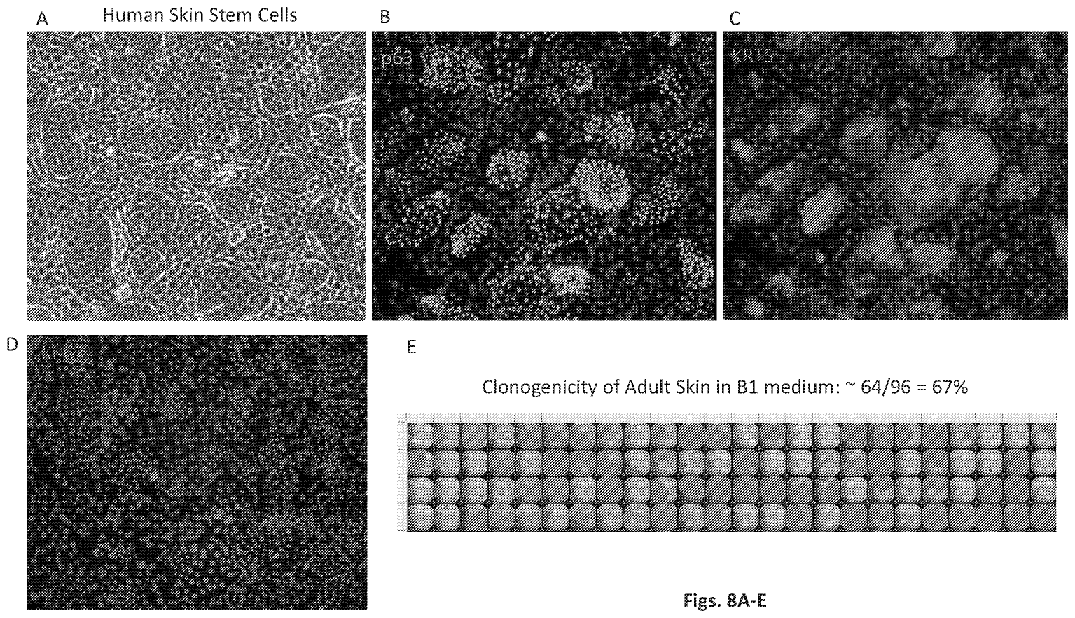

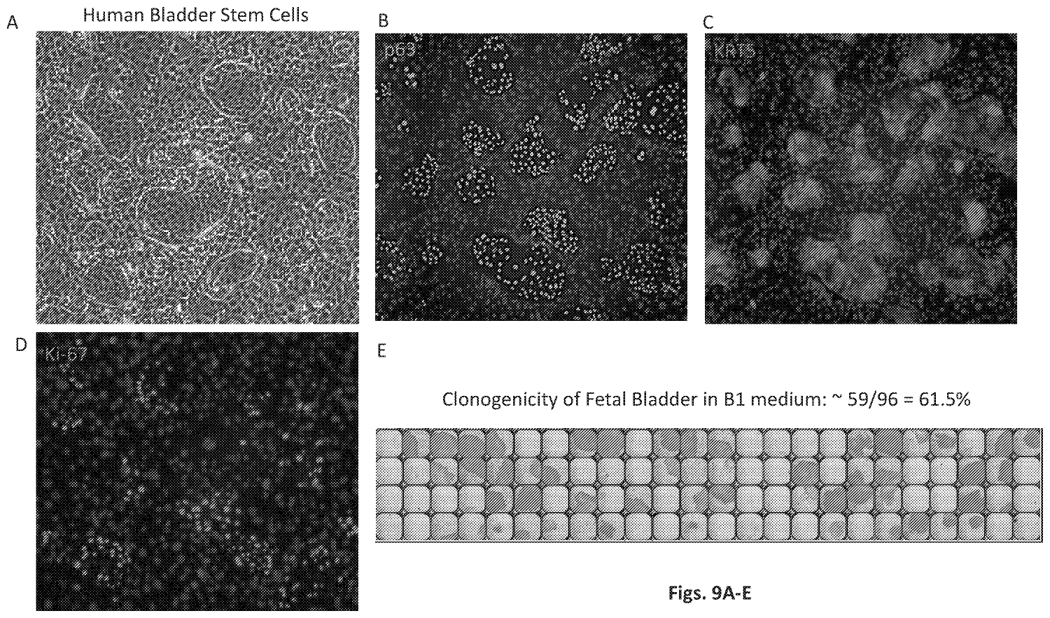

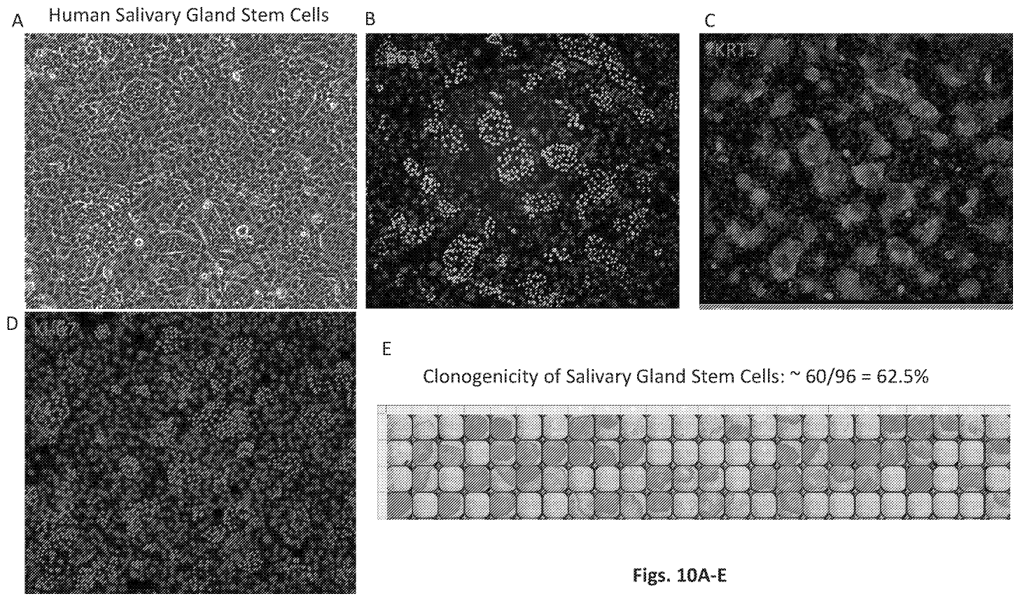

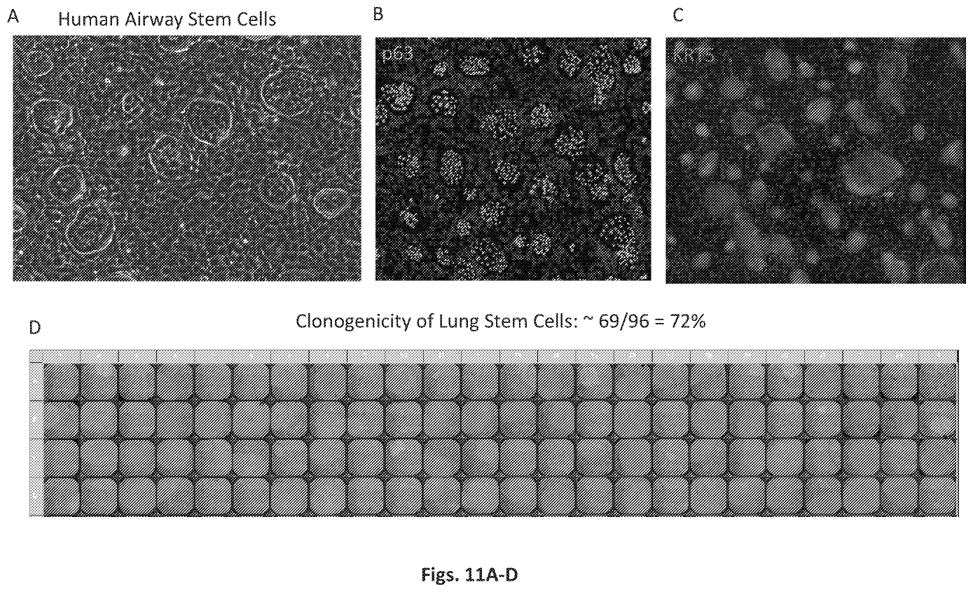

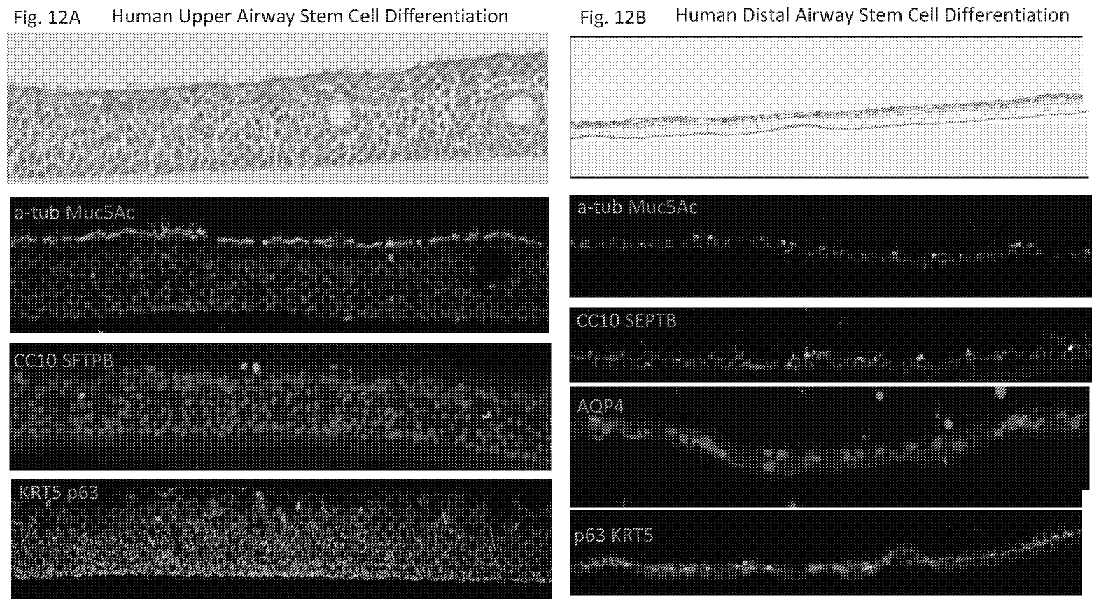

The media described herein have been tested and proven to support robust growth of epithelial stem cells derived from stratified epithelial tissues from human and other mammals. For example, lung stem cells, bladder stem cells and esophagus stem cells have been cloned (see FIGS. 1 and 2).

An illustrative system is a medium referred to as SQM. The SQM medium has been tested and proven to support robust growth of epithelial stem cells derived from human tissues or other mammals in the presence of the irradiate feeder of mouse fibroblast cells (3T3-J2). For example, lung stem cells, esophagus stem cell, bladder stem cells and ovarian cancer stem cells, can all grow robustly in this culture system that comprises SQM medium, along with irradiated 3T3-J2 feeders in the illustrated example.

The base medium comprises DMEM, F12, FBS, L-glutamine, Adenine, Pen/Strep, Insulin, T3, Hydrocortisone, EGF and is further added a Rock inhibitor, TGF-beta inhibitor, BMP4 inhibitor, VEGF inhibitor, TrkA inhibitor, Ponatinib and FGF10. Inclusion of jagged1 and nicotinamide in the medium was observed to induce the abortion of some epithelial stem cell (lung, esophagus etc) self-renewal, and so the those two components are not used in present media or other embodiments of the media of the present invention. R-spondin was found not required to support the self-renewal and multipotency of stratified epithelial stem cells. Epithelial stem cells from a variety of different tissues, including lung and Esophagus, have been passaged in the present medium for more than twenty-five passages and maintain self-renewal ability and multi-potent differentiation ability both in vitro and in xenograft model using NSG mice.

Another illustrative system is a medium referred to as SGM-63+. The SGM-63+ medium has been tested and proven to support robust growth of epithelial stem cells derived from human tissues or other mammals in the absence of the irradiate feeder of mouse fibroblast cells (3T3-J2). For example, lung stem cells, esophagus stem cell, bladder stem cells and ovarian cancer stem cells, can all grow robustly in this culture system that comprises SGM-63+ medium without the need of irradiated 3T3-J2 feeders in the illustrated example.

The base medium comprises DMEM, F12, FBS, L-glutamine, Adenine, Pen/Strep, Insulin, T3, Hydrocortisone, EGF and is further added a Rock inhibitor, TGF-beta inhibitor, BMP4 inhibitor, VEGF inhibitor, TrkA inhibitor, Ponatinib, CK2 inhibitor, Syk inhibitor, LPA Receptor antagonist, Oct 4-activating compound 1, GSK3 inhibitor and FGF10. Inclusion of jagged1 and nicotinamide in the medium was observed to induce the abortion of some epithelial stem cell (lung, esophagus etc) self-renewal, and so the those two components are not used in present media or other embodiments of the media of the present invention. R-spondin was found not required to support the self-renewal and multipotency of stratified epithelial stem cells. Epithelial stem cells from a variety of different tissues, including lung and Esophagus, have been passaged in the present medium for more than twenty-five passages and maintain self-renewal ability and multi-potent differentiation ability both in vitro and in xenograft model using NSG mice.

### SQM Medium (1 Liter)

| | | |
|---|---|---|
| DMEM | 645 | ml |
| F12 | 215 | ml |
| FBS | 100 | ml |
| L-glutamine | 10 | ml |
| Adenine | 10 | ml |
| Pen/Strep | 10 | ml |
| Insulin | 1 | ml |
| T3 | 1 | ml |
| Hydrocortisone | 2 | ml |
| EGF | 1 | ml |
| Noggin | 1 | ml |
| Y-27632 | 1 | ml |
| SB431542 | 1 | ml |
| hFGF10 | 1 | ml |
| Tivozanib | 500 | uL |
| Potatinib | 500 | uL |
| GW441756 | 500 | uL |

Filter and store at 4° C.

### Components

### DMEM

(Invitrogen 11960)
High glucose (4.5 g/L), no L-glutamine, no sodium pyruvate

### F-12 Nutrient Mixture (Ham)

(Invitrogen 11765)
Contains L-glutamine

### Adenine

(Calbiochem 1152 10 g)
Add 243 mg of adenine to 100 ml of 0.05 M HCl (dilute 0.4 ml of concentrated HCl in 100 ml of distilled H₂O)
Stir for about one hour at RT to dissolve
Filter sterilize
Divide into 10.0 ml aliquots
Final Concentration: 1.8×10−4M
Store at −20° C.

### FBS

(Hyclone SH30910.03 500 mL)
DO NOT heat inactivate serum
Thaw and aliquot serum into 50 ml/tube and store at −20° C.

### L-Glutamine

(GIBCO 25030-081 100 ml)
Thaw and divide into 10.0 ml aliquots
Store at −20° C.

### Penicillin/Streptomycin

(GIBCO 15140-122 100 mL)

### Fungizone

(GIBCO, 15290-018) only apply to primary culture

### Gentamicine

(GIBCO, 15710-064) only apply to primary culture

### Insulin

(Sigma I-5500 50 mg)
Dissolve 50 mg in 10 ml of 0.005N HCl (stock 5 mg/ml)
Distribute in 1 ml aliquots and store at −20° C.
Final concentration 5 ug/ml

### T3 (3,3″,5-Triiodo-L-Thyronine)

(Sigma T-2752 100 mg)
Dissolve 13.6 mg in 15 ml of 0.02N NaOH
Make volume up to 100 ml with PBS (concentrated stock 2×10−4 M)
Distribute in 10 ml aliquots and store at −20° C.
Take 0.1 ml concentrated stock, make volume up to 10 ml with PBS
Distribute in 1 ml aliquots and store at −20° C. (stock 2×10−6 M)
Final concentration 2×10−9 M

### Hydrocortisone

(Sigma H-0888 1 g or Calbiochem/EMD 386698)
Dissolve 25 mg in 5 ml 95% ETOH (concentrated stock 5 mg/ml)
Store at −20° C.
Take 0.4 ml of concentrated stock, make up to 10 ml with serum-free SBM medium
Distribute in 1 ml aliquots and store at −20° C. (stock 200 μg/ml)
Final concentration 0.4 ug/ml

### EGF

(Upstate Biotechnology 01-107)

### Preparation of 0.1% BSA:

100 mg BSA (Sigma A-2058; IgG-free, cell culture tested 5 g)
Dissolve in 100 ml distilled H2O
Sterile filter through 0.22μ Nalgene
Store at either 4° C. or −20° C., depending on frequency of use

### Preparation of EGF:

Dissolve 1 mg EGF in 1 ml 0.1% BSA
Distribute in 100 μl aliquots and store at −80° C. (concentrated stock 100 μg/100 μl)
Bring 100 μg concentrated stock to 10 ml with 0.1% BSA
Sterile filter using 0.22μ Millipore Millex-GV
Distribute in 1 ml aliquots and store at −20° C. (stock 10 μg/ml)
Final concentration 10 ng/ml

### Human Noggin

(Cat. 6057-NG, R&D systems; Final concentration: 100 ng/ml, stock: 100 ug/ml)
(Dissolve 10 mg in 100 ml sterile PBS as stock, aliquot 1 ml/vial)

### Y-27632

(Cat. 688000, Calbiochem; Final concentration: 2.5 uM, stock: 2.5 mM)
(Dissolve 5 mg in 5.912 ml sterile H2O as stock, aliquot 1 ml/vial)

### SB431542

(Cat. 13031, Cayman chemical company; Final concentration: 2 uM, stock: 2 mM)
(Dissolve 5 mg in 6.5 ml DMSO as stock, aliquot 1 ml/vial)

### FGF10

(Cat. 345-FG, R&D systems; Final concentration: 100 ng/ml, stock: 100 ug/ml)
(Dissolve 1 mg in 10 ml sterile PBS as stock, aliquot 1 ml/vial)

### Tivozanib (AV-951)

(Cat. S1207, Selleckchem; Final concentration: 500 nM, stock: 10 mM)

### Potatinib (AP24534)

(Cat. S1490, Selleckchem; Final concentration: 500 nM, stock: 10 mM)
GW441756 (Cat. S2891, Selleckchem; Final concentration: 500 nM, stock: 10 mM)

### SGM-63+ Medium (1 Liter)

DMEM: 645 ml
F12: 215 ml
FBS: 100 ml
L-glutamine: 10 ml
Adenine: 10 ml
Pen/Strep: 10 ml
Insulin: 1 ml
T3: 1 ml
Hydrocortisone: 2 ml
EGF: 1 ml
Noggin: 1 ml
Y-27632: 1 ml
SB431542: 1 ml
hFGF10: 1 ml
Tivozanib (AV-951) Final concentration: 500 nM
Potatinib (AP24534) Final concentration: 500 nM
GW441756 Final concentration: 500 nM
Silmitasertib (CX-4945) Final concentration: 1 microM
R406 Final concentration: 1 microM
Ki16198 Final concentration: 1 microM
OAC1 Final concentration: 1 microM
AZD1080 Final concentration: 1 microM
   Filter and store at 4° C.

### Components

### DMEM (Invitrogen 11960)

High glucose (4.5 g/L), no L-glutamine, no sodium pyruvate

### F-12 NUTRIENT MIXTURE (Ham) (Invitrogen 11765)

Contains L-glutamine

### Adenine (Calbiochem 1152 10 g)

Add 243 mg of adenine to 100 ml of 0.05 M HCl (dilute 0.4 ml of concentrated HCl in 100 ml of distilled H2O)
Stir for about one hour at RT to dissolve
Filter sterilize
Divide into 10.0 ml aliquots
Final Concentration: 1.8×10−4M
Store at −20° C.

### FBS (Hyclone SH30910.03 500 mL)

DO NOT heat inactivate serum
Thaw and aliquot serum into 50 ml/tube and store at −20° C.

### L-Glutamine (GIBCO 25030-081 100 ml)

Thaw and divide into 10.0 ml aliquots
Store at −20° C.

### Penicillin/Streptomycin (GIBCO 15140-122 100 mL)

Fungizone (Gibco, 15290-018) only apply to primary culture
Gentamicine (Gibco, 15710-064) only apply to primary culture

### Insulin (Sigma I-5500 50 mg)

Dissolve 50 mg in 10 ml of 0.005N HCl (stock 5 mg/ml)
Distribute in 1 ml aliquots and store at −20° C.
Final concentration 5 ug/ml

### T3 (3,3″,5-Triiodo-L-Thyronine) (Sigma T-2752 100 mg)

Dissolve 13.6 mg in 15 ml of 0.02N NaOH
Make volume up to 100 ml with PBS (concentrated stock 2×10−4 M)
Distribute in 10 ml aliquots and store at −20° C.
Take 0.1 ml concentrated stock, make volume up to 10 ml with PBS
Distribute in 1 ml aliquots and store at −20° C. (stock 2×10−6 M)
Final concentration 2×10−9 M

### Hydrocortisone (Sigma H-0888 or Calbiochem/EMD 386698)

Dissolve 25 mg in 5 ml 95% ETOH (concentrated stock 5 mg/ml)
Store at −20° C.
Take 0.4 ml of concentrated stock, make up to 10 ml with serum-free SBM medium
Distribute in 1 ml aliquots and store at −20° C. (stock 200 μg/ml)
Final concentration 0.4 ug/ml

### EGF (Upstate Biotechnology 01-107)

### Preparation of 0.1% BSA:

100 mg BSA (Sigma A-2058; IgG-free, cell culture tested 5 g)
Dissolve in 100 ml distilled H2O
Sterile filter through 0.22μ Nalgene
Store at either 4° C. or −20° C., depending on frequency of use

### Preparation of EGF:

Dissolve 1 mg EGF in 1 ml 0.1% BSA
Distribute in 100 μl aliquots and store at −80° C. (concentrated stock 100 μg/100 μl)
Bring 100 μg concentrated stock to 10 ml with 0.1% BSA
Sterile filter using 0.22μ Millipore Millex-GV
Distribute in 1 ml aliquots and store at −20° C. (stock 10 μg/ml)
Final concentration 10 ng/ml
   Human Noggin (Cat. 6057-NG, R&D Systems; Final Concentration: 100 ng/ml, Stock: 100 ug/ml)
(Dissolve 10 mg in 100 ml sterile PBS as stock, aliquot 1 ml/vial)

### Y-27632 (Cat. 688000, Calbiochem; Final Concentration: 2.5 uM, Stock: 2.5 mM)

(Dissolve 5 mg in 5.912 ml sterile H2O as stock, aliquot 1 ml/vial)

### SB431542 (Cat. 13031, Cayman Chemical Company; Final Concentration: 2 uM, Stock: 2 mM)

(Dissolve 5 mg in 6.5 ml DMSO as stock, aliquot 1 ml/vial)
   FGF10 (Cat. 345-FG, R&D Systems; Final Concentration: 100 ng/ml, Stock: 100 ug/ml)
(Dissolve 1 mg in 10 ml sterile PBS as stock, aliquot 1 ml/vial)
   Tivozanib (AV-951) (Cat. S1207, Selleckchem; Final concentration: 500 nM, stock: 10 mM)
   Potatinib (AP24534) (Cat. S1490, Final concentration: 500 nM, stock: 10 mM)
   GW441756 (Cat. S2891, Selleckchem; Final concentration: 500 nM, stock: 10 mM)
   Silmitasertib (CX-4945) (Cat. S2248, Selleckchem; Final concentration: 1 microM, stock: 10 mM)
   R406 (Cat. S2194, Selleckchem; Final concentration: 1 microM, stock: 10 mM)
   Ki16198 (Cat. S2906, Selleckchem; Final concentration: 1 microM, stock: 10 mM)
   OAC1 (Cat. S7217, Selleckchem; Final concentration: 1 microM, stock: 10 mM)
   AZD1080 (Cat. S7145, Selleckchem; Final concentration: 1 microM, stock: 10 mM)

### B1 Medium (Feeder Dependent)

### For 1L

DMEM: 645 ml
F12: 215 ml
FBS: 100 ml
L-glutamine: 10 ml
Adenine: 10 ml
Pen/Strep: 10 ml
Insulin: 1 ml
T3: 1 ml
Hydrocortisone: 2 ml
Cholera Enterotoxin (optional): 1 ml
EGF: 1 ml
Gentamicin: 5 ml
Fungizone (optional): 1 ml

### Y-27632

Working Concentration: 2.5 uM
Stock Concentration: 25 mM in H2O
Molecular Weight: 320.26
To make 25 mM,
10 mg in 1.249 ml of H2O
1:10,000 so 100 ul for 1L medium

### SB431542

M.W.: 384
Final concentration: 2 uM
Stock Concentration: 20 mM
10 mg in 1.3008 ml DMSO
1:10,000 so 100 ul for 1L medium

### AV-951

Working Concentration: 500 nM
Stock solution: 10 mM
1:20,000 so 50 ul for 1L medium

### Ponatinib

Working Concentration: 500 nM
Stock solution: 10 mM
1:20,000 so 50 ul for 1L medium

### GW441756

Working Concentration: 500 nM
Stock solution: 10 mM
1:20,000 so 50 ul for 1L medium

### ClonoVision™ B1F Medium (Feeder Free)

### For 1L

DMEM: 645 ml
F12: 215 ml
FBS: 100 ml
L-glutamine: 10 ml
Adenine: 10 ml
Pen/Strep: 10 ml
Insulin: 1 ml
T3: 1 ml
Hydrocortisone: 2 ml
Cholera Enterotoxin (optional): 1 ml
EGF: 1 ml
Gentamicin: 5 ml
Fungizone (optional): 1 ml

### Y-27632

Working Concentration: 2.5 uM
Stock Concentration: 25 mM in H2O
Molecular Weight: 320.26
To make 25 mM,
10 mg in 1.249 ml of H2O
1:10,000 so 100 ul for 1L medium

### SB431542

M.W.: 384
Final concentration: 2 uM
Stock Concentration: 20 mM
10 mg in 1.3008 ml DMSO
1:10,000 so 100 ul for 1L medium

### AV-951

Working Concentration: 500 nM
Stock solution: 10 mM
1:20,000 so 50 ul for 1L medium

### Ponatinib

Working Concentration: 500 nM
Stock solution: 10 mM
1:20,000 so 50 ul for 1L medium

GW441756
Working Concentration: 500 nM
Stock solution: 10 mM
1:20,000 so 50 ul for 1L medium

### Silmitasertib (CX-4945)

Work Concentration: 1 uM
Stock solution: 10 mM
1:10,000 so 100 ul for 1L medium

### R406

Work Concentration: 1 uM
Stock solution: 10 mM
1:10,000 so 100 ul for 1L medium

### Ki16198

Work Concentration: 1 uM
Stock solution: 10 mM
1:10,000 so 100 ul for 1L medium

### OAC1

Working Concentration: 1 uM
Stock solution: 10 mM
1:10,000 so 100 ul for 1L medium

### AZD1080

Working Concentration: 1 uM
Stock solution: 10 mM
1:10,000 so 100 ul for 1L medium

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A defined culture medium for isolating and stably maintaining the epigenetics of stratified epithelial stem cells through a plurality of passaging in culture, the medium comprising: a basal medium; and each of a ROCK (Rho Kinase) inhibitor, a mitogenic growth factor, insulin or IGF, a TrkA Inhibitor, and an Oct4-activating agent, which culture media includes at least one of a VEGF inhibitor, a tyrosine kinase inhibitor and/or FGF10 or an FGF10 agonist, and which culture media optionally further includes a TGFβ signaling pathway inhibitor, and/or a Bone Morphogenetic Protein (BMP) antagonist, wherein the culture media supports the epigenetically stable growth and proliferation of stem cells of stratified epithelial tissue origin in culture.

2. The defined culture medium of claim 1, which permits the passaging of the cells in a feeder free manner and further includes a SYK Inhibitor, an LPA receptor antagonist, a GSK3 inhibitor, and a CK2 inhibitor.

3. The defined culture medium of claim 1, wherein the epithelial stem cells are in contact with extracellular matrix or other biomatrix.

4. The defined culture medium of claim 1, wherein the stem cell is isolated from a tissue sample taken from normal epithelial tissue.

5. The defined culture medium of claim 1, wherein the stem cell is isolated from a tissue sample taken from diseased epithelial tissue, such as from an inflammatory or autoimmune patient.

6. The defined culture medium of claim 1, wherein the stem cell is isolated from a tissue sample taken from a tumor.

7. A clonal stratified epithelial stem cell isolated utilizing the defined culture medium of claim 1.
